(19) United States
(12) Patent Application Publication
(10) Pub. No.: US 2022/0267469 A1
(43) Pub. Date: Aug. 25, 2022

(54) NOVEL BSSL ANTIBODIES

(71) Applicant: LIPUM AB, UMEÅ (SE)

(72) Inventors: Olle HERNELL, UMEÅ (SE); Susanne LINDQUIST, RÖBÄCK (SE); Lennart LUNDBERG, BILLDAL (SE); Helena PERSSON LOTSHOLM, SIGTUNA (SE)

(21) Appl. No.: 17/625,527

(22) PCT Filed: Jul. 10, 2020
(86) PCT No.: PCT/SE2020/050728

§ 371 Date: Jan. 7, 2022

(30) Foreign Application Priority Data
Jul. 12, 2019 (SE) 1950888-6

(51) Int. Cl. C07K 16/40; C12N 9/20; A61P 29/00

(52) U.S. Cl. CPC C07K 16/40; C12N 9/20; A61P 29/00; C07K 2299/00; C07K 2317/24; C07K 2317/33; C07K 2317/52; C07K 2317/622; C07K 2317/76; C07K 2317/92; C07K 2317/94; C07K 2317/565

(57) ABSTRACT

The present invention relates to novel isolated antibodies and antigen-binding fragments thereof that bind to human Bile Salt-Stimulated Lipase (hBSSL). The antibodies and antigen-binding fragments thereof bind to a previously uncharacterized epitope, situated in the N-terminal part of hBSSL and identified as comprising the amino acid residues 7-12 and the amino acid residues 42-55. The 5 present invention also relates to the medical uses of the antibodies and/or the antigen-binding fragments thereof, in particular in treatment of inflammatory conditions, and to related pharmaceutical compositions.

### TECHNICAL FIELD

The invention relates to novel isolated antibodies, and antigen-binding fragments thereof, that bind to a previously uncharacterized epitope of Bile Salt-Stimulated Lipase (BSSL) situated in the N-terminal part of the BSSL protein. The present document also relates to the medical uses of the antibodies, and antigen-binding fragments thereof, in particular in treatment of inflammatory conditions, and to related pharmaceutical compositions. The present document also discloses the use of the antibodies, or antigen-binding fragments thereof, as molecular tools in the detection of BSSL and/or for diagnosing BSSL related diseases.

### BACKGROUND

Inflammatory conditions, including autoimmune and autoinflammatory diseases, remain a significant threat to human health. Despite advancements in the treatment of inflammatory conditions, improved therapies are still being sought.

Inflammatory conditions include a vast array of disorders and diseases that are characterized by inflammation, including autoimmune diseases and autoinflammatory diseases. Inflammation can e.g., occur as a response to infections, injuries, allergens and/or toxins, or as a response to the body itself, e.g., autoimmune processes. An autoimmune disease occurs when the body's immune system attacks and destroys healthy body tissue by mistake. It is reported that there are more than approximately 80 known autoimmune diseases.

Some inflammatory conditions are chronic. Chronic inflammation occurs when an inflammation response lingers, leaving the body in a constant state of alert. Examples of inflammatory diseases and conditions that include chronic inflammation are rheumatoid arthritis (RA), juvenile idiopathic arthritis (JIA), psoriatic arthritis (PsA) and inflammatory bowel disease (IBD), such as ulcerative colitis (UC) and Crohn's disease (CD).

RA is a chronic, inflammatory, systemic autoimmune disease. Current therapies for RA include non-steroid anti-inflammatory drugs (NSAIDs) for pain treatment, disease modifying antirheumatic drugs (DMARDs) and biological agents that target specific proinflammatory cytokines, or cell surface receptors of various cell types.

JIA, also known as juvenile rheumatoid arthritis (JRA), is the most common form of arthritis in children and adolescents. JIA has an onset before age 16 and the cause of JIA is largely unknown. The major emphasis of treatment for JIA is to help the child regain normal level of physical and social activities. Most children are treated with NSAIDs and intra-articular corticosteroid injections. Methotrexate, a DMARD is a powerful drug which helps suppress joint inflammation in the majority of JIA patients with polyarthritis, though it has been reported to be less useful in systemic arthritis, and many children receive TNFα inhibitor drugs, such as etanercept.

IBD is term used to describe disorders that involve chronic inflammation in the digestive tract. IBD includes UC and CD.

The goal of IBD treatment is to reduce the inflammation that triggers the signs and symptoms. In the best cases, this may lead not only to symptom relief but also to long-term remission and reduced risks of complications. IBD treatment usually involves either drug therapy, such as anti-inflammatory drugs (NSAIDs), immune system suppressors and/or biological agents as well as surgery.

Bile Salt-Stimulated Lipase (BSSL), also known as Bile Salt-Dependent Lipase (BSDL), Carboxyl Ester Lipase (CEL) or Bile Salt-Activated Lipase (BAL) is a lipolytic enzyme encoded by the CEL gene and expressed in the exocrine pancreas and secreted into the intestinal lumen in all species so far investigated and aids in the digestion of lipids.

In some species, including humans, primates, dogs, cats and mice, BSSL is also expressed in lactating mammary gland and secreted in the milk. Moreover, BSSL has been found in low, but significant levels in serum of healthy individuals and to be involved in lipoprotein metabolism and modulation of atherosclerosis. BSSL has also been found to have a role in inflammatory processes.

BSSL may be isolated from a suitable tissue such as human milk. Alternatively recombinant BSSL can be produced using standard methods through the isolation of DNA encoding BSSL.

DNA encoding BSSL may be conveniently isolated from commercially available RNA, cDNA libraries, genomic DNA, or genomic DNA libraries using conventional molecular biology techniques such as library screening and/or Polymerase Chain Reaction (PCR).

Methods for purification of BSSL from different tissues and transfected cell-lines are known in the art [1].

Document [2] describes antigen-binding compounds that bind BSSL or Feto-Acinar Pancreatic Protein (FAPP). The compounds are disclosed to recognize a C-terminal peptide (J28 epitope) of BSSL. FAPP is an oncofetal form of BSSL characterized by the J28 carbohydrate dependent epitope. The antigen-binding compounds are said to induce apoptosis and/or slow the proliferation of tumor cells expressing a BSSL or FAPP polypeptide. Document [2] describes compounds that are able to directly target tumor cells, particularly BSSL- or FAPP-expressing pancreatic tumor cells, and cause their death via apoptosis and/or halt their proliferation.

Documents [3, 4] describe the discovery that BSSL has a role in inflammatory processes and that inhibition or elimination of BSSL protects from development of chronic arthritis in animal models. Documents [3, 4] disclose that the BSSL protein is present in inflammatory cells and inflamed tissue and that BSSL deficient mice are protected from development of inflammatory disease, exemplified by collagen-induced arthritis (CIA).

Although therapies for inflammatory conditions, such as autoinflammatory disease and autoimmune disease, have improved considerably over the years by the introduction of new drugs and drug classes, such as antibodies, most regimens and drugs still have in common that they aim at suppressing the immune system, as is e.g., the case with all TNFα inhibitor drugs and corticosteroids. This in turn increases the risk for secondary infections and complications.

Consequently, there is still a significant clinical need for new selective, preferably biological, drugs for the treatment, prophylaxis and prevention of inflammatory diseases, which are directed towards different and/or novel targets involved in inflammatory signaling and processes, and which do not primarily act through suppressing the immune system, and which, thus, are expected to have fewer and/or less severe adverse effects.

### SUMMARY

It is a general objective to provide antibodies, or antigen-binding fragments thereof, that bind specifically to a Bile Salt Stimulated Lipase (BSSL), such as human BSSL (hBSSL).

This and other objectives are met by embodiments as disclosed herein.

The present invention is defined in the independent claims. Further embodiments of the invention are defined in the dependent claims.

An aspect of the invention relates to an isolated antibody, or antigen-binding fragment thereof, comprising three complementary determining regions (CDRs) of a heavy chain variable region (HCVR), denoted HCDR, and three CDRs of a light chain variable region (LCVR), denoted LCDR. In this aspect, the first HCDR comprises, preferably consists of, an amino acid sequence according to SEQ ID NO: 7, or an amino acid sequence having at least 87% identity to SEQ ID NO: 7, the second HCDR comprises, preferably consists of, an amino acid sequence according to SEQ ID NO: 8, or an amino acid sequence having at least 75% identity to SEQ ID NO: 8 and the third HCDR comprises, preferably consists of, an amino acid sequence according to SEQ ID NO: 9, or an amino acid sequence having at least 83% identity to SEQ ID NO: 9. Furthermore, the first LCDR comprises, preferably consists of, an amino acid sequence according to SEQ ID NO: 10, or an amino acid sequence having at least 80% identity to SEQ ID NO: 10, the second LCDR comprises, preferably consists of, the amino acid sequence ATS, or an amino acid sequence having at least 66% identity to the amino acid sequence ATS, preferably AAS, and the third LCDR comprises, preferably consists of, an amino acid sequence according to SEQ ID NO: 11, or an amino acid sequence having at least 87% identity to SEQ ID NO: 11.

Another aspect of the invention relates to an isolated antibody, or antigen-binding fragment thereof, comprising a heavy chain variable region (HCVR) consisting of an amino acid sequence selected from ZH1-[GYTFTSYN]-ZH2-[X₅₃GVIX₅₇PGDGX₆₄TSYX₆₈QKFX_{72]}-ZH₃-[ARDYYGSSPLGY]-ZH4 and a light chain variable region (LCVR) consisting of an amino acid sequence selected from ZL1 -[X₂₄ASX₂₇SISYX₃₉N]ZL2-[AX₅₇SX₆₆LX_{68]}-ZL3-[HQRSSX₁₁₅PT]-ZL4. In this aspect, each of ZH1, ZH2, ZH3 and ZH4 independently represents zero, one or several independently selected amino acid residues, X₅₃ is selected from I and M, X₅₇ is selected from N and Y, X₆₄ is selected from A and S, X₆₈ is selected from A and N, and X₇₂ is selected from K and Q. Furthermore, each of ZL1, ZL2, ZL3 and ZL4 independently represents zero, one or several independently selected amino acid residues, X₂₄ is selected from S and R, X27 is selected from S and P, X39 is selected from M and L, X₅₇ is selected from A and T, X₆₆ is selected from K and S, X₆₈ is selected from A and P, and X₁₁₅ is selected from S, T and Y.

A further aspect of the invention relates to an isolated antibody, or an antigen-binding fragment thereof, that specifically binds to an epitope of a BSSL, preferably hBSSL. The epitope comprises a first surface comprising an amino acid sequence according to SEQ ID NO: 1, or an amino acid sequence having at least 80%, preferably at least 83%, identity to SEQ ID NO: 1, and a second surface comprising an amino acid sequence according to SEQ ID NO: 2, or an amino acid sequence having at least 80%, preferably at least 85% or at least 92%, identity to SEQ ID NO: 2.

Yet another aspect of the invention relates to a pharmaceutical composition comprising an isolated antibody and/or an antigen-binding fragment thereof according to above and a pharmaceutically acceptable carrier or excipient.

Further aspects of the invention relates to an isolated antibody, or an antigen-binding fragment thereof, according to above, or a pharmaceutical composition according to above, for use as a medicament, and for use in the treatment and/or prevention of an inflammatory disease.

A related aspect of the invention defines the use of an isolated antibody, or antigen-binding fragment thereof, according to above, or a pharmaceutical composition according to above, for the manufacture of a medicament for the treatment and/or prevention of an inflammatory disease.

Another related aspect of the invention defines a method for treating and/or ameliorating and/or preventing and/or prophylaxis of an inflammatory disease. The method comprises administering a therapeutically effective amount of an isolated antibody, or an antigen-binding fragment thereof, according to above or a pharmaceutical composition according to above to a subject in need thereof.

Additional aspects of the invention relates to a polynucleotide encoding an antibody, or antigen-binding fragment thereof, according to above, an expression vector comprising such a polynucleotide and a cell comprising an antibody, or antigen-binding fragment thereof, according to above, a polynucleotide according to above and/or an expression vector according to above.

Another aspect of the invention relates to a method for detecting the presence or absence of BSSL and/or quantifying the amount of BSSL in a sample. The method comprises contacting the sample with an isolated antibody, or an antigen-binding fragment thereof, according to above and detecting the presence or absence of BSSL in the sample and/or quantifying the amount of BSSL in the sample based on an amount of isolated antibody, or antigen-binding fragment thereof bound to BSSL.

A further aspect of the invention relates to a method for diagnosis of a BSSL-related disorder. The method comprises contacting a sample from a subject with an isolated antibody, or an antigen-binding fragment thereof, according to above and detecting the presence or absence of BSSL and/or quantifying the amount of BSSL in the sample based on an amount of isolated antibody, or antigen-binding fragment thereof bound to BSSL. The method also comprises concluding, based on the results from the detection and/or quantification, whether the subject is suffering from a BSSL-related disorder or not.

Yet another aspect of the invention relates to a BSSL epitope comprising a first surface comprising an amino acid sequence according to SEQ ID NO: 1, or an amino acid sequence having at least 80%, preferably at least 83%, identity to SEQ ID NO: 1, and a second surface comprising an amino acid sequence according to SEQ ID NO: 2, or an amino acid sequence having at least 80%, preferably at least 85% or at least 92%, identity to SEQ ID NO: 2.

The antibodies, and antigen-binding fragments thereof, of the present invention bind to a previously uncharacterized epitope in hBSSL distinct from the active site of the enzyme. The antibodies, and antigen-binding fragments thereof, can therefore bind to hBSSL without competing with the enzymatic activity of hBSSL. The antibodies, and antigen-binding fragments thereof, of the present invention are useful in the treatment and/or prevention of inflammatory conditions, while alleviating the abovementioned and other drawbacks of current therapies.

### BRIEF DESCRIPTION OF THE DRAWINGS

The embodiments, together with further objects and advantages thereof, may best be understood by making reference to the following description taken together with the accompanying drawings, in which:

FIG. 1 shows interaction between hBSSL and immobilized AS20 mlgG1. Sensorgram fitted to the 1:1 binding model. Due to the good fit, the sensorgram and fitted line cannot be distinguished.

FIG. 2 shows steady-state analysis of the interaction between mBSSL and immobilized AS20 mlgG1. The K_{D} is taken from half the maximum response where max has automatically been corrected for high bulk effect with an offset of -173 RU (from the top). The bulk effect is not removed in the figure.

FIG. 3a shows a graph illustrating AS20 scFv ELISA binding to non-biotinylated and biotinylated human BSSL. The displayed absorbance values (y-axis) are averages of duplicates. FIG. 3b shows a graph illustrating AS20 scFv ELISA binding to mouse and human BSSL, as well as to a non-relevant protein. The displayed absorbance values (y-axis) are averages of duplicates.

FIG. 4 shows a bar graph of the results of a multi-plexed bead assay (LUMINEX®) analyzing the ability of AS20 scFv (left bars) to bind to human BSSL and to 30 different non-relevant proteins. A positive control scFv, non-relevant scFv_1 (right bars), expected to bind b-non-relevant protein_1 was also included in the assay.

FIG. 5 shows the results of a HTRF based competition assay analyzing the binding of AS20 scFv to mouse and human BSSL.

FIG. 6 is a sequence comparison between AS20, AS20 CDR graft and AS20 humanization library scaffold. * indicates a stop in the reading frame, introduced in LCDR3 to warrant that only clones mutagenized in this region are displayed on phage. X indicates positions mutagenized in the AS20 humanization library. Boundaries for CDRs are as defined by Kabat and residue numbering are as defined by the IMGT nomenclature [5]. eHCDR2, eLCDR1 and eLCDR2 indicates extended HCDR2, LCDR1 and LCDR2 regions including an amino acid position outside of the respective CDR region in accordance with IMGT.

FIG. 7 are graphs showing size exclusion chromatography data trends at A) +40° C. and B) +4° C. as described in Example 12.

FIG. 8 are graphs showing the average Tm1 (circle) and Tm2 (square) values plotted for each candidate. Bars indicate standard deviation; a) +4° C. b) +40° C.

FIG. 9 shows results from DLS analysis showing intensity vs. size for the candidates. Samples were analyzed after storage for 30 days at -80° C. (hatched arrow), +4° C. (dotted arrow) and +40° C. (full arrow) respectively.

FIG. 10 is a graphical representation of the structure of BSSL with indicated epitope regions of the prenominated antibody candidates (aa 7-12 for S-SL048-11, S-SL048-46, S-SL048-106, S-SL048-116, S-SL048-118; aa 42-55 for S-SL048-11, S-SL048-46, S-SL048-106, S-SL048-116, S-SL048-118; aa 84-101 for S-SL048-46; aa 174-180 for S-SL048-116; aa 283-295 for S-SL048-11).

FIG. 11 is as representation of the S-SL048-106 svFv with potential post translational liabilities highlighted.

FIG. 12 is a cartoon representation of t-hBSSL showing the “oven glove” view and the epitope circled in dashed line and colored light grey around the back of the glove. Strands are shown as arrows and helices as spirals.

FIG. 13 shows, in the left panel, t-hBSSL in dark grey surface representation with the sequences that interact with AS20-Fab in light grey. The variable regions of the heavy and light chain are shown in ribbon representation (light chain colored grey, and heavy chain colored black). The right panel shows the same view but t-hBSSL is represented in “sticks” with the active site triad highlighted in black. In the left panel the active site is hidden under the surface.

FIG. 14 is a table showing the differences in amino acid sequence between the 38 candidate scFv.

FIGS. 15A and 15B are a summary of the design of combinatorial scFv library for the heavy chain variable region as described in Example 5.

FIGS. 16A and 16B are a summary of the design of combinatorial scFv library for the light chain variable region as described in Example 5.

FIG. 17 are graphs showing the BSSL activity assays according to Example 20. A) and B) show the results from the triglyceride hydrolysis assay and C) and D) shown the results from the cholesterol ester hydrolysis assay. Note that chimeric AS20 is indicated as AS20 in the figure.

FIG. 18 illustrates arthritis severity. CAIA development in mice following CIA-MAB-50 injection and treatment of (A) isotype control anti-NP hIgG1 LALA-PG (90 mg/kg), AS20 hIgG1 LALA-PG (90, 30 and 10 mg/kg) every 4^{th} day from day −1 until day 15. (B) Isotype control and AS20 hIgG1 LALA-PG 90 mg/kg. (C) Isotype control and AS20 IgG1 LALA-PG 30 mg/kg. (D) Isotype control and AS20 hIgG1 LALA-PG 10 mg/kg. Two animals, one in the AS20 hIgG1 LALA-PG 10 mg/kg group and one in the isotype control group, were removed pre-termination (day 12) for ethical reasons (high score). These animals are included in the results until day of removal. Data is presented as mean±SEM. *p<0.05; **p<0.01.

FIG. 19 illustrates disease parameter graphs. CAIA disease parameters including animals treated i.p. with isotype control anti-NP hIgG1 LALA-PG (90 mg/kg), AS20 hIgG1 LALA-PG (90, 30 and 10 mg/kg) every 4^{th} day from day −1 until day 15. (A) Mean CAIA score (sum of score during experiment divided by number of scoring days). (B) Maximum CAIA score. (C) Total disease burden (AUC). (D) Percentage inhibition. Two animals, one in the AS20 hIgG1 LALA-PG 10 mg/kg group and one in the isotype control group, were removed pre-termination (day 12) for ethical reasons (high score). These animals are only included in the maximum score. Data is presented as mean±SEM. *p<0.05; **p<0.01.

FIG. 20 illustrates graphs of cellular subset in total number. Data is presented as mean±SD.

FIG. 21 illustrates graphs of cellular subset in percentage. Data is presented as mean±SD. FIG. 22 illustrates structure of the Fab-BSSL complex drawn as cartoon. Dimeric complex of S-SL048-116 Fab with light chain and heavy chain and BSSL. The same complex is turned 180° to the right.

FIG. 23 interactions between BSSL and S-SL048-116 Fab. Variable Ig domains of the Fab with light chain and the heavy chain interacting with BSSL. Two vital amino acids for the epitope Arg 176 and Gln 52 are drawn as ball and stick.

FIG. 24 illustrates arthritis severity. CAIA development in mice following CIA-MAB-50 injection and treatment of (A) vehicle and S-SL048-116 (SOL-116) (90, 30 and 10 mg/kg) every 4^{th} day from day −1 until day 15. (B) Vehicle and S-SL048-116, 90 mg/kg. (C) Vehicle and S-SL048-116, 30 mg/kg. (D) Vehicle and S-SL048-116, 10 mg/kg. Three animals were removed pre-termination for ethical reasons, two in the vehicle group (day 7 and day 15) and one in the 90 mg/kg group (day 12). These animals are included in the results until day of removal, the animal removed day 7 is completely excluded from data. Data is presented as mean±SEM.

FIG. 25 illustrates CAIA disease parameters including animals treated i.p. with vehicle and S-SL048-116 (SOL-116) at different doses (90, 30 and 10 mg/kg). (A) Mean CAIA score (sum of score during experiment divided by number of scoring days). (B) Maximum CAIA score. (C) Total disease burden (AUC). (D) Percentage inhibition. Three animals were removed pre-termination for ethical reasons, two in the vehicle group (day 7 and day 15) and one in the 90 mg/kg group (day 12). These animals are only included in the maximum score. Data is presented as mean±SEM.

FIG. 26 illustrates total number of leukocytes in (A) spleen and (B) mesenteric lymph nodes. Data is presented as mean±SEM. **p<0.01.

FIG. 27 illustrates proportion of NK cells out of CD45+ cells in (A) spleen, (B) blood and (C) mesenteric lymph nodes. Data is presented as mean±SEM. ****p<0.001.

### DETAILED DESCRIPTION

### Definitions

The term “isolated” when used in connection with antibodies, such as in the expression “isolated antibody” and the like, means the antibody has been removed from its original environment. An isolated antibody, as used herein, is intended to refer to an antibody that is substantially free of other antibodies having different antigenic specificities, e.g., an isolated antibody that specifically binds BSSL, in particular human BSSL (hBSSL), is substantially free of antibodies that specifically bind antigens other than BSSL. An isolated antibody that specifically binds hBSSL may, however, have cross-reactivity to other antigens, such as BSSL molecules from other species, such as mouse or murine BSSL (mBSSL). Moreover, an isolated antibody may be substantially free of other cellular material and/or chemicals. For example, the isolated antibody, or an antigen-binding fragment thereof, may purified to greater than 95% or 99% purity as determined by, for example, electrophoretic, e.g., sodium dodecyl sulfate-polyacrylamide gel electrophoresis (SDS-PAGE), isoelectric focusing (IEF), capillary electrophoresis, or chromatographic, e.g., ion exchange or reverse-phase high-performance liquid chromatography (HPLC). The skilled person will appreciated that isolated antibodies, or antigen-binding fragments thereof, are referred to herein even though the word “isolated” is not explicitly mentioned each time the term “antibodies, or antigen-binding fragments thereof” and the like are used.

The term “isolated humanized antibody” and the like as used herein refers to an isolated antibody which has been humanized.

The term “antigen-binding fragment” is in the context of the present document intended to mean a fragment or part of an antibody, which substantially retains antigen-binding properties. An antigen-binding fragment is a portion or region of an antibody molecule, or a derivative thereof, that retains all or a significant part of the antigen binding of the corresponding full-length antibody. An antigen-binding fragment may comprise one or more complementarity-determining region (CDR) sequences of the antibody or part of these CDR sequences, part or all of the heavy chain variable region (HCVR), part or all of the light chain variable region (LCVR), or a combination thereof. In an embodiment, an antigen-binding fragment of an antibody may be composed of a consecutive amino acid sequence of the antibody it is obtained from or may be composed of different parts of the antibody's amino acid sequence, joined together with or without linker(s). Examples of antigen-binding fragments are single-chain variable fragments (scFv), Fab fragments, F(ab′)₂ fragments, F(ab′)₃ fragments, Fab′ fragments, Fd fragments, Fv fragments, dAb fragments, isolated complementarity determining regions (CDRs) and nanobodies.

A “single chain fragment variable” or “single-chain variable fragment” (“scFv”) is a fusion protein of the variable regions of the heavy and light chains of immunoglobulins, connected with a short linker peptide of typically about 10 to 25 amino acids. scFvs of the same or different type (having an affinity for the same or different epitopes) may be combined in different ways as is known to the person skilled in the art. Non-limiting examples of such combinations are tandem di-scFv, diabodies, tandem tri-scFv or tri(a)bodies.

The term “epitope” refers to the part of an antigen that is recognized by the immune system, such as by antibodies. Epitope is also referred to as antigenic determinant.

The term “paratope” refers to the part of an antibody that binds to the epitope.

As used herein, the terms “that binds to”, “having affinity for”, “affinity” and the like refer to the property of an antibody, or an antigen-binding fragment thereof, of binding to a target molecule. Standard assays to evaluate the binding ability of an antibody or an antigen-binding fragment towards a target molecule include for example, enzyme immunoassays (EIA), such as enzyme-linked immunosorbent assay (ELISA), Western blot, radioimmunoassay (RIA), surface plasmon resonance (SPR), LUMINEX® Multiplex Assay and flow cytometry analysis. As is exemplified in the experimental section, the binding kinetics, e.g., binding affinity, of antibodies also can be assessed by standard assays known in the art, such as by the BIACORE® system analysis.

By “specifically binds to”, “specifically binding to” and the like it is meant that the molecule in question, such as an antibody, or an antigen-binding fragment thereof, specifically binds to the target antigen without any significant binding to other molecules. The specificity of an antibody, or an antigen-binding fragment thereof, can be determined based on affinity and/or avidity. The affinity, represented by the equilibrium constant for the dissociation of an antigen with the antibody, or the antigen-binding fragment thereof, (K_{D}) is a measure for the binding strength between an antigenic determinant, i.e., epitope, and an antigen-binding site on the antibody, or the antigen-binding fragment thereof. The lower the value of K_{D}, the stronger the binding strength between the antigenic determinant and the antibody, or the antigen-binding fragment thereof. Alternatively, the affinity can also be expressed as the affinity constant (K_{A}), which is 1/K_{D}. As will be clear to the skilled person, affinity can be determined in a manner known per se, depending on the specific antigen of interest.

Typically, antibodies, or antigen-binding fragments thereof, will bind to their antigen with an equilibrium dissociation constant (K_{D}) of 10⁻⁵ to 10⁻¹² moles/liter (M) or less, and preferably 10⁻⁷ to 10⁻¹² M or less and more preferably 10⁻⁸ to 10⁻¹² M, i.e., with an affinity constant (K_{A}) of 10⁵ to 10¹² M⁻¹ or more, and preferably 10⁷ to 10¹² M⁻¹ or more and more preferably 10⁸ to 10¹² M⁻¹. Generally, any K_{D} value greater than 10⁻⁴ M (or any K_{A} value lower than 10⁴ M⁻¹) is considered to indicate non-specific binding. Preferably, an antibody, or an antigen-binding fragment thereof, of the embodiments will bind to a BSSL with an affinity less than 500 nM, preferably less than 200 nM, more preferably less than 10 nM, such as less than 5 nM.

The term “detection”, “detecting” and the like includes any means of detecting, including direct and indirect detection.

Herein, all amino acids in the variable regions, including the herein described CDRs, are consequently numbered according to the IMGT unique numbering as defined by Marie-Paule Lefranc [5].

The term “Kabat numbering” and the like refers to a scheme for the numbering of amino acid residues in antibodies based upon the variable regions.

The term “monoclonal antibody”, “monoclonal antibodies” and the like as used herein refers to an antibody/antibodies having monovalent affinity, meaning that each antibody molecule in a sample of the monoclonal antibody binds to the same epitope on the antigen. Monoclonal antibodies are made by identical immune cells that are clones of a unique parent cell, for example a hybridoma cell line.

The term “polyclonal antibodies” as used herein refers to a collection of antibodies that react against a specific antigen, but in which collection there may be different antibody molecules for example identifying different epitopes on the antigen. Polyclonal antibodies are typically produced by inoculation of a suitable mammal and are purified from the mammal's serum.

The term “human antibody derivatives” refers to any modified form of the human antibody, e.g., a conjugate of the antibody and another agent or antibody.

The term “full-length antibody” as used herein refers to an antibody of any class, such as immunoglobulin D (IgD), IgE, IgG, IgA, IgM or IgY, or any sub-class thereof. The subunit structures and three-dimensional configurations of different classes of antibodies are well known.

The term “chimeric antibody” as used herein, refers to a recombinant or genetically engineered antibody, such as, for example, mouse monoclonal antibody, which contain polypeptides or domains from a different species, for example human, introduced to reduce the immunogenicity of the antibody.

As used herein, the term “at least one” is to be interpreted as one or more.

As is understood by one skilled in the art, reference to “about” a value or parameter herein includes (and describes) embodiments that are directed to that value or parameter per se. For example, description referring to “about X” includes description of “X”.

The terms “polynucleotide” and “nucleic acid,” are used interchangeably herein and refer to polymers of nucleotides of any length, and include deoxyribonucleic acid (DNA) and ribonucleic acid (RNA). The nucleotides can be deoxyribonucleotides, ribonucleotides, modified nucleotides or bases, and/or their analogs, or any substrate that can be incorporated into a polymer by DNA or RNA polymerase, or by a synthetic reaction.

As used herein, a “host cell” includes an individual cell or cell culture, which can be or has been a recipient of any vector of this document. Host cells include progeny of a single host cell, and the progeny may not necessarily be completely identical, in morphology or in total DNA complement, to the original parent cell due to natural, accidental, or deliberate mutation and/or change. A host cell includes cells transfected or infected with a vector comprising a nucleic acid of the present document. Host cells may be prokaryotic or eukaryotic cells.

The term “isolated” when used in connection with polynucleotides, polypeptides and the like means that the molecule or polypeptide has been removed from its original environment.

As used herein, the terms “% identity” or “% identical” as used herein may determined using methods well known in the art. For example, % identity be calculated as follows. The query sequence is aligned to the target sequence using the CLUSTAL W algorithm [6]. A comparison is made over the window corresponding to the shortest of the aligned sequences. The shortest of the aligned sequences may in some instances be the target sequence. In other instances, the query sequence may constitute the shortest of the aligned sequences. The amino acid residues or nucleotides at each position are compared and the percentage of positions in the query sequence that have identical correspondences in the target sequence is reported as % identity.

A “therapeutically effective amount” of an agent, as used herein refers to an amount effective, at dosages and for periods of time necessary, to achieve the desired therapeutic or prophylactic result.

It is understood that aspect and embodiments described herein include “consisting” and/or “consisting essentially” of aspects and embodiments. As used herein, the singular form “a”, “an”, and “the” includes plural references unless indicated otherwise.

“hIgG1 LALA-PG” as described in [7].

“hIgG4 S228P, hIgG4 S241P” as described in [8].

“G-SP140-8”, SP140-binding clone, negative control.

“expiHEK293 cells”, human cells derived from the 293 cell line, and a core component of the Expi293TM Expression System.

It is an object of the present invention to provide antibodies, and/or antigen-binding fragments thereof, that are useful in the treatment and/or prevention of inflammatory conditions, while alleviating the previously mentioned and other drawbacks of current therapies. Further it is an object of the present disclosure to provide agents to be used in diagnostics of inflammatory conditions and for studying the Bile Salt-Stimulated Lipase (BSSL) protein.

In more detail, the invention relates to novel isolated antibodies, and antigen-binding fragments thereof, that bind to a previously uncharacterized epitope of BSSL situated in the N-terminal part of the BSSL protein. The present document also relates to the medical uses of the antibodies, and antigen-binding fragments thereof, in particular in treatment of inflammatory conditions, and to related pharmaceutical compositions. The present document also discloses the use of the antibodies, or antigen-binding fragments thereof, as molecular tools in the detection of BSSL and/or for diagnosing BSSL related diseases.

In an embodiment, whenever BSSL is referred to, this also includes human BSSL (hBSSL), unless it is made clear from the context that hBSSL is not intended to be included.

The present disclosure describes a novel group of antibodies against BSSL, including antigen-binding fragments thereof, which bind to a formerly unrecognized epitope on the hBSSL. The antibodies may be humanized or their CDR sequences grafted onto a non-human backbone. The antibodies, or antigen-binding fragments thereof, may also bind to mouse or murine BSSL (mBSSL) although the affinity for hBSSL and mBSSL may differ due to amino acid difference(s) in one of the epitopes that the antibodies and/or antigen-binding fragments thereof bind to.

As is well known, antibodies are immunoglobulin molecules capable of specific binding to a target (an antigen), such as a carbohydrate, polynucleotide, lipid, polypeptide or other, through at least one antigen recognition site located in the variable region of the immunoglobulin molecule. An antibody is a glycoprotein comprising at least two heavy (H) chains (HC) and two light (L) chains (LC) inter-connected by disulfide bonds. The heavy chain variable regions (HCVRs) and the light chain variable regions (LCVRs) contain a binding domain that interacts with an antigen. The constant regions of the antibodies may mediate the binding of the immunoglobulin to host tissues or factors, including various cells of the immune system, e.g., effector cells, and the first component of the classical complement system, i.e., complement component 1 q (C1q).

The antibodies, or antigen-binding fragments thereof, disclosed herein may be used to inhibit or reduce at least some of biological activities of the BSSL protein when bound thereto. The binding may, for instance, significantly or completely inhibit some of the biological activities of the BSSL protein. These effects of the antibodies, or the antigen-binding fragments thereof, of the invention were highly surprising given that the antibodies, or the antigen-binding fragments thereof, do not bind to the active site of BSSL. Thus, the antibodies, or the antigen-binding fragments thereof, of the invention preferable do not significantly inhibit or reduce the enzymatic activity of BSSL, such as do not significantly inhibit or reduce the capability of BSSL to hydrolyze cholesterol esters (EC 3.1.1.13).

The antibodies, or antigen-binding fragments thereof, can be used to reduce pro-inflammatory effects of BSSL in subjects in need thereof. Thus, the antibodies, or antigen-binding fragments thereof, can be used in treatment and/or prevention of various inflammatory diseases as is further described herein. These medical uses of the antibodies, or antigen-binding fragments thereof, of the invention can be achieved without blocking the enzymatic activity of BSSL. Hence, the antibodies, or antigen-binding fragments thereof, of the invention do not contribute to negative effects caused by inhibition of the enzymatic activity of BSSL that other anti-BSSL antibodies binding to or in connection with the active site of BSSL may have.

Also, the herein disclosed antibodies, or antigen-binding fragments thereof, may be used for diagnostic purposes for diagnosing BSSL related conditions, such as BSSL-related inflammatory conditions.

As is demonstrated in the experimental section, several approaches were used to try to develop a humanized BSSL antibody, or antigen-binding fragment thereof, but despite an initial number of about 1,000,000 candidate scFv, a surprisingly low number were found to show a sufficient binding affinity to the hBSSL protein. Of the 1,000,000 candidates, 68 initial candidates were identified, which was then reduced to 38 candidates and eventually down to 5 candidates chosen for further evaluation and characterization. Accordingly, anti-BSSL antibodies, or antigen-binding fragments thereof, cannot easily be humanized using standard protocols and still have sufficient binding affinity to hBSSL. Thus, humanization of anti-BSSL antibodies, or antigen-binding fragments, has been a big challenge that was overcome as described herein to achieve humanized antibodies, or antigen-binding fragments thereof, that still present sufficient binding affinity to hBSSL. These antibodies, or antigen-binding fragments thereof, of the present invention share common structural and functional features as described elsewhere herein.

### Generation of Antibodies and Antigen-Binding Fragments

The antibodies, and antigen-binding fragments thereof, of the present invention were generated in a multistep method with an aim to find antibodies, and antigen-binding fragments thereof, which had a sufficiently good binding affinity for hBSSL. It was also an object to provide humanized antibodies, and antigen-binding fragments thereof. Some of the identified antibodies, and antigen-binding fragments thereof, were also found to bind with a sufficient binding affinity to mBSSL. Although the present invention is not limited to humanized antibodies binding to BSSL, humanized BSSL-binding antibodies, and antigen-binding fragments thereof, are disclosed herein.

The antibodies, and antigen-binding fragments thereof, were generated based on the sequence of a non-human monoclonal mBSSL antibody. DNA encoding the heavy and light chain immunoglobulins were obtained from a non-human hybridoma expressing this antibody and engineered to contain non-murine, e.g., human, immunoglobulin sequences using standard molecular biology techniques.

However, it was surprisingly found that a CDR-graft constructed as disclosed in Example 6 did not have as high binding affinity as would have been expected based on the mBSSL antibody that was used to provide the CDR sequences. Thus, in order to obtain antibodies, and antigen-binding fragments thereof, with high enough binding affinity to hBSSL, further modifications to the framework (FW) of the antibody had to be performed by introducing mutations into both the CDR and the adjacent FW regions as disclosed in Example 5. This was achieved by constructing a humanization library used for selection and isolation of scFv fragments binding mouse and human BSSL using phage display. Such phage display methods for isolating human antibodies are established in the art, see for example U.S. Pat. Nos. 5,223,409; 5,403,484; 5,571,698; 5,427,908; 5,580,717; 5,969,108; 6,172,197; 5,885,793; 6,521,404; 6,544,731; 6,555,313; 6,582,915 and 6,593,081.

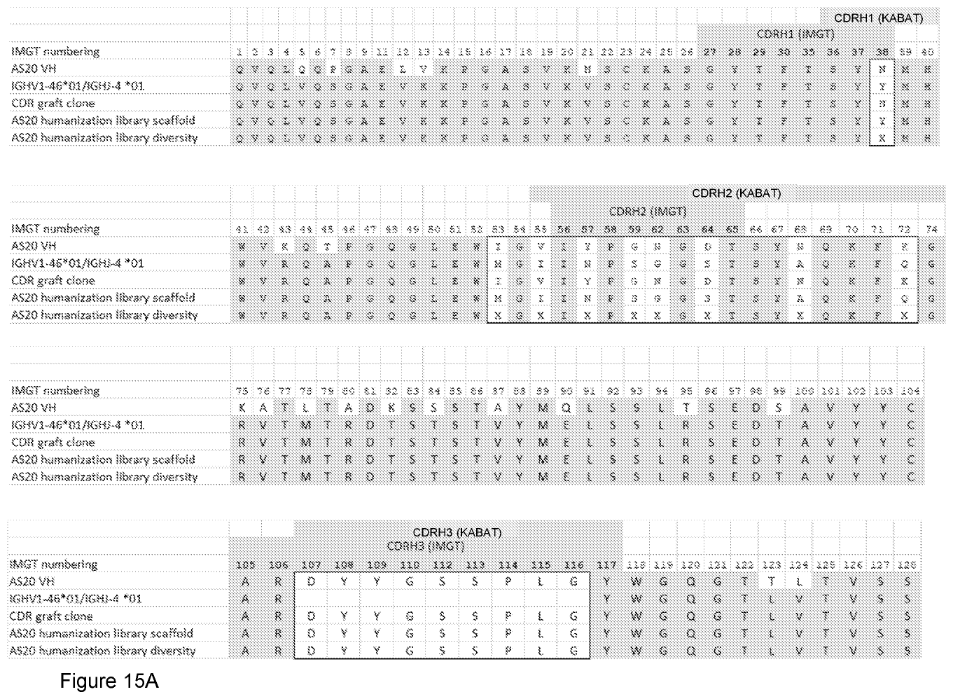

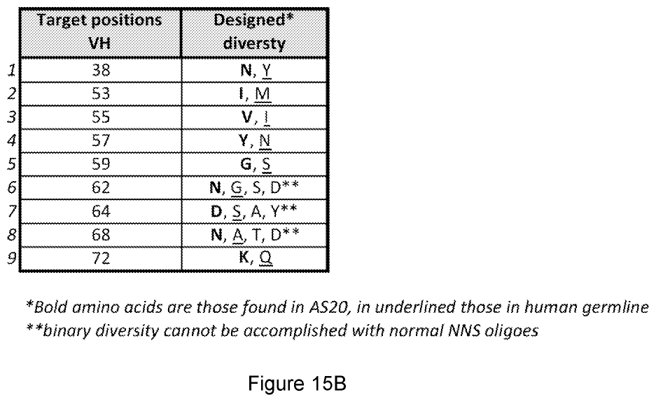

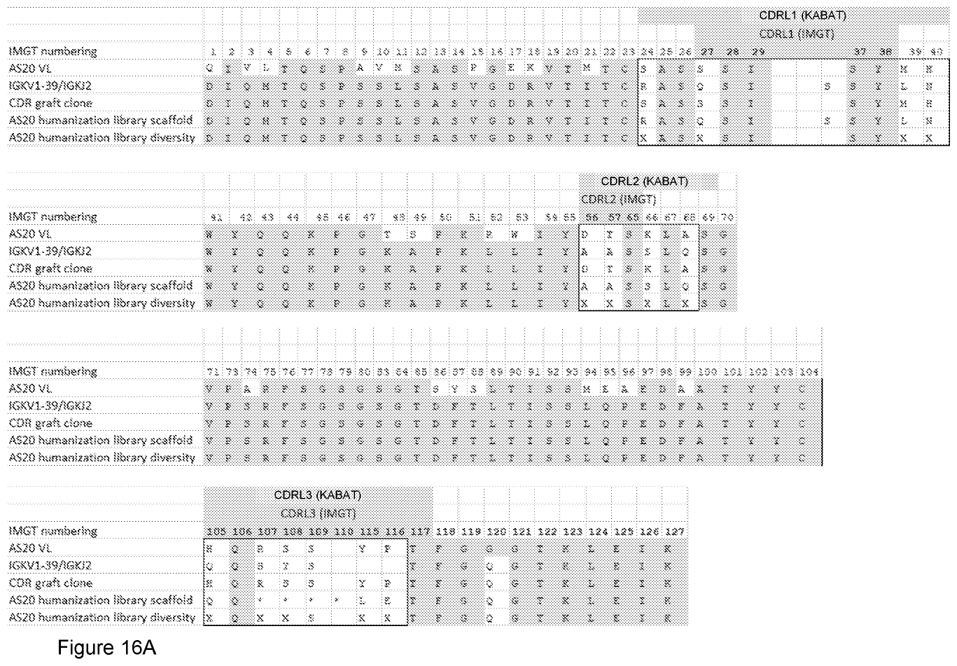

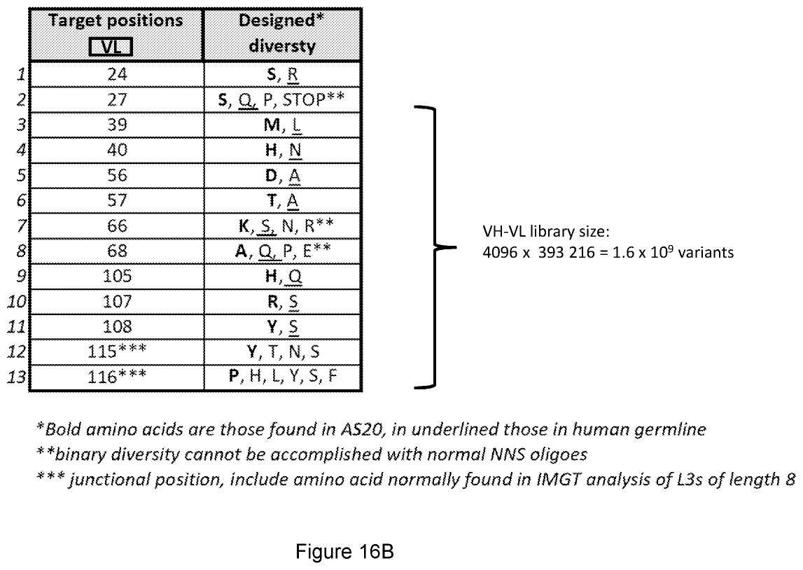

The resulting isolated antibody and antigen-binding fragment thereof had a minimized animal-derived CDR content wherein only essential non-human germ-line residues were allowed. The rest of the CDRs were converted to the human v-gene sequence, with the exception of a few novel variations by introduction of species-neutral essential de-novo residues, such as in IMGT amino acid residues 62, 64, 68, 27, 66, 68, 115 and 116 (see FIGS. 15 and 16). These CDR sequences can be grafted onto a human or non-human framework to prepare a humanized or non-humanized antibody and/or antigen-binding fragment thereof, depending on the intended use of the antibody.

In humanized antibodies, the constant regions and part of the variable regions, except for the CDR sequences, have a framework, which is derived from human germline immunoglobulin sequences. However, in such humanized antibodies, the CDR sequences are derived from the germline of another mammalian species, such as a mouse, that have been grafted onto the human framework sequences. Such humanized antibodies may in the context of the present invention also be denoted CDR-grafts. An advantage with the use of humanized antibodies is that they decrease the risk for immunogenic reactions, which may occur when a framework from another species is used if the antibody is injected into a human subject. This opens up for their use for medical applications in humans. The antibodies, or antigen-binding fragments thereof, disclosed herein are useful in diagnostic applications and for the detection of the BSSL protein, such as hBSSL, in different kinds of samples as disclosed in more detail elsewhere herein.

The present document, thus, also discloses a method for producing an isolated antibody, or an antigen-binding fragment thereof, according to the present invention. The method comprises culturing a host cell expressing an antibody, or an antigen-binding fragment thereof, under conditions permissive of expression of the antibody, or antigen-binding fragment thereof, from an expression vector comprised in the host cell and comprising a polynucleotide encoding the antibody, or the antigen-binding fragment thereof. The method also comprises isolating the antibody, or antigen-binding fragment thereof, from the host cell or from a culture medium, in which the host cell is cultured.

### Epitope Binding of the Antibodies or Antigen-Binding Fragments Thereof

The isolated antibodies, or antigen-binding fragments thereof, have been found to bind to previously unrecognized epitope of the human BSSL protein, which is located in the N-terminal part of the BSSL protein. The epitope may form a conformational epitope of BSSL.

It was surprisingly found that the antibodies, and antigen-binding fragments thereof, generated according to the present invention bind to a previously unrecognized epitope of the hBSSL protein.

Even more surprisingly, the epitope was found to not being located near the active site of BSSL for lipid metabolism but rather in the N-terminal part of BSSL. This has several advantageous effects. One is that the antibodies, or antigen-binding fragments thereof, are less likely to cause negative side effects as they do not significantly affect the enzymatic lipase activity of BSSL. Another advantage is that the antibodies, or antigen-binding fragments thereof, are suitable for studying the BSSL protein and its lipase activity as this is not significantly affected by the antibodies, or antigen-binding fragments thereof, of the present invention.

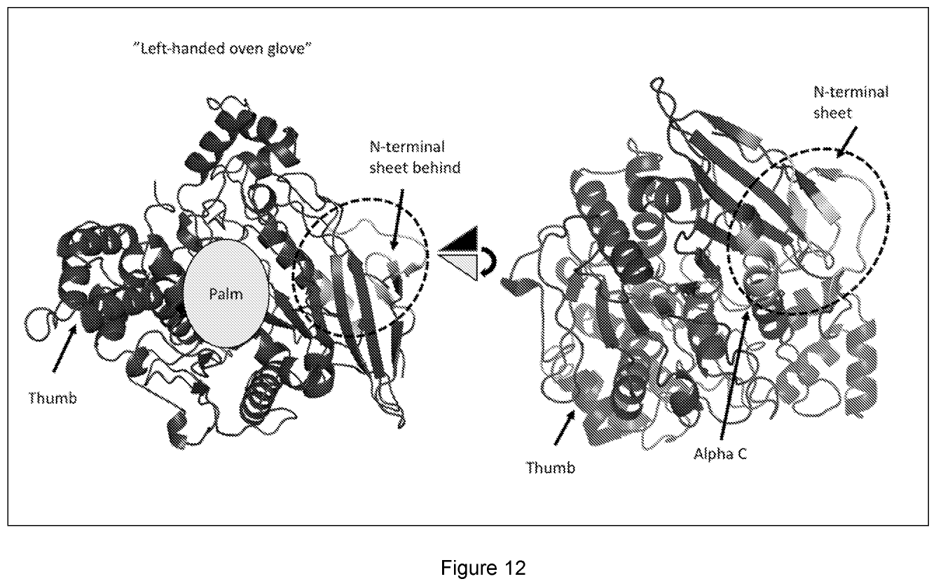

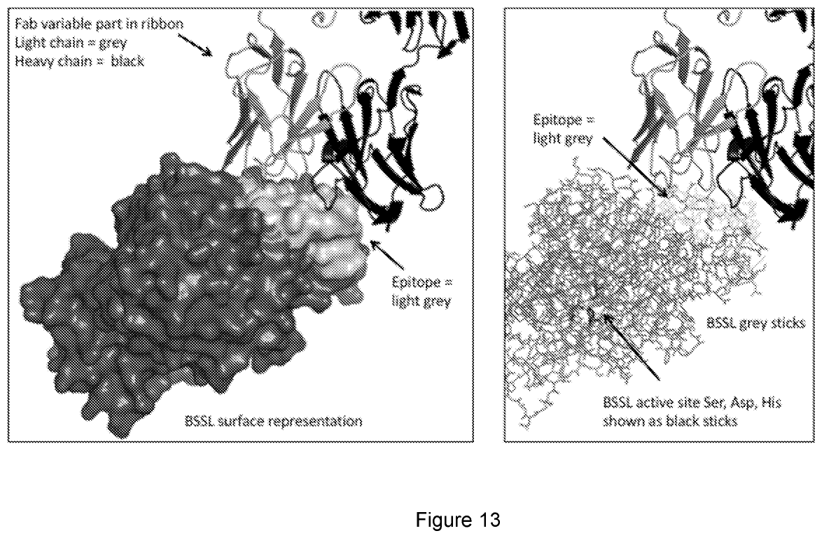

The BSSL structure has been described as a having a large core region consisting of a twisted, 11-stranded beta-sheet surrounded by alpha helices and connecting loops ([9], FIG. 12). At the N-terminus there is a smaller 3-stranded beta-sheet. The structure has been likened to a left-handed oven-glove with the palm containing the active site triad close to the “thumb”. With this likeness, the small N-terminal beta-sheet is located on the back of the hand close to the “little finger”, see FIG. 12. The part of the BSSL structure, which interacts with the Fab molecule is located to the small N-terminal beta-sheet and the C-terminal part of alpha C, the third alpha helix in the structure, see FIG. 13. In other words, the binding region for the antibody is not close to the active site of BBSL but on the opposite side of BSSL.

The epitope region now identified comprises residues 7-12 (strand 1 and 2) and 42-55 (loop region leading into strand 3 of the sheet). The epitope is rather flat with only a few characteristic residues sticking out, namely Tyr7, Phe12 and GIn52 (main interactions listed in Table 25). The loop region of 47-54 is well defined and forms a uniform surface. Proline 47 is important for a stacking interaction with Tyr31 of the antibody but as a whole the surface is flat here. In a preferred embodiment, the epitope region also comprises residues 174-180 (the C-terminal end of alpha C).

An aspect of the invention relates to an isolated antibody, or antigen-binding fragment thereof, that specifically binds to an epitope of BSSL, preferably hBSSL. The epitope comprises a first surface comprising, or defined by, an amino acid sequence according to SEQ ID NO: 1 or an amino acid sequence having at least 80%, such as at least 83%, identity to SEQ ID NO: 1. The epitope also comprises a second surface comprising, or defined by, an amino acid sequence according to SEQ ID NO: 2, or an amino acid sequence having at least 80%, such as at least 85% or at least 92%, identity to SEQ ID NO: 2.

A first peptide comprising the amino acid sequence of SEQ ID NO: 1 defines the first surface of the epitope in BSSL and a second peptide comprising the amino acid sequrence of SEQ ID NO: 2 defines the second surface of the epitope in BSSL. Hence, the first surface comprises, or rather is defined by, the amino acid sequence of SEQ ID NO: 1 and the second surface comprises, or rather is defined by, the amino acid sequence of SEQ ID NO: 2.

In an embodiment, the first peptide comprises an amino acid sequence according to SEQ ID NO: 3, or an amino acid sequence having at least 80%, preferably at least 83%, and more preferably at least 91% identity to SEQ ID NO: 3. SEQ ID NO: 3 is a longer amino acid sequence comprising as a part of it the amino acid sequence according to SEQ ID NO: 1.

The isolated antibody, or antigen-binding fragment thereof, may further specifically bind to another peptide and surface, i.e., a third peptide and a third surface, of BSSL, such as hBSSL. In an embodiment, this third peptide comprises an amino acid sequence according to SEQ ID NO: 5, or an amino acid sequence having at least 80%, preferably at least 85%, identity to SEQ ID NO: 5. In another embodiment, the third peptide comprise an amino acid according to SEQ ID NO: 4, or an amino acid sequence having at least 80%, preferably at least 83%, more preferably at least 88%, such as at least 94%, identity to SEQ ID NO: 4. In a further embodiment, the third peptide comprises an amino acid sequence according to SEQ ID NO: 6, or an amino acid sequence having at least 80%, preferably at least 84%, and more preferably at least 92%, identity to SEQ ID NO: 6.

In an embodiment, the isolated antibody, or antigen-binding fragment thereof, may specifically bind to a first peptide comprising, such as consisting of, SEQ ID NO: 1, a second peptide comprising, such as consisting of, SEQ ID NO: 2 and a third peptide comprising, such as consisting of, SEQ ID NO: 4, or an amino acid sequence having the herein defined respective identities thereto. Alternatively, instead of binding to the shorter amino acid sequence according to SEQ ID NO: 1, such an antibody, or antigen-binding fragment thereof, may bind to the longer amino acid sequence according to SEQ ID NO: 3, or an amino acid sequence having the herein specified identity thereto.

In another embodiment, the isolated antibody, or antigen-binding fragment thereof, binds specifically to a first peptide comprising, such as consisting of, SEQ ID NO: 1, a second peptide comprising, such as consisting of SEQ ID NO: 2, and a third peptide comprising, such as consisting of, SEQ ID NO: 5, or an amino acid sequence having the herein defined respective identities thereto. Alternatively, instead of binding to the shorter amino acid sequence according to SEQ ID NO: 1, such an antibody, or antigen-binding fragment thereof, may specifically bind to the longer amino acid sequence according to SEQ ID NO: 3, or an amino acid sequence having the herein defined identity thereto.

In a further embodiment, the isolated antibody, or antigen-binding fragment thereof, specifically binds to a first peptide comprising, such as consisting of SEQ ID NO: 1, a second peptide comprising, such as consisting of SEQ ID NO: 2, and a third peptide comprising, such as consisting of, SEQ ID NO: 6, or an amino acid sequence having the herein defined respective specified identities thereto. Alternatively, instead of binding to the shorter amino acid sequence according to SEQ ID NO: 1, such an antibody, or antigen-binding fragment thereof, may bind to the longer amino acid sequence according to SEQ ID NO: 3 or an amino acid sequence having the herein defined specified identity thereto.

Another aspect of the invention is directed to a BSSL epitope, such as a hBSSL epitope, comprising a first peptide comprising, such as consisting of, an amino acid sequence according to SEQ ID NO: 1, or an amino acid sequence having at least 80%, preferably at least 83%, identity to SEQ ID NO: 1. The BSSL epitope also comprises a second peptide comprising, such as consisting of, an amino acid sequence according to SEQ ID NO: 2, or an amino acid sequence having at least 80%, preferably at least 85% or at least 92%, identity to SEQ ID NO: 2.

In an embodiment, the first peptide comprises, such as consists of, an amino acid sequence according to SEQ ID NO: 3, or an amino acid sequence having the herein defined identity thereto.

The epitope may further comprise a third peptide comprising, such as consisting of, an amino acid sequence according to SEQ ID NO: 4, or an amino acid sequence having the herein defined identity thereto, an amino acid sequence according to SEQ ID NO: 5, or an amino acid sequence having the herein defined identity thereto, or an amino acid sequence according to SEQ ID NO: 6, or an amino acid sequence having the herein defined identity thereto.

Such epitopes may be useful for the development of antibodies, or antigen-binding fragments thereof, binding to the BSSL protein, such as hBSSL, e.g., for use for the treatment and/or prevention of BSSL related conditions and/or for use as molecular tools to study the BSSL protein. The present invention is therefore also directed to the use of such epitopes for the development of an antibody, or antigen-binding fragment thereof. As these epitopes are not located at the BSSL proteins lipase catalytic center, it is an attractive target to develop anti-BSSL antibodies, or antigen-binding fragments, against.

The isolated antibody, or antigen-binding fragment thereof, according to the present invention may specifically bind to an epitope(s) as defined herein.

### Antigen-Binding Parts of the Antibodies and Antigen-Binding Fragments Thereof

A full-length antibody comprises two heavy chains and two light chains inter-connected by disulfide bonds. Each heavy chain contains a heavy chain variable region (HVCR) and first, second and third constant regions (C_{H}1, C_{H}2 and C_{H}3). In this disclosure the terms V_{H}, VH and HCVR are used interchangeably. Each light chain contains a light chain variable region (LVCR), and a light chain constant region (C_{L}). In this disclosure the terms VL, VL and LCVR are used interchangeably.

The HCVR and LCVR regions can be further subdivided into regions of hypervariability, termed complementarity-determining regions (CDR), interspersed with regions that are more conserved, termed framework regions (FR or FW). Each HCVR and LCVR is composed of three CDRs and four FRs/FWs, arranged from N-terminus to C-terminus in the following order: FW1, CDR1, FW2, CDR2, FW3, CDR3, FW4.

Extended CDR (eCDR) as used herein relates to an amino acid sequence that comprises at least one additional amino acid residue beyond the amino acids of the CDR as defined according to the IMGT nomenclature.

It is well known in the art that the paratope, also known as the antigen-binding site, is the part of an antibody, or antigen-binding fragment thereof, which recognizes and binds to an antigen. It is a small region of the antibody's Fv region, and contains parts of the antibody's heavy and light chains. Each arm of the Y shape of an antibody monomer is tipped with a paratope, which is the set of 6 CDRs. The paratope is made up of three light chain CDRs (LCDRs) and three heavy chains CDRs (HCDRs), which extend from the fold of antiparallel beta sheets.

In the following sections, the isolated antibody, or antigen-binding fragment thereof, of the present invention is defined by the structural features of its CDRs, in other words by the amino acid sequence of its HCDRs and/or LCDRs, or the amino acid structure of regions comprising the HCDRs and/or LCDRs. The skilled person will appreciate that minor variations, such as substitutions of one, two, three, four or even more amino acid residues, in the amino acid sequence may occur without affecting the functional properties, such as its binding capacity or binding affinity to BSSL, such as hBSSL, of the isolated antibody, or antigen-binding fragment thereof. It will be understood that the first HCDR, second HCDR, third HCDR, first LCDR, second LCDR and third LCDR may be independently selected from the amino acid sequences recited.

An aspect of the invention, thus, relates to an isolated antibody, or antigen-binding fragment thereof comprising three CDRs of a HCVR (HCDRs) and three CDRs of a LCHV (LCDRs). In this aspect, the first HCDR comprises, preferably consists of, an amino acid sequence according to SEQ ID NO: 7, or an amino acid sequence having at least 87%, such as at least 87.5%, identity to SEQ ID NO: 7, the second HCDR comprises, preferably consists of, an amino acid sequence according to SEQ ID NO: 8, or an amino acid sequence having at least 75% identity to SEQ ID NO: 8, and the third HCDR comprises, preferably consists of, an amino acid sequence according to SEQ ID NO: 9, or an amino acid sequence having at least 83%, such as at least 91.6%, identity to SEQ ID NO: 9. Furthermore, in this aspect the first LCDR comprises, preferably consists of, an amino acid sequence according to SEQ ID NO: 10, or an amino acid sequence having at least 80% identity to SEQ ID NO: 10, the second LCDR comprises, preferably consists of, the amino acid sequence ATS, or an amino acid sequence having at least 66% identity to the amino acid sequence ATS, such as AAS, and the third LCDR comprises, preferably consists of, an amino acid sequence according to SEQ ID NO: 11, or an amino acid sequence having at least 87% identity to SEQ ID NO: 11.

Experimental data as presented in Example 22, indicates that the second LCDR may not be that important for interaction with BSSL. Hence, in embodiment, the isolated antibody, or antigen-binding fragment thereof comprising a first HCDR comprising, preferably consisting of, an amino acid sequence according to SEQ ID NO: 7, or an amino acid sequence having at least 87%, such as at least 87.5%, identity to SEQ ID NO: 7, a second HCDR comprising, preferably consisting of, an amino acid sequence according to SEQ ID NO: 8, or an amino acid sequence having at least 75% identity to SEQ ID NO: 8, a third HCDR comprising, preferably consisting of, an amino acid sequence according to SEQ ID NO: 9, or an amino acid sequence having at least 83%, such as at least 91.6%, identity to SEQ ID NO: 9, a first LCDR comprising, preferably consisting of, an amino acid sequence according to SEQ ID NO: 10, or an amino acid sequence having at least 80% identity to SEQ ID NO: 10, and a third LCDR comprising, preferably consisting of, an amino acid sequence according to SEQ ID NO: 11, or an amino acid sequence having at least 87% identity to SEQ ID NO: 11.

In an embodiment, the first HCDR comprises, preferably consists of, the amino acid sequence according to SEQ ID NO: 7, the second HCDR comprises, preferably consists of, an amino acid sequence selected from the group consisting of SEQ ID NO: 8, SEQ ID NO: 18 and SEQ ID NO: 19, and the third HCDR comprises, preferably consists of, the amino acid sequence according to SEQ ID NO: 9. In this embodiment, the first LCDR comprises, preferably consists of, an amino acid sequence selected from the group consisting of SEQ ID NO: 10 and SEQ ID NO: 20, the second LCDR comprises, preferably consists of, an amino acid sequence selected from the group consisting of ATS and AAS, and the third LCDR comprises, preferably consists of, the amino acid sequence selected from the group consisting of SEQ ID NO: 11, SEQ ID NO: 21 and SEQ ID NO: 22.

In an embodiment, the first HCDR comprises, preferably consists of, an amino acid sequence according to SEQ ID NO: 7, the second HCDR comprises, preferably consists of, the amino acid sequence according to SEQ ID NO: 8 and the third HCDR comprises, preferably consists of, the amino acid sequence according to SEQ ID NO: 9. In this embodiment, the first LCDR comprises, preferably consists of, the amino acid sequence according to SEQ ID NO: 10, the second LCDR comprises, preferably consists of, the amino acid sequence ATS and the third LCDR comprises, preferably consists of, the amino acid sequence according to SEQ ID NO: 11.

In a particular embodiment, the isolated antibody, or antigen-binding fragment thereof, comprises an extended second HCDR comprising, preferably consisting of, an amino acid sequence according to SEQ ID NO: 12, an extended first LCDR comprising, preferably consisting of, an amino acid sequence according to SEQ ID NO: 14, and an extended second LCDR comprising, preferably consisting of, an amino acid sequence according to SEQ ID NO: 15.

In another particular embodiment, the isolated antibody, or antigen-binding fragment thereof, comprises an extended second HCDR comprising, preferably consisting of, an amino acid sequence according to SEQ ID NO: 12, an extended first LCDR comprising, preferably consisting of, an amino acid sequence according to SEQ ID NO: 16 and an extended second LCDR comprising, preferably consisting of, the amino acid sequence according to SEQ ID NO: 17.

In an embodiment, the first HCDR comprises, preferably consists of, the amino acid sequence according to SEQ ID NO: 7, the second HCDR comprises, preferably consists of, the amino acid sequence according to SEQ ID NO: 18 and the third HCDR comprises, preferably consists of, the amino acid sequence according to SEQ ID NO: 9. In this embodiment, the first LCDR comprises, preferably consists of, the amino acid sequence according to SEQ ID NO: 10, the second LCDR comprises, preferably consists of, the amino acid sequence ATS and a third LCDR comprises, preferably consists of, an amino acid sequence according to SEQ ID NO: 21.

In a particular embodiment, the isolated antibody, or antigen-binding fragment thereof, comprises an extended second HCDR comprising, preferably consisting of, an amino acid sequence according to SEQ ID NO: 23, an extended first LCDR comprising, preferably consisting of, an amino acid sequence according to SEQ ID NO: 16 and an extended second LCDR comprising, preferably consisting of, an amino acid sequence according to SEQ ID NO: 15.

In an embodiment, the first HCDR comprises, preferably consists of, the amino acid sequence according to SEQ ID NO: 7, the second HCDR comprises, preferably consists of, an amino acid sequence according to SEQ ID NO: 8 and the third HCDR comprises, preferably consists of, the amino acid sequence according to SEQ ID NO: 9. In this embodiment, the first LCDR comprises, preferably consists of, the amino acid sequence according to SEQ ID NO: 20, the second LCDR comprises, preferably consists of, the amino acid sequence AAS and the third LCDR comprises, preferably consists of, the amino acid sequence according to SEQ ID NO: 11.

In a particular embodiment, the isolated antibody, or antigen-binding fragment thereof, comprises an extended second HCDR comprising, preferably consisting of, an amino acid sequence according to SEQ ID NO: 24, an extended first LCDR comprising, preferably consisting of, an amino acid sequence according to SEQ ID NO: 27 and an extended second LCDR comprising, preferably consisting of, an amino acid sequence according to SEQ ID NO: 29.

In an embodiment, the first HCDR comprises, preferably consists of, the amino acid sequence according to SEQ ID NO: 7, the second HCDR comprises, preferably consists of, the amino acid sequence according to SEQ ID NO: 19 and the third HCDR comprises, preferably consists of, the amino acid sequence according to SEQ ID NO: 9. In this embodiment, the first LCDR comprises, preferably consists of, the amino acid sequence according to SEQ ID NO: 20, the second LCDR comprises, preferably consists of, the amino acid sequence ATS and the third LCDR comprises, preferably consists of, the amino acid sequence according to SEQ ID NO: 22.

In a particular embodiment, the isolated antibody, or antigen-binding fragment thereof, comprises an extended second HCDR comprising, preferably consisting of, an amino acid sequence according to SEQ ID NO: 25, an extended first LCDR comprising, preferably consisting of, an amino acid sequence according to SEQ ID NO: 26, and an extended second LCDR comprising, preferably consisting of, the amino acid sequence according to SEQ ID NO: 28.

In an embodiment, the isolated antibody, or antigen-binding fragment thereof, comprises a first HCDR comprising, preferably consisting of, the amino acid sequence according to SEQ ID NO: 7, an extended second HCDR comprising, preferably consisting of, one amino acid sequence selected from the group consisting of SEQ ID NO: 12, 23, 24 and 25, and a third HCDR comprising, preferably consisting of, the amino acid sequence according to SEQ ID NO: 9. In this embodiment, the isolated antibody, or antigen-binding fragment thereof, an extended first LCDR comprising, preferably consisting of, one amino acid sequence selected from the SEQ ID NO: 14, SEQ ID NO: 16, SEQ ID NO: 26 and SEQ ID NO: 27, an extended second LCDR comprising, preferably consisting of, an amino acid sequence selected from the group consisting of SEQ ID NO: 15, SEQ ID NO: 17, SEQ ID NO: 28 and SEQ ID NO: 29, and a third LCDR comprising, preferably consisting of, an amino acid sequence selected from the group consisting of SEQ ID NO: 11, SEQ ID NO: 21 and SEQ ID NO: 22.

In an embodiment, when the first HCDR comprises, preferably consists of, the amino acid sequence according to SEQ ID NO: 7, this also includes an amino acid sequence which is at least 87.5% identical to SEQ ID NO: 7.

In an embodiment, when the second HCDR comprises, preferably consists of, the amino acid sequence according to SEQ ID NO: 8, this also includes an amino acid sequence which is at least 75%, such at least 87%, or at least 87.5%, identical to SEQ ID NO: 8.

In an embodiment, when the third HCDR comprises, preferably consists of, the amino acid sequence according to SEQ ID NO: 9, this also includes an amino acid sequence which is at least 83%, such as at least 91.6% identical to SEQ ID NO: 9.

In an embodiment, when the first LCDR comprises, preferably consists of, the amino acid sequence according to SEQ ID NO: 10, this also includes an amino acid sequence which is at least 80% identical to SEQ ID NO: 10.

In an embodiment, when the second LCDR comprises, preferably consists of, the amino acid sequence ATS or AAS, this also includes an amino acid sequence which is at least 66% identical to either one of the amino acid sequences ATS and AAS.

In an embodiment, when the third LCDR comprises, preferably consists of, the amino acid sequence according to SEQ ID NO: 11, this also includes an amino acid sequence which is at least 87.5% identical to SEQ ID NO: 11.

In an embodiment, when the third LCDR comprises, preferably consists of, an amino acid sequence according to SEQ ID NO: 21, this also includes an amino acid sequence which is at least 75%, such as at least 87.5% identical to SEQ ID NO: 21.

In an embodiment, when the third LCDR comprises, preferably consists of, the amino acid sequence according to SEQ ID NO: 22, this also includes an amino acid sequence which is at least 75%, such as at least 87.5% identical to SEQ ID NO: 22.

In an embodiment, when the extended second HCDR comprises, preferably consists of, the amino acid sequence according to SEQ ID NO: 12, this also includes an amino acid sequence which is at least 77.8%, such as at least 83%, such as at least 83.3%, such as at least 88%, such as at least 88.9%, such as at least 94%, such as at least 94.4% identical to SEQ ID NO: 12.

In an embodiment, when the extended second HCDR comprises, preferably consists of, the amino acid sequence according to SEQ ID NO: 18, this also includes an amino acid sequence which is at least 75%, such as at 87.5%, identical to SEQ ID NO: 18.

In an embodiment, when the extended second HCDR comprises, preferably consists of, the amino acid sequence according to SEQ ID NO: 19, this also includes an amino acid sequence which is at least 75%, such as at 87.5%, identical to SEQ ID NO: 19.

In an embodiment, when the extended second HCDR comprises, preferably consists of, the amino acid sequence according to SEQ ID NO: 23, this also includes an amino acid sequence which is at least 77.8%, such as at least 83%, such as at least 83.3%, such as at least 88%, such as at least 88.9%, such as at least 94%, such as at least 94.4% identical to SEQ ID NO: 23.

In an embodiment, when the extended second HCDR comprises, preferably consists of, the amino acid sequence according to SEQ ID NO: 24, this also includes an amino acid sequence which is at least 77.8%, such as at least 83%, such as at least 83.3%, such as at least 88%, such as at least 88.9%, such as at least 94%, such as at least 94.4% identical to SEQ ID NO: 24.

In an embodiment, when the second HCDR comprises, preferably consists of, the amino acid sequence according to SEQ ID NO: 25, this also includes an amino acid sequence which is at least 77.8%, such as at least 83%, such as at least 83.3%, such as at least 88%, such as at least 88.9%, such as at least 94%, such as at least 94.4% identical to SEQ ID NO: 25.

In an embodiment, when the extended first LCDR comprises, preferably consists of, the amino acid sequence according to SEQ ID NO: 14, this also includes an amino acid sequence which is at least 80%, such as at least 90% identical to SEQ ID NO: 14.

In an embodiment, when the extended first LCDR comprises, preferably consists of, the amino acid sequence according to SEQ ID NO: 16, this also includes an amino acid sequence which is at least 80%, such as at least 90% identical to SEQ ID NO: 16.

In an embodiment, when the extended first LCDR comprises, preferably consists of, the amino acid sequence according to SEQ ID NO: 20, this also includes an amino acid sequence which is at least 80% identical to SEQ ID NO: 20.

In an embodiment, when the extended first LCDR comprises, preferably consists of, the amino acid sequence according to SEQ ID NO: 26, this also includes an amino acid sequence which is at least 80%, such as at least 90% identical to SEQ ID NO: 26.

In an embodiment, when the extended first LCDR comprises, preferably consists of, the amino acid sequence according to SEQ ID NO: 27, this also includes an amino acid sequence which is at least 80%, such as at least 90% identical to SEQ ID NO: 27.

In an embodiment, when the extended second LCDR comprises, preferably consists of, an amino acid sequence according to SEQ ID NO:15 this also includes an amino acid sequence which is at least 66.7%, such as least 83%, such as at least 83.3%, identical to SEQ ID NO: 15.

In an embodiment, when the extended second LCDR comprises, preferably consists of, the amino acid sequence according to SEQ ID NO: 17, this also includes an amino acid sequence which is at least 66.7%, such as least 83%, such as at least 83.3%, identical to SEQ ID NO: 17.

In an embodiment, when the extended second LCDR comprises, preferably consists of, the amino acid sequence according to SEQ ID NO: 28, this also includes an amino acid sequence which is at least 66.7%, such as least 83%, such as at least 83.3%, identical to SEQ ID NO: 28.

In an embodiment, when the extended second LCDR comprises, preferably consists of, the amino acid sequence according to SEQ ID NO: 29, this also includes an amino acid sequence which is at least 66.7%, such as least 83%, such as at least 83.3%, identical to SEQ ID NO: 29.

The isolated antibodies, or antigen-binding fragments thereof, as disclosed herein may also, or alternatively, be structurally described by the amino acid sequence of their HCVRs and/or the LCVRs. The skilled person will appreciate the HCVRs and LCVRs may be independently selected from the recited amino acid sequences. As explained above, the skilled person will appreciate that minor variations, such as substitutions, including deletion or addition of amino acids, of one, two, three, four or even more amino acid residues, in the amino acid sequence may occur without affecting the functional properties, such as its ability to bind to hBSSL, of the isolated antibody, or antigen-binding fragment thereof. The variation may be in the amino acid sequence of the CDRs, in the amino acid sequence outside the CDR regions, which is herein referred to as the framework regions, or both in the amino acid sequence of the CDRs and in the amino acid sequence outside the CDR regions of the HCVRs or LCVRs.

Hence, in an embodiment, the antibody, or antigen-binding fragment thereof, comprises a HCVR comprising, preferably consisting of, an amino acid sequence selected the group consisting of SEQ ID NO: 30, SEQ ID NO: 32, SEQ ID NO: 34 and SEQ ID NO: 36, and an amino acid sequence having at least 96%, such at least 97%, such as at least 98%, such as at least 99%, identity to any one of SEQ ID NO: 30, SEQ ID NO: 32, SEQ ID NO: 34 and SEQ ID NO: 36.

In a particular embodiment, the amino acid sequence of the HVCR is selected from the group consisting of SEQ ID NO: 30, SEQ ID NO: 34 and SEQ ID NO: 36, and an amino acid sequence which is at least 96%, such at least 97%, such as at least 98%, such as at least 99%, identical to any one of SEQ ID NO: 30, SEQ ID NO: 34 and SEQ ID NO: 36. In another particular embodiment, the amino acid sequence of the HVCR is selected from the group consisting of SEQ ID NO: 34 and SEQ ID NO: 36, and an amino acid sequence which is at least 96%, such at least 97%, such as at least 98%, such as at least 99%, identical to any one of SEQ ID NO: 34 and SEQ ID NO: 36. For example, the HCVR may comprise an amino acid sequence according to SEQ ID NO: 36 or an amino acid sequence which is at least 96%, such as at least 97%, such as at least 98%, such as at least 99% identical to any one of SEQ ID NO: 36.

In an embodiment, the antibody, or antigen-binding fragment thereof, comprises a LCVR comprising an amino acid sequence selected the group consisting of SEQ ID NO: 31, SEQ ID NO: 33, SEQ ID NO: 35, SEQ ID NO: 37 and SEQ ID NO: 38 and an amino acid sequence which is at least 96%, such at least 97%, such as at least 98%, such as at least 99% identical to any one of SEQ ID NO: 31, SEQ ID NO: 33, SEQ ID NO: 35, SEQ ID NO: 37 and SEQ ID NO: 38.

In a particular embodiment, the amino acid sequence of the LVCR is selected from the group consisting of SEQ ID NO: 31, SEQ ID NO: 35, SEQ ID NO: 37 and SEQ ID NO: 38 and an amino acid sequence which is at least 96%, such at least 97%, such as at least 98%, such as at least 99% identical to any one of SEQ ID NO: 31, SEQ ID NO: 35, SEQ ID NO: 37 and SEQ ID NO: 38. In another particular embodiment, the amino acid sequence of the LVCR is selected from the group consisting of SEQ ID NO: 35, SEQ ID NO: 37 and SEQ ID NO: 38 and an amino acid sequence which is at least 96%, such at least 97%, such as at least 98%, such as at least 99% identical to any one of SEQ ID NO: 35, SEQ ID NO: 37 and SEQ ID NO: 38; such as selected from the group consisting of SEQ ID NO: 37 and SEQ ID NO: 38 and an amino acid sequence which is at least 96%, such at least 97%, such as at least 98%, such as at least 99% identical to any one of SEQ ID NO: 37 and SEQ ID NO: 38. For example, the HCVR may comprise an amino acid sequence according to SEQ ID NO: 37 and an amino acid sequence which is at least 96%, such at least 97%, such as at least 98%, such as at least 99% identical to any one of SEQ ID NO: 37.

In an embodiment, the antibody, or antigen-binding fragment thereof, comprises a HCVR comprising an amino acid sequence selected the group consisting of SEQ ID NO: 30, SEQ ID NO: 32, SEQ ID NO: 34 and SEQ ID NO: 36 and an amino acid sequence which is at least 96%, such at least 97%, such as at least 98%, such as at least 99% identical to any one of SEQ ID NO: 30, SEQ ID NO: 32, SEQ ID NO: 34 and SEQ ID NO: 36. The antibody, or antigen-binding fragment thereof, also comprises a LCVR comprising an amino acid sequence independently selected the group consisting of SEQ ID NO: 31, SEQ ID NO: 33, SEQ ID NO: 35, SEQ ID NO: 37 and SEQ ID NO: 38 and an amino acid sequence which is at least 96%, such at least 97%, such as at least 98%, such as at least 99% identical to any one of SEQ ID NO: 31, SEQ ID NO: 33, SEQ ID NO: 35, SEQ ID NO: 37 and SEQ ID NO: 38.

In a particular embodiment, the isolated antibody, or antigen-binding fragment thereof, comprises an HCVR comprising, preferably consisting of, an amino acid sequence according to SEQ ID NO: 36 and a LCVR comprising, preferably consisting of, an amino acid sequence according to SEQ ID NO: 37.

In another particular embodiment, the isolated antibody, or antigen-binding fragment thereof, comprises a HCVR comprising, preferably consisting of, an amino acid sequence according to SEQ ID NO: 36 and a LCVR comprising, preferably consisting of, an amino acid sequence according to SEQ ID NO: 38.

In a further embodiment, the isolated antibody, or antigen-binding fragment thereof, comprises a HCVR comprising, preferably consisting of, an amino acid sequence according to SEQ ID NO: 30 and a LCVR comprising, preferably consisting of, an amino acid sequence according to SEQ ID NO: 31.

In yet another embodiment, the isolated antibody, or antigen-binding fragment thereof, comprises a HCVR comprising, preferably consisting of, an amino acid sequence according to SEQ ID NO: 32 and a LCVR comprising, preferably consisting of, an amino acid sequence according to SEQ ID NO: 33.

In an embodiment, the isolated antibody, or antigen-binding fragment thereof, comprises a HCVR comprising, preferably consisting of, an amino acid sequence according to SEQ ID NO: 34, and a LCVR comprising, preferably consisting of, an amino acid sequence according to SEQ ID NO: 35.

An aspect of the invention relates to an isolated antibody, or antigen-binding fragment thereof, comprising a HCVR consisting of an amino acid sequence selected from i) ZH1-[GYTFTSYN]-ZH2-[X₅₃GVIX₅₇PGDGX₆₄TSYX₆₈QKFX_{72]}-ZH3-[ARDYYGSSPLGY]-ZH4, or an amino acid sequence having at least 92% identity to the sequence defined in i), such as 93% or greater, such as 94% or greater, such as 95% or greater, such as 96% or greater, such as 97% or greater, such as 98% or greater, such as 99% or greater identity to the sequence defined in i), and a LCVR consisting of an amino acid sequence selected from ii) ZL1-[X₂₄ASX₂₇SISYX₃₉N]ZL2-[AX₅₇SX₆₆LX_{68]}-ZL3-[HQRSSX₁₁₅PT]-ZL4, or an amino acid an amino acid sequence having at least 87% identity to the sequence defined in ii), such as 88% or greater, such as 89% or greater, such as 90% or greater, such as 91% or greater, such as 92% or greater, such as 93% or greater, such as 94% or greater, such as 95% or greater, such as 96% or greater, such as 97% or greater, such as 98% or greater, such as 99% or greater, identity to the sequence defined in ii).

In this aspect, each of ZH1, ZH2, ZH3 and ZH4 independently represents zero, one or several independently selected amino acid residues and each of ZL1, ZL2, ZL3 and ZL4 independently represents zero, one or several independently selected amino acid residues. X₅₃ is selected from I and M, X₅₇ is selected from N and Y, X₆₄ is selected from A and S, X₆₈ is selected from A and N, X₇₂ is selected from K and Q, X₂₄ is selected from S and R, X₂₇ is selected from S and P, X₃₉ is selected from M and L, X₅₇ is selected from A and T, X₆₆ is selected from K and S, X₆₈ is selected from A and P, and X₁₁₅ is selected from S, T and Y. The numbering of amino acid residues is in accordance with the IMTG numbering standard, i.e., for a position “Xₙ,” in sequence i) or ii) as defined above, n is an integer and denotes the position of the amino acid residue X according to IMGT numbering.

GYTFTSYN is presented in SEQ ID NO: 7, X₅₃GVIX₅₇PGDGX₆₄TSYX₆₈QKFX₇₂ is presented in SEQ ID NO: 169, ARDYYGSSPLGY is presented in SEQ ID NO: 9, X₂₄ASX₂₇SISYX₃₉N is presented in SEQ ID NO: 170, AX₅₇SX₆₆LX₆₈ is presented in SEQ ID NO: 171 and HQRSSX₁₁₅PT is presented in SEQ ID NO: 172.

Herein are provided antibodies, or antigen-binding fragments thereof, wherein Xₙ in sequence i) is independently selected from a group of possible residues listed below in list A. The skilled person will appreciate that Xₙ may be selected from any one of the listed groups of possible residues and that this selection is independent from the selection of amino acids in Xₘ, wherein n≠m. Thus, any of the listed possible residues in position Xₙ may be independently combined with any of the listed possible residues any other variable position according to list A.

List A: Listing of possible amino acid residues in sequence i)

X₅₃ may bel;

X₅₃ may be M;

X₅₇ may be N;

X₅₇ may be Y;

X₅₉ may be G;

X₅₉ may be S;

X₆₄ may be A;

X₆₄ may be 5;

X₆₈ may be selected from A and T;

X₆₈ may be selected from A and N;

X₆₈ may be selected from T and N;

X₆₈ may be A;

X₆₈ may be N;

X₆₈ may be T;

X₇₂ may be K; and

X₇₂ may be Q.

In a similar manner, herein are provided antibodies, or antigen-binding fragments, thereof wherein Xₖ in sequence ii) herein is independently selected from a group of possible residues according to the list below in list B. The skilled person will appreciate that Xₖ may be selected from any one of the listed groups of possible residues and that this selection is independent from the selection of amino acids in X_{I}, wherein k≠I. Thus, any of the listed possible residues in position Xₖ in list A may be independently combined with any of the listed possible residues any other variable position according to list B.

List B: Listing of possible amino acid residues in sequence ii)

X₂₄ may be S;

X₂₄ may be R;

X₂₇ may be S;

X₂₇ may be P;

X₃₉ may be M;

X₃₉ may be L;

X₄₀ may be H;

X₄₀ may be N;

X₅₇ may be A;

X₅₇ may be T;

X₆₆ may be selected from K and S;

X₆₆ may be selected from R and S;

X₆₆ may be selected from R and K;

X₆₆ may be K;

X₆₆ may be R;

X₆₆ may be S;

X₆₈ may be selected from A and P;

X₆₈ may be selected from A and Q;

X₆₈ may be selected from P and Q;

X₆₈ may be A;

X₆₈ may be P;

X₆₈ may be Q;

X₁₀₅ may be H;

X₁₀₅ may be Q;

X₁₁₅ may be selected from S and T;

X₁₁₅ may be selected from S and Y;

X₁₁₅ may be selected from T and Y;

X₁₁₅ may be S;

X₁₁₅ may be Y; and

X₁₁₅ may be T.

To clarify, the selection of amino acid residues for amino acid positions in sequence i) from list A is independent from the selection of amino acid residues for amino acid positions in sequence ii) from list B. For avoidance of doubt, list A and list B each discloses several specific and individualized examples according to the present disclosure and the listed examples may be freely combined.

As defined above, each of ZH1, ZH2, ZH3 and ZH4 may represent zero, one or several independently selected amino acid residues. The identity and number of amino acid residues in each of said ZH1, ZH2, ZH3 and ZH4 may be independently selected.

Similarly, each of ZL1, ZL2, ZL3 and ZL4 may represent zero, one or several independently selected amino acid residues. The identity and number of amino acid residues in each of the ZL1, ZL2, ZL3 and ZL4 may be independently selected. Additionally, ZL1 may be connected via an amino acid linker or other linker to ZH1 or ZH4. Also, ZL4 may be connected via an amino acid linker or other linker to ZH1 or ZH4. Thus, the sequence as defined in i) and the sequence as defined in ii) may be the part of one amino acid sequence, in other words may be part of one polypeptide.

In an embodiment, ZH1 comprises, preferably consisting of, an amino acid sequence according to SEQ ID NO: 39, or an amino acid sequence which is at least 90%, such as at least 91%, such as at least 92%, such as at least 93%, such as at least 94%, such as at least 95%, such as at least 96%, such as at least 97%, such as at least 98%, such as at least 99% identical to SEQ ID NO: 39.

In an embodiment, ZH2 comprises, preferably consists of, an amino acid sequence according to SEQ ID NO: 40, or an amino acid sequence which is at least 90%, such as at least 91%, such as at least 92%, such as at least 93%, such as at least 94%, such as at least 95%, such as at least 96%, such as at least 97%, such as at least 98%, such as at least 99% identical to SEQ ID NO: 40.

In an embodiment, ZH3 comprises, preferably consists of, an amino acid sequence according to SEQ ID NO: 41, or an amino acid sequence which is at least 90%, such as at least 91%, such as at least 92%, such as at least 93%, such as at least 94%, such as at least 95%, such as at least 96%, such as at least 97%, such as at least 98%, such as at least 99% identical to SEQ ID NO: 41.

In an embodiment, ZH4 comprises, preferably consists of, an amino acid sequence according to SEQ ID NO: 42, or an amino acid sequence which is at least 90%, such as at least 91%, such as at least 92%, such as at least 93%, such as at least 94%, such as at least 95%, such as at least 96%, such as at least 97%, such as at least 98%, such as at least 99% identical to SEQ ID NO: 42.

In an embodiment, ZL1 comprises, preferably consists of, an amino acid sequence according to SEQ ID NO: 43, or an amino acid sequence which is at least 90%, such as at least 91%, such as at least 92%, such as at least 93%, such as at least 94%, such as at least 95%, such as at least 96%, such as at least 97%, such as at least 98%, such as at least 99% identical to SEQ ID NO: 43.

In an embodiment, ZL2 comprises, preferably consists of, an amino acid sequence according to SEQ ID NO: 44, or an amino acid sequence which is at least 90%, such as at least 91%, such as at least 92%, such as at least 93%, such as at least 94%, such as at least 95%, such as at least 96%, such as at least 97%, such as at least 98%, such as at least 99% identical to SEQ ID NO: 44.

In an embodiment, ZL3 comprises, preferably consists of, an amino acid sequence according to SEQ ID NO: 45, or an amino acid sequence which is at least 90%, such as at least 91%, such as at least 92%, such as at least 93%, such as at least 94%, such as at least 95%, such as at least 96%, such as at least 97%, such as at least 98%, such as at least 99% identical to SEQ ID NO: 45.

In an embodiment, ZL4 comprises, preferably consists of, an amino acid sequence according to SEQ ID NO: 46 or an amino acid sequence which is at least 90%, such as at least 91%, such as at least 92%, such as at least 93%, such as at least 94%, such as at least 95%, such as at least 96%, such as at least 97%, such as at least 98%, such as at least 99% identical to SEQ ID NO:46.

The skilled person will appreciate that the % identity of each of ZH1, ZH2, ZH3, ZH4, ZL1, ZL2, ZL3 and ZL4 to the amino acid sequence according to the respective SEQ ID NO: recited above is independent of the % identity of any other of ZH1, ZH2, ZH3, ZH4, ZL1, ZL2, ZL3 and ZL4 to its respective SEQ ID NO:. Thus for example, ZH1 may exhibit 95% identity to SEQ ID NO: 39 and ZH2 may exhibit 99% identity to SEQ ID NO: 40.

During the humanization process leading to the presently presented novel anti-BSSL antibodies, or antigen-binding fragments thereof, several mutations have been introduced into both CDR and adjacent FW regions. For the purpose of the present disclosure, each antibody HCVR and LCVR is composed of three CDRs, of which one, two or three CDRs may be extended CDRS (eCDRs), and four FRs/FWs, arranged from N-terminus to C-terminus in the following order as defined in Table 1.

TABLE 1
IMGT numbering

| | IMGT numbering |
|---|---|
| VH-FW1 | 1, 2, 3, 4, 5, 6, 7, 8, 9, 11, 12, 13, 14, |
| | 15, 16, 17, 18, 19, 20, 21, 22, 23, 24, 25, 26 |
| CDR-H1 | 27, 28, 29, 30, 35, 36, 37, 38 |
| VH-FW2 | 39, 40, 41, 42, 43, 44, 45, 46, 47, 48, 49, |
| | 50, 51, 52, 53, 54, 55 |
| eCDR-H2 | 53, 54, 55, 56, 57, 58, 59, 62, 63, 64, 65, |
| | 66, 67, 68, 69, 70, 71, 72 |
| CDR-H2 | 56, 57, 58, 59, 62, 63, 64, 65 |
| VH-FW3 | 66, 67, 68, 69, 70, 71, 72, 74, 75, 76, 77, |
| | 78, 79, 80, 81, 82, 83, 84, 85, 86, 87, 88, |
| | 89, 90, 91, 92, 93, 94, 95, 96, 97, 98, 99, |
| | 10, 101, 102, 103, 104 |
| CDR-H3 | 105, 106, 107, 108, 109, 110, 112, 113, |
| | 114, 115, 116, 117 |
| VH-FW4 | 118, 119, 120, 121, 122, 123, 124, 125, |
| | 126, 127, 128 |
| VL-FW1 | 1, 2, 3, 4, 5, 6, 7, 8, 9, 10, 11, 12, 13, 14, |
| | 15, 16, 17, 18, 19, 20, 21, 22, 23, 24, 25, 26 |
| eCDR-L1 | 24, 25, 26, 27, 28, 29, 37, 38, 39, 40 |
| CDR-L1 | 27, 28, 29, 37, 38 |
| VL-FW2 | 39, 40, 41, 42, 43, 44, 45, 46, 47, 48, 49, |
| | 50, 51, 52, 53, 54, 55 |
| eCDR-L2 | 56, 57, 65, 66, 67, 68 |
| CDR-L2 | 56, 57, 65 |
| VL-FW3 | 66, 67, 68, 69, 70, 71, 73, 74, 75, 76, 77, |
| | 78, 79, 80, 83, 84, 85, 86, 87, 88, 89, 90, |
| | 91, 92, 93, 94, 95, 96, 97, 98, 99, 100, |
| | 101, 102, 103, 104 |
| CDR-L3 | 105, 106, 107, 108, 109, 115, 116, 117 |
| VL-FW4 | 118, 119, 120, 121, 122, 123, 124, |
| | 125, 126, 127 |

The three CDRs in the HCRV are flanked by framework regions, which may be the regions ZH1, ZH2, ZH3 and ZH4 described above. The three CDRs in LCRV are flanked by framework regions, which may be ZL1, ZL2, ZL3 and ZL4 as described above.

HVCR of antibodies, or antigen-binding fragments thereof, constructed herein are listed among the amino acid sequences in the group consisting of SEQ ID NO: 30, 32, 34, 36 and 47-84. Correspondingly, LVCR of antibodies, or antigen-binding fragments thereof, constructed herein are listed among the amino acid sequences in the group consisting of SEQ ID NO: 31, 33, 35, 37, 38 and 86-123. Particular examples of these antibodies, or antigen-binding fragments thereof, comprise, preferably consists of, the following combinations of HVCR and LVCR: SEQ ID NO: 30 and 31; SEQ ID NO: 32 and 33; SEQ ID NO: 34 and 35; SEQ ID NO: 36 and 37; SEQ ID NO: 36 and 38; SEQ ID NO: 47 and 81; SEQ ID NO: 48 and 82; SEQ ID NO: 49 and 83; SEQ ID NO: 50 and 84; SEQ ID NO: 51 and 85; SEQ ID NO: 52 and 86; SEQ ID NO: 53 and 87; SEQ ID NO: 54 and 88; SEQ ID NO: 55 and 89; SEQ ID NO: 56 and 90; SEQ ID NO: 57 and 91; SEQ ID NO: 58 and 92; SEQ ID NO: 59 and 93; SEQ ID NO: 60 and 94; SEQ ID NO: 61 and 95; SEQ ID NO: 62 and 96; SEQ ID NO: 63 and 97; SEQ ID NO: 64 and 98; SEQ ID NO: 65 and 99; SEQ ID NO: 66 and 100; SEQ ID NO: 67 and 101; SEQ ID NO: 68 and 102; SEQ ID NO: 69 and 103; SEQ ID NO: 70 and 104; SEQ ID NO: 71 and 105; SEQ ID NO: 72 and 106; SEQ ID NO: 73 and 107; SEQ ID NO: 74 and 108; SEQ ID NO: 85 and 109; SEQ ID NO: 86 and 110; SEQ ID NO: 77 and 111; SEQ ID NO: 78 and 112; SEQ ID NO: 79 and 113 or SEQ ID NO: 80 and SEQ ID NO: 114.

The skilled person will understand that various modifications and/or additions can be made to an antibody, or antigen-binding fragment thereof, as disclosed herein in order to tailor the antibody, or antigen-binding fragment thereof, to a specific application without departing from the scope of the present disclosure. For example, the antibody, or antigen-binding fragment thereof, may have an amino acid sequence that has been extended by and/or comprises additional amino acids at the C-terminus and/or the N-terminus, for example at the C terminus and/or N terminus of the its heavy or light chain. Thus, the antibody, or antigen-binding fragment thereof, may comprise any suitable number of additional amino acid residues, for example at least one additional amino acid residue. Each additional amino acid residue may individually or collectively be added in order to, for example, improve and/or simplify production, purification, stabilization in vivo or in vitro, coupling or detection of the polypeptide. Such additional amino acid residues may comprise one or more amino acid residues added for the purpose of chemical coupling. An example is the addition of a cysteine residue. Additional amino acid residues may also provide a “tag” for purification or detection of the antibody, or antigen-binding fragment thereof, such as a Hiss tag, a (HisGlu)₃ tag, a “myc” (c-myc) tag or a FLAG tag.

The isolated antibody, or antigen-binding fragment thereof, of the invention may be selected from full-length antibodies, combinations of CDR sequences, single-chain variable fragments, Fab fragments, F(ab′)₂ fragments, F(ab′)₃ fragments, Fab′ fragments, Fd fragments, Fv fragments, dAb fragments, isolated complementarity determining regions (CDRs) and nanobodies although not limited thereto.

In an embodiment, the antibody, or antigen-binding fragment thereof is selected from the group consisting of a human antibody, a humanized antibody and a chimeric antibody, or an antigen-binding fragment thereof.

It may be desirable to reduce or eliminate effector function by antibodies, or antigen-binding fragments thereof, for example, to prevent target cell death or unwanted cytokine secretion. This may be in particular suitable when the antibodies, or antigen-binding fragments thereof, are intended to engage cell surface receptors and prevent receptor-ligand interactions, i.e., antagonists. Other examples where reduced effector function may be warranted include preventing antibody-drug conjugates from interacting with Fc receptors (FcyRs) leading to off-target cytotoxicity.

Hence, in an embodiment, the isolated antibody, or antigen-binding fragment thereof, comprises at least one Fc silencing mutation inhibiting interaction with FcyRs. For example, an antibody, or antigen-binding fragment thereof, based on IgG1 isotype class may comprise at least one, preferably at least two and more preferably all three of the Fc silencing mutations L234A, L235A and P329G.

Also, antibodies, or antigen-binding fragments thereof, of the IgG4 isotype are considered potential candidates for immunotherapy when reduced effector functions are desirable. IgG4 antibodies are known to be dynamic molecules able to undergo a process known as Fab arm exchange (FAE) and, without being bound by theory, this is thought to result in functionally monovalent, bispecific antibodies (bsAbs) with unknown specificity and, hence, potentially, reduced therapeutic efficacy. This may introduce undesired pharmacodynamics unpredictability for human immunotherapy.

Hence, in an embodiment, the isolated antibody, or antigen-binding fragment thereof comprises, at least one stabilizing mutation which prevents or reduces in vivo Fab arm exchange. For example, it has been suggested in the field that a single amino acid mutation (S228P) in the IgG4 core-hinge region is sufficient to prevent the in vivo FAE [8]. In a particular embodiment, the isolated antibody, or antigen-binding fragment thereof, is of IgG4 isotype subclass and the at least one stabilizing mutation is S228P.

In an embodiment, the isolated antibody, or antigen-binding fragment thereof, has an isotype class selected from the group consisting of IgG, IgA, IgM, IgD and IgE. In a particular embodiment, the isotype class is IgG. For example, the isolated antibody, or antigen-binding fragment thereof, may be selected from the group consisting of isotype subclass IgG1 and IgG4.

In an embodiment, the isolated antibody, or antigen-binding fragment thereof, is a monoclonal antibody, or an antigen-fragment thereof. The monoclonal antibody, or antigen-binding fragment thereof, is preferably a humanized monoclonal antibody, or an antigen-binding fragment thereof.

Currently preferred antibodies, or antigen-binding fragments thereof are denoted S-SL048-11 (herein also denoted clone 11, heavy chain SEQ ID NO: 119 and light chain SEQ ID NO: 120), S-SL048-46 (herein also denoted clone 46, heavy chain SEQ ID NO: 121 and light chain SEQ ID NO: 122), S-SL048-106 (herein also denoted clone 106, heavy chain SEQ ID NO: 123 and light chain SEQ ID NO: 124), S-SL048-116 (herein also denoted clone 116, heavy chain SEQ ID NO: 125 and light chain SEQ ID NO: 126) and S-SL048-118 (herein also denoted clone 118, heavy chain SEQ ID NO: 127 and light chain SEQ ID NO: 128).

The stability of these five candidates was evaluated by size exclusion chromatography (SEC), SDS-PAGE, nano Differential Scanning Fluorimetry (nano-DSF), and differential light scattering. By combining these data to an overall ranking, it was concluded that the most stable candidates in hIgG4 S228P format were candidates 106, 118 and 116.

### Binding Affinities

In an embodiment, the isolated antibody, or antigen-binding fragment thereof, according to the present document may have an affinity to hBSSL of no more than K_{D} 1×10⁻⁻⁷M, preferably of no more than K_{D} 1×10⁻⁸ M. For example, the isolated antibody, or antigen-binding fragment thereof, may have an affinity to hBSSL of no more than K_{D} 5 nM, such as no more than K_{D} 3 nM. As illustrated in the experimental section, an isolated antibody, or antigen-binding fragment thereof, may have an affinity to hBSSL of no more than K_{D} 1.7 nM, such as no more than K_{D} 1.7, 1.6, 1.5, 1.4, 1.3, 1.2, 1.1, 1.0, 0.9, 0.8, 0.7 or no more than 0.6 nM. In an example, the isolated antibody, or antigen-binding fragment thereof, that binds to hBSSL according to the present disclosure has an affinity to hBSSL of between K_{D} 0.6-1.7 nM, such as between K_{D} 0.6-1.0, 0.7-0.9, 0.8-1.6, 0.9-1.5, 1.0-1.7, 1.1-1.6, 1.2-1.7, 1.3-1.5, 1.0-1.4, 0.7-1.5, 0.7-1.6 or 1.0-1.7 nM.

### Pharmaceutical Compositions

The term “pharmaceutical composition” refers to a preparation, which is in such form as to permit the biological activity of an active ingredient contained therein to be effective, and which contains no additional components which are unacceptably toxic to a subject to which the formulation would be administered. The pharmaceutical compositions of the present document comprise antibody, and/or an antigen-binding fragment thereof, such as a scFv, as defined herein and a pharmaceutically acceptable carrier or excipient.

The antibody, or antigen-binding fragment thereof, such as a scFv, as defined herein, may be formulated by means known in the art into the form of, for example, tablets, capsules, aqueous or oily solutions, suspensions, emulsions, creams, ointments, gels, nasal sprays, suppositories, finely divided powders or aerosols for inhalation, and for parenteral use (including intravenous, subcutaneous or intramuscular infusion), sterile aqueous or oily solutions or suspensions or sterile emulsions.

Thus, herein is provided a composition comprising an isolated antibody, or antigen-binding fragment thereof, as described herein and at least one pharmaceutically acceptable excipient or carrier. For example, the excipient may be a diluent. In one example, the pharmaceutical composition may further comprise at least one additional active agent, such as at least two additional active agents, such as at least three additional active agents. Non-limiting examples of additional active agents that may prove useful in such combination are immune response modifying agents.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer or preservative.

As used herein, pharmaceutically acceptable carriers includes any and all solvents, dispersion media, coatings, antibacterial and antifungal agents, isotonic and absorption delaying agents, and the like that are physiologically compatible. Preferably, the carrier is suitable for oral as well as intravenous, intramuscular, subcutaneous, spinal or epidermal administration (e.g., by injection or infusion).

A pharmaceutical composition as disclosed herein also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxy anisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and non-aqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of presence of microorganisms may be ensured both by sterilization procedures, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art.

Also provided herein is a kit of parts comprising an antibody, or antigen-binding fragment thereof, or a pharmaceutical composition according to the present inventions, means for administering the antibody, or antigen-binding fragment thereof, or a pharmaceutical composition, and optionally a package insert comprising instructions for use. Means for administering the antibody, or antigen-binding fragment thereof, or a pharmaceutical composition may e.g., be a syringe. The term “package insert” is used to refer to instructions customarily included in commercial packages of therapeutic products, that contain information about the indications, usage, dosage, administration, combination therapy, contraindications and/or warnings concerning the use of such therapeutic products.

Medical uses of the Isolated Antibodies or Antigen-Binding Fragments Thereof

An antibody, or antigen-binding fragment thereof, according to the present invention may be used to effectively reduce a pro-inflammatory effect of BSSL in a subject, such as a human.

An advantage with the antibodies, and antigen-binding fragments thereof, of the present invention is that they do not bind to the active site on the BSSL protein that is responsible for the lipase activity of BSSL. This is demonstrated and further elaborated on in the experimental section. Thus, the risk for negative side effects is decreased as the lipase activity is not significantly affected.

The present invention is, thus, directed to an isolated antibody, or antigen-binding fragment thereof, such as a scFv, or a pharmaceutical composition, as defined herein, for use as a medicament.

The present document is also directed to an isolated antibody, or antigen-binding fragment thereof, or a pharmaceutical composition, as defined herein, for use in the treatment and/or prevention of an inflammatory disease. The present invention is also directed to the use of an isolated antibody, or antigen-binding fragment thereof, such as a scFv, or a pharmaceutical composition, as defined herein, for the manufacture of a medicament for the treatment and/or prevention of an inflammatory disease. The present document is also directed to a method for treating and/or ameliorating and/or preventing and/or prophylaxis of an inflammatory disease. This method comprises administering a therapeutically effective amount of an isolated antibody, or antigen-binding fragment thereof, such as a scFv, or a pharmaceutical composition, to a subject in need thereof.

Different in vivo models may be used to predict the antibodies' and antigen-binding fragments' effect in treating and/or preventing inflammatory disease. Typically mice are used in these models and different substances are injected to elicit an immune response. The effect of the antibodies, or antigen-binding fragments thereof, on such an immune response can thus be studied after administration of the antibodies/antigen-binding fragments.

One model that may be used is the so called “Collagen induced arthritis” (CIA) model. In this model, collagen type II (CII) in complete Freund's adjuvant (CFA) is injected typically with a boost of CII in incomplete Freund's adjuvant (IFA) at day 21. This model induces autoimmune arthritis. Arthritis typically appears after 21-28 days after the first injection with CII. This model is B and T cell dependent (adaptive immunity).

Another model is the “Collagen antibody induced arthritis” (CAIA). In this model, a cocktail of CII antibodies is injected, typically with a boost of lipopolysaccharides (LPS) on day 5. This model is B and T cell independent (innate immunity). A protocol for testing the in vivo efficacy of the antibodies and antigen-binding fragments thereof of the present document in the treatment and/or prevention of inflammatory disease using the CAIA model is disclosed in the experimental section of this document.

Yet another model is the “Glucose-6-phosphate isomerase induced arthritis” model. Here a peptide corresponding to a sequence in the glucose-6-phosphate isomerase is injected to elicit an immune response. This model is T cell dependent.

Another model is the “Pristane induced arthritis” (PIA) model where pristan is injected. This model is T cell dependent.

Also, the “Dextran sulphate sodium induced colitis” model may be used, wherein dextran sulphate sodium (DSS) is given in drinking water.

All of the above methods are well-known to a person skilled in the art. The methods may be used to test the in vivo efficiency of the antibodies, or antigen-binding fragments thereof, of the present document.

The inflammatory disease to be treated and/or prevented according to the present document may e.g., be a chronic inflammatory disease. The inflammatory disease may be a local or a systemic inflammatory disease.

The inflammatory disease may e.g., be an autoimmune disease or an autoinflammatory disease. Another type of inflammatory disease is a natural killer (NK) cell mediated inflammatory disease. Such NK-cell mediated inflammatory diseases include rheumatoid arthritis (RA), systemic juvenile idiopathic arthritis (sJIA), macrophage activation syndrome (MAS), systemic lupus erythematosus (SLE), systemic sclerosis, multiple sclerosis (MS), Sjogren's syndrome and inflammatory bowel disease (IBD).

In an embodiment, the inflammatory disease is selected from the group consisting of RA, JIA, psoriatic arthritis, an IBD, such as Crohn's disease or ulcerative colitis (UC), hepatic steatosis, also referred to as liver steatosis, and hyperinflammation.

In a particular embodiment, the inflammatory disease is an inflammatory condition induced by a pathogen, such as a bacteria or a virus. Examples of such viruses include coronaviruses, such as severe acute respiratory syndrome coronavirus 1 (SARS-CoV-1) or SARS-CoV-2. The latter virus causes coronavirus disease 2019 (COVID-19). Severe COVID-19 patients often suffer from systemic hyperinflammation including elevated levels of various inflammatory cytokines, such as interleukin 2 (IL-2), IL-7, IL-6, granulocyte-macrophage colony-stimulating factor (GM-CSF), interferon-γ inducible protein 10 (IP-10), monocyte chemoattractant protein 1 (MCP-1), macrophage inflammatory protein 1-α (MIP-1a), and TNF-α.

RA primarily affects joints but may also be a systemic inflammatory disease that may give extraarticular manifestations in several organs. RA may therefore be considered a systemic inflammatory disease.

Furthermore, the herein disclosed isolated antibody, or antigen-binding fragment thereof, such as a scFv fragment, or pharmaceutical composition may be an alternative to current biological treatments for patients not responding, or responding transiently to current tumor necrosis factor alpha (TNFa) inhibitors, which reduces the need of administering corticosteroids and/or immunosuppressing agents and or pharmaceuticals. Thus, the use of a herein disclosed isolated antibody or antigen-binding fragment thereof, such as a scFv fragment, prohibits and/or reduces adverse effects and/or side-effects of alternative treatment regimens in patients, which is a key issue in qualitative care in general, and in particular important in young patients and children, as well as in immune-suppressed patients and/or elderly patients.

The treatment and/or prevention using an isolated antibody and/or antigen-binding fragment thereof or a pharmaceutical composition as disclosed herein is typically a passive immunotherapy, meaning that an antibody, or antigen-binding fragment thereof, or a pharmaceutical composition comprising such antibodies, and/or antigen-binding fragments thereof, is administered to a subject in need thereof. However, other types of immunotherapeutic methods may also be employed, such as gene therapy wherein, instead of administering an antibody, or antigen-binding fragment thereof, directly, a gene construct being able to express such an antibody, or antigen-binding fragment thereof, is administered to a subject.

A subject according to the present disclosure may be any human or non-human animal. The term “non-human animal” includes all vertebrates, e.g., mammals and non-mammals, such as non-human primates, sheep, dogs, cats, horses, cows, chickens, amphibians, reptiles, etc. Mammals include, but are not limited to, domesticated animals (e.g., cows, sheep, cats, dogs, and horses), primates (e.g., humans and non-human primates, such as monkeys), rabbits, and rodents (e.g., mice and rats). The term “subject” may be used interchangeably with the term “patient” in the present document. The subject may be a human.

As used herein, “treatment” (and grammatical variations thereof such as “treat” or “treating”) refers to clinical intervention in an attempt to alter the natural course of the diseases of the individual being treated, and can be performed either for prophylaxis or during the course of clinical pathology. Desirable effects of treatment include, but are not limited to, preventing occurrence or recurrence of disease, alleviation of symptoms (improvement of quality of life), diminishment of any direct or indirect pathological consequences of the disease, preventing metastasis, decreasing the rate of disease progression, amelioration or palliation of the disease state, and remission or improved prognosis. An antibody, or antigen-binding fragment thereof, according to the present invention may be used to delay development of a disease or to slow the progression of a disease.

By “reduce” or “inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated. Reduce or inhibit also encompass delaying the onset of a disease, in particular an inflammatory disease.

### Administration Modes

An isolated antibody, or antigen-binding fragment thereof, such as a scFv, or a pharmaceutical composition according to the present invention may be administered in standard manner for the condition that it is desired to treat and/or prevent, for example by oral, topical, parenteral, intravenous, subcutaneous, buccal, nasal, or rectal administration or by inhalation. For example, the antibody, or antigen-binding fragment thereof, such as a scFv, or the pharmaceutical composition for use as described herein may be formulated for parenteral administration, such as intravenous or subcutaneous administration, in particular subcutaneous administration.

Typically, the isolated antibody, or antigen-binding fragment thereof, such as a scFv, or a pharmaceutical composition, as defined herein, is systemically administered. The administration mode may e.g., be parenteral, such as by intravenous or subcutaneous administration, in particular subcutaneous administration.

Although the administration regimen may be adjusted to the particular disease and subject to be treated, typically the isolated antibody, or antigen-binding fragment thereof, such as a scFv, or a pharmaceutical composition, as defined herein, is administered 1-3 times per week, such as 1-2 times per week, such as 1 time a week although other administration regimes are also possible.

The antibodies, antigen-binding fragments thereof, and/or pharmaceutical compositions of the present invention may also be administered in combination therapy, i.e., combined with other agents. For example, the combination therapy can include an antibody, or antigen-binding fragment thereof, such as a scFv, according to the present invention, combined with at least one other anti-inflammatory or immunosuppressant agent. It will be understood that the combination therapy encompasses sequential as well as concurrent administration. The term “concurrent” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

Dosage regimens may be adjusted to provide the optimum desired response, e.g., a therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage.

The therapeutically effective amount of an antibody, or antigen-binding fragment thereof, such as a scFv, may vary according to factors such as the disease state, age, sex, and weight of the individual, and the ability of the antibody, or antigen-binding fragment thereof, such as a scFv, to elicit a desired response in the subject. A therapeutically effective amount is also one, in which any toxic or detrimental effects of the administered substance are outweighed by the therapeutically beneficial effects. When the antibody, or antigen-binding fragment thereof, such as a scFv, is used for the prevention of a condition (prophylactic purposes), typically but not necessarily, the prophylactically effective amount is less than the therapeutically effective amount as the prophylactic dose is used in subjects prior to or at an earlier stage of disease.

A pharmaceutically effective amount, i.e., the dose, of an antibody, or antigen-binding fragment thereof, such as a scFv, according to the present invention is typically in the range of from about 0.0001 to 100 mg/kg, and more usually 0.01 to 5 mg/kg, of the host body weight. However, the exact dose has to be adjusted depending on e.g., the condition to be treated or prevented, the age and/or the sex of the subject and whether it is intended to treat or prevent a condition.

### Expression Systems

The present invention also relates to a polynucleotide, such as an isolated polynucleotide, where the polynucleotide encodes an antibody, or antigen-binding fragment thereof, according to the present invention.

Examples of polynucleotides according to the embodiments are shown in SEQ ID NO: 174 to 202 showing the DNA sequences encoding for the HC and LC of five different antibody fragments: S-SL048-11 HC (SEQ ID NO: 174; 185; 196) and LC (SEQ ID NO: 175; 186; 197), S-SL048-46 HC (SEQ ID NO: 176; 187) and LC (SEQ ID NO: 177; 188), S-SL048-106 HC (SEQ ID NO: 178; 189; 198) and LC (SEQ ID NO: 179; 190; 199), S-SL048-116 HC (SEQ ID NO: 180; 191; 200) and LC (SEQ ID NO: 181; 192; 201), and S-SL048-118 HC (SEQ ID NO: 180; 191; 200) and LC (SEQ ID NO: 182; 193; 202), and of AS20 HC SEQ ID NO: 183) and LC (SEQ ID NO: 184) and of CDR graft HC (SEQ ID NO: 194) and LC (SEQ ID NO: 195).

Hence, in an embodiment, the polynucleotide is selected from the group consisting of SEQ ID NO: 174 to 202, and any combination and/or variant thereof. A variant of any of SEQ ID NO: 174 to 202 as used herein include a polynucleotide encoding for the same antibody, or antingen-binding fragment thereof, as the polynucleotide as defined in any of SEQ ID NO: 174 to 202 but may have at least one synonymous substitution, i.e., substitution of at least one base for another such that the produced amino acid sequence is not modified. Hence, such a synonymous substitution changes at least one base in a codon in the polynucleotide into another codon, which both encode for the amino acid residue. For instance, a polynucleotide according to any of SEQ ID NO: 174 to 202, or a combination thereof, could be codon optimization for expression in a particular host cell.

The polynucleotide encoding an antibody, or antigen-binding fragment thereof, as disclosed herein may be introduced into an expression vector. The expression vector allows the propagation of the polynucleotide introduced therein. The vector may be a self-replicating nucleic acid structure as well as a vector incorporated into the genome of a host cell into which it has been introduced. The present invention is, thus, also directed to such an expression vector comprising a polynucleotide encoding an antibody, or antigen-binding fragment thereof.

The expression vector preferably comprises the polynucleotide encoding an antibody, or antigen-binding fragment thereof, operatively linked to at least one regulatory element. In an embodiment, the regulatory element is or comprises a promoter. A promoter is a sequence of DNA, to which proteins bind that initate transcription of a RNA molecule from the DNA (gene) downstream of it. Another example of a regulatory element is an enhancer. An enhancer is a short region of DNA that can be bound by activators to increase the likelihood that transcription of a particular gene will occur.

Examples of expression vectors include a DNA molecule, an RNA molecule, a plasmid, an episomal plasmid and a virus vector. Non-limiting, but illustrative, examples of virus vectors include a lentiviral vector, an adenoviral vector, an adeno-associated viral vector, a retroviral vector, a Semliki Forest virus, a polio virus and a hybrid vector.

The expression vector may be introduced into a host cell for expression and/or propagation of the vector comprising the polynucleotide. In particular, the expression vector is for use in the treatment and/or prevention of an inflammatory disease by being expressed in the subject to thereby produce antibodies, or antigen-binding fragments thereof, in the subject.

Also provided herein is, thus, a host cell comprising the expression vector. The host cell used can be any type of host cell, including both eukaryotic and prokaryotic host cells. Host cells include “transformants” and “transformed cells,” which include the primary transformed cell and progeny derived therefrom without regard to the number of passages.

The invention also relates to a cell comprising an antibody, or antigen-binding fragment thereof, according to the invention, a polynucleotide according to the invention and/or an expression vector according to the invention.

The cell may be an isolated cell, including a cell of a cell line. The cell can be selected from a bacterial cell, an eukaryotic cell, such as a yeast cell, a mammalian cell, a human cell or a non-human cell.

The antibodies, or antigen-binding fragments thereof, may be produced by introducing their sequence into an expression vector and allowing the expression vector to express the antibody or antigen-binding fragment thereof in a host cell after which the produced antibodies or antigen-binding fragments thereof are isolated/purified before use e.g., for medical treatment purposes or for diagnostic purposes as disclosed elsewhere herein. Also, the vector itself may be introduced into a subject for direct expression of the antibody or antigen-binding fragment thereof in the subject to be treated. The expression vector then preferably comprises a promoter controlling expression of the polynucleotide encoding the antibody, or antigen-binding fragment thereof.

Hence, the invention also relates to a method of producing an antibody, or an antigen-binding fragment thereof. The method comprises culturing a cell according to the invention comprising an expression vector according to the invention under conditions where the antibody, or antigen-binding fragment thereof, is expressed by the cell. In an embodiment, the method optionally comprises isolating the antibody, or antigen-binding fragment thereof, from the cell or a culture medium, in which the cell is cultured.

### Diagnostic use of the Isolated Antibodies or Antigen-Binding Fragments Thereof

The antibodies, or antigen-binding fragments thereof, of the present invention can also be used for the detection of BSSL, such as hBSSL, in a sample using standard techniques, such as, but not limited to, ELISAs, Western blots, RIAs, surface plasmon resonance (SPR) and flow cytometry analysis.

An advantage with using the antibodies, or antigen-binding fragments thereof, of the present invention is that they do not bind to the active site of the BSSL and thereby do not inhibit the lipase activity of the protein. Thus, it is possible to use the antibodies, or antigen-binding fragments thereof, to study the BSSL protein without significantly affecting the lipase activity as is demonstrated in the experimental section. The antibodies, or antigen-binding fragments, thereof are, thus, useful as molecular tools when studying the BSSL protein and/or its enzymatic activity in vitro/ex vivo and/or in vivo.

The present invention, thus, discloses a method for detecting the presence or absence of BSSL and/or for quantifying the amount of BSSL, such as hBSSL, in a sample. The method comprises contacting a sample with an isolated antibody, or antigen-binding fragment thereof, according to the invention. The method also comprises detecting the presence or absence of BSSL and/or quantifying the amount of BSSL in the sample based on an amount of isolated antibody, or antigen-binding fragment thereof, bound to BSSL. The detection or quantification may, for instance, be performed by using ELISA, Western blot, RIA, surface plasmon resonance (SPR), proximity ligation assay (PLA) or flow cytometry analysis. One or multiple, i.e., at least two, of the antibodies, or antigen-binding fragments thereof, of the invention may be used in such a detection.

The above described method may be in the form of an ex vivo or in vitro method. In such a case, the method comprises contacting, ex vivo or in vitro, the sample with the isolated antibody, or antigen-binding fragment thereof, according of the invention. In an embodiment, the method also comprises providing a sample potentially containing BSSL.

The present invention also discloses a method for diagnosis of a BSSL related disorder. The method comprises a) contacting a sample with an isolated antibody, or antigen-binding fragment thereof, according to the invention and b) detecting the presence or absence of BSSL and/or quantifying the amount of BSSL in the sample based on an amount of isolated antibody, or antigen-binding fragment thereof, bound to BSSL. The detection or quantification may, for instance, be performed by using ELISA, Western blot, RIA, SPR, PLA or flow cytometry analysis. The method also comprises c) concluding, based on the results in step b), whether the subject is diagnosed with a BSSL related disorder or not.

In an embodiment, the method also comprises providing a sample from a subject suspected of suffering from a BSSL related disorder.

In a particular embodiment, the method comprises comparing the quantified amount of BSSL in the sample with a threshold value. In such a particular embodiment, step c) comprises concluding whether the subject is diagnosed with the BSSL related disorder or not based on the comparison between the quantified amount of BSSL in the sample and the threshold value. For instance, if the amount of BSSL in the BSSL exceeds the threshold value, the subject is concluded to be diagnosed with the BSSL related disorder or not.

The value of the threshold value depends on the particular BSSL related disorder and can be defined by quantifying the amount of BSSL in samples taken from subjects already diagnosed with the particular BSSL related disorder and/or by quantifying the amount of BSSL in samples taken from healthy subjects that are not suffering from the particular BSSL related disorder. The threshold value could then be determined based on these quantified amounts of BSSL from subjects suffering from the particular BSSL related disorder and preferably based on the quantified amounts of BSSL from the healthy subjects.

The BSSL related disorder is typically an inflammatory condition as disclosed elsewhere herein. The inflammatory condition may e.g., be a chronic or a systemic inflammatory condition, such as an inflammatory disease, auto-inflammatory disease and/or autoimmune disease. The inflammatory condition may e.g., be rheumatoid arthritis, juvenile arthritis, psoriatic arthritis, atherogenesis, Crohn's disease, or ulcerative colitis.

The sample potentially containing BSSL may be any kind of sample such as a sample obtained from a subject. Hence, in an embodiment, the sample is a biological sample. An example of such a biological sample is a body fluid sample, e.g., a blood sample, a blood plasma sample or a serum sample. Another example of a biological sample is a body tissue sample, such as a biopsy. The sample may be a natural sample or an in vitro sample potentially containing BSSL. The methods for detecting BSSL and/or diagnosis of BSSL related conditions include both in vitro methods and in vivo methods, such as in situ hybridization.

As mentioned elsewhere herein, the antibodies, or antigen-binding fragments thereof, may be humanized or their CDR sequences (or parts of them) grafted onto a non-human backbone. The latter may be advantageous when using the antibodies, or antigen-binding fragments thereof, as a molecular tool to study the BSSL protein in other species than humans in order to e.g., decrease negative immunogenic reactions to the antibodies and/or antigen-binding fragments thereof.

### Illustrative Embodiments

An embodiment relates to an isolated antibody, or antigen-binding fragment thereof, that specifically binds to Bile Salt Stimulated Lipase (BSSL), such as human BSSL (hBSSL). The antibody, or antigen-binding fragment thereof, binds to at least one of a first epitope and a second epitope identified on BSSL. The first epitope comprises, preferably consists of, an amino acid sequence according to SEQ ID NO: 1 or an amino acid sequence having at least 80%, such as 85%, 90%, 95%, 96%, 97%, 98% or 99% identity to SEQ ID NO: 1 and the second epitope comprises an amino acid sequence according to SEQ ID NO: 2, or an amino acid sequence having at least 80%, such as 85%, 90%, 95%, 96%, 97%, 98% or 99% identity to SEQ ID NO: 2.

In an embodiment, the first epitope comprises, preferably consists of, an amino acid sequence according to SEQ ID NO: 3 or an amino acid sequence having at least 80%, such as 85%, 90%, 95%, 96%, 97%, 98% or 99% identity to SEQ ID NO: 3.

In an embodiment, the antibody, or antigen-binding fragment thereof, specifically binds to both the first epitope and the second epitope.

In an embodiment, the isolated antibody, or antigen-binding fragment thereof, further specifically binds to an amino acid sequence according to SEQ ID NO: 4 or an amino acid sequence having at least 80%, such as 85%, 90%, 95%, 96%, 97%, 98% or 99% identity thereto.

In an embodiment, the isolated antibody, or antigen-binding fragment thereof, further specifically binds to an amino acid sequence according to SEQ ID NO: 5 or an amino acid sequence having at least 80%, such as 85%, 90%, 95%, 96%, 97%, 98% or 99% identity thereto.

In an embodiment, the isolated antibody, or antigen-binding fragment thereof, further specifically binds to an amino acid sequence according to SEQ ID NO: 6 or an amino acid sequence having at least 80%, such as 85%, 90%, 95%, 96%, 97%, 98% or 99% identity thereto.

An embodiment relates to an isolated antibody, or antigen-binding fragment thereof. The isolated antibody, or antigen-binding fragment thereof, comprises three complementary determining regions (CDRs) of a heavy chain variable region (HCVR) (HCDR). A first HCDR comprises or consists of an amino acid sequence according to SEQ ID NO: 7, or an amino acid sequence which is at least 87% identical to SEQ ID NO: 7, a second HCDR comprises or consists of an amino acid sequence according to SEQ ID NO: 8, or an amino acid sequence which is at least 75% identical to SEQ ID NO: 8, and a third HCDR comprises or consists of an amino acid sequence according to SEQ ID NO: 9 or an amino acid sequence which is at least 90% identical to SEQ ID NO: 9. The isolated antibody, or antigen-binding fragment thereof, comprises three CDRs of a light chain variable region (LCVR) (LCDR). A first LCDR comprises or consists of an amino acid sequence according to SEQ ID NO: 10, or an amino acid sequence which is at least 80% identical to SEQ ID NO: 10, a second LCDR comprises or consists of the amino acid sequence ATS, or an amino acid sequence which is at least 66% identical to the amino acid sequence ATS, such as AAS, and a third LCDR comprises or consists of an amino acid sequence according to SEQ ID NO: 11 or an amino acid sequence which is at least 87% identical to SEQ ID NO: 11.

In an embodiment, the first HCDR comprises, preferably consists of, an amino acid sequence according to SEQ ID NO: 7, the second HCDR comprises, preferably consists of, an amino acid sequence selected from the group consisting of SEQ ID NO: 8, 18 and 19, and the third HCDR comprises, preferably consists of, an amino acid sequence according to SEQ ID NO: 9. In this embodiment, the first LCDR comprises, preferably consists of, an amino acid sequence selected from the group consisting of SEQ ID NO: 10 and 20, the second LCDR comprises, preferably consists of, an amino acid sequence selected from the group consisting of ATS and AAS, and the third LCDR comprises, preferably consists of, one amino acid sequence selected from the group consisting of SEQ ID NO: 11, 21 and 22.

In an embodiment, the isolated antibody, or antigen-binding fragment thereof, comprises three HCDRs of a HCVR. A first HCDR comprises, preferably consists of, an amino acid sequence according to SEQ ID NO: 7, a second HCDR comprises, preferably consists of, an amino acid sequence according to SEQ ID NO: 18 and a third HCDR comprises, preferably consists of, an amino acid sequence according to SEQ ID NO: 9. In this embodiment, the isolated antibody, or antigen-binding fragment thereof, comprises three LCDRs of a LCVR. A first LCDR comprises, preferably consists of, an amino acid sequence according to SEQ ID NO: 10, a second LCDR comprises, preferably consists of, an amino acid sequence ATS and a third LCDR comprises, preferably consists of, an amino acid sequence according to SEQ ID NO: 21.

In an embodiment, the isolated antibody, or antigen-binding fragment thereof, comprises three HCDRs of a HCVR. A first HCDR comprises, preferably consists of, an amino acid sequence according to SEQ ID NO: 7, a second HCDR comprises, preferably consists of, an amino acid sequence according to SEQ ID NO: 8 and a third HCDR comprises, preferably consists of, an amino acid sequence according to SEQ ID NO: 9. In this embodiment, the isolated antibody or antigen-binding fragment thereof comprises three LCDRs of a LCVR. A first LCDR comprises, preferably consists of, an amino acid sequence according to SEQ ID NO: 10, a second LCDR comprises, preferably consists of, an amino acid sequence ATS and a third LCDR comprises, preferably consists of, an amino acid sequence according to SEQ ID NO: 11.

In an embodiment, the isolated antibody, or antigen-binding fragment thereof, comprises three HCDRs of a HCVR. A first HCDR comprises, preferably consists of, an amino acid sequence according to SEQ ID NO: 7, a second HCDR comprises, preferably consists of, an amino acid sequence according to SEQ ID NO: 19 and a third HCDR comprises, preferably consists of, an amino acid sequence according to SEQ ID NO: 9. In this embodiment, the isolated antibody, or antigen-binding fragment thereof, comprises three LCDRs of a LCVR. A first LCDR comprises, preferably consists of, an amino acid sequence according to SEQ ID NO: 20, a second LCDR comprises, preferably consists of, an amino acid sequence ATS and a third LCDR comprises, preferably consists of, an amino acid sequence according to SEQ ID NO: 22.

In an embodiment, the antibody, or antigen-binding fragment thereof, comprises three HCDRs of a HCVR. A first HCDR comprises, preferably consists of, an amino acid sequence according to SEQ ID NO: 7, a second HCDR comprises, preferably consists of, an amino acid sequence according to SEQ ID NO: 8 and a third HCDR comprises, preferably consists of, an amino acid sequence according to SEQ ID NO: 9. In this embodiment, the isolated antibody, or antigen-binding fragment thereof, comprises three LCDRs of a LCVR. A first LCDR comprises, preferably consists of, an amino acid sequence according to SEQ ID NO: 20, a second LCDR comprises, preferably consists of, an amino acid sequence AAS and a third LCDR comprises, preferably consists of, an amino acid sequence according to SEQ ID NO: 11.

In an embodiment, the isolated antibody, or antigen-binding fragment thereof, comprises three HCDRs of a HCVR. A first HCDR comprises, preferably consists of, an amino acid sequence according to SEQ ID NO: 7, or an amino acid sequence which is at least 87% identical to SEQ ID NO: 7, a second HCDR comprises, preferably consists of, an amino acid sequence according to SEQ ID NO: 23, or an amino acid sequence which is at least 77% identical to SEQ ID NO: 23, and a third HCDR comprises, preferably consists of, an amino acid sequence according to SEQ ID NO: 9, or an amino acid sequence which is at least 83% identical to SEQ ID NO: 9. In this embodiment, the isolated antibody, or antigen-binding fragment thereof, comprises three LCDRs of a LCVR. A first LCDR comprises, preferably consists of, an amino acid sequence according to SEQ ID NO: 16, or an amino acid sequence which is at least 80% identical to SEQ ID NO: 16, a second LCDR comprises, preferably consists of, the amino acid sequence according to SEQ ID NO: 15, or an amino acid sequence which is at least 66% identical to SEQ ID NO: 15, and a third LCDR comprises, preferably consists of, an amino acid sequence according to SEQ ID NO: 21, or an amino acid sequence which is at least 87% identical to SEQ ID NO: 21.

In an embodiment, the isolated antibody, or antigen-binding fragment thereof, comprises three heavy chain complementary determining regions (HCDRs) of a heavy chain variable region (HCVR). A first HCDR comprises, preferably consists of, an amino acid sequence according to SEQ ID NO: 7, or an amino acid sequence which is at least 87% identical to SEQ ID NO: 7, a second HCDR comprises, preferably consists of, an amino acid sequence according to SEQ ID NO: 24, or an amino acid sequence which is at least 77% identical to SEQ ID NO: 24, and a third HCDR comprises, preferably consists of, an amino acid sequence according to SEQ ID NO: 9, or an amino acid sequence which is at least 83% identical to SEQ ID NO: 9. In this embodiment, the isolated antibody, or antigen-binding fragment thereof, comprises three CDRs of a light chain variable region (LCVR). A first LCDR comprises, preferably consists of, an amino acid sequence according to SEQ ID NO: 27, or an amino acid sequence which is at least 70% identical to SEQ ID NO: 27, a second LCDR comprises, preferably consists of, the amino acid sequence according to SEQ ID NO: 29, or an amino acid sequence which is at least 50% identical to SEQ ID NO: 29, and a third LCDR comprises, preferably consists of, an amino acid sequence according to SEQ ID NO: 11, or an amino acid sequence which is at least 87% identical to SEQ ID NO: 11.

In an embodiment, the isolated antibody, or antigen-binding fragment thereof, comprises three HCDRs of a HCVR. A first HCDR comprises, preferably consists of, an amino acid sequence according to SEQ ID NO: 7, or an amino acid sequence which is at least 87% identical to SEQ ID NO: 7, a second HCDR comprises, preferably consists of, an amino acid sequence according to SEQ ID NO: 25, or an amino acid sequence which is at least 83% identical to SEQ ID NO: 25, and a third HCDR comprises, preferably consists of, an amino acid sequence according to SEQ ID NO: 9 or an amino acid sequence which is at least 83% identical to SEQ ID NO: 9. In this embodiment, the isolated antibody, or antigen-binding fragment thereof, comprises three LCDRs of a LCVR. A first LCDR comprises, preferably consists of, an amino acid sequence according to SEQ ID NO: 26, or an amino acid sequence which is at least 90% identical to SEQ ID NO: 26, a second LCDR comprises, preferably consists of, the amino acid sequence according to SEQ ID NO: 28, or an amino acid sequence which is at least 66% identical to SEQ ID NO: 28, and a third LCDR comprises, preferably consists of, an amino acid sequence according to SEQ ID NO: 22 or an amino acid sequence which is at least 87% identical to SEQ ID NO: 22.

In an embodiment, the isolated antibody, or antigen-binding fragment thereof, comprises three HCDRs of a HCVR. A first HCDR comprises, preferably consists of, an amino acid sequence according to SEQ ID NO: 7, or an amino acid sequence which is at least 87% identical to SEQ ID NO: 7, a second HCDR comprises, preferably consists of, an amino acid sequence according to SEQ ID NO: 12, or an amino acid sequence which is at least 77% identical to SEQ ID NO: 12, and a third HCDR comprises, preferably consists of, an amino acid sequence according to SEQ ID NO: 9, or an amino acid sequence which is at least 83% identical to SEQ ID NO: 9. In this embodiment, the isolated antibody, or antigen-binding fragment thereof, comprises three LCDRs of a LCVR. A first LCDR comprises, preferably consists of, an amino acid sequence according to SEQ ID NO: 14, or an amino acid sequence which is at least 70% identical to SEQ ID NO: 14, a second LCDR comprises, preferably consists of, the amino acid sequence according to SEQ ID NO: 15, or an amino acid sequence which is at least 66% identical to SEQ ID NO: 15, and a third LCDR comprises, preferably consists of, an amino acid sequence according to SEQ ID NO: 11, or an amino acid sequence which is at least 87% identical to SEQ ID NO: 11.

In an embodiment, the isolated antibody, or antigen-binding fragment thereof, comprises three HCDRs of a HCVR. A first HCDR comprises, preferably consists of, an amino acid sequence according to SEQ ID NO: 7, or an amino acid sequence which is at least 87% identical to SEQ ID NO: 7, a second HCDR comprises, preferably consists of, an amino acid sequence according to SEQ ID NO: 12, or an amino acid sequence which is at least 77% identical to SEQ ID NO: 12, and a third HCDR comprises, preferably consists of, an amino acid sequence according to SEQ ID NO: 9, or an amino acid sequence which is at least 83% identical to SEQ ID NO: 9. In this embodiment, the isolated antibody, or antigen-binding fragment thereof, comprises three LCDRs of a LCVR. A first LCDR comprises, preferably consists of, an amino acid sequence according to SEQ ID NO: 16, or an amino acid sequence which is at least 80% identical to SEQ ID NO: 16, a second LCDR comprises, preferably consists of, the amino acid sequence according to SEQ ID NO: 17, or an amino acid sequence which is at least 50% identical to SEQ ID NO: 17, and a third LCDR comprises, preferably consists of, an amino acid sequence according to SEQ ID NO: 11, or an amino acid sequence which is at least 87% identical to SEQ ID NO: 11.

In an embodiment, the isolated antibody, or antigen-binding fragment thereof, comprises three HCDRs of a HCVR. A first HCDR comprises, preferably consists of, an amino acid sequence according to SEQ ID NO: 7, a second HCDR comprises, preferably consists of, one amino acid sequence selected from the group consisting of SEQ ID NO: 12, 23, 24 and 25, and a third HCDR comprises, preferably consists of, an amino acid sequence according to SEQ ID NO: 9. In this embodiment, the isolated antibody, or antigen-binding fragment thereof, comprises three LCDRs of a LCVR. A first LCDR comprises, preferably consists of, one amino acid sequence selected from the SEQ ID NO: 14, 16, 26 and 27, and a second LCDR comprises, preferably consists of, one amino acid sequence selected from the group consisting of SEQ ID NO: 15, 17, 28 and 29, and a third LCDR comprises, preferably consists of, one amino acid sequence selected from the group consisting of SEQ ID NO: 11, 21 and 22.

In an embodiment, the isolated antibody, or antigen-binding fragment thereof, comprises three HCDRs of a HCVR. A first HCDR comprises, preferably consists of, an amino acid sequence according to SEQ ID NO: 7, a second HCDR comprises, preferably consists of, an amino acid sequence according to SEQ ID NO: 23 and a third HCDR comprises, preferably consists of, an amino acid sequence according to SEQ ID NO: 9. In this embodiment, the isolated antibody or antigen-binding fragment thereof comprises three LCDRs of a LCVR. A first LCDR comprises, preferably consists of, an amino acid sequence according to SEQ ID NO: 16, a second LCDR comprises, preferably consists of, an amino acid sequence according to SEQ ID NO: 15 and a third LCDR comprises, preferably consists of, an amino acid sequence according to SEQ ID NO: 21.

In an embodiment, the isolated antibody, or antigen-binding fragment thereof, comprises three HCDRs of a HCVR. A first HCDR comprises, preferably consists of, an amino acid sequence according to SEQ ID NO: 7, a second HCDR comprises, preferably consists of, an amino acid sequence according to SEQ ID NO: 24 and a third HCDR comprises, preferably consists of, an amino acid sequence according to SEQ ID NO: 9. In this embodiment, the isolated antibody, or antigen-binding fragment thereof, comprises three LCDRs of a LCVR. A first LCDR comprises, preferably consists of, an amino acid sequence according to SEQ ID NO: 27, a second LCDR comprises, preferably consists of, an amino acid sequence according to SEQ ID NO: 29 and a third LCDR comprises, preferably consists of, an amino acid sequence according to SEQ ID NO: 11.

In an embodiment, the isolated antibody, or antigen-binding fragment thereof, comprises three HCDRs of a HCVR. A first HCDR comprises, preferably consists of, an amino acid sequence according to SEQ ID NO: 7, a second HCDR comprises, preferably consists of, an amino acid sequence according to SEQ ID NO: 25 and a third HCDR comprises, preferably consists of, an amino acid sequence according to SEQ ID NO: 9. In this embodiment, the isolated antibody, or antigen-binding fragment thereof, comprises three LCDRs of a LCVR. A first LCDR comprises, preferably consists of, an amino acid sequence according to SEQ ID NO: 26, a second LCDR comprises, preferably consists of, an amino acid sequence according to SEQ ID NO: 28 and a third LCDR comprises, preferably consists of, an amino acid sequence according to SEQ ID NO: 22.

In an embodiment, the isolated antibody, or antigen-binding fragment thereof, comprises three HCDRs of a HCVR. A first HCDR comprises, preferably consists of, an amino acid sequence according to SEQ ID NO: 7, a second HCDR comprises, preferably consists of, an amino acid sequence according to SEQ ID NO: 12 and a third HCDR comprises, preferably consists of, an amino acid sequence according to SEQ ID NO: 9. In this embodiment, the isolated antibody, or antigen-binding fragment thereof, comprises three LCDRs of a LCVR. A first LCDR comprises, preferably consists of, an amino acid sequence according to SEQ ID NO: 14, a second LCDR comprises, preferably consists of, an amino acid sequence comprising the SEQ ID NO: 15 and a third LCDR comprises, preferably consists of, an amino acid sequence according to SEQ ID NO: 11.

In an embodiment, the isolated antibody, or antigen-binding fragment thereof, comprises three HCDRs of a HCVR. A first HCDR comprises, preferably consists of, an amino acid sequence according to SEQ ID NO: 7, a second HCDR comprises, preferably consists of, an amino acid sequence according to SEQ ID NO: 12 and a third HCDR comprises, preferably consists of, an amino acid sequence according to SEQ ID NO: 9. In this embodiment, the isolated antibody, or antigen-binding fragment thereof, comprises three LCDRs of a LCVR. A first LCDR comprises, preferably consists of, an amino acid sequence according to SEQ ID NO: 16, a second LCDR comprises, preferably consists of, an amino acid sequence comprising the SEQ ID NO: 17 and a third LCDR comprises, preferably consists of, an amino acid sequence according to SEQ ID NO: 11.

In an embodiment, the HCVR comprises, preferably consists of, one amino acid sequence selected from the group consisting of SEQ ID NO: 30, 32, 34, and 36, or an amino acid sequence which is at least 98% identical thereto; such as the group consisting of SEQ ID NO: 30, 34 and 36, or an amino acid sequence which is at least 96% identical thereto; such as the group consisting of SEQ ID NO: 34 and 36, or an amino acid sequence which is at least 96% identical thereto.

In an embodiment, the HCVR comprises, or consist of, an amino acid sequence according to SEQ ID NO: 36, or an amino acid sequence which is at least 96% identical thereto.

In an embodiment, the LCVR comprises, preferably consists of, one amino acid sequence selected the group consisting of SEQ ID NO: 31, 33, 35, 37 and 38 or an amino acid sequence which is at least 96% identical thereto; such as the group consisting of SEQ ID NO: 31, 35, 37 and 38, or an amino acid sequence which is at least 96% identical thereto; such as the group consisting of SEQ ID NO: 35, 37 and 38, or an amino acid sequence which is at least 96% identical thereto; such as the group consisting of SEQ ID NO: 37 and 38, or an amino acid sequence which is at least 96% identical thereto.

In an embodiment, the LCVR comprises, preferably consists of, an amino acid sequence according to SEQ ID NO: 37, or an amino acid sequence which is at least 96% identical thereto.

In an embodiment, the HCVR comprises, preferably consists of, an amino acid sequence independently selected the group consisting of SEQ ID NO: 30, 32, 34 and 36, or an amino acid sequence which is at least 96% identical thereto, and the LCVR comprises an amino acid sequence independently selected from the group consisting of SEQ ID NO: 31, 33, 35, 37 and 38, or an amino acid sequence which is at least 96% identical thereto. In a particular embodiment, the HCVR comprises, preferably consists of, an amino acid sequence independently selected the group consisting of SEQ ID NO: 30, 34 and 36, or an amino acid sequence which is at least 96% identical thereto and the LCVR comprises, preferably consists of, an amino acid sequence independently selected the group consisting of SEQ ID NO: 31, 35, 37 and 38, or an amino acid sequence which is at least 96% identical thereto. In another particular embodiment, the HCVR comprises, preferably consists of, an amino acid sequence independently selected the group consisting of SEQ ID NO: 34 and 36, or an amino acid sequence which is at least 96% identical thereto, and the LCVR comprises, preferably consists of, an amino acid sequence independently selected the group consisting of SEQ ID NO: 35, 37 and 38, or an amino acid sequence which is at least 96% identical thereto. In a further particular embodiment, the HCVR comprises, preferably consists of, an amino acid sequence according to SEQ ID NO: 36, or an amino acid sequence which is at least 96% identical thereto, and the LCVR comprises, preferably consists of, an amino acid sequence independently selected the group consisting of SEQ ID NO: 37 and 38, or an amino acid sequence which is at least 96% identical thereto.

In an embodiment, the isolated antibody, or antigen-binding fragment comprises, preferably consists of, a HCVR comprising an amino acid sequence according to SEQ ID NO: 36, or an amino acid sequence which is at least 96% identical thereto, and a LCVR comprising an amino acid sequence according to SEQ ID NO: 37 or 38, or an amino acid sequence which is at least 96% identical thereto.

In an embodiment, the isolated antibody, or antigen-binding fragment thereof, comprises a HCVR and a LCVR. The HCVR and LCVR are an amino acid sequence pair which is at least 96% identical to an amino acid sequence pair selected from the group consisting of the amino acid sequence pair SEQ ID NO: 30 and 31; the amino acid sequence pair SEQ ID NO: 32 and 33; the amino acid sequence pair SEQ ID NO: 34 and 35; the amino acid sequence pair SEQ ID NO: 36 and 37; and the amino acid sequence pair SEQ ID NO: 36 and 38; such as amino acid sequence pair selected from the group consisting of the amino acid sequence pair SEQ ID NO: 36 and 37; and the amino acid sequence pair SEQ ID NO: 36 and 38.

In an embodiment, the isolated antibody, or antigen-binding fragment thereof, comprises a heavy chain variable region and a light chain variable region. The heavy chain variable region comprises an amino acid sequence ZH1-[CDR-H1]-ZH2-[eCDR-H2] ZH3-[CDR-H3]-ZH4, wherein each of ZH1, ZH2, ZH3 and ZH4 represents zero, one or several independently selected amino acid residues. In an embodiment, the heavy chain variable region consists of an amino acid sequence selected from i) ZH1 -[GYTFTSYN]-ZH2-[X₅₃GVIX₅₇PGDGX₆₄TSYX₆₈Q K FX_{72]}-ZH3-[ARDYYGSSPLGY]-ZH4, wherein, independently from each other, X₅₃ is selected from I and M; X₅₇ is selected from N and Y; X₆₄ is selected from A and S; X₆₈ is selected from A and N; and X₇₂ is selected from K and Q, and ii) an amino acid sequence which has at least 92% identity to the sequence defined in i). The light chain variable region comprises an amino acid sequence ZL1-[eCDR-L1]-ZL2-[eCDR-L2]-ZL3-[CDR-L3]-ZL4, wherein each of ZL1, ZL2, ZL3 and ZL4 represents zero, one or several independently selected amino acid residues. In an embodiment, the light chain variable region consists of an amino acid sequence selected from iii) ZL1-[X₂₄ASX₂₇SISYX₃₉N] -ZL2-[AX₅₇SX₆₆LX_{68]} -ZL2- [HQRSSX₁₁₅PT]-ZL4, wherein, independently from each other, X₂₄ is selected from S and R; X₂₇ is selected from S and P; X₃₉ is selected from M and L; X₅₇ is selected from A and T; X₆₆ is selected from K and S; X₆₈ is selected from A and P; and X₁₁₅ is selected from S, T and Y, and iv) an amino acid sequence which has at least 87% identity to the sequence defined in iii).

In an embodiment, ZH1 comprises, preferably consists of, an amino acid sequence according to SEQ ID NO: 39 or an amino acid sequence which is at least 90% identical to SEQ ID NO: 39

In an embodiment, ZH2 comprises, preferably consists of, an amino acid sequence according to SEQ ID NO: 40 or an amino acid sequence which is at least 90% identical to SEQ ID NO: 40.

In an embodiment, ZH3 comprises, preferably consists of, an amino acid sequence according to SEQ ID NO: 41 or an amino acid sequence which is at least 90% identical to SEQ ID NO: 41.

In an embodiment, ZH4 comprises, preferably consists of, an amino acid sequence according to SEQ ID NO: 42 or an amino acid sequence which is at least 90% identical to SEQ ID NO: 42.

In an embodiment, ZL1 comprises, preferably consists of, an amino acid sequence according to SEQ ID NO: 43 or an amino acid sequence which is at least 90% identical to SEQ ID NO: 43.

In an embodiment, ZL2 comprises, preferably consists of, an amino acid sequence according to SEQ ID NO: 44 or an amino acid sequence which is at least 90% identical to SEQ ID NO: 44.

In an embodiment, ZL3 comprises, preferably consists of, an amino acid sequence according to SEQ ID NO: 45 or an amino acid sequence which is at least 90% identical to SEQ ID NO: 45.

In an embodiment, ZL4 comprises, preferably consists of, an amino acid sequence according to SEQ ID NO: 46 or an amino acid sequence which is at least 90% identical to SEQ ID NO:46.

In an embodiment, the antibody is a full-length antibody.

In an embodiment, the antibody is selected from the group consisting of human antibodies, humanized antibodies and chimeric antibodies.

In an embodiment, the antigen-binding fragment is an antigen-binding fragment, such as a single chain fragment variable, a Fab fragment, F(ab′)₂ fragment, a F(ab′)₃ fragment, a Fab′ fragment, a Fd fragment, a Fv fragment, a dAb fragment, an isolated complementarity determining region (CDR) and a nanobody. In a particular embodiment, the antigen-binding fragment is a scFv fragment.

In an embodiment, the isolated antibody, or antigen-binding fragment thereof, is a monoclonal antibody or an antigen-fragment thereof. In a particular embodiment, the monoclonal antibody, or antigen-binding fragment thereof, is a humanized monoclonal antibody or antigen-binding thereof.

In an embodiment, the isolated antibody or antigen-binding fragment thereof is selected from the group consisting of isotype class IgG, IgA, IgM, IgD and IgE; such as IgG. In a particular embodiment, the isolated antibody, or antigen-binding fragment thereof, is selected from the group consisting of isotype subclass IgG1 and IgG4.

In an embodiment, the isolated antibody, or antigen-binding fragment thereof, comprises one or more Fc silencing mutations. In a particular embodiment, the IgG1 comprises the Fc silencing mutations L234A, L235A and P329G.

In an embodiment, the isolated antibody, or antigen-binding fragment thereof, comprises one or more stabilizing mutations which prevent or reduce in vivo Fab arm exchange. In a particular embodiment, the IgG4 comprises the stabilizing mutation S228P.

In an embodiment, the isolated antibody, or antigen-binding fragment thereof, is a single chain fragment variable (scFv) that specifically binds to hBSSL and which comprises an HCVR domain comprising a first HCDR, a second HCDR and a third HCDR comprising or consisting of amino acid sequences which are at least 80% identical to SEQ ID NO: 7, SEQ ID NO: 8, and SEQ ID NO: 9, respectively, and an LCVR domain comprising a first LCDR, a second LCDR and a third LCDR comprising or consisting of amino acid sequences at least 80% identical to SEQ ID NO: 10, the amino acid sequence ATS, and SEQ ID NO: 11, respectively.

In an embodiment, the first HCDR, the second HCDR and the third HCDR consist of amino acid sequences according to SEQ ID NO: 7, SEQ ID NO: 8, and SEQ ID NO: 9, respectively, and the first LCDR, the second LCDR and the third LCDR consist of amino acid sequences according to SEQ ID NO: 10, the amino acid sequence ATS, and SEQ ID NO: 11, respectively.

In an embodiment, the antibody is a humanized antibody.

In an embodiment, the isolated antibody, or antigen-binding fragment thereof, has an affinity to hBSSL of no more than K_{D} 1.7 nM.

In an embodiment, the isolated antibody, or antigen-binding fragment thereof, is capable of displacing binding of hBSSL to monocytes, preferably CD14+monocytes.

An embodiment relates to a pharmaceutical composition comprising an isolated antibody and/or an antigen-binding fragment thereof according to the invention and a pharmaceutically acceptable carrier or excipient.

An embodiment relates to an isolated antibody and/or antigen-binding fragment thereof, or a pharmaceutical composition according to the invention, for use as a medicament.

An embodiment relates to an isolated antibody and/or antigen-binding fragment thereof, or a pharmaceutical composition according to the invention, for use in the treatment and/or prevention of an inflammatory disease.

An embodiment relates to use of an isolated antibody and/or antigen-binding fragment thereof, or a pharmaceutical composition according to the invention, for the manufacture of a pharmaceutical composition for the treatment and/or prevention of an inflammatory disease.

An embodiment relates to a method for treating and/or ameliorating and/or preventing and/or prophylaxis of an inflammatory disease. In this method, a therapeutically effective amount of an isolated antibody and/or antigen-binding fragment thereof, or a pharmaceutical composition according to the invention, is administered to a subject in need thereof.

In an embodiment, the inflammatory disease is a chronic inflammatory disease.

In an embodiment, the inflammatory disease is a systemic inflammatory disease.

In an embodiment, the inflammatory disease is an autoimmune disease. In a particular embodiment, the autoimmune disease is rheumatoid arthritis or juvenile rheumatoid arthritis. In another particular embodiment, the autoimmune disease is inflammatory bowel disease (IBD), such as Crohn's disease or ulcerative colitis.

In an embodiment, inflammatory disease is an autoinflammatory disease. In a particular embodiment, the autoinflammatory disease is psoriatic arthritis.

In an embodiment, the inflammatory disease is liver steatosis.

In an embodiment, the isolated antibody and/or antigen-binding fragment thereof or the pharmaceutical composition is systemically administered.

In an embodiment, the isolated antibody and/or antigen-binding fragment thereof or the pharmaceutical composition is parenterally administered, such as subcutaneously administered. Hence, in a particular embodiment, the isolated antibody and/or antigen-binding fragment thereof or the pharmaceutical composition is formulated for parenteral administration, such as subcutaneous administration.

In an embodiment, the isolated antibody and/or antigen-binding fragment thereof or the pharmaceutical composition is administered 1-3 times per week, such as 1-2 times per week, such as 1 time a week.

In an embodiment, the treatment and/or prevention is by passive immunotherapy.

Embodiments relate to a polynucleotide encoding an isolated antibody or antigen-binding fragment thereof as defined according to the invention, an expression vector comprising a polynucleotide according to the invention and a host cell comprising an expression vector according to the invention.

An embodiment relates to a method of producing an isolated antibody or antigen-binding fragment thereof according to the invention. The method comprises culturing a host cell according to the invention under conditions permissive of expression of the antibody, or antigen-binding fragment thereof, and isolating the antibody, or antigen-binding fragment thereof.

An embodiment relates to a method for detecting the presence or absence of BSSL and/or quantifying the amount of BSSL in a sample. The method comprises the steps of a) providing a sample potentially containing BSSL, b) contacting the sample with an isolated antibody, or antigen-binding fragment thereof, according to the invention, and c) detecting the presence or absence of BSSL and/or quantifying the amount of BSSL in said sample.

An embodiment relates to a method for diagnosis of a BSSL related disorder. The method comprises the steps of a) providing a sample from a subject suspected of suffering from a BSSL related disorder, b) contacting said sample with an isolated antibody, or antigen-binding fragment thereof, according to the invention, c) detecting the presence or absence of BSSL and/or quantifying the amount of BSSL in the sample, and d) concluding based on the results in step c) whether the subject is diagnosed with a BSSL related disorder or not.

In an embodiment, the BSSL related disorder is an inflammatory disease, such as a chronic inflammatory disease; a systemic inflammatory disease; an autoimmune disease, such as rheumatoid arthritis, juvenile rheumatoid arthritis, inflammatory bowel disease, such as Crohn's and ulcerative colitis; an autoinflammatory disease, such as psoriatic arthritis; or liver steatosis.

An embodiment relates to a method for determining the enzymatic activity of BSSL. The method comprises the steps of: a) providing a sample containing BSSL, b) contacting the sample with an isolated antibody, or antigen-binding fragment thereof according to the invention, and c) determining the enzymatic activity of the BSSL in the sample.

An embodiment relates to a BSSL epitope comprising or consisting of a first epitope and a second epitope. The first epitope comprises or consists of an amino acid sequence according to SEQ ID NO: 1 or an amino acid sequence having at least 80%, such as 85%, 90%, 95%, 96%, 97%, 98% or 99% identity to SEQ ID NO: 1. The second epitope consists of a second surface comprising or consisting of an amino acid sequence according to SEQ ID NO: 2, or an amino acid sequence having at least 80%, such as 85%, 90%, 95%, 96%, 97%, 98% or 99% identity to SEQ ID NO: 2.

In an embodiment, the first epitope comprises, preferably consists of, an amino acid sequence according to SEQ ID NO: 3, or an amino acid sequence having at least 80%, such as 85%, 90%, 95%, 96%, 97%, 98% or 99% identity thereto.

In an embodiment, the epitope further comprises an amino acid sequence according to SEQ ID NO: 4, or an amino acid sequence having at least 80%, such as 85%, 90%, 95%, 96%, 97%, 98% or 99% identity thereto.

In an embodiment, the epitope further comprises an amino acid sequence according to SEQ ID NO: 5, or an amino acid sequence having at least 80%, such as 85%, 90%, 95%, 96%, 97%, 98% or 99% identity thereto.

In an embodiment, the epitope further comprises an amino acid sequence according to SEQ ID NO: 6, or an amino acid sequence having at least 80%, such as 85%, 90%, 95%, 96%, 97%, 98% or 99% identity thereto.

While the invention has been described with reference to various exemplary aspects and embodiments, it will be understood by those skilled in the art that various changes may be made and equivalents may be substituted for elements thereof without departing from the scope of the invention. In addition, many modifications may be made to adapt a particular situation or molecule to the teachings of the invention without departing from the essential scope thereof. Therefore, it is intended that the invention not be limited to any particular embodiment contemplated, but that the invention will include all embodiments falling within the scope of the appended claims.

### EXAMPLES

TABLE 2
BSSL material used in the Examples

| | Short | | | SEQ |
|---|---|---|---|---|
| Reagent | name | Characteristics | Source/Supplier | ID NO: |
| Human | hBSSL | Native | Astra Hässle AB | 138 |
| BSSL | | | | |
| Biotinylated | b-hBSSL | Biotinylated on | Astra Hässle AB, | 138 |
| human | | glycosacarides, | biotinylated | |
| BSSL | | 30-40 | in house | |
| | | biotin/BSSL | | |
| Biotinylated | hBSSL-b | Human, | Astra Hässle AB, | 138 |
| human | amine | biotinylated on | biotinylated | |
| BSSL | | free amine, ≈3 | in house | |
| | | biotin/BSSL | | |
| Mouse | mBSSL | Recombinant | R&D Systems | 137 |
| BSSL | | | (Cat. no: | |
| | | | 5658-CE) | |

### Example 1—Binding of AS20 IgG to Human and Mouse BSSL (SPR)

In this Example, the binding of antibody AS20 mouse-IgG1 (AS20 mlgG) to both human and mouse BSSL was investigated by surface plasmon resonance (SPR). AS20 mlgG (heavy chain variable region (HCVR) SEQ ID NO: 80 and light chain variable region (LCVR) SEQ ID NO: 114) has been raised in mouse against the full-length BSSL protein (SEQ ID NO: 138), purified from human milk. The reactivity to mouse BSSL was unknown.

### Material and Methods

The hBSSL and mBSSL (Table 2) were used together with AS20 mouse IgG1 (AS20 mlgG1) (in house produced, HCVR SEQ ID NO: 80 and LCVR SEQ ID NO: 114) in the experiments described in this Example.

SPR measurements were performed with a BIACORE® T200 instrument (GE Healthcare). In order to minimize avidity effects, the antibody was immobilized on the sensor surface and BSSL was injected as the analyte. Immobilization of AS20 mlgG1 was performed by amine coupling to a BIACORE® CM5 (carboxylated dextran surface) sensor chip. The chips were activated by injection of a 1:1 mixture of 0.2 M N-ethyl-N′-[(dimethylamino)peopyl]carbodiimide (EDC) and 0.05 M N-hydroxysucciminimde (NHS) with a contact time of 7 minutes. The antibody was injected for 0.2-2.8 minutes diluted to 14-50 μg/ml in 10 mM acetate-HCl pH 5.0 (set one) or pH 6.0 (set two) to reach final immobilization levels of between 560 and 830 RU. Remaining activated carboxyl groups on the sensor surface were deactivated with an injection of 1 M ethanolamine for 7 minutes.

The running buffer in the first set of experiments was PBS buffer pH 7.4 (10 mM phosphate, 2.5 mM KCl, 137 mM NaCl) with addition of 0.05% (v/v) Tween 20. In the second set of experiments the running buffer was 25 mM TrisHCl, pH 7.5, 150 mM NaCl. 146 mM H₃PO₄ was used as the standard regeneration solution. The kinetic studies and the non-linear regression analysis were made according to the Single Cycle Kinetics (SCK) method of the BIACORE® T200 instrument and evaluation software. The interaction between mBSSL and AS20 mlgG1 was analyzed with steady state affinity model.

In experiment set one, three SCK experiments were made with the highest hBSSL concentrations in the concentration series being 300, 100 and 50 nM, respectively. The concentration series were made with 1:3, 1:3.16 (half-log) and 1:2 dilutions.

In the second set of experiments, hBSSL was diluted to 20 nM starting concentration in the running buffer, followed by a 1:1 serial dilution in the same buffer, resulting in 5-points concentrations ranging from 20 nM to 1.25 nM. mBSSL was diluted to 2000 nM starting concentration in the running buffer, followed by a 1:1 serial dilution in the same buffer, resulting in 5-points concentrations, ranging from 2000 nM to 125 nM.

### Results

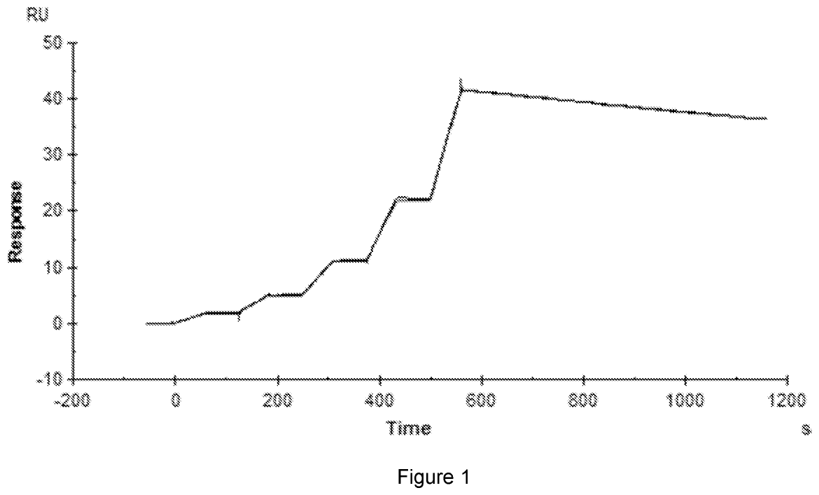

AS20 mlgG1 was found to bind to both human and mouse BSSL. The affinity to human BSSL was strong with low nanomolar affinity. The interaction was well characterized by a 1:1 binding model (FIG. 1). The association and dissociation rate constants and the equilibrium dissociation constant from the non-linear regression analysis of the SCK experiments are presented in Table 3. In the first set of experiments, the measurements were performed in triplicate and thus the averages and standard deviations are presented.

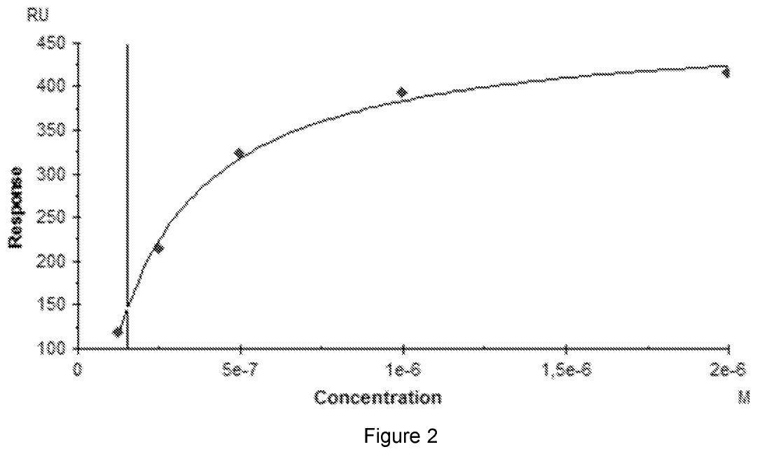

Mouse BSSL was also seen to interact with immobilized AS20 mlgG1, however, with almost 100 times weaker affinity. A steady state analysis was performed in order to determine the affinity (FIG. 2 and Table 3).

TABLE 3
Kinetic parameters of the interaction between
AS20 mIgG1 and hBSSL, and AS20 mIgG1 and mBSSL

| AS20 mIgG | kₒₙ | k_{off} | K_{D} |
|---|---|---|---|
| interaction with: | (M⁻¹s⁻¹) | (s⁻¹) | (nM) |
| hBSSL*¹ | (9.7 ± 0.9) × 10⁴ | (2.7 ± 0.1) × 10⁻⁴ | 2.8 ± 0.2 |
| hBSSL*² | 2.3 × 10⁵ | 2.5 × 10⁻⁴ | 1.1 |
| mBSSL**² | N.D | N.D | 155 |

*Single cycle kinetics
**Steady state analysis
N.D Not determined
¹First set of experiments
²Second set of experiments

### Example 2—Production and Binding Characterization of AS20 scFv to Human and Mouse BSSL (ELISA, SPR and LUMINEX®)

In this Example, a single-chain variable fragment (scFv) version based on AS20 mlgG1, denoted AS20 scFv (comprising HCVR SEQ ID NO: 80 and LCVR SEQ ID NO: 114), was generated with retained binding towards human BSSL as assessed by enzyme-linked immunosorbent assay (ELISA), SPR and LUMINEX®.

### Material and Methods

### Small-Scale Production and Purification

By fusing the HCVR (SEQ ID NO: 80) to the LCVR (SEQ ID NO: 114) via a glycine-serine linker, a gene encoding the corresponding scFv construct was formed. The scFv gene was sub-cloned into the pHAT-6 screening vector (SciLifeLab, Stockholm, Sweden), providing a signal for secretion of the scFv along with a triple-FLAG tag and a hexahistidine (His) tag at the C-terminus. The construct was subsequently transformed into TOP10 Escherichia coli. Bacterial supernatant of lysed cells was purified using α-FLAG antibody conjugated magnetic beads (Sigma Aldrich, #M8823). Purified scFv was analyzed by gel electrophoresis under reducing conditions to determine its purity and integrity, and protein concentration was determined by the BCA (Bicinchoninic Acid) assay kit (Pierce).

### ELISA

Non-biotinylated human BSSL (hBSSL) and biotinylated human BSSL (b-hBSSL) was either directly coated or coated through streptavidin, into a 384-well ELISA plate at two different concentrations, 1 μg/ml and 0.5 μg/ml in PBS at 4° C. overnight. Purified AS20 scFv was serial diluted 3-fold in blocking buffer (phosphate buffered saline (PBS) supplemented with 0.5% bovine serum albumin (BSA) and 0.05% Tween20) with concentrations ranging from 1 μg/ml to 4 ng/ml. Detection of binding was enabled through a horseradish peroxidase (HRP)-conjugated α-FLAG M2 antibody (Sigma-Aldrich), followed by incubation with chromogen substrate Ultra 3,3′,5,5′-Tetramethylbenzidine (TMB) ELISA. Signal development was stopped by addition of 1 M sulphuric acid and absorbance was measured at 450 nm.

In a second ELISA assay, 1 μg/ml of human and mouse BSSL, as well as a negative control protein, were directly coated into a 384-well ELISA plate and incubated at 4° C., overnight. Purified AS20 scFv and a negative control scFv were added in two different concentrations, 1 μg/ml and 0.2 μg/ml. Detection of binding signal was performed as described above. All samples, in both ELISA set ups, were assayed in duplicates. hBSSL, b-hBSSL and mBSSL (Table 2) were used together with AS20 scFv (in house produced, HCVR SEQ ID NO: 80 and LCVR SEQ ID NO: 114) in the experiments conducted in this Example.

SPR Measurements

Affinity rating of the scFv clones were performed by SPR using BIACORE® T200 (GE Healthcare). An α-FLAG M2 antibody was immobilized onto a CM5 S chip through primary amine coupling using NHS-EDC chemistry, allowing capture of AS20 scFv through its 3xFLAG tag. A 3-fold dilution series comprised of five different concentrations, 200 nM to 2 nM, of hBSSL and b-hBSSL was sequentially injected over the flow cells, allowing binding to captured AS20 scFv. Regeneration of the surface was accomplished under acidic conditions using 10 mM glycin-HCl at pH 2.2. Obtained single cycle kinetic data was fitted to a 1:1 Langmuir binding model and kinetic parameters, ka (1/Ms), k_{d} (1/s) and K_{D} (M) was retrieved using software BIAevaluation.

### LUMINEX® Analyses

Biotinylated hBSSL was incubated with neutravidin-coupled LUMINEX® beads and mixed with 30 different beads ID, each conjugated to a non-relevant protein. The mixed bead pool was incubated with AS20 scFv present in bacterial supernatant diluted 1:10 in assay buffer (PBS supplemented with 3% BSA, 0.05% Tween20 and 10 μg/ml neutravidin). One positive scFv control was also included, i.e., a scFv expected to bind to beads coated with one of the non-relevant proteins. Binding of scFv clones to a particular protein-conjugated bead was enabled through a R-PE-conjugated anti-FLAG M2 antibody followed by analyses on a FlexMAP 3D instrument.

### Results

### Small-Scale Production and Purification

Gel electrophoresis of bacterially expressed and purified AS20 scFv showed a high purity with one main protein band corresponding well to the expected molecular weight of a scFv (data not shown).

### ELISA

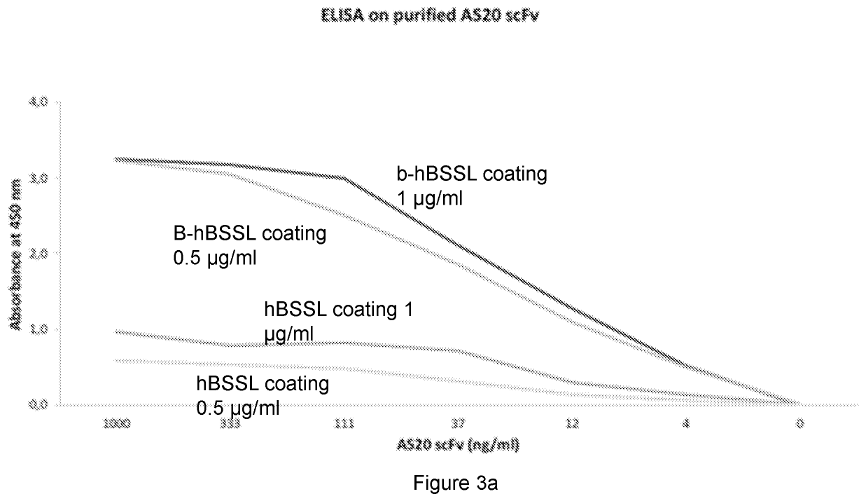

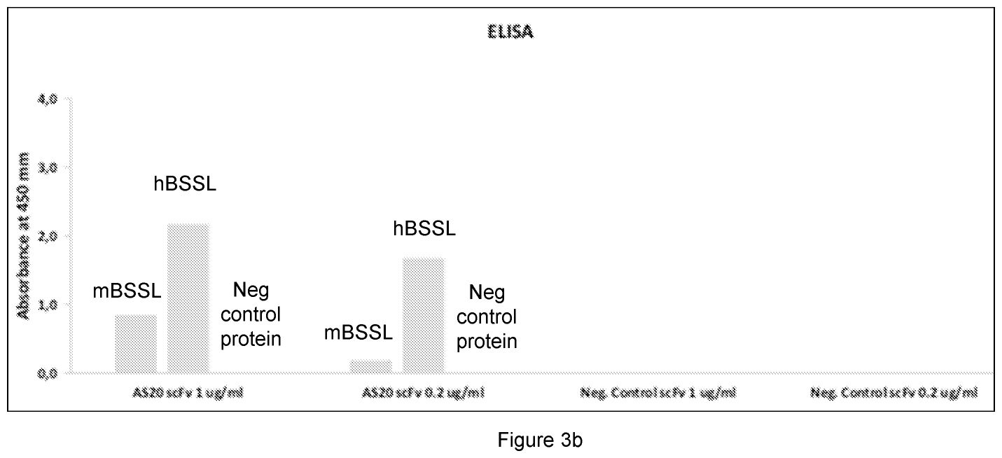

AS20 scFv displayed a concentration-dependent binding towards both non-biotinylated and biotinylated human BSSL (FIG. 3a). Signal intensity at a particular scFv concentration was much higher towards biotinylated BSSL then towards non-biotinylated BSSL, which may be due to differences in coating conditions.

AS20 scFv also displayed binding towards mouse BSSL but the signal intensity was much weaker than towards human BSSL (FIG. 3b), which may indicate a weaker affinity of AS20 scFv towards mouse BSSL. No binding of AS20 scFv to the negative control was detected.

SPR measurements

A single cycle kinetic approach was used to determine the affinity of AS20 scFv to non-biotinylated and biotinylated human BSSL. AS20 scFv displayed very similar affinities to non-biotinylated human BSSL (K_{D}=0.6 nM) and to biotinylated human BSSL (K_{D}=0.8 nM) (Table 4).

TABLE 4
Kinetic parameters of the interaction between
AS20 scFv and hBSSL, native and biotinylated

| AS20 scFv | kₒₙ | k_{off} | K_{D} |
|---|---|---|---|
| interaction with: | (M⁻¹s⁻¹) | (s⁻¹) | (nM) |
| hBSSL | 1.9 × 10⁵ | 1.2 × 10⁻⁴ | 0.6 |
| b-hBSSL | 5.5 × 10⁴ | 4.4 × 10⁻⁵ | 0.8 |

### Luminex Analyses

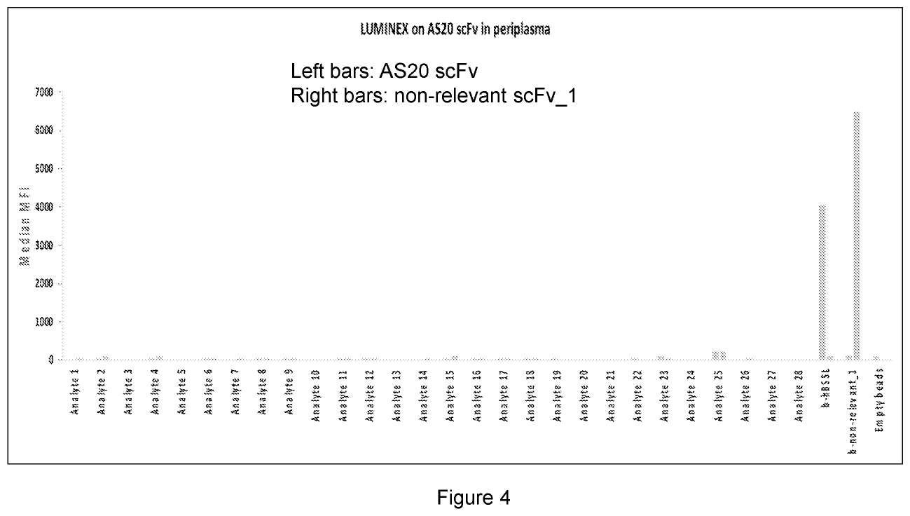

To determine if AS20 scFv is prone to non-specific interaction with non-relevant proteins, a Luminex assay was performed in which AS20 scFv was analyzed on 30 different non-relevant proteins, as well as to its cognate target. AS20 scFv only displayed binding to human BSSL with no or very low binding to all other proteins included in the assay (FIG. 4).

### Conclusions

The AS20 scFv was found to bind to both non-biotinylated human BSSL and biotinylated human BSSL with similar affinities (similar K_{D} values), in the sub-nanomolar range. The obtained K_{D}-value for non-biotinylated BSSL is well in line with what has been reported in Example 1 for the full-length IgG antibody. AS20 scFv also displayed low off-target binding to 30 non-relevant proteins when assayed in a Luminex-based approach. As indicated by the ELISA results, AS20 scFv shows binding towards mouse BSSL, which was shown for the full-length IgG in Example 1.

### Example 3—Binding Characterization of AS20 scFv to Mouse BSSL by HTRF

Homogeneous Time-Resolved Fluorescence (HTRF) is a spectrophotometric method based on the phenomenon of fluorescence resonance energy transfer (FRET) between two different molecules, which are known as donor and acceptor, respectively. This Example describes the development and use of an HTRF based competition assay as an alternative method to characterize the interaction between the AS20 scFv and mouse BSSL.

### Material and Methods

In order to study the interaction between AS20 scFv and native mouse BSSL, a competition assay was developed. Detection of binding was enabled through donor molecule terbium-conjugated α-FLAG antibody (Cisbio #611FG2TL), which interacts with the C-terminal located FLAG-tag of the scFv, and acceptor molecule streptavidin-conjugated XL665 (Cisbio #610SAXL), which interacts with the biotin-moieties on human BSSL. The experiments were carried out using a range of concentrations for the non-biotinylated proteins; 0-500 nM for mBSSL and 0-80 nM for hBSSL. hBSSL, b-hBSSL and mBSSL (Table 2) were used together with AS20 scFv (in house produced, HCVR SEQ ID NO: 80 and LCVR SEQ ID NO: 114) in the experiments conducted in this Example.

2.5 nM of AS20 scFv was pre-incubated for 2 hours with the mouse or human orthologue of BSSL, after which 5 nM b-hBSSL and the FRET donor and acceptor molecules were added. The mixture was finally incubated at room temperature for 16 hours and the binding signal (665 nm) and background/noise signal (615 nm) was measured using EnVision (PerkinElmer). The experimental output, Delta R, was calculated for each point using four replicates and two blanks.

### Results

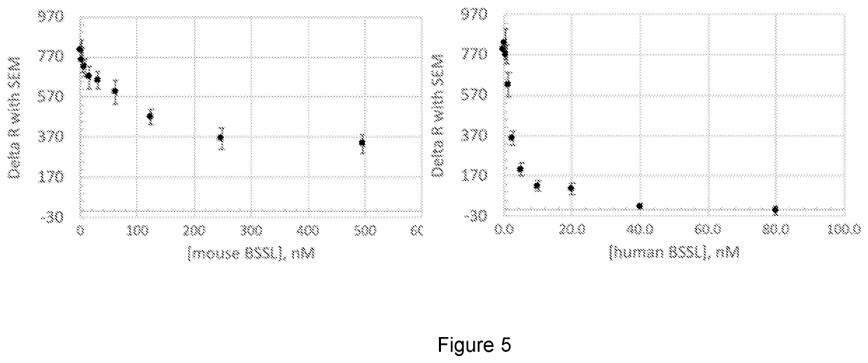

Data indicated that mouse BSSL competed, in a concentration dependent manner, with human BSSL-biotin for binding to the AS20 scFv (FIG. 5). The same observation was seen for human native BSSL. A 50% reduction in Delta R was achieved with 150-200 nM of mouse BSSL and roughly 2 nM of human BSSL, suggesting that the AS20 scFv has a roughly 100-fold lower affinity for mouse BSSL compared to the human orthologue.

### Conclusions

The obtained data indicated that AS20 scFv had a roughly 100-fold lower affinity for mouse BSSL compared to the human orthologue. This was very much in line with the affinities that were obtained for full length antibody formats of the same clone using SPR; AS20 mlgG1 (Example 1) and chimeric AS20 (Example 4).

### Example 4—Production of Chimeric AS20

In this Example, a chimeric AS20 is produced. More specifically, a chimer of the human IgG4 subclass was constructed.

### Materials and Methods

Production of material was outsourced to GenScript (Piscataway, N.J., USA). Sequences of the variable domains of the heavy (VH) and light (VL) chains of the mouse AS20 antibody, corresponding to SEQ ID NO: 80 and SEQ ID NO: 114, respectively were used. Genes encoding the heavy and light chains were synthesized and cloned into a vector encoding the human IgG4 subclass. The constructs were transfected and transiently expressed in FreeStyle 293-F cells. The expressed antibody was subsequently purified by affinity chromatography using protein A followed by preparative size exclusion chromatography (SEC), purity determined by high-performance liquid chromatograph (HPLC) and concentration determined spectrophotometrically at 280 nm.

### Results

The purity was determined by HPLC to >98%. The sequence of the chimeric AS20 was determined to be SEQ ID NO: 139 for the heavy chain and SEQ ID NO: 140 for the light chain.

The chimeric AS20 retained the same binding affinity (data not shown) to mouse and human BSSL as the AS20 mouse IgG1 antibody in Example 1.

### Example 5—Design and Construction of AS20 CDR-Graft Antibody and of AS20 Humanization Library

AS20 is a mouse antibody, AS20 mlgG, see Example 1. Non-human antibodies have been shown to induce human immune responses, which can result in neutralization of the administered antibody and in turn limits the effect of the antibody in treatment of disease. To overcome this potential problem, humanization of the antibody was performed. This Example, described two strategies for humanization of AS20, namely complementary determining regions (CDR) grafting and a library-based approach. The resulting CDR graft antibody is referred to as AS20 CDR graft or CDR graft herein and the generated library is referred to as AS20 humanization library herein. The library was subsequently used for selection and isolation of AS20 binding scFv fragments using phage display (see Example 6).

### Materials and Methods

FIGS. 15A and 15B are a summary of the design of combinatorial scFv library for the heavy chain variable region and FIGS. 16A and 16B is a summary of the design of combinatorial scFv library for the light chain variable region.

The scFv format was chosen as scaffold for both the CDR graft and the humanization library. Data presented in Example 2 showed that the AS20 scFv fully retained the binding ability of its full-length parental IgG counterpart, suggesting that the scFv gene will be a good scaffold format for both the CDR graft and to build combinatorial libraries on.

The human immunoglobulin heavy chain variable region germline gene (IHGV) with highest sequence homology to the AS20 heavy chain variable region, according to IMGT/DomainGapAlign, (http://www.imgt.org/3Dstructure-DB/cgi/DomainGapAlign.cgi), IGHV1-46, with a homology of 73.5% (reside 1-104), was chosen as the IHGV framework. For choice of light chain sequence, homology was considered but also heavy and light chain pairings with favorable biophysical properties were taken into account [10]. Taken together this resulted in choosing the human germline gene IGKV1-39. Also based on the IMGT/DomainGapAlign domain search, IGHJ4 and IKVJ2 were chosen as joining fragments to obtain full variable heavy and light chain domains, respectively.

The AS20 CDR graft was obtained by grafting the six mouse CDR loops into the human germline genes. For the heavy chain the following regions were grafted into the IGHV1-46 framework: heavy chain complementarity-determining region 1 (HCDR1) (SEQ ID NO: 7); extended HCDR2 (eHCDR2) (SEQ ID NO: 141); HCDR3 (SEQ ID NO: 9). This resulted in the CDR graft with the HCVR according to SEQ ID NO: 144. For the light chains the following regions extended light chain complementarity-determining region 1 (eLCDR1) (SEQ ID NO: 142), eLCDR2 (SEQ ID NO: 143) and LCDR3 (SEQ ID NO: 21) were grafted into the IGKV1-39 framework resulting in an LCVR according to SEQ ID NO: 145. By fusing the HCVR to the LCVR via a glycine-serine linker ((Gly₄Ser)₃), a gene encoding the corresponding scFv construct was formed. Two additional amino acids (Arg and Thr, both part of the CL domain) were added at the end of LCVR in order to include the BsiW restriction site. Synthesis and sub-cloning of the scFv gene was out-sourced to GenScript (Piscataway, NJ, USA). After synthesis, the scFv gene was cloned into an in-house phagemid using restriction enzymes Sfil and BsiWll.

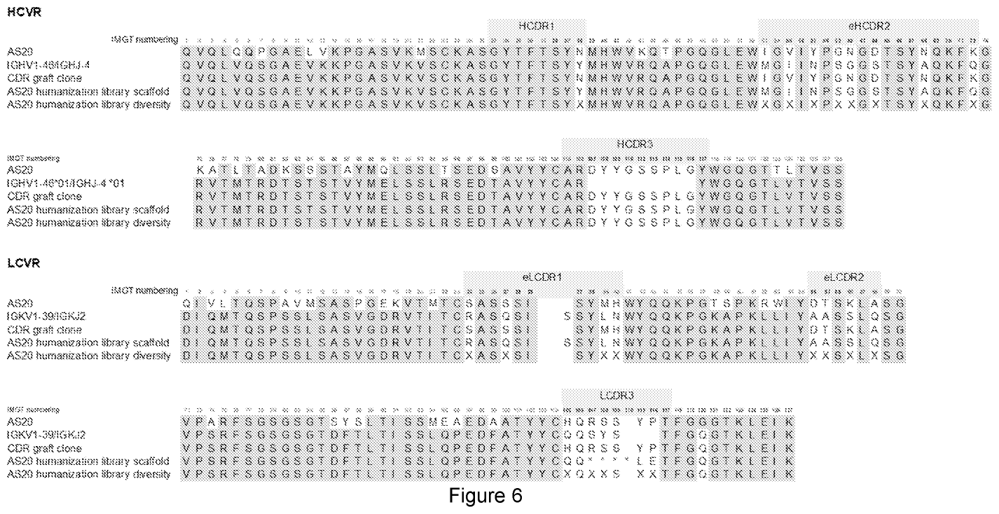

The same HCVR and LCVR framework as used in the CDR graft was used to construct the AS20 humanization library scaffold (FIG. 6). The mutagenesis strategy for the AS20 humanization library is summarized in Table 5. The HCDR3 is considered the most important region for antigen binding. It was reasoned that this loop is most likely also important for the AS20-BSSL interaction and was therefore kept constant. Instead, the 22 positions differing between AS20 and the AS20 humanization scaffold in the other five CDR regions were chosen for variation (FIG. 6). Here, primarily a dual diversity was attempted, i.e., allowing the residues found in AS20 and in the human germline genes that build up the humanization scaffold at a particular position. However, not all pairs of amino acids can be established by NNS oligoes without the introduction of additional amino acids and extra chemical diversity was therefore added in six positions (three in VH: 62, 64, 68 and three in VL: 27, 66, 68). In LCDR3, an alternative strategy was taken. Here, diversity found in rearranged functional antibodies encoded by germline gene IGVK1-39/IKV1D-39 as found in the IMGT database (http://www.imgt.org/ligmdb/) was taken into account. More specifically, sequences of antibodies having an LCDR3 length of 8 amino acids, the length of LCDR3 in AS20, was used as guide for what diversity to introduce. The obtained consensus sequence was QQSYSTPT (aa 105-117, SEQ ID NO: 173). Based on the mouse AS20 and the human consensus sequences, a dual diversity was introduced in position 105, 107 and 108. In position 115, again due to the limitation of NNS oligoes, four amino acids were introduced. As a result of the VJ gene joining process, most diversity in LCDR3 is found in position 116. In an attempt to mimic this variability, but also being limited by the use of NNS codons, six amino acids were allowed here (P, H, L, Y, S and F). This strategy allows us to capture >50% of the diversity found among antibodies in this position. Altogether, the above procedure creates a combinatorial theoretical diversity of approximately 1.2×10⁹ different variants.

TABLE 5
Positions targeted for mutagenesis in the AS20 humanization library.
Positions in HCVR and LCVR are listed in the upper and lower
part of the table, respectively. Amino acids marked in bold
are those found in AS20, whereas underlined amino acids are
corresponding diversity found in the human germline gene. Numbering
is as defined by the IMGT nomenclature and for codon definition
the IUPAC nucleotide code is used. One primer each for the five
targeted regions (HCDR1, HCDR2, LCDR1, LCDR2 and LCDR3) was
used to introduce the diversity (Table 6).

| | Designed diversity | Codon |
|---|---|---|
| Target positions HCVR | | |
| 38 | N, Y | WAT |
| 53 | I, M | ATR |
| 55 | V, I | RTA |
| 57 | Y, N | WAC |
| 59 | G, S | RGT |
| 62 | N, G, S, D | RRT |
| 64 | D, S, A, Y | KMC |
| 68 | N, A, T, D | RMT |
| 72 | K, Q | MAG |
| Target positions LCVR | | |
| 24 | S, R | AGK |
| 27 | S, Q, P, STOP | YMG |
| 39 | M, L | WTG |
| 40 | H, N | MAT |
| 56 | D, A | GMT |
| 57 | T, A | RCA |
| 66 | K, S, N, R | ARS |
| 68 | A, Q, P, E | SMA |
| 105 | H, A | CAS |
| 107 | R, S | AGK |
| 108 | S, Y | TMT |
| 115 | Y, T, N, S | WMT |
| 116 | P, H, L, Y, S, F | YHC |

The diversity was introduced into the library scaffold gene using an optimized Kunkel mutagenesis methodology basically as described in [11], making use of the AS20 humanization library scaffold gene (FIG. 6) together with the five mutagenic oligonucleotides (Table 6). To assess whether the intended diversity had been incorporated, TOP10 E. coli cells were chemically transformed with a small aliquot of the DNA generated by the Kunkel mutagenesis methodology and 96 clones were picked and sent for sequencing (GATC, Germany). The remaining DNA was subsequently electroporated into SS320 cells (Lucigen, Middleton, Wis., USA), yielding a highly diverse library containing approximately 1.7×10¹⁰ clones, as measured by the number of bacterial colonies obtained after transformation. The transformed SS320 cells were harvested and stored with 15% glycerol at −80° C. The bacterial glycerol stock was used to inoculate a total of 600 ml 2×YT with antibiotics selective for both the phagemid and the F″ episome. The bacteria were grown until exponential phase and then infected by M13KO7 helper phages (New England Biolabs, Ipswich, Mass., USA) using a multiple of infection of five. The culture was propagated overnight and scFv displaying phages were harvested by standard polyethylene glycol PEG/NaCl precipitation. The final library stock was dissolved in PBS supplemented with 0.5% BSA, 0.05% Tween-20.

TABLE 6
Oligonucleotide primers used for the construction of the AS20 humanization
library. Sequences are formatted using the IUPAC nucleotide code

| | | SEQ ID |
|---|---|---|
| Primer name | NT sequence | NO: |
| SL048_hum_AS20_H1 | GATACACCTTCACCAGCTACWATATGCACTGGGTGCG | 164 |
| SL048_hum_AS20_H2 | GACAAGGGCTTGAGTGGATRGGARTAATCWACCCTRGTRRTGGTKMCACA | 165 |
| | AGCTACRMTCAGAAGTTCMAGGGCCGCGTCACC | |
| SL048_hum_AS20_L1 | CGTCACCATCACCTGCAGKGCAAGTYMGAGCATTAGCTATWTGMA | 166 |
| | TTGGTATCAGCAGAAAC | |
| SL048_hum_AS20_L2 | CCTAAGCTCCTGATCTATGMTRCATCCARSTTGSMAAGTGGGGTC | 167 |
| | CCATCAC | |
| SL048_hum_AS20_L3 | GATTTTGCAACTTATTACTGTCASCAGAGKTMTAGTWMTYHCACT | 168 |
| | TTTGGCCAGGGG | |

### Results

The genes encoding the AS20 CDR graft and AS20 humanization library scaffold were synthesized and cloned into the pHAT4 phagemid vector. The AS20 humanization library was constructed by the use of an optimized Kunkel procedure giving rise to 1.7×10¹⁰ of transformants. Sequencing of 96 randomly picked clones confirmed the introduction of the intended diversity (data not shown).

### Conclusion

The AS20 CDR graft and AS20 humanization library were successfully constructed. In both cases, IGHV1-46 and IGKV1-39 were used as human framework scaffold genes. The binding of the AS20 CDR graft to BSSL was assessed both in scFv (Example 6) and IgG format (Example 9 and 11). The AS20 humanization library was used for isolation of humanized BSSL binding scFv fragments by phage display and various binding screen assays (Example 6). Several of the selected clones showed binding with affinity and specificity equivalent to the parental IgG to the cognate target (human BSSL), and even better affinity to the mouse orthologue (Example 9). When analyzing the sequences of selected clones (Example 11) it is clear that there is an enrichment of specific residues in certain positions. Interestingly, in a few positions (e.g. VH: 62, 64 and VL: 115) there is a preferential selection for amino acids beyond the dual diversity, indicating that the introduction of additional diversity was a successful strategy.

### Example 6—Phage Display Selections on Human and Mouse BSSL and Subsequent Screening and Sequencing

In this Example, phage display selections were performed to enable isolation of scFv fragments specific for human and mouse BSSL.

### Material and Methods

### Antigens

During phage display selection, as target antigens, mouse BSSL and non-biotinylated and biotinylated human BSSL were used. More specifically, two variants with different degrees of biotinylation and coupling chemistry were made. These were the BSSL-b amine and the BSSL-b glyco. hBSSL, b-hBSSL, hBSSL-b amine and mBSSL (Table 2) were used as target for phage display selection in this Example.

### Phage Display Selections

For all antigens phage display was performed using four rounds of enrichment employing two in-house constructed human synthetic scFv phage libraries, SciLifeLib2 and AS20 humanization library (see Example 5). SciLifeLib2 is a naive human synthetic scFv library, similar in design and construction to previously reported [12]. The selection pressure was increased by gradually decreasing the antigen amount and by increasing the number and intensity of washes between the different rounds. For the two biotinylated samples of human BSSL, the selection was performed by immobilizing them on streptavidin-coated paramagnetic beads (Dynabeads M-280, ThermoFisher Scientific, #11206D), and most of the steps in the selection process were automated and performed with a Kingfisher Flex robot. The selection on native antigens was carried out by coating them on a 96-well plate (NUNC Maxisorp #442404). In some of the tracks, in order to preferentially select for cross-species reactive scFv, the antigen was alternated between human and mouse BSSL in the different rounds. Elution of phages was performed with trypsin or by competitive elution using mouse BSSL for selection on human BSSL and vice versa. The combination of these different parameters resulted in a scheme covering a total of five different selection tracks for ScilifeLib2 and nine for the AS20 Humanization library. Recovered phages were propagated in Top1OF′ E. coli, either on agar plates at 37° C. overnight (Round 1 and 2) or in solution at 30° C. overnight (Rounds 3 and 4). Phage stocks were made by infecting with an excess of M13K07 helper phage (New England Biolabs, #N0315S) and scFv expression induced by the addition of IPTG. The overnight cultures were PEG/NaCl-precipitated, resuspended in selection buffer and used for the next round of selection. Table 7 summarizes the phage display selection tracks.

TABLE 7
Phage display tracks

| | | | | | | Pre- |
|---|---|---|---|---|---|---|
| Track | Round 1 | Round 2 | Round 3 | Round 4 | Library | adsorption |
| 1 | 200 pmol | 50 pmol | 12.5 nMI | 2.5 nM | SciLifeLib | R1&R2 SAV |
| | hBSSL¹ | hBSSL¹ | hBSSL¹ | hBSSL¹ | 2 (scFv) | |
| 2 | 200 pmol | 50 pmol | 12.5 nM | 2.5 nM | | |
| | hBSSL² | hBSSL² | hBSSL² | hBSSL² | | |
| 3 | | 50 pmol | 12.5 nM | 2.5 nM | | |
| | | hBSSL^{2,4} | hBSSL^{2,4} | hBSSL^{2,4} | | |
| 4 | | mBSSL³ | 12.5 nM | mBSSL³ | | |
| | | | hBSSL² | | | |
| 5 | mBSSL³ | mBSSL³ | mBSSL³ | mBSSL³ | | |
| 6 | 200 pmol | 50 pmol | 12.5 nMI | 2.5 nM | AS20 | |
| | hBSSL¹ | hBSSL¹ | hBSSL¹ | hBSSL¹ | (scFv) | |
| 7 | 200 pmol | 50 pmol | 12.5 nM | 2.5 nM | | |
| | hBSSL² | hBSSL² | hBSSL² | hBSSL² | | |
| 8 | | 50 pmol | 12.5 nM | 2.5 nM | | |
| | | hBSSL^{2,4} | hBSSL^{2,4} | hBSSL^{2,4} | | |
| 9 | | mBSSL³ | 12.5 nM | mBSSL³ | | |
| | | | hBSSL² | | | |
| 10 | mBSSL³ | mBSSL³ | mBSSL³ | mBSSL³ | | |
| 11 | hBSSL³ | hBSSL³ | hBSSL³ | hBSSL³ | | |
| 12 | | hBSSL^{3,4} | hBSSL^{3,4} | hBSSL^{3,4} | | |
| 13 | | mBSSL^{3,4} | mBSSL^{3,4} | mBSSL^{3,4} | | |
| 14 | | mBSSL³ | mBSSL³ | hBSSL³ | | |

¹selections performed on biotinylated-BSSL (amine-coupled, 10X) coupled to magnetic SAV beads (M280)
²selections performed on biotinylated-BSSL (on carbohydrates, BH8520) coupled to magnetic SAV beads (M280)
³selections performed on native antigen coated onto the surface of immunotubes
⁴phage elution using competition by other species of BSSL (mouse or human) (trypsin will be used for elution in all other cases)

### Re-Cloning

To allow production of soluble scFv, phagemid DNA from the third and fourth round of each selection track was isolated. In pools, the genes encoding the scFv fragments were sub-cloned into a screening vector, providing a signal for secretion of the scFv along with a triple-FLAG tag and a hexahistidine (His) tag at the C-terminus. The constructs were subsequently transformed into TOP10 E. coli.

### Primary ELISA and Sequencing

For each selection track, between 89 and 222 colonies in total from round 3 and 4 were picked and cultured in 96-well plates and grown over-night. Expressed scFv (supernatants) were screened with ELISA for binding towards the native form of the respective selection target. The experiments in this screening process included the AS20 scFv that had previously been constructed, produced and characterized with respect to binding towards both human and mouse BSSL (see Example 2). Also the AS20 CDR graft as scFv was included (see Example 5). Clones considered positive in ELISA were subjected to DNA sequencing (GATC Biotech, Cologne, Germany).

### Secondary ELISA and HTRF

All sequence unique clones identified for each selection track were further analyzed in a second screening with ELISA. Here, the number of antigens were increased to include native human and mouse BSSL as well as biotinylated human BSSL. Streptavidin was used as a non-relevant antigen. The binding of all of the scFv was also assessed by HTRF (Homogeneous Time Resolved Fluorescence).

### Results

A total of 14 phage selection tracks were performed in parallel on the four forms of BSSL using SciLifeLib 2 and AS20 humanization library. Between 89 and 222 clones were picked and analyzed from each of the 14 tracks. ELISA and HTRF binding screens and sequencing resulted in a total of 68 unique scFv clones capable of binding to the orthologue of BSSL that they were selected for. Unexpectedly, no binding of the AS20 CDR graft scFv was detectable.

### Conclusion

A primary screen of a total of 2365 clones by ELISA resulted in a total of 467 scFv fragments with potential binding affinity for human and mouse BSSL being sent for sequencing. A secondary ELISA screen followed by HTRF and re-sequencing resulted in a total of 68 sequence unique scFv clones. The binding data of these suggests that their relative binding to human and mouse BSSL can be divided into three groups with the characteristics of recognizing either one, or both, of these orthologues.

64 of the isolates scFv originated in the AS20 humanization library, while only four of them originated in SciLifeLib2. This indicated that BSSL is a challenging target for phage display selections using a naive antibody library.

### Example 7—ELISA and Affinity Ranking by SPR of 68 Anti-BSSL scFvs

In this Example, the 68 unique scFvs generated in Example 6 were analyzed in ELISA and furthermore ranked based on affinity using SPR. Together with earlier binding data (ELISA and HTRF), the results were used as decision point for selecting candidates for further development.

### Material and Methods

hBSSL and mBSSL (Table 2) were used as BSSL reagents in this Example.

### ELISA

Human and mouse BSSL were coated at 4° C. overnight, 1 μμg/ml in PBS, into a 384-ELISA well plate. Two negative control proteins, streptavidin and BSA, were also included. FLAG-tagged scFv clones present in bacterial supernatant were diluted 1:2 and 1:20 in assay buffer (PBS +0.5% BSA+0.05% Tween20) and allowed to bind to coated proteins. All samples were assayed in duplicates. Detection of binding was enabled through an HRP-conjugated α-FLAG M2 antibody (Sigma-Aldrich #A8592) followed by incubation with TMB ELISA substrate (ThermoFisher Scientific #34029). The colorimetric-signal development was stopped by adding 1 M sulfuric acid and the plate was analyzed at 450 nm.

### SPR

The kinetic screen was performed on a BIACORE® T200 biosensor instrument (GE Healthcare). An α-FLAG M2 antibody (Sigma-Aldrich #F1804), functioning as a capture ligand, was immobilized onto all 4 surfaces of a CM5-S amine sensor chip according to manufacturer's recommendations.

FLAG-tagged scFv clones present in bacterial supernatant were injected and captured onto the chip surface, followed by injection of either human or mouse BSSL at 50 nM and 200 nM, respectively. The surface was regenerated with 10 mM glycin-HCl pH 2.2. All experiments were performed at 25° C. in running buffer (PBS+0.1% BSA +0.05% Tween20 pH 7.5 for human BSSL and 25 mM Tris-HCl+150 mM NaCl pH 7.5 for mouse BSSL).

### Results

### ELISA

Binding of 68 scFv-clones to directly coated human and mouse BSSL could be confirmed for the majority of clones. As observed in Example 6, BSSL-binding clones could be divided into three groups with the characteristics of recognizing either human BSSL, mouse BSSL, or both human and mouse BSSL. The majority of clones display preferential binding towards human BSSL (data not shown).

### SPR

Analysis of data was performed by visual inspection of the sensorgrams (not shown). When studying these, it was clear that the majority of clones had a considerable higher affinity, lower K_{D}-value (M), towards human BSSL than towards mouse BSSL. Inspection of the sensorgrams also showed that many of the humanized clones showed affinities in the same range as observed for the AS20 scFv. Examples of such clones are S-SL048-11 (comprising HCVR SEQ ID NO: 30 and LCVR SEQ ID NO: 31), S-SL048-14 (comprising HCVR SEQ ID NO: 50 and LCVR SEQ ID NO: 84), S-SL048-106 (comprising HCVR SEQ ID NO: 34 and LCVR SEQ ID NO: 35), S-SL048-108 (comprising HCVR SEQ ID NO: 72, LCVR SEQ ID NO: 106), S-SL048-109 (comprising HCVR SEQ ID NO: 73 and LCVR SEQ ID NO: 107), S-SL048-116 (comprising HCVR SEQ ID NO: 36 and LCVR SEQ ID NO: 37) and S-SL048-125 (comprising HCVR SEQ ID NO: 77, LCVR SEQ ID NO: 111). The affinities for mouse BSSL for these particular clones were also similar to what is seen for AS20.

A few clones derived from phage display selection tracks panned against mouse BSSL displayed preferential binding towards mouse BSSL than human BSSL, exemplified by clone S-SL048-66 (comprising HCVR SEQ ID NO: 61 and LCVR SEQ ID NO: 95)

### Conclusion

In this Example, the binding of the previously identified 68 scFv-clones to BSSL was confirmed by ELISA. A kinetic screen was also performed on all 68 clones, using a single concentration of human and mouse BSSL. As was found for AS20, the far majority of clones displayed a higher estimated binding affinity, defined as equilibrium of dissociation constant, K_{D} (M), towards human BSSL than towards mouse BSSL. Estimated affinities towards human BSSL was in the low nanomolar to nanomolar range, with reference AS20 scFv displaying a K_{D}-value of 2 nM. A small set of scFv clones displayed a higher affinity to mouse BSSL than to human BSSL, with the highest affinity corresponding to a K_{D}-value of 43 nM.

From all data collected, 38 clones (comprising HCVR SEQ ID NO: 30, 32, 34, 36 and 47-79 and LCVR SEQ ID NO: 31, 33, 35, 37, 38 and 81-113) and reference AS20 scFv (HCVR SEQ ID NO: 80 and LCVR SEQ ID NO: 114), were selected for conversion into human IgG4 S228P. All of candidate clones originate in the AS20 humanization library and none in SciLifeLib.

### Example 8—Conversion to hIgG4 S228P Format and Small-Scale Transient Expression of 38 Humanized BSSL-Specific Antibodies

In this Experiment, 38 of the most promising humanized scFv clones (comprising HCVR SEQ ID NO: 30, 32, 34, 36 and 47-79 and LCVR SEQ ID NO: 31, 33, 35, 37, 38 and 81-113) from the phage selection and subsequent binding screening in Example 7 were converted to the human IgG4 S228P antibody format. In addition, AS20 (parental clone) (comprising HCVR SEQ NO: 80 and LCVR SEQ ID NO: 114) and AS20 CDR graft (comprising HCVR SEQ NO: 144 and LCVR SEQ ID NO: 145) were similarly converted to IgG4 S228P.

Briefly, human IgG4 is considered to be the most Fc-silent natural IgG subclass in man, i.e., it does not mediate major effector functions via the Fc-part of the antibody. Similar to IgG1, IgG4 has a serum half-life of 21 days. However, IgG4 naturally tends to dissociate in vivo into half-IgG4 molecules and can then combine with other circulating IgG4 molecules. This half molecule exchange can be avoided by the introduction of a stabilizing mutation in the hinge region, namely S228P (Eu numbering; this is identical to Kabat numbering S241P) [13].

The genes encoding VH and VL of the 38 scFv clones, AS20 and AS20 CDR graft were successfully transferred into a vector encoding the human IgG4 S228P subclass. ExpiHEK293 cells were transiently transfected, antibodies expressed in small scale (4 ml) and protein A purified. The purity and integrity/monomeric content were analyzed in SDS-PAGE and analytical size exclusion chromatography (SEC).

### Material and Methods

Sequence analysis

The amino acid sequences of the HCVR and LCVR of scFv chosen for IgG conversion are presented in the sequence listing and for clarity in Table 8 together with anti-hapten (4-hydroxy-3-nitrophenyl acetyl, NP) antibody (anti-NP).

TABLE 8
Antibodies used in this Example

| | | | SEQ | SEQ |
|---|---|---|---|---|
| | | | ID NO: | ID NO: |
| | | | hIgG4 | hIgG4 |
| | SEQ | SEQ | S228 P | S228 P |
| | ID NO: | ID NO: | heavy | light |
| | HCVR | LCVR | chain (HC) | chain (LC) |
| S-SL048-10 | 47 | 81 | | |
| S-SL048-11 | 30 | 31 | 119 | 120 |
| S-SL048-12 | 48 | 82 | | |
| S-SL048-13 | 49 | 83 | | |
| S-SL048-14 | 50 | 84 | | |
| S-SL048-17 | 51 | 85 | | |
| S-SL048-18 | 52 | 86 | | |
| S-SL048-38 | 53 | 87 | | |
| S-SL048-40 | 54 | 88 | | |
| S-SL048-41 | 55 | 89 | | |
| S-SL048-43 | 56 | 90 | | |
| S-SL048-45 | 57 | 91 | | |
| S-SL048-46 | 32 | 33 | 121 | 122 |
| S-SL048-47 | 58 | 92 | | |
| S-SL048-48 | 59 | 93 | | |
| S-SL048-65 | 60 | 94 | | |
| S-SL048-66 | 61 | 95 | | |
| S-SL048-74 | 62 | 96 | | |
| S-SL048-75 | 63 | 97 | | |
| S-SL048-79 | 64 | 98 | | |
| S-SL048-81 | 65 | 99 | | |
| S-SL048-86 | 66 | 100 | | |
| S-SL048-89 | 67 | 101 | | |
| S-SL048-103 | 68 | 102 | | |
| S-SL048-104 | 69 | 103 | | |
| S-SL048-105 | 70 | 104 | | |
| S-SL048-106 | 34 | 35 | 123 | 124 |
| S-SL048-107 | 71 | 105 | | |
| S-SL048-108 | 72 | 106 | | |
| S-SL048-109 | 73 | 107 | | |
| S-SL048-110 | 74 | 108 | | |
| S-SL048-112 | 75 | 109 | | |
| S-SL048-115 | 76 | 110 | | |
| S-SL048-116 | 36 | 37 | 125 | 126 |
| S-SL048-118 | 36 | 38 | 127 | 128 |
| S-SL048-125 | 77 | 111 | | |
| S-SL048-131 | 78 | 112 | | |
| S-SL048-134 | 79 | 113 | | |
| AS20 | 80 | 114 | 129 | 130 |
| AS20 CDRgraft | 144 | 145 | 131 | 132 |
| anti-NP | | | 133 | 134 |

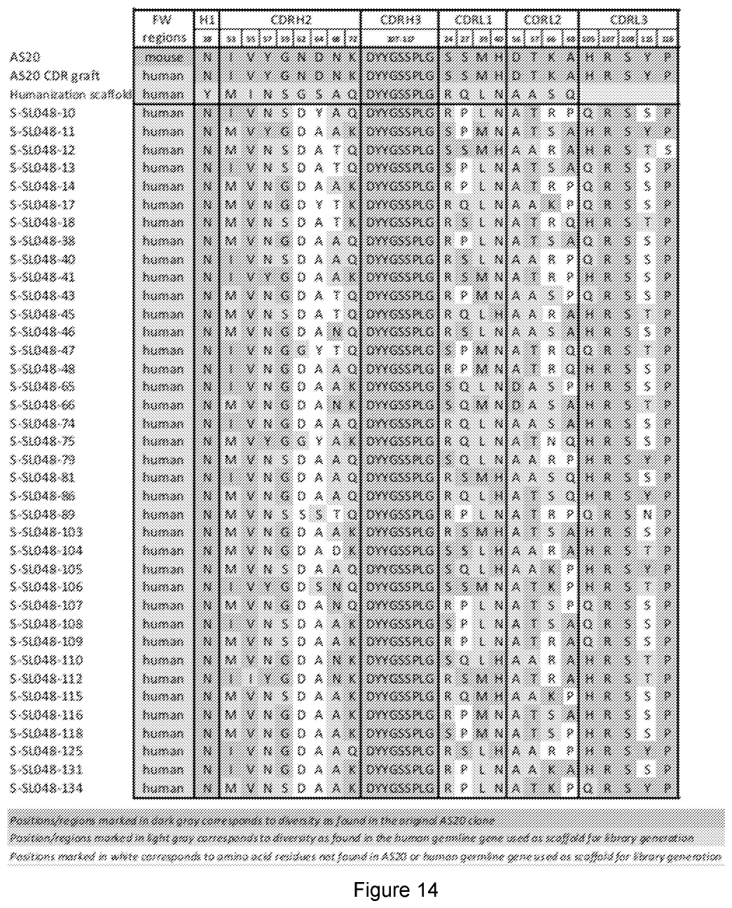

FIG. 14 illustrates sequence differences between the 38 humanized clones converted to hIgG4 S228P. In the AS20 humanization library, a total of 20 positions were targeted for diversification in CDR1 and CDR2 of the heavy chain and CDR1, CDR2 and CDR3 of the light chain. For comparison, AS20 and the CDR graft construct are included in the figure.

In-Fusion cloning

Plasmid DNA of the 38 BSSL-specific scFv and AS20 was purified from bacterial culture by a standard miniprep procedure. The gene for the AS20 CDR graft was synthesized by Genscript. The VH and VL regions were PCR amplified and inserted into in house constructed vector pHAT-hIgG4-S241P using the In-Fusion HD Plus Cloning Kit (Clontech #638909). One representative example of a resulting full-length IgG sequence is that of S-SL048-11 hIgG4 S228P heavy Chain (VH-CH1-hinge-CH2-CH3) corresponding to SEQ ID NO: 119 and S-SL048-11 hIgG4 S228P light Chain (VL-CL) corresponding to SEQ ID NO: 120.

Transfection into HEK293, Expression and Purification

Transfection of plasmid DNA into expiHEK293 cells was performed using an ExpiFectamine™ 293 Transfection Kit (ThermoFisher Scientific #A14525) in 4 ml cultures in a 24 deep well plate. After 5 days of cultivation at 37° C., 6% CO₂, 80% rH and 400 rpm, the media supernatant was mixed with Protein A conjugated magnetic beads and purified on a KingFisher Flex instrument. Immediately following elution in 0.1 M Glycine, pH 2.7, neutralization was performed by addition of 1 M Tris-HCl, pH 8.8, and a buffer exchange to PBS was performed by the use of a 96 well spin desalting plate. SDS-PAGE was performed to determine purity and integrity of the purified IgG and concentrations determined using an Implen NP80 UV-Vis Spectrophotometer (Fisher Scientific).

### Results

### In-Fusion Cloning

The 38 unique scFvs, AS20 and AS20 CDR graft were successfully converted to full-length human IgG4 antibodies, as confirmed by sequencing.

Transfection into HEK293, Expression and Purification

The antibodies were expressed in expiHEK293 cells and purified from the supernatant by Protein A purification. The purity and integrity of the purified IgG was confirmed by SDS-PAGE (data not shown).

### Conclusion

All BSSL-binding antibodies were successfully re-cloned to hIgG4 format, expressed in HEK293 cells and purified by Protein A-conjugated magnetic beads on a Kingfisher Flex instrument. All demonstrated acceptable level of purity, as evaluated by SDS-PAGE.

### Example 9—Binding of 38 hIgG4 S228P Clones to Human and Mouse BSSL

This Example describes the target binding analysis of the 38 hIgG4 S228P clones (Example 8, Table 8) by surface plasmon resonance (SPR), which was performed in order to verify that binding to human and mouse BSSL was retained after conversion from scFv to IgG format.

### Material and Methods

Kinetic parameters of the hIgG4 S228P clones were determined by SPR using BIACORE® T200 (GE Healthcare). Single cycle kinetics was used to measure the affinity of the purified hIgG4 molecules to human and mouse BSSL. An anti-Fab antibody (GE Healthcare, #28958325) was immobilized on a CM5 S sensor chip by primary amine coupling using NHS-EDC chemistry. hIgG4 was captured by the anti-Fab antibody, and subsequently, five different concentrations of hBSSL (1:5 dilutions starting from 50 nM) or mBSSL (1:5 dilutions starting from 500 nM) were injected over the surface. The sensor chip surface was regenerated with 10 mM glycine-HCl pH 2.1. The BSSL reagents as listed in Table 2 were used. For binding to human BSSL, single cycle kinetic data was fitted to a 1:1 binding model and kinetic parameters were retrieved using software BlAevaluation. For mouse BSSL, a steady state analysis was performed by plotting the response level at equilibrium against each concentration, and K_{D} values were retrieved by the BlAevalution software.

### Results

A single cycle kinetic approach was used to determine the affinity of the converted antibodies to non-biotinylated human BSSL and mouse BSSL. The obtained equilibrium dissociation constants (K_{D}) are listed in Table 9. For hBSSL, the data was, in general, successfully fitted to a 1:1 binding model. For mouse BSSL, the data did not always fit the 1:1 binding model very well. Instead the data was analyzed using steady state analysis. The determined K_{D} values for binding to human as well as murine BSSL were in the same range as the K_{D} values determined for the same clones in scFv format (see Example 7). For the AS20 CDR graft clone, a low degree of binding to human BSSL was observed (data not shown). However, the model fitting was not considered accurate and therefore a K_{D} value was not retrieved for this particular clone.

TABLE 9
Measured equilibrium dissociation constants (K_{D}) In a few
cases, the quality of the data was considered too low
to be reliable, denoted here as “n.d.” (not determined)

| | K_{D} (nM), hBSSL | K_{D} (nM), mBSSL |
|---|---|---|
| S-SL048-10 | 2.2 | 57 |
| S-SL048-11 | 0.7 | 24 |
| S-SL048-12 | 6.4 | 57 |
| S-SL048-13 | 0.9 | 57 |
| S-SL048-14 | 0.9 | 89 |
| S-SL048-17 | 4.3 | 68 |
| S-SL048-18 | 2.3 | 68 |
| S-SL048-38 | 1.0 | 67 |
| S-SL048-40 | 1.0 | 87 |
| S-SL048-41 | 1.2 | 79 |
| S-SL048-43 | 1.2 | 137 |
| S-SL048-45 | 9.6 | 82 |
| S-SL048-46 | 3.7 | 24 |
| S-SL048-47 | 8.5 | n.d. |
| S-SL048-48 | 0.8 | 41 |
| S-SL048-65 | n.d. | 30 |
| S-SL048-66 | 8.0 | 83 |
| S-SL048-74 | 4.8 | 21 |
| S-SL048-75 | n.d | 164 |
| S-SL048-79 | 5.0 | 48 |
| S-SL048-81 | 4.4 | 60 |
| S-SL048-86 | 6.8 | 167 |
| S-SL048-89 | 6.0 | n.d. |
| S-SL048-103 | 1.7 | 199 |
| S-SL048-104 | 3.3 | 111 |
| S-SL048-105 | 6.0 | 237 |
| S-SL048-106 | 1.2 | 111 |
| S-SL048-107 | 1.2 | 98 |
| S-SL048-108 | 1.3 | 149 |
| S-SL048-109 | 1.4 | 353 |
| S-SL048-110 | 5.9 | 168 |
| S-SL048-112 | 5.0 | n.d. |
| S-SL048-115 | 6.0 | 186 |
| S-SL048-116 | 0.6 | 52 |
| S-SL048-118 | 0.9 | 95 |
| S-SL048-125 | 2.1 | 123 |
| S-SL048-131 | 2.7 | 63 |
| S-SL048-134 | 1.8 | n.d. |
| AS20 | 0.6 | 161 |
| AS20 CDRgraft | n.d. | n.d. |

### Conclusion

Overall, the results indicate that the binding affinity of the antibodies towards human and murine BSSL was not affected by the re-cloning into hIgG4 format. However, the AS20 CDR graft behaved differently. As shown in Example 6, the AS20 CDR graft did not show any binding to BSSL when expressed in the scFv format. However, in the IgG format a binding signal was observed to human BSSL.

### Example 10—Functional Test of 28 hIgG4 S228P Clones using Flow Cytometry Displacement Assay

In this Example, the 28 hIgG4 S228P antibodies with highest binding affinity towards human and/or mouse BSSL in Example 9 (comprising HCVR SEQ ID NO: 30, 32, 34, 36, 47, 50-56, 59-65, 68, 69, 71-73, 75, 77 and 78 and LCVR SEQ ID NO:31, 33, 35, 37, 38, 81, 84-90, 93-99, 102, 103,105-107, 109, 111 and 112) were tested for their capacity to block binding of human BSSL to CD14+ monocytes using a flow cytometry-based displacement assay. Five antibodies were included as references; AS20 mlgG1 (HC SEQ ID NO: 135 and LC SEQ ID NO: 136), AS20 hIgG4 (HC SEQ ID NO: 129 and LC SEQ ID NO: 130), AS20 CDR graft (HC SEQ ID NO: 131 and LC SEQ ID NO: 132), anti-human alpha-synuclein mlgG1 and anti-NP hIgG4 (HC SEQ ID NO: 133 and LC SEQ ID NO: 134).

### Material and Methods

### Preparation of Buffy Coat

Human blood was drawn from one single healthy donor in vacutainer tubes supplemented with citrate anti-coagulant (BD Vacutainer). The buffy coat, consisting of white blood cells and platelets, was isolated after centrifugation at 1300×g for 10 min. at room temperature in a swing-out bucket rotor.

### Flow Cytometry Displacement Assay

The 28 BSSL-specific hIgG4 antibodies and the five reference antibodies at different concentrations (ranging from 0.5 μg to 3.0 μg per reaction; see Table 10) were added to b-hBSSL (1 μg per reaction, see Table 2) in round-bottom polystyrene tubes, lx PBS (pH 7.4) was added to a final volume of 20 μl and the antibody/b-hBSSL mixtures were incubated for 30 min. at +4° C. to facilitate binding of antibodies to BSSL. Buffy coat (50 μl) was then added to each antibody/b-hBSSL mixture and incubation continued for another 30 min at +4° C. Thereafter, 2 ml of FACS lysing solution (BD Biosciences) was added and the cells were incubated for 10 min at room temperature in order to lyse erythrocytes and fix the white blood cells. The cells were then centrifuged for 5 min. at 200×g, the resulting pellets were washed by adding 2 ml of FACS buffer (1×PBS supplemented with 1% FCS and 0.1% NaN3) and centrifuged again. Finally, the supernatants were discarded and the cells were resuspended in the last drop of liquid, approximately 50 μl.

5 μl of BV421-labelled anti-human CD14 (BD Biosciences) and 5 μl of BB515-labelled streptavidin (BD Biosciences) was added to each tube and incubated for 30 min, at +4° C., protected from light. Thereafter, cells were washed twice with FACS buffer, resuspended in 500 μl FACS buffer, and run on BD LSRII flow cytometer (BD Biosciences). Finally, data were analyzed using FlowJo software (BD Biosciences).

Results

The CD14+cells were first gated out to delineate the monocyte population. Then, binding of b-hBSSL to gated CD14+monocytes was detected by BB515-labelled streptavidin and quantified as median fluorescence intensity (MFI) in the BB515 channel. The capacity of BSSL-specific hIgG4 and reference antibodies to displace binding of b-hBSSL to CD14+monocytes was quantified as a reduction in BB515 MFI in monocytes following incubation with increased concentrations of BSSL-specific hIgG4 or reference antibodies.

TABLE 10
The 28 BSSL-specific hIgG4 S228P and reference antibodies capacity
to block BSSL binding to human CD14⁺ monocytes. Note
that not all antibodies were tested at all concentrations.

| | Capacity to displace binding of | | | | |
|---|---|---|---|---|---|
| | b-hBSSL to monocytes (percentage | | | | |
| | reduction relative to binding | | | | |
| | without competing antibody) | | | | |
| Antibody | 0.5 μg | 1.0 μg | 1.5 μg | 2.0 μg | 3.0 μg |
| S-SL048-10 | | | 34 | | 42 |
| S-SL048-11 | 1 | 9 | 60 | | 68 |
| S-SL048-14 | 11 | 32 | 48 | | 56 |
| S-SL048-17 | | | 16 | | 36 |
| S-SL048-18 | | | 58 | | 61 |
| S-SL048-38 | 6 | 44 | 52 | | 51 |
| S-SL048-40 | 20 | 28 | 44 | | 53 |
| S-SL048-41 | 12 | 29 | 51 | 71 | 72 |
| S-SL048-43 | | | 37 | | 55 |
| S-SL048-46 | | | 43 | | 61 |
| S-SL048-48 | 28 | 23 | 50 | 61 | 68 |
| S-SL048-65 | 5 | 23 | 21 | 35 | 39 |
| S-SL048-66 | 12 | 12 | 39 | 43 | 51 |
| S-SL048-74 | | | 29 | | 52 |
| S-SL048-75 | | | 3 | | 0 |
| S-SL048-79 | | | 20 | | 52 |
| S-SL048-81 | | | 31 | | 62 |
| S-SL048-103 | 12 | −1 | 26 | | 69 |
| S-SL048-104 | | | 2 | | 44 |
| S-SL048-106 | | | 52 | | 61 |
| S-SL048-107 | | | 37 | | 49 |
| S-SL048-108 | | | 41 | | 48 |
| S-SL048-109 | | | 38 | | 51 |
| S-SL048-112 | | | 28 | | 54 |
| S-SL048-116 | | | 58 | | 64 |
| S-SL048-118 | | | 23 | | 58 |
| S-SL048-125 | | | 9 | | 33 |
| S-SL048-131 | | | 31 | | 58 |
| AS20 CDRgraft | 8 | −13 | | −8 | |
| AS20 IgG4 | 19 | 21 | | 58 | |
| Anti-NP hIgG4 | −5 | 0 | −2 | 4 | 3 |
| AS20 mIgG1 | 31 | 66 | 72 | 74 | 80 |
| α-synuclein mIgG1 | −8 | −2 | −4 | −1 | −7 |

Conclusion

The molecular mass of hBSSL (76 kD) is approximately half of the IgG molecule (150 kD). Hence, in this example 1 μg of BSSL and 2 μg of IgG corresponded roughly to a 1:1 molar ratio. With the highest antibody concentration used (3 μg per reaction) we showed that 9 out of the 28

BSSL-specific hIgG4 S228P antibodies inhibited (displaced) at least 60% of BSSL (1 μg per reaction) from binding to monocytes. The most effective antibody to displace binding was AS20 mlgG1, whereas the AS20 CDR graft hIgG4, anti-NP hIgG4 and anti-a-synuclein mlgG1 did not influence binding at all.

### Example 11—Production of 5 Prenominated hIgG4 S228P Antibodies and Controls

Based on results obtained from binding assays performed in Example 9 and in vitro functional studies in Example 10 and sequence content, five humanized hIgG4 S228P clones were chosen for larger scale production (10 mg), namely S-SL048-11, S-SL048-46, S-SL048-106, S-SL048-116 and S-SL048-118.

Also, two controls were included; AS20, and the AS20 CDR graft clone. In addition, an isotype control was included. For this purpose, the anti-hapten (4-hydroxy-3-nitrophenyl acetyl, NP) antibody (clone B1-8) was ordered from Absolute Antibody. The chosen subclass format hIgG4 S228P is the same as previously used for assessment of 38 BSSL specific clones in Example 8.

### Material and Methods

Production of Antibodies

Production of material was outsourced to Absolute Antibody (Oxford, UK). Sequence information of VH and VL of seven of the clones was sent to the company, where the genes were synthesized and cloned into vectors encoding the human IgG4-S228P subclass. The antibodies were transiently expressed in mammalian HEK293 cells and subsequently purified by affinity chromatography using protein A. The purity and integrity were assessed by SDS-PAGE and endotoxin levels determined by LAL chromogenic endotoxin assay.

The amino acid sequences of the eight antibodies correspond to SEQ ID NOs as shown in Table 11.

TABLE 11
Antibodies used in this Example

| Antibody name | HC SEQ ID NO: | LC SEQ ID NO: |
|---|---|---|
| S-SL048-11 hIgG4 S228P | 119 | 120 |
| S-SL048-46 hIgG4 S228P | 121 | 122 |
| S-SL048-106 hIgG4 S228P | 123 | 124 |
| S-SL048-116 hIgG4 S228P | 125 | 126 |
| S-SL048-118 hIgG4 S228P | 127 | 128 |
| AS20 hIgG4 S228P | 129 | 130 |
| AS20 CDR graft hIgG4 S228P | 131 | 132 |
| Anti-NP hIgG4 S228P | 133 | 134 |

### Surface Plasmon Resonance (SPR)

Kinetic parameters of the hIgG4 S228P clones were determined by SPR using BIACORE® T200 (GE Healthcare). An anti-Fab antibody (GE #28958325) was immobilized on a CM5 S sensor chip by primary amine coupling using NHS-EDC chemistry. The hIgG4 antibodies were captured by the anti-Fab antibody, and subsequently, five different concentrations of hBSSL (1:5 dilutions, 0.08-50 nM) or mBSSL (1:2 dilutions, 50-800 nM) were injected over the surface. The sensor chip surface was regenerated with 10 mM glycine-HCl pH 2.1. For binding to human BSSL, single cycle kinetic data was fitted to a 1:1 binding model and kinetic parameters were retrieved using software BlAevaluation. For mouse BSSL, a steady state analysis was performed by plotting the response level at equilibrium against each concentration, and K_{D} values were retrieved by the BlAevalution software.

### Results

### Production of Antibodies

Ten milligrams at a concentration of 4.6-5 mg/ml of the eight antibodies in 25 mM histidine, 150 mM NaCl, 0.02% P80 (pH6.0) was received from Absolute Antibody. The purity was determined by SDS-PAGE to >98% and endotoxin levels determined by LAL chromogenic endotoxin assay to <0.05 EU/mg. The monomeric content of each of the clones were determined by analytical size exclusion chromatography to >98%.

### SPR

SPR was used to determine the affinity of antibodies to human and mouse BSSL. Table 12 summarizes the different K_{D} values of the prenominated clones for hBSSL and mBSSL binding. For comparison, the K_{D} values determined for the same clones produced in-house are also included (these experiments are described in Example 9).

TABLE 12
Summary of results of SPR analyses of antibodies in hIgG4
S228P format. *These values were obtained from experiments
reported in Example 9. Numbers marked with # are
considered unreliable and should be viewed with care. In
some cases, the quality of the data was considered too
low to be reliable, denoted here as “n.d.” (not determined).

| | |
|---|---|
| In-house | Absolute Antibody |
| production batch* | protein batch |

| | hBSSL | mBSSL | hBSSL | mBSSL |
|---|---|---|---|---|
| | K_{D} | K_{D} | K_{D} | K_{D} |
| Clone | (nM) | (nM) | (nM) | (nM) |
| S-SL048-11 | 0.7 | 24^{#} | 0.6 | 65 |
| S-SL048-46 | 3.7 | 24 | 3.8 | 55 |
| S-SL048-106 | 1.2 | 111^{#} | 1.2 | >500 |
| S-SL048-116 | 0.6 | 52 | 0.5 | 40 |
| S-SL048-118 | 0.9 | 95 | 0.5 | 70 |
| AS20 | 0.6 | 161 | 0.6 | 223 |
| CDRgraft | n.d. | n.d. | 61 | n.d. |

### Conclusion

Five prenominated candidates and three controls produced by Absolute Antibody were analyzed in ELISA (data not shown) and SPR for BSSL binding. The results showed that the binding to BSSL was, in general, very similar to that observed for the respective clones produced in-house in the same IgG format (see Example 9). The results also showed that the isotype control anti-NP hIgG4 S228P did not bind to BSSL, and should therefore be suitable to use as a negative control in future analyses. The functionality of these antibody batches was also assessed in the displacement assay (Example 10). Very similar results were obtained for the different clones as reported in Example 10 (data not shown).

In this Example we could for the first time measure the affinity of the CDR-graft. Here, the AS20 CDR graft hIgG4 S228P (comprising HC SEQ ID NO: 131 and LC SEQ ID NO: 132) exhibited a clear binding to hBSSL at higher concentrations, although the binding was greatly reduced compared to AS20 hslgG S228P (comprising HC SEQ ID NO: 129 and LC SEQ ID NO: 130). The reduction in affinity as measured here was around a 100-fold for hBSSL, and for mBSSL the affinity was too low to be determined. Thus, the affinity for the target was significantly reduced when the CDR was grafted onto a new framework. The observed stronger BSSL binding of the AS20 CDR graft hIgG4 S228P compared to the scFv AS20 CDR graft (comprising HCVR SEQ ID NO: 144 and LCVR SEQ ID NO: 145, see Example 6) might be due to the different antibody formats.

### Example 12—Stability Studies of Five Candidate Antibodies

This Example describes the stability study of S-SL048-11, S-SL048-46, S-SL048-106, S-SL048-116 and S-SL048-118 in hIgG4 S228P format in order to investigate the biophysical stability of the antibodies. For comparison, AS20 both as hIgG4 S228P and hIgG1 LALA-PG (see Example 17) were included. Also, the AS20 CDR graft hIgG4 S228P and the anti-NP hIgG1 LALA-PG isotype control were included. Analysis was performed by SDS-PAGE, analytical SEC, nano-DSF, and DLS.

### Materials and Methods

The nine different antibodies included in the stability study and their corresponding SEQ ID NOs are listed in Table 13. The antibodies were aliquoted into vials and incubated at −80° C., +4° C. and +40° C. at 5 mg/ml in 25 mM Histidine, 150 mM NaCl, 0.02% P80, pH 6.0. Samples were withdrawn and analyzed according the schedule in Table 14 where day 0 is the start day.

TABLE 13
Antibodies used in this Example

| Antibody name | HC SEQ ID NO: | LC SEQ ID NO: |
|---|---|---|
| S-SL048-11 hIgG4 S228P | 119 | 120 |
| S-SL048-46 hIgG4 S228P | 121 | 122 |
| S-SL048-106 hIgG4 S228P | 123 | 124 |
| S-SL048-116 hIgG4 S228P | 125 | 126 |
| S-SL048-118 hIgG4 S228P | 127 | 128 |
| AS20 hIgG4 S228P | 129 | 130 |
| AS20 CDR graft hIgG4 S228P | 131 | 132 |
| AS20 hlgG1 LALA-PG | 115 | 116 |
| Anti-NP hlgG1 LALA-PG | 117 | 118 |

TABLE 14
Schedule of sample analysis

| | Temp | Day | | | | | | |
|---|---|---|---|---|---|---|---|---|
| Analysis | (° C.) | 0 | 1 | 2 | 9 | 16 | 22 | 30 |
| SEC HPLC | −80 | | X | | | | | X |
| | +4 | X | X | X | X | X | X | X |
| | +80 | | X | X | X | X | X | X |
| SDS PAGE | −80 | | | | X | | | X |
| | +4 | X | | | X | | | X |
| | +80 | | | | X | | | X |
| DSF | −80 | | | | | | | X |
| | +4 | X | X | X | X | X | X | X |
| | +80 | | X | X | X | X | X | X |
| DLS | −80 | | | | | | | X |
| | +4 | | | | | | | X |
| | +80 | | | | | | | X |

Analytical size exclusion chromatography (SEC) was performed using a BioSEC column (300A, 7.8×300 mm; πP.N. 5190-2511, Agillent) connected to an Agilent 1100 system and using a running buffer of 0.15 M sodium phosphate (NaₓH_{y}PO₄) pH 6.8 at a flow rate of 1 ml/min. Proteins were detected by measuring the absorbance at A₂₈₀ and A₂₂₀. 20 μg of each sample was loaded on to the column. SDS-PAGE was performed using NuPAGE 4-12% Bis-Tris gels (InVitrogen NP0321BOX) using NuPAGE MES SDS Running buffer (InVItrogen NP000202) according to the manufacturer's instructions. Samples were either run under reducing or non-reducing conditions. Bands were visualized using SimplyBlue stain (Invitrogen LC6065) and bands quantified using a densitometric scanner (Oddyssey, Li-cor). 5 μg of each sample was loaded per well.

Nano-DSF (differential scanning fluorimetry) was performed using a Prometheus instrument (NanoTemper) applying a temperature gradient from 20-95° C. with a ramping of 1 degree/minute. The fluorescence emission at 300 nm and 350 nm was recorded as well as the back-scatter signal. 10 μL of sample with the concentration of 5 mg/ml was loaded for each sample. Data was analyzed using the PR.Stability analysis v1.02 from Nanotemper.

Dynamic light scattering was performed using a Zetasizer Pro (Malvern). The scattered light was recorded and analyzed using ZS Explorer software v 1.0.0.436 and the built-in algorithm. Samples were analyzed at 5 mg/ml.

Results

Size exclusion Chromatography (SEC)

Chromatograms were obtained for each sample according to schedule presented in Table 14. It was noted that the additional peaks appearing over time were of lower molecular weight suggesting a degradation of the material. Plotting the total integrated area over time indicated that no material was lost in the prefilter or on the column.

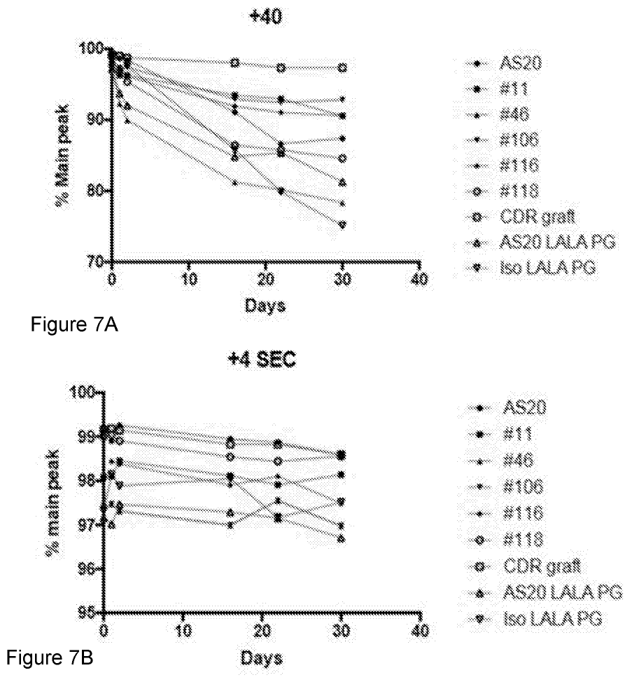

The size exclusion chromatography data is presented in FIG. 7. Looking at the percentage of the total area made up of the main peak only small changes could be observed at +4° C. At +40° C. more pronounced effects could be seen.

The highest reduction was observed for S-SL048-46 hIgG4 S228P and anti-NP hIgG1 LALA-PG. S-SL048-118 hIgG4 S228P, AS20 in hIgG4 S228P and AS20 hIgG1 LALA-PG showed intermediate reduction while the CDR graft showed virtually no reduction.

### DS-PAGE

Samples were analyzed under reducing and non-reducing conditions points as outlined in Table 14. The -80° C. samples showed no additional bands except for S-SL048-46 hIgG4 S228P and anti-NP hIgG1 LALA-PG, which showed one weak band each constituting less than 1% under reduced conditions. This was close to identical to what was observed for the 0 day samples. In fact the 0 day sample showed additional bands not seen in the −80° C. analyzed on day 30 reflecting the variability of the analysis. The non-reduced samples were close to identical to the 0 day samples. At +4° C. no additional significant band could be seen at day 9 or day 30 under reducing or non-reducing conditions. At +40° C. all samples showed additional bands appearing at day 9 and day 30. Under reducing condition all candidates showed lower molecular weight bands i.e., lower than 25 kD whereas AS20 hIgG4 S228P, AS20 hIgG1 LALA-PG and anti-NP hIgG1 LALA-PG all displayed higher Mw bands (i.e., larger than 50 kDa at day 9 and at day 30). In both cases these bands became more pronounced over time. Under non-reducing conditions clear differences could be seen with additional bands appearing in all samples. The results suggest that candidates S-SL048-46 and S-SL048-106 may be more prone to degradation.

### Nano-DSF

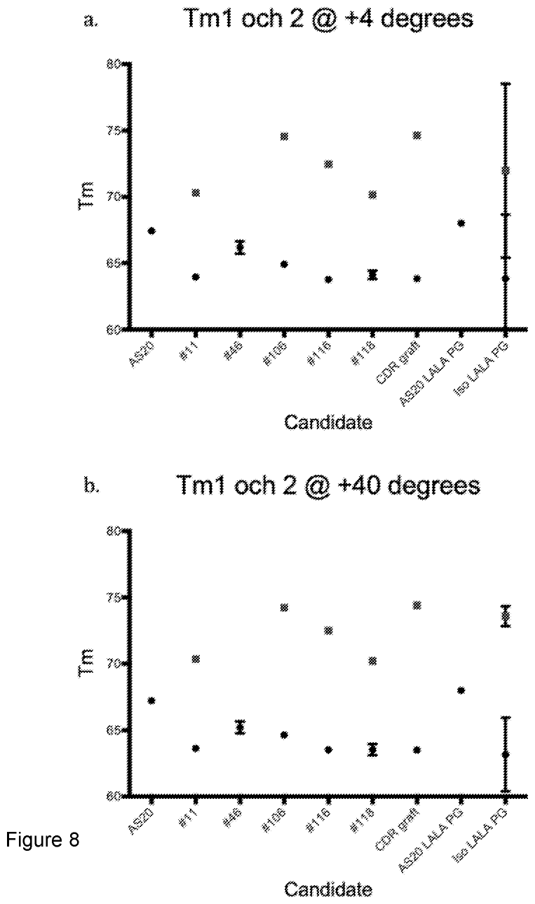

Samples were analyzed according to the schedule in Table 14 and the results are presented in FIG. 8. The main observation that could be made from the nano-DSF data was the changes occurring in the AS20 hIgG4 S228P, AS20 hIgG1 LALA-PG format and anti-NP hIgG1 LALA-PG, where major melting temperature Tₘ shifts were visible upon storage at +40° C.

For the candidates the main observation was the reduced amplitude of the second Tₘ (data not shown), which may reflect the main contribution from the Fab part of the antibody. Candidates S-SL048-46 and S-SL048-106 showed a slightly larger change and S-SL048-11, S-SL048-116 and 5-SL048-118 showed similar but slightly smaller effects. These differences are considered minor for all the candidates. The CDR graft appeared to be the most stable of the samples tested.

### Dynamic Light Scattering (DLS)

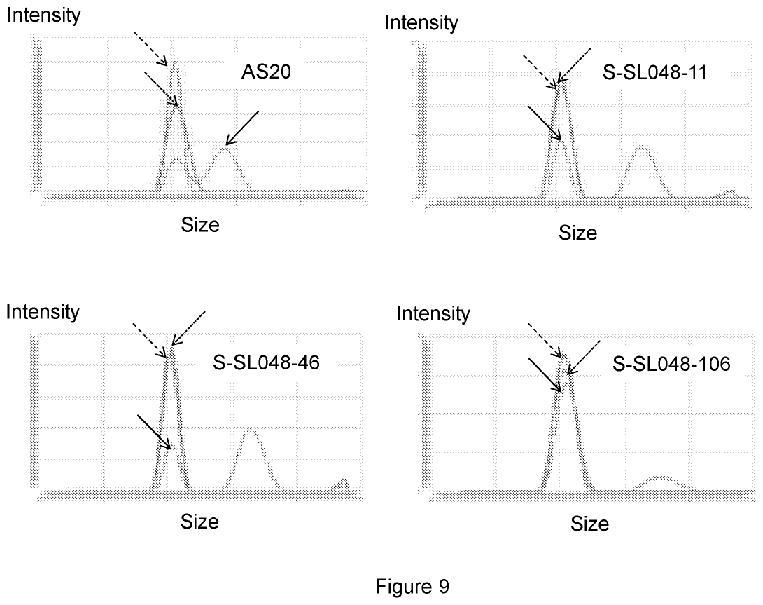

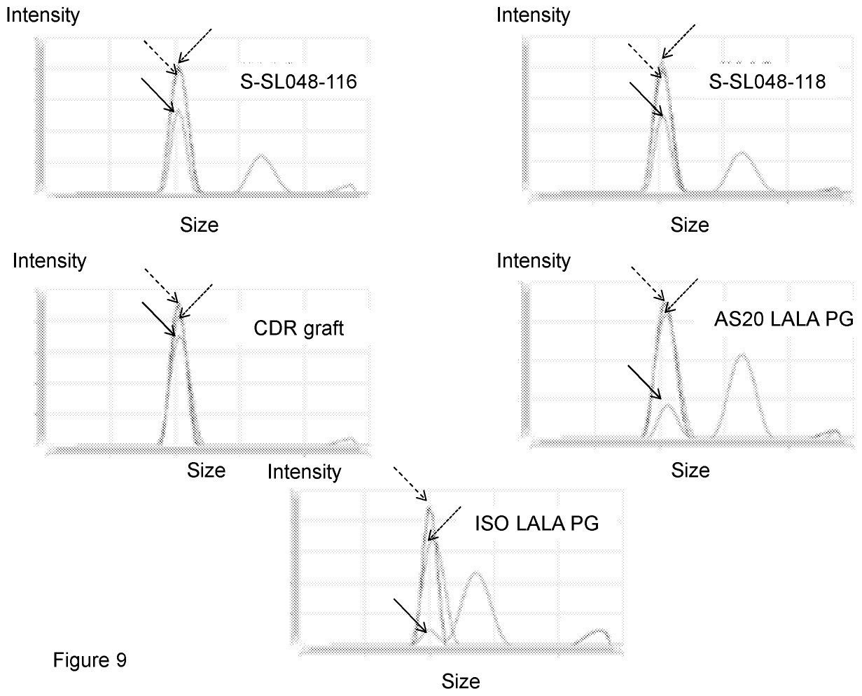

The samples were analyzed by DLS at day 30. The results at the three different temperatures for each sample are shown in FIGS. 9. At −80 and +4 ° C. all sample displays one peak with apparent diameters of around 10-11 nm in line with expected results for monomeric antibodies. No larger particles could be observed in any of the samples i.e., only protein molecules could be detected.

At +40° C. larger particles are clearly seen at varying degrees. Using the Z average mean and polydispersity numbers as indicators of size homogeneity (data not shown) S-SL048-106 exhibited the lowest amount of higher Mw particles. In contrast, AS20 hIgG1 LALA-PG and anti-NP hIgG1 LALA-PG showed the highest number of higher Mw particles indicating that these two molecules were more prone to aggregation.

### Conclusion

In conclusion, combining the data to an overall ranking the most stable candidates in hIgG4 S228P format appear to be: S-SL048-106, S-SL048-118 and S-SL048-116 in that order.

### Example 13—Epitope Mapping of AS20 by HDX-MS

In this Example, hydrogen deuterium exchange mass spectrometry (HDX-MS) was performed on the chimeric AS20 IgG4 antibody together with human BSSL in order to map its epitope.

### Methods and Materials

40 μL of a 2 mg/mL human BSSL (SEQ ID NO: 138) solution in PBS was mixed with 92.5 μL of 1.7 mg/ml chimeric AS20 hIgG4 antibody (comprising HC SEQ ID NO: 139 and LC SEQ ID NO: 140) for a 1:1 molar ratio. The BSSL/antibody complex was concentrated to 40 μL using a 10K Centrifugal filter units (Amicon Ultra, Merck). In parallel, a sample containing BSSL only, without the addition of antibody, was prepared analogously. The samples were analyzed in an automated HDX-MS system (CTC PAL/Biomotif HDX) in which samples were automatically labeled, quenched, digested, cleaned and separated at 2° C. More specifically, samples were labeled by mixing 3 μL of BSSL (or BSSL/antibody complex) with 22 μL of deuterated PBS and incubated at 4° C. for four labeling time points: 5 min, 30 min, 90 min and 180 min. The labelling reaction was stopped/quenched by decreasing the pH to ˜2.3 and temperature to ˜4° C. through the addition of 20 μL of a solution containing 6 M Urea, 100 mM TCEP and 0.5% TFA. Samples were digested using an immobilized pepsin column (2.1 column (2.1×30 mm) at 250 μl/min, followed by an on-line desalting step using a 2 mm I.D×10 mm length C-18 pre-column (ACE HPLC Columns, Aberdeen, UK) using 0.05% TFA at 350 μl/min for 3 min. Peptic peptides were then separated by a 18 min 8-55% linear gradient of ACN in 0.1% formic acid using a 2 mm I.D x 50 mm length HALO C18/1.8 μm analytical column operated at 95 μL/min. An Orbitrap Q Exactive mass spectrometer (Thermo Fisher Scientific) operated at 70,000 resolution at m/z 400 was used for analysis. The software Mascot was used for peptide identification and HDExaminer (Sierra Analytics, USA) was used to process all HDX-MS data. Statistical analysis was done using a 95% confidence interval.

### Results

The deuteration kinetics of 66 peptides was followed by HDX-MS covering 57.9% of the protein. Deuterium labeling (5, 30, 90 and 180 min) and differential deuteration uptake kinetics between the BSSL alone and in the presence of AS20 were calculated. Several peptides close to the N-terminus showed statistically lower deuterium uptake in the presence of AS20 antibody, clearly mapping the epitope to this region. By considering overlapping peptides the spatial resolution could be reduced, resulting in the three peptide regions shown in Table 15.

TABLE 15
A list of the AS20 epitope regions suggested
by the HDX-MS experiment

| Epitope | Start-End | | SEQ ID |
|---|---|---|---|
| regions | amino acid (aa) | Sequence | NO: |
| 1 | aa 7-12 | YTEGGF | 1 |
| 2 | aa 42-55 | LENPQPHPGWQGTL | 2 |
| 3 | aa 172-180 | AWVKRNIAA | 13 |

### Conclusion

Taken together, the AS20 epitope was successfully mapped to the N-terminal region of BSSL. A cluster of three discontinuous peptide regions was identified with significantly lower deuterium uptake in presence of AS20. These regions map together in the three-dimensional structure of BSSL, indicating that these peptides together makes up a conformational epitope of AS20. In Example 21 we describe the crystal structure of the AS20 Fab fragment in complex with hBSSL. The analysis of the three-dimensional structure confirms the results from the HDX-MS data and can specifically define the amino acids involved in the interaction.

### Example 14—Epitope Mapping of Prenominated Antibody Candidates by HDX-MS

In this Example, hydrogen deuterium exchange mass spectrometry (HDX-MS) was performed to map the BSSL epitopes of the five prenominated antibody candidates S-SL048-11 hIgG4 S228P (mAb11, HC SEQ ID NO: 119, LC SEQ ID NO: 120), S-SL048-46 hIgG4 S228P (mAb46, HC SEQ ID NO: 121, LC SEQ ID NO: 122), S-SL048-106 hIgG4 S228P (mAb106, HC SEQ ID NO: 123, LC SEQ ID NO: 124), S-SL048-116 hIgG4 S228P (mAb116, HC SEQ ID NO: 125, LC SEQ ID NO: 126) and S-SL048-118 hIgG4 S228P (mAb11,8 HC SEQ ID NO: 127, LC SEQ ID NO: 128).

### Methods and Materials

A 2 mg/mL BSSL solution in PBS was mixed with the different antibodies (in 5 mg/mL in 25 mM histidine, 150 mM NaCl, 0.02% P80, pH 6.0) for a 1:1 molar ratio. The samples were concentrated and the buffer exchanged to PBS using a 10K Centrifugal filter unit (Amicon Ultra, Merck). In parallel, a sample containing BSSL only, without the addition of antibody, was subjected to the same procedure.

The samples were analyzed in an automated HDX-MS system (CTC PAL/Biomotif HDX) in which samples were automatically labeled, quenched, digested, cleaned and separated at 2° C. More specifically, samples were labeled by mixing 3 μL of BSSL (or BSSL/antibody complex) with 22 μL of deuterated PBS and incubated at 4° C. for four labeling time points: 5 min, 30 min, 90 min and 180 min. The labelling reaction was stopped/quenched by decreasing the pH to ˜2.3 and temperature to ˜4° C. through the addition of 20 μL of a solution containing 2 M Urea, 100 mM TCEP and 0.5% TFA. Samples were digested using an immobilized pepsin column (2.1 column (2.1×30 mm) at 250 μl/min, followed by an on-line desalting step using a 2 mm I.D×10 mm length C-18 pre-column (ACE HPLC Columns, Aberdeen, UK) using 0.05% TFA at 350 μl/min for 3 min. Peptic peptides were then separated by a 15 min 8-60% linear gradient of ACN in 0.1% formic acid using a 2 mm I.D×50 mm length HALO C18/1.8 pm analytical column operated at 95 μL/min. An Orbitrap Q Exactive mass spectrometer (Thermo Fisher Scientific) operated at a resolution of 70,000 resolution and m/z 400 was used for analysis. The software Mascot was used for peptide identification and HDExaminer (Sierra Analytics, USA) was used to process all HDX-MS data. Statistical analysis was done using a 95% confidence interval.

### Results

The deuteration kinetics of 54 peptides were followed by HDX-MS covering 64% of the protein. Deuterium labeling (5, 30, 90 and 180 min) and differential deuteration uptake kinetics between the BSSL alone and in the presence of any of the five antibodies was calculated. It was observed that several peptides near the N-terminus of BSSL showed statistically lower deuterium uptake in the presence of the antibody for each of the prenominated candidates. By considering overlapping peptides the spatial resolution could be reduced, resulting in the two peptide regions shown in Table 16.

TABLE 16
A list of the common epitope regions suggested
by the HDX-MS experiment

| Epitope | Start-End | | |
|---|---|---|---|
| regions | amino acid (aa) | Sequence | SEQ ID NO: |
| 1 | aa 1-12 | AKLGAVYTEGGF | 3 |
| 2 | aa 42-55 | LENPQPHPGWQGTL | 2 |

In addition to the epitope regions common to all prenominated antibodies, one extra peptide was identified as having statistically lower deuterium uptake in three of the antibodies, namely peptide 10 (aa 84-101; NIWVPQGRKQVSRDLPVM (SEQ ID NO: 4)) for S-SL048-46, peptide 24 for S-SL048-116 (aa 174-180; VKRNIAA (SEQ ID NO: 5)) and peptide 39 (aa 283-295; HYVGFVPVIDGDF (SEQ ID NO: 6)) for S-SL048-11 (Table 17). However, the signals of these were relatively week.

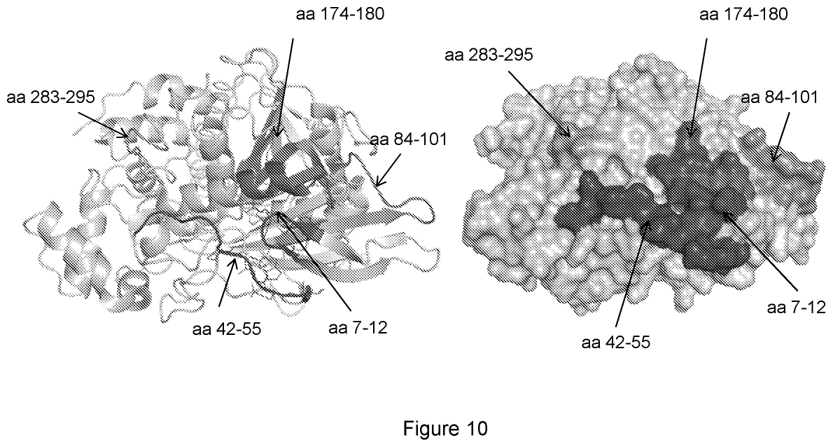

It can be noted, although spread out in the primary sequence, the two epitope regions common to all five antibodies (aa 1-12 and aa 42-55), cluster together in the three-dimensional structure (FIG. 10). Peptide 24 (aa 174-180) is relatively close to this core region, whereas peptides 10 (aa 84-101) and 39 (aa 283-295) are more spread out.

TABLE 17
Summary of peptides having differential deuteration
uptake kinetics between BSSL alone and in the presence
of any of the five antibodies

| Peptide | | | Sequence | Statistical significance | | | | |
|---|---|---|---|---|---|---|---|---|
| no. | Start | End | (SEQ ID NO:) | mAb46 | mAb116 | mAb106 | mAb118 | mAb11 |
| 1 | 1 | 7 | AKLGAVY | ++ | ++ | | | + |
| | | | (148) | | | | | |
| 2 | 1 | 11 | AKLGAVYTEGG | ++ | ++ | + | | ++ |
| | | | (149) | | | | | |
| 3 | 1 | 12 | AKLGAVYTEGGF | ++ | ++ | + | + | ++ |
| | | | (3) | | | | | |
| 4 | 1 | 25 | AKLGAVYTEGGFVE | ++ | ++ | | | ++ |
| | | | GVNKKLGLLGD | | | | | |
| | | | (150) | | | | | |
| 5 | 1 | 26 | AKLGAVYTEGGFVE | + | + | | | ++ |
| | | | GVNKKLGLLGDS | | | | | |
| | | | (151) | | | | | |
| 6 | 1 | 28 | AKLGAVYTEGGFVE | ++ | ++ | + | + | ++ |
| | | | GVNKKLGLLGDSVD | | | | | |
| | | | (152) | | | | | |
| 9 | 42 | 55 | LENPQPHPGWQGTL | ++ | ++ | +++ | +++ | +++ |
| | | | (2) | | | | | |
| 10 | 84 | 101 | NIWVPQGRKQVSRD | + | | | | |
| | | | LPVM | | | | | |
| | | | (4) | | | | | |
| 24 | 174 | 180 | VKRNIAA | | + | | | |
| | | | (5) | | | | | |
| 39 | 283 | 295 | HYVGFVPVIDGDF | | | | | + |
| | | | (6) | | | | | |

### Conclusion

The epitopes of the five prenominated antibody candidates were successfully mapped to the N-terminal region of BSSL. Two discontinuous peptide regions were identified with significantly lower deuterium uptake in presence of all five antibodies.

Overlapping peptides can allow for a reduction of the spatial resolution. For example, in the AS20 experiment (Example 13) the peptide corresponding to aa 1-6 was found to have no change in deuterium exchange, whereas the longer overlapping peptide did change (AKLGAVYTEGGF, aa 1-12, SEQ ID NO: 3). In other words, the epitope residues could be reduced to YTEGGF (aa 7-12) (SEQ ID NO: 1). In the current example, in contrast, such reduction could not be done since peptide corresponding to aa 1-6 was not detected.

In addition to the two common epitope regions, one extra peptide each was identified as potential epitope region in three of the prenominated candidates (S-SL048-46, S-SL048-116 and S-SL048-11). Although statistically significant, the magnitude of signal changes of these peptides was relatively low and may be less important for the interaction. Still, the fact that the peptide VKRNIAA (aa 174-180, SEQ ID NO: 5) clusters to the core region in three-dimensional space may suggest that this region is also part of the epitope. Of note, this peptide also overlaps with one peptide detected in the original mapping of AS20. Signal change of the two more peripheral peptide 10 (aa 84-101, SEQ ID NO: 4) and peptide 39 (aa 283-295, SEQ ID NO: 6) can possible be explained by stabilization upon antibody binding (“allosteric” effects).

Taken together, the data strongly suggest a shared common core, consisting of two peptide regions somewhere within the sequences 1-12 (SEQ ID NO: 3) and 42-55 (SEQ ID NO: 2) for the all prenominated candidates. These regions were also identified in previous epitope mapping experiments of AS20 as described in Example 13 and are seen to be important for the interaction in the crystal structure of the AS20 complex, described in Example 21.

### Example 15—Immunogenicity Assessment

This Example describes the immunigenicity assessment of the candidates SL048-11 (HC SEQ ID NO: 119 and LC SEQ ID NO: 120), S-SL048-46 (HC SEQ ID NO: 121 and LC SEQ ID NO: 122), S-SL048-106 (HC SEQ ID NO: 123 and HC SEQ ID NO: 124), S-SL048-116 (HC SEQ ID NO: 125 and LC SEQ ID NO: 126) and S-SL048-118 (HC SEQ ID NO: 127 and HC SEQ ID NO: 128) and AS20 hIgG4 S228P (HC SEQ ID NO: 129 and LC SEQ ID NO: 130) performed by Abzena.

### Methods and Materials

Using Abzena's iTope™ MHC Class II prediction in silico algorithm, sequences provided by Lipum were analysed for immunogenicity potential. The iTope™ software predicts favourable interactions between amino acid side chains of a peptide and specific binding pockets (pocket positions; p1, p4, p6, p7 and p9) within the open-ended binding grooves of 34 human MHC class II alleles. These alleles represent the most common HLA-DR alleles found world-wide with no weighting attributed to those found most prevalently in any ethnic population. Twenty of the alleles contain the ‘open’ p1 configuration and 14 contain the ‘closed’ configuration where glycine at position 83 is replaced by a valine. The location of key binding residues is achieved by the in silico generation of 9-mer peptides that overlap by eight amino acids spanning the test protein sequence.

Results should be assessed in the light of the fact that all predictive methods for MHC class II binding inherently over-predict the number of T cell epitopes since they do not allow for other important processes during antigen presentation such as protein/peptide processing, recognition by the T cell receptor or T cell tolerance to the peptide.

The position of p1 anchor residues (comprising the first residue of a MHC class II core 9-mer ligand) were highlighted in the S-SL048-11, S-SL048-46, S-SL048-106, S-SL048-116, S-SL048-118 and AS20 in hIgG4 S228P format.

If ≥50% of the MHC class II binding peptides (i.e., ≥17 out of 34 alleles) had a high binding affinity (score >0.6), such peptides were defined as “promiscuous high affinity” MHC class II binding peptides.

MHC class II binding peptides binding 50% of alleles with a score >0.55 (but without the majority >0.6) were defined as “promiscuous moderate affinity”.

These criteria were altered in the case of a large aromatic amino acid (i.e., F, W, Y) occurring in the p1 anchor position where the open p1 pocket of 20 of the 34 alleles allows the binding of a large aromatic residue. Where this occurs, a promiscuous peptide is defined as binding to 10 or more of the subset of 20 alleles.

The p1 anchor positions of germline moderate and high affinity binding peptides were also analysed and these would not be expected to be problematic in healthy individuals due to T cell tolerance.

Positive iTope™ hits were BLAST searched against the TCED™ database of known positive peptides and regions representing closely homologous peptides from the T cell epitope database (i.e., peptides known to induce T cell activation in the ex vivo EpiScreen™ T cell epitope mapping assay) were indicated. Kabat numbering was used for labelling antibody sequences.

### Results

TCED™ analysis of peptides previously tested in ex vivo EpiScreen™ T cell assays revealed strong homology to a number of iTope™ MHC binding peptides. In Table 18, +indicates mismatched residues where substitutions are amino acids with similar physiochemical properties and - indicates other mismatched residues. The positions of key MHC class II pocket positions for both iTope™ and TCED™ sequences are shown in the bottom row in Table 18.

TABLE 18
Results from TCED ™ analysis

| | Anchor | iTope ™ residues | TCED ™ homology |
|---|---|---|---|
| HC sequence | residue | (SEQ ID NO:) | match |
| AS20 | V12 | VKPGASVKM (153) | VKPGASVKM |
| S-SL048-11, -46, -106, | Y32 | YNMHWVRQA (154) | +-MHWVRQA |
| -118 | | | |
| S-SL048-11, -116, | Y59 | YAQKFKGRV (155) | YAQK++GRV |
| -118 | | | |
| AS20 | W103 | WGQGTTLTV (156) | WGQGT-+TV |
| MHC class II pocket position | | P1 4 67 9 | P1 4 67 9 |
| AS20 | V19 | VTMTCSASS (157) | VTMTC-ASS |
| S-SL048-46 | V19 | VTITCRASS (158) | VT-TCRASS |
| S-SL048-106 | V19 | VTITCSASS (159) | VTITC-AS- |
| S-SL048-106 | L46 | LLIYATSKL (160) | LLIYA-SKL |
| S-SL048-11, -116 | I48 | IYATSSLAS (161) | IYATS-LAS |
| S-SL048-46 | I48 | IYAASSLAS (162) | IYA+S-LAS |
| A520 | F98 | FGGGTKLEI (163) | FG-GTKLEI |

| | | |
|---|---|---|
| MHC class II pocket position | P1 4 67 9 | P1 4 67 9 |

Table 19 shows total number of iTope™ promiscuous moderate and high affinity MHC class II binding peptides and TCED™ hits for each candidate sequence (AS20 is shown for reference only).

TABLE 19
Summary results of immunogenicity analysis

| | Non-germinline | Non-germline | |
|---|---|---|---|
| | promiscuous | promiscuous | |
| | moderate | high | |
| | affinity MHC | affinity MHC | |
| | class II | class II | TCED ™ |
| Sequence | binding peptides | binding peptides | hits |
| AS20 hIgG4 S228P | 8 | 13 | 4 |
| S-SL048-116 hIgG4 | 5 | 7 | 3 |
| S228P | | | |
| S-SL048-11 hIgG4 | 6 | 6 | 3 |
| S228P | | | |
| S-SL048-118 hIgG4 | 6 | 6 | 2 |
| S228P | | | |
| S-SL048-46 hIgG4 | 3 | 6 | 3 |
| S228P | | | |
| S-SL048-106 hIgG4 | 5 | 5 | 3 |
| S228P | | | |

All five candidate humanised clones contained fewer non-germline promiscuous MHC class II peptides when compared to AS20 hIgG4 S228P. All variants sequences contained at least two TCED™ hits, with partial (a minimum 6 out of 9 positions) homology to peptides known to induce T activation in EpiScreen™ ex vivo assays. Out of the five variants, S-SL048-106 hIgG4 S228P represented the lowest immunogenicity risk based on the number of non-germline promiscuous high and moderate affinity MHC class II binding peptides. The candidates were ranked according to the following: fewer promiscuous hits 106<46<11=118<116 more promiscuous hits.

It is to be noted that in silico immunogenicity analysis is inherently over-predictive.

### Example 16—Manufacturability Assessment

This Example describes the in silico manufacturability assessment of the candidates S-SL048-11 (HC SEQ ID NO: 119 and LC SEQ ID NO: 120), S-SL048-46 (HC SEQ ID NO: 121 and LC SEQ ID NO: 122), S-SL048-106 (HC SEQ ID NO: 123 and LC SEQ ID NO: 124), S-SL048-116 (HC SEQ ID NO: 125 and LC SEQ ID NO: 126) and S-SL048-118 (HC SEQ ID NO: 127 and LC SEQ ID NO: 128) in hIgG4 S228P format performed by Abzena.

### Methods and Materials

Amino acid sequences of S-SL048-11, S-SL048-46, S-SL048-106, S-SL048-116 and S-SL048-118 in hIgG4 S228P format were analysed using Abzena's in silico liability prediction algorithm. Liabilities identified are subsequently analysed in a structural context. Briefly, for the sequence of each V domain the following was analysed:
Presence of deamidation sites;
Presence of isomerisation sites;
Potential oxidation sites;
Presence of N-linked glycosylation sites
Presence of free cysteines.

Herein, IMGT CDR definitions and numbering is used throughout unless otherwise indicated.

### Presence of Deamidation Sites

Deamidation of asparagine residues can lead to structural changes, changes in pharmacokinetics and efficacy and potential immunogenicity. Asparagine residues as potential deamidation sites were analysed in the context of both the amino and carboxy adjacent amino acids using a method based on [14].

### Presence of Isomerisation Sites

Aspartate isomerisation sites were predicted by analysing the sequence for known isomerisation motifs (DG, DS, DT or DD), focussing on CDR regions.

### Potential Oxidation Sites

Structural models of both the heavy and light chain variable domains were generated and methionine and tryptophan residues were identified and assessed in order to determine whether they are likely to be surface exposed and therefore candidates for oxidation. The oxidation of individual residues may impact the biological activity of antibodies and may have biological consequences for example, reduced efficacy or altered pharmacokinetics.

### Presence of N-Linked Glycosylation Sites

VH and V_{K} sequences were analysed based on the consensus N-linked glycosylation motif: —N—X—S/T —where X can be any amino acid except proline.

Presence of Free Cysteines

Sequences were analysed to identify unpaired cysteine residues. VH and VK domains typically each contain two canonical cysteines which form an intra-chain disulphide bond in the folded molecule. Additional cysteines would be expected to be detrimental to folding and potentially cause issues such as aggregation.

### Results

Analysis was performed and no potential N-linked glycosylation sites were identified within either the VH or V_{K} sequences for the five candidate antibodies; no unpaired cysteines were identified within either the VH or VK sequences for the five candidate antibodies; no potential oxidation sites were identified within the VH or V_{K} sequences for the five candidate antibodies and no sites with high (t_{1/2}<10 days) or medium (t_{1/2}<25 days) potential for deamidation were identified within the VH or V_{K} for any of the five candidate antibodies.

Two potential isomerisation sites were identified within the VH for all five clones in regions at or close to CDRs.

Asp 54 (DG) is located within VH CDR2 so isomerisation may have an effect on antigen binding;

Asp72 is located close to the CDRs in a region sometimes referred to as “CDR4”, and so isomerisation may have an effect on antigen binding.

TABLE 20
Sequence Liability Summary

| Candidate | Identified features |
|---|---|
| S-SL048-11 | Two potential aspartate isomerisation |
| hIgG4 S228P | sites were identified: |
| | Asp 54 (VH CDR2) |
| | and Asp 72 (FW2, adjacent to the CDR region) |
| S-SL048-46 | Two potential aspartate isomerisation |
| hIgG4 S228P | sites were identified: |
| | Asp 54 (VH CDR2) and |
| | Asp 72 (FW2, adjacent to the CDR region) |
| | One potential deamidation site was |
| | identified at Asn 60 within VH CDR2 |
| S-SL048-106 | Two potential aspartate isomerisation |
| hIgG4 S228P | sites were identified |
| | Asp 54 (VH CDR2) |
| | and Asp 72 (FW2, adjacent to the CDR region) |
| | One potential deamidation site was |
| | identified at Asn 60 within VH CDR2 |
| S-SL048-116 | Two potential aspartate isomerisation |
| hIgG4 S228P | sites were identified: |
| | Asp 54 (VH CDR2) |
| | and Asp 72 (FW2, adjacent to the CDR region) |
| S-SL048-118 | Two potential aspartate isomerisation |
| hIgG4 S228P | sites were identified: |
| | Asp 54 (VH CDR2) |
| | and Asp 72 (FW2, adjacent to the CDR region) |

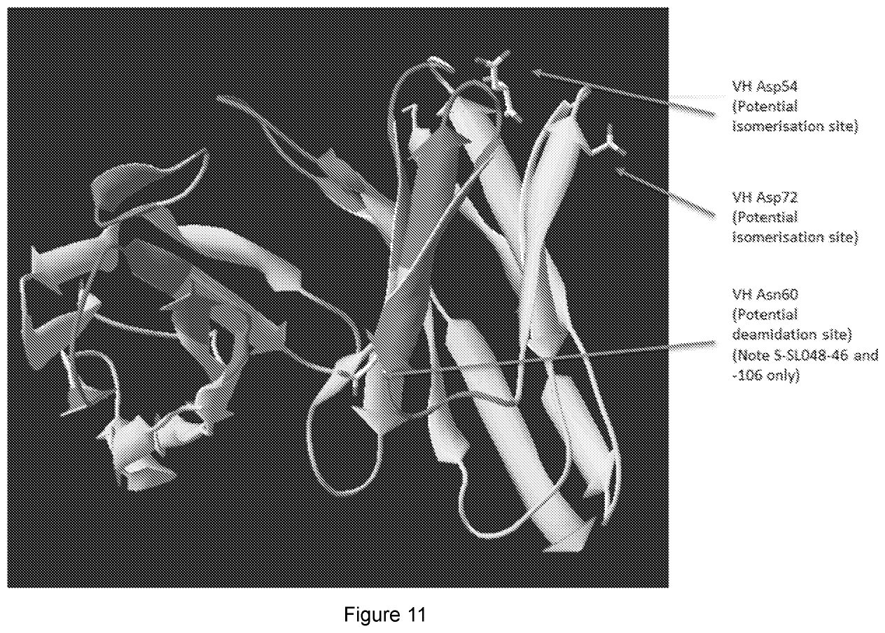

A representation of the S-SL048-106 Fv with potential post translational liabilities highlighted is shown in FIG. 11.

In summary, no free cysteines, oxidation sites or N-linked glycosylation sites were identified within any of the five lead candidates. No high potential deamidation sites were identified in S-SL048-11, S-SL048-116 or S-SL048-118. S-SL048-46 and S-SL048-106 contain a potential deamidation site within VH CDR2. Two potential isomerisation sites were identified within the heavy chain of each of the five lead variants.

### Example 17—Production and Binding Characterization of AS20 hIgG1 LALA-PG and Anti-NP hIgG1 LALA-PG

In this Example, the production and binding characterization of AS20 and anti-NP (clone B1-8) antibodies of human IgG1-LALA-PG subclass, hereafter called AS20 hIgG1 LALA-PG (comprising HC SEQ ID NO: 115 and LC SEQ ID NO: 116) and anti-NP hIgG1 LALA-PG (comprising HC SEQ ID NO: 117 and LC SEQ ID NO: 118) is described. The anti-NP antibody was included as isotype control.

### Material and Methods

Expression and purification

IgG4 is the most Fc inert natural human subclass. However, several publications have shown that IgG4 can interact with FcR as well as complement in mice [15] as well as humans. Therefore, the human IgG1 with mutations in three positions, namely L234A, L235A and P329G (hIgG1 LALA-PG for short), was chosen in this study as it has been reported to be the most Fc silent variant available [7, 16], i.e., having no immune effector functions.

Production of material was outsourced to Absolute Antibody (Oxford, UK). Sequence information of VH and VL of AS20 was sent to the company, where the genes were synthesized and cloned into vectors encoding the human IgG1-LALA-PG subclass. The antibodies were transiently expressed in mammalian HEK293 cells and subsequently purified by affinity chromatography using protein A. The purity and integrity were assessed by SDS-PAGE and endotoxin levels determined by LAL chromogenic endotoxin assay.

### Surface Plasmon Resonance (SPR)

Single cycle kinetics was used to measure the affinity of the antibodies. AS20 hIgG1 LALA-PG and anti-NP hIgG1 LALA-PG were immobilized on a CM5 S sensor chip by primary amine coupling using NHS-EDC chemistry. Five different concentrations of antigen (1:3 dilutions starting from 50 nM for hBSSL and 1:2 dilutions starting from 800 nM for mBSSL) were injected over the surface. The sensor chip surface was regenerated with 10 mM glycine-HCl pH 2.1. Obtained single cycle kinetic data was fitted to a Langmuir 1:1 binding model and kinetic parameters were retrieved using the software BlAevaluation. For mBSSL, a steady state analysis was also performed by plotting the response level at equilibrium against each concentration, and K_{D} values were retrieved by the BlAevalution software.

### Results

AS20 hIgG1 LALA-PG and anti-NP hIgG1 LALA-PG was successfully produced (data not shown).

A single cycle kinetic SPR approach was used to determine the affinity of the IgG. For binding of AS20 hIgG1 LALA-PG to hBSSL, an equilibrium dissociation constant (K_{D}) of 0.91 nM was calculated. For mBSSL, the K_{D} was determined to 361 nM. As expected, no binding to BSSL was seen for anti-NP hIgG1 LALA-PG.

### Conclusion

This Example describes a quality check performed in order to verify that AS20 hIgG1 LALA-PG is functional, i.e., that the binding to BSSL is comparable to that of AS20 in the hIgG4 S228P format.

SPR analysis of AS20 hIgG1 LALA-PG showed that the binding to BSSL was very similar to that observed for AS20 in other IgG formats. The K_{D} for hBSSL was determined by SPR to 0.91 nM, which is comparable to K_{D} values of 0.6nM -3 nM, which had been determined for other IgG formats of AS20 (as described in Example 1, 2 and 4). The affinity of AS20 hIgG1 LALA-PG for mBSSL was estimated in a steady state affinity analysis to a K_{D} value of 361 nM, which is in the same order of magnitude as the K_{D} previously estimated by the same analysis (155 nM, Example 1), and the appearance of the sensorgrams were similar (not shown).

Thus, the data showed that the binding of AS20 hIgG1 LALA-PG was unaffected by the change in sub-class. The results also showed that the isotype control anti-NP hIgG1 LALA-PG did not bind to BSSL, and could therefore be suitable to use as a negative control in future analyses.

### Example 18—Efficacy Validation of AS20 hIgG1 LALA-PG in a Mouse Model of Rheumatoid Arthritis

In this Example, the effect of AS20 hIgG1 LALA-PG (heavy chain SEQ ID NO: 115 and light chain SEQ ID NO: 116) was investigated in an in vivo mouse model of rheumatoid arthritis (RA), i.e., collagen antibody induced arthritis (CAIA). The anti-NP hIgG1 LALA-PG antibody (heavy chain SEQ ID NO: 117 and light chain SEQ ID NO: 118) was included in the study as isotype control.

### Model Description—CAIA in Mouse

Collagen antibody induced arthritis (CAIA) in mice is an arthritis model independent of both B and T cells. Disease is induced with antibodies to collagen type II (CII) administered intravenously (i.v.), followed by intraperitoneal (i.p.) administration of LPS after 3-5 days in order to boost disease development. The injected antibodies bind to cartilage, thereby activating the immune system and recruiting macrophages and granulocytes to the joints. Boost injection of LPS is required to reach significant severity and incidence of disease development. The disease course is highly predictable and has an onset after LPS boost. The disease reaches maximum severity around day 15 and is thereafter decreasing in severity until eventually healing out. The study was approved by the local animal ethic committee Malmö/Lund, Sweden (M118-15).

### Material and Methods

### Disease Induction

DBA/1 mice (males, 8-9 weeks) were injected i.v. with 2 mg/mouse of a cocktail of monoclonal anti-CII antibodies (CIA-MAB-50, MD Bioproducts) on day 0. Day 5 the mice were injected with LPS (50 μg/mouse) i.p. in order to boost the disease.

### Experimental Groups and Administration of Test Items

AS20 hIgG1 LALA-PG and isotype control antibodies were delivered at a concentration of 5 mg/ml and further diluted in vehicle (25 mM Histidine, 150 mM NaCl, 0.02% P80, pH 6.0). Test items (antibodies) were administered i.p. every 4^{th} day starting one day prior to disease induction (day −1) and then day 3, 7, 11 and 15. AS20 hIgG1 LALA-PG was administered at three different doses, i.e., 10, 30 and 90 mg/kg and the isotype control (anti-NP hIgG1 LALA-PG) at 90 mg/kg, based on mean weight of the animals at day -2. The relatively high doses were chosen to compensate for AS20 hIgG1 LALA-PG's low affinity for mouse BSSL compared to human BSSL, as described in Example 17. The experimental groups are outlined in Table 21. The first administration at day −1 was a bolus dose, i.e., all test items were given as double doses divided into two injections, the first injection was given in the morning and the second injection in the afternoon. The following administrations (day 3, 7, 11 and 15) were given as single doses. The animals were weighed before each administration and the dose volume, 20 ml/kg was based on the individual weight of the animals.

TABLE 21
Experimental groups

| | | Dose | Route of | Time of | No. of |
|---|---|---|---|---|---|
| Group | Treatment | (mg/kg) | admin | term | animals |
| 1 | Isotype | 90 | i.p. | 96 hours after | 14 |
| | Ctrl | | | last dose | |
| | anti-NP | | | | |
| | hIgG1 | | | | |
| | LALA-PG | | | | |
| 2 | AS20 | 90 | i.p. | 96 hours after | 16* |
| | hIgG1 | | | last dose | |
| | LALA-PG | | | | |
| 3 | AS20 | 30 | i.p. | 96 hours after | 16* |
| | hIgG1 | | | last dose | |
| | LALA-PG | | | | |
| 4 | AS20 | 10 | i.p. | 96 hours after | 16* |
| | hIgG1 | | | last dose | |
| | LALA-PG | | | | |

*Two satellite animals per group.

### Disease Evaluation

Disease was evaluated daily from day 3 in a blinded fashion using a macroscopic scoring system of the four limbs ranging from 0 to 15 (1 point for each swollen or red toe, 1 point for a swollen or red mid foot digit or knuckle, 5 points for a swollen ankle) resulting in a maximum total score of 60 for each mouse. Due to ethical restrictions, animals with a score exceeding 45 were removed from the experiment.

### Blood Sampling

Blood was collected from all mice on study discontinuation day 19 by cardiac puncture under deep anesthesia (96 hours after the last injection on day 15) into micro tubes containing LiHeparin. The samples were immediately put on ice and centrifuged (2000×g for 5 min, at 4° C.) within 20 minutes from sampling. Plasma samples were aliquoted and frozen on dry ice. The aliquots were stored at −80° C. until analysis of AS20 IgG1 LALA-PG exposure and anti-drug antibodies (ADA) (all groups, n=70) and a panel of safety biomarkers (group 1-3, n=42).

### PK-Sampling of Satellite Animals

In order to get information about the PK-profile and ADA, there were two satellite animals included per dosing group of antibody-treated animals. Blood samples were taken from sublingual vein 1 h prior to administration on day 7 and 24 hours after administration (i.e., on day 8) (two animals per dosing group). Blood samples were also taken 1 h prior to dosing on day 15 and 24 hours after administration (i.e., day 16) (two animals per dosing group).

### Tissue Collection

At study discontinuation spleens from all animals were dissected out and weighed. The 14 spleens from isotype control-treated group (group 2) and from the 14 animals in the 90 mg/kg AS20 hIgG1 LALA-PG group (group 3) were homogenized and analyzed by FACS as described in Example 19.

### Drug Exposure in Plasma

Exposure of AS20 IgG1 LALA-PG was analyzed in plasma samples collected from the satellite animals (see above) and from all animals at study discontinuation (day 19) using liquid chromatography-tandem mass spectrometry (LC-MS/MS).

### Anti-Drug Antibodies (ADA)

Immunogenicity assessment was performed by analyzing ADA in plasma samples collected from the satellite animals (see above) and from all animals at study discontinuation (day 19).

ADA was measured using an enzyme-linked immunosorbent assay (ELISA). In brief, maxisorp plates were coated with AS20 hIgG1 LALA-20 or isotype control antibodies and blocked with 5% BSA, 0.05% Tween-20 in 1×PBS. Plasma samples, or mouse plasma spiked with positive control antibodies, were added and the plates were incubated for 2 h, at room temp. Plates were washed, the secondary antibody peroxidase AffiniPure Goat Anti-mouse IgG+IgM (H+L) (Jackson ImmunoResearch) was added and after incubation 1 h at room temp, plates were thoroughly washed and finally TMD substrate (Sigma-Aldrich) was used for detection.

### Measurement of Safety Biomarkers

Plasma samples collected from group 1 (vehicle control), group 2 (isotype control, 90 mg/kg) and group 3 (AS20 hIgG1 LALA-PG, 90 mg/kg) were analyzed using clinical chemistry methods for 14 safety biomarkers (albumin, alanine aminotransferase, alkaline phosphatase, amylase, bilirubin, blood urea nitrogen, calcium, creatinine, globulin, glucose, phosphate, potassium, sodium and total protein). The analyses were performed at the unit of Chemical and pharmaceutical Safety, RISE, Sodertalje, Sweden, using Abaxis Vetscan system with cassette #500-0038.

### Results

### Arthritis Severity

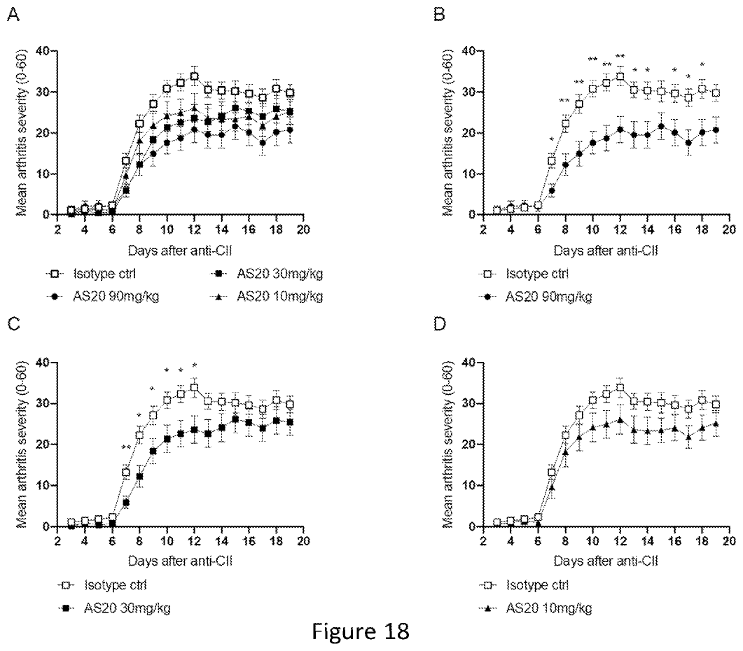

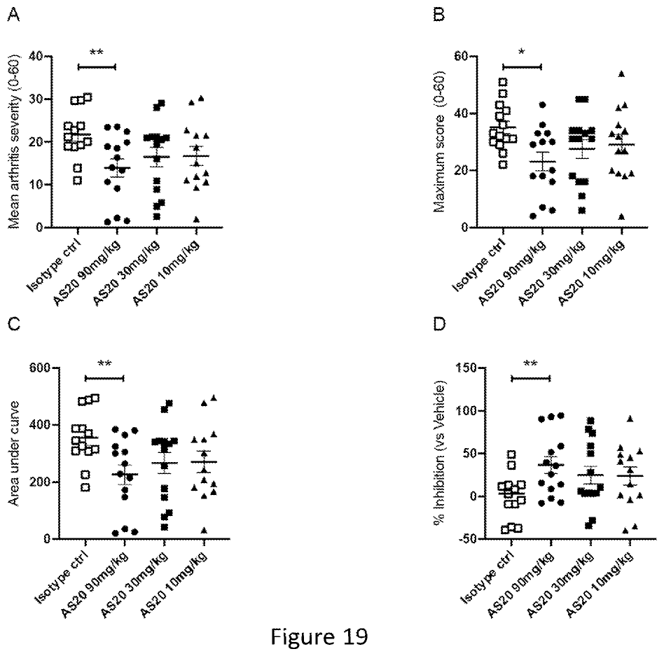

The CAIA induced and vehicle treated animals developed a moderate to severe disease with 100% incidence. Similar results were seen with the isotype control treated animals. The efficacy of AS20 hIgG1 LALA-PG, dosed i.p. every fourth day from day −1 until termination was evaluated in three doses (10, 30 and 90 mg/kg). AS20 hIgG1 LALA-PG at 90 mg/kg and 30 mg/kg showed significantly ameliorating effect on disease compared with isotype control on days 7-14, 16, 18 and 7-12, respectively. A small decrease in disease severity was seen with AS20 hIgG1 LALA-PG dosed at 90 mg/kg when comparing with vehicle, albeit not statistically significant (FIGS. 18 and 19).

Two animals, one in the AS20 hIgG1 LALA-PG 10 mg/kg group and one in the isotype control group, were removed pre-termination (day 12) for ethical reasons (high score). These animals were included in the maximum score but excluded from mean, AUC and % inhibition (FIG. 19, Table 22).

### Disease Parameter Statistics are Outlined in Table 22.

TABLE 22
CAIA severity parameter statistics

| Group | Incidence¹ | MES² | Max | AUC³ | % Inhibition⁴ |
|---|---|---|---|---|---|
| Iso Ctrl hIgG1 | 100% | 21.8 ± 1.6 | 35.1 ± 8.2 | 355.0 ± 26.5 | 0.0 ± 7.5 |
| LALA-PG90 mg/kg | | | | | |
| AS20 hIgG1 | 100% | 13.9 ± 2.1** | 23.1 ± 3.2** | 226.0 ± 34.7* | 36.4 ± 9.8** |
| LALA-PG 90 mg/kg | | | | | |
| AS20 hIgG1 | 100% | 16.5 ± 2.3 | 27.5 ± 3.2 | 267.0 ± 36.9 | 24.8 ± 10.4 |
| LALA-PG 30 mg/kg | | | | | |
| AS20 hIgG1 | 100% | 16.7 ± 2.3 | 29.1 ± 3.4 | 270.8 ± 37.3 | 23.71 ± 10.5 |
| LALA-PG 10 mg/kg | | | | | |

¹Cumulative incidence. A mouse was considered to have developed disease if scored a point of 1 or higher on two consecutive scoring days.
²Mean CAIA score for all scoring time points.
³Area under curve.
⁴Percent change in overall disease burden in each animal relative to isotype Ctrl treated group. Calculated by determining the difference between the isotype Ctrl group mean AUC and the AUC for each individual animal, divided by the isotype Ctrl group AUC and multiplied by 100 *(−1).
*p < 0.05;
**p < 0.01 compared with isotype Ctrl.

### Health Evaluation

General health assessment was performed daily in conjunction with evaluation of disease from day 3 until termination. The mice were weighed two times weekly as part of the general health assessment. No adverse effects from treatments were observed in the animals (data not shown).

### Drug Exposure in Plasma

The plasma exposure of AS20 hIgG1 LALA-PG at the end of the study on day 19 was overall in expected concentration range based on prior single dose pharmacokinetic assessment. The average concentration in the 10 mg/kg dose group was determined to 199 μg/mL (-1.3 μM, 58-309 pg/mL), for the 30 mg/kg dose group to 614 μg/mL (-4.1 μM, 229-970 μg/mL) and for the 90 mg/mL dose group the corresponding average concentration was 1408 μg/mL (˜9.4 μM, 520-2557 μg/mL). A slightly non-linear dose-exposure relationship was thus observed. No sign of altered plasma exposure over treatment time due to immunogenicity was detected. The average concentration of hIgG1 LALA-PG in the plasma samples from mice receiving isotype control was determined to 1815 (˜12 μM, 1224-2511 μg/mL),

### Anti-Drug Antibodies (ADA)

No alarming ADA was detected in the study samples. Although the majority of samples was tested positive, the titers was not higher in the AS20 hIgG1 LALA-PG groups than in the control groups. Considering the early onset of the response (˜day 7) and the unaltered drug exposure over time, it is possible that the detected anti-drug signal in the samples were caused mainly by unspecific/low affinity IgMs.

### Safety Biomarkers

Glucose increased in plasma following treatment with both AS20 hIgG1 LALA-PG and the isotype control antibody. None of the liver injury biomarkers were increased by the high dose AS20 hIgG1 LALA-PG. There was a tendency of increased plasma creatinine in the AS20 hIgG1 LALA-PG treated group compared to isotype control group, albeit not statistically significant (p<0.06). Other kidney markers, i.e. blood urea, electrolytes or total protein, did not differ between AS20 hIgG1 LALA-PG and isotype control treated mice.

### Conclusion

The CAIA induced isotype control treated mice developed a moderate to severe disease with 100% incidence. The same results were seen with the vehicle treated mice.

AS20 hIgG1 LALA-PG dosed at 90 mg/kg and 30 mg/kg showed significantly ameliorating effect on disease compared with isotype control on days 7-14, 16-18 and 7-12, respectively. A small decrease in disease severity was seen with AS20 hIgG1 LALA-PG dosed at 90 mg/kg when comparing with vehicle, albeit not statistically significant.

The plasma exposure of AS20 IgG1 LALA-PG at the end of the study was overall in expected concentration range. No signs of altered plasma exposure over treatment time due to immunogenicity was detected. No alarming ADA was detected in the study samples.

None of the liver injury biomarkers were increased by the high dose AS20 IgG1 LALA-PG. There was a tendency of increased plasma creatinine in the AS20 hIgG1 LALA-PG treated group, albeit not statistically significant. None of the other kidney markers, i.e., blood urea, electrolytes or total protein, differed between AS20 hIgG1 LAL

### Example 19—FACS Analysis of Spleen from CAIA Experiment

In this Example, the effect of AS20 hIgG1 LALA-PG (heavy chain SEQ ID NO: 115 and light chain SEQ ID NO: 116) on cellular subsets in spleen from mice with collagen antibody induced arthritis (CAIA) was investigated by fluorescence-activated cell sorting (FACS). The anti-NP hIgG1 LALA-PG (heavy chain SEQ ID NO: 117 and light chain SEQ ID NO: 118) was included as isotype control.

### Material and Methods

This study is part of the experimental in vivo study described above (Example 18). In brief, CAIA was induced in DBA/1 mice (males, 8-9 weeks) by intravenous (i.v.) administration of antibodies to collagen type II, followed by intraperitoneal (i.p.) administration of LPS to boost disease development. Mice were treated by i.p. administrations of AS20 hIgG1 LALA-PG or isotype control antibodies (anti-NP hIgG1 LALA-PG) every 4^{th} day, starting one day prior to disease induction (day -1). At study discontinuation (day 19), spleens from animals treated with the highest dose AS20 hIgG1 LALA-PG (90 mg/kg) or isotype control (90 mg/kg) were dissected out, weighed and homogenized as described below. A FACS panel was designed to identify T cells, B cells, NKT cells, Neutrophils, Eosinophils, NK cells, Dendritic cells, Monocytes and Macrophages.

### Single Cell Suspensions

Spleens were passed through a 70-μm cell strainer in RPMI-1640 culture media (Thermo Fisher, HyClone) to get single cell suspensions. The cells were pelleted and resuspended in 1 ml MilliQ water to lyse erythrocytes (10 sec), 1 ml of 2×PBS was added, followed by 10 ml 1×PBS. The cells were washed with 10 ml HBSS (Thermo Fisher, HyClone), and finally resuspended in an appropriate volume of HBSS to get 10×10⁶ cells/ml.

### FACS Protocol

1×10⁶ cells were seeded into each well of a 96-well round bottom plate and centrifuged for 1 min at 850×g, 4° C., washed with FACS buffer (1×PBS, 3% FBS, 2 mM EDTA) and pelleted again. Purified rat anti-mouse CD16/CD32 (Fc block) diluted 1:50 in FACS buffer was added to each well and the cells were incubated for 15 min, thereafter washed again with FACS buffer. An antibody mix was prepared by adding the following antibodies to an appropriate volume of FACS buffer:

FITC Hamster Anti-Mouse CD3ε (1:100)

PE Rat Anti-mouse CD86 (1:100)

PE-CF594 Rat Anti-Mouse Ly-6C (1:50)

PE-Cy7 Rat Anti-Mouse Ly-6G (1:200)

Biotin Hamster Anti-Mouse CD49b (1:200)

APC-Cy7 Hamster Anti-Mouse CD11 c (1:50)

Alexa Fluor 647 Rat anti-Mouse I-A/I-E (MHClI) (1:200)

Alexa Fluor 700 Mouse anti-Mouse CD45.2 (1:100)

BV421 Rat anti-Mouse Siglec-F (1:200)

BV605 Rat anti-CD11 b (1:200)

BV650 Rat anti-Mouse CD19 (1:100) BV786 Hamster anti-Mouse CD80 (1:200) eF506 Fixable Viability Dye (1:400) 25 μl of the antibody mix was added to the cells in each well and incubated for 20 min, on ice, protected from light. The cells were washed with FACS buffer, 25 μl secondary antibody mix (PerCP-Cy5.5 Streptavidin, diluted 1:200 in FACS buffer) was added and the cells were incubated for 20 min, on ice, protected from light. Then, cells were washed twice with FACS buffer, resuspended in 250 μl FACS buffer, transferred to FACS tubes and finally analyzed on CytoFLEX flow cytometer platform (Beckman Coulter).

### Gating Strategy

The cellular subsets were identified as follows:

T cells: CD45.2+, CD11b−, CD3+

B cells: CD45.2+, CD11b−, CD19+

NKT cells: CD45.2+, CD11b−, CD3+, CD49b+

Neutrophils: CD45.2+, CD11b+, Ly6G+

Eosinophils: CD45.2+, CD11 b+, Ly6G−, Siglec F+

NK cells: CD45.2+, CD11b+, Ly6G−, Siglec F−, CD49b+

Dendritic cells: CD45.2+, CD11b+, Ly6G−, Siglec F−, CD11c+, MHClI+

Monocytes: CD45.2+, CD11b+, Ly6G−, Siglec F−, CD110−, Ly6C+

Macrophages: CD45.2+, CD11b+, Ly6G−, Siglec F−, CD110−, Ly6C−, MHClI+

Moreover, expression of CD80 and CD86 on dendritic cells were analyzed.

### Results

There was no significant difference in body weight between the AS20 hIgG1 LALA-PG treated group (group 3, Example 18) and the isotype control group (group 2, Example 18), neither two days before disease induction (AS20, 23.9±1.4 g; isotype control, 23.8±1.2 g) nor at study discontinuation day 19 (AS20, 22.5±1.7 g; isotype control, 22.1±1.0 g). However, the spleen weights were significantly higher in the AS20 hIgG1 LALA-PG treated group compared to the isotype control group (110±23 mg vs. 88±21 mg; p=0.02), and the total number of CD45+ leukocytes was also higher in the AS20 group compared to the isotype control group (44.5×10⁶ vs. 39.0×10⁶; p=0.008).

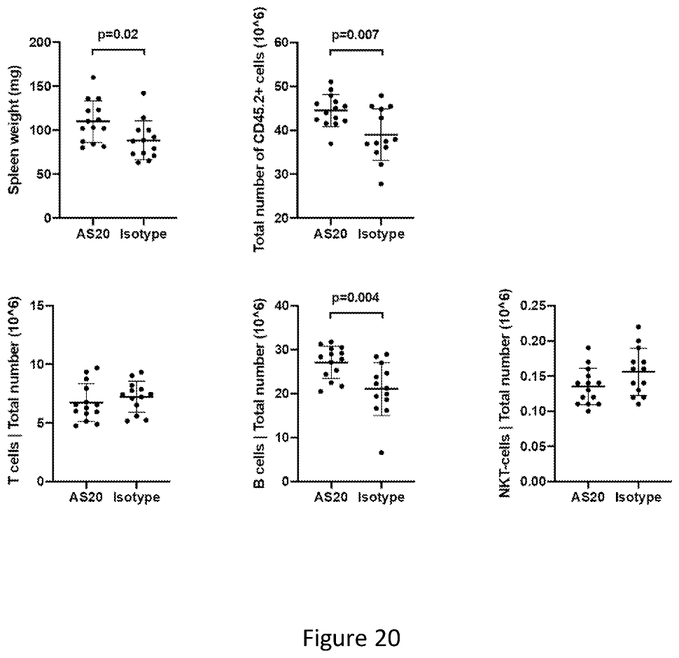

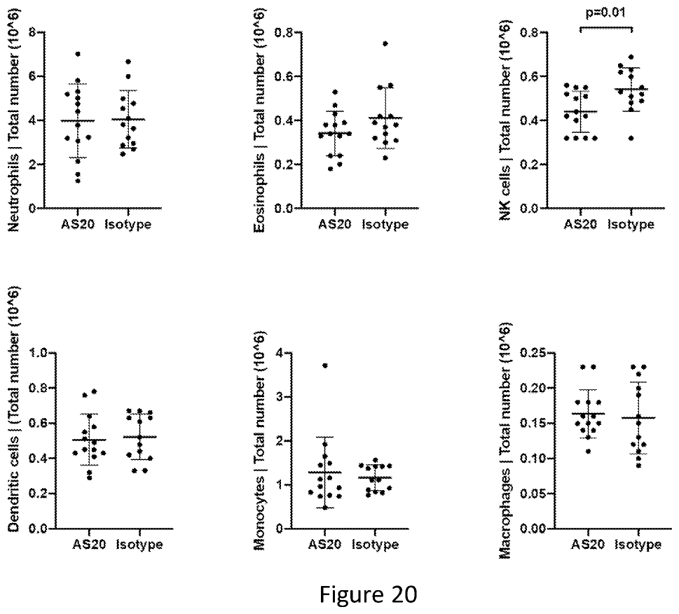

The total number of B cells in the spleen was higher in AS20 hIgG1 LALA-PG treated compared isotype control treated mice (27.1±3.5×10⁶ vs. 21.0±5.8×10⁶; p=0.004). In contrast, the total number of NK cells was reduced in AS20 hIgG1 LALA-PG treated compared to isotype control treated mice (0.44±0.09×10⁶ vs. 0.54±0.09×10⁶; p=0.01). The total number of NKT cells was also reduced in AS20 hIgG1 LALA-PG treated compared to isotype control treated mice, albeit not statistically significant (0.14±0.03×10⁶ vs. 0.16±0.03×10⁶; p=0.08). No significant difference was found in the total number of neutrophils, eosinophils, monocytes, macrophages, dendritic cells or T cells between AS20 IgG1 LALA-PG treated and isotype control treated mice (FIG. 20).

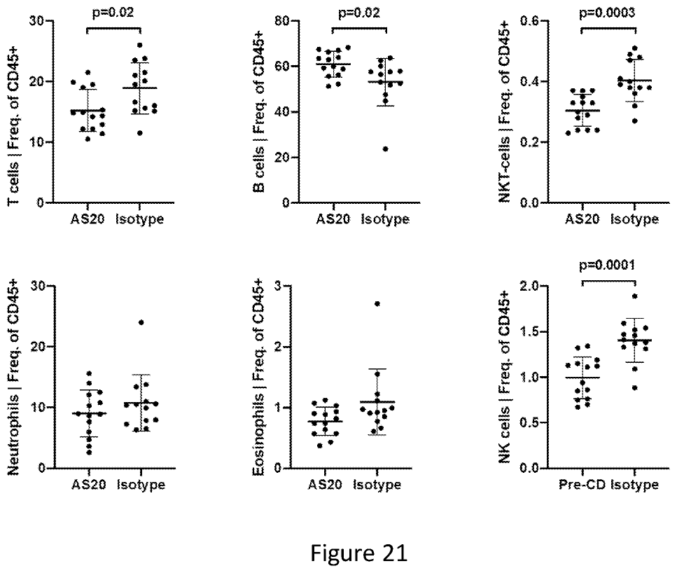

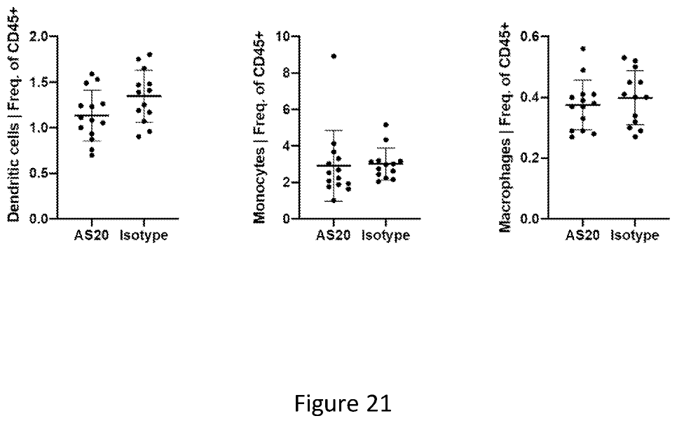

The proportion of B cells out of CD45+ cells in spleen was higher in AS20 IgG1 LALA-PG treated compared to isotype control treated mice (60.9±5.4% vs. 53.0±10.0%, p=0.02). In contrast, the proportion of T cells, NKT cells and NK cells out of CD45+ cells were found to be lower in AS20 hIgG1 LALA-PG treated compared to isotype control treated mice (T cells: 15.2±3.3% vs. 18.9±4.1%, p=0.02; NKT cells: 0.31±0.05% vs. 0.40±0.07%, p=0.0003; NK cells: 0.99±0.22% vs. 1.4±0.23%, p=0.0001). No significant difference in the proportion of neutrophils, eosinophils, monocytes, macrophages or dendritic cells out of CD45+ leukocytes were found between AS20 IgG1 LALA-PG treated and isotype control treated mice (FIG. 21). There was no significant difference between AS20 IgG1 LALA-PG treated and isotype control treated mice in cells expressing CD80 or CD86, neither in total number nor proportion out of CD45+ cells (data not shown).

### Conclusion

Treatment with AS20 hIgG1 LALA-PG (90 mg/kg dosage) significantly reduced the total number of NK cells and the proportion of T cells, NKT cells and NK cells out of CD45+ cells in spleen of mice with CAIA.

### Example 20—Analysis of the Effect of Prenominated Antibody Candidates on BSSL Enzyme Activity

In this Example, the effect of the five prenominated antibody candidates, namely S-SL048-11 (heavy chain SEQ ID NO: 119 and light chain SEQ ID NO: 120), S-SL048-46 (heavy chain SEQ ID NO: 121 and light chain SEQ ID NO: 122), S-SL048-106 (heavy chain SEQ ID NO: 123 and light chain SEQ ID NO: 124), S-SL048-116 (heavy chain SEQ ID NO: 125 and light chain SEQ ID NO: 126) and S-SL048-118 (heavy chain SEQ ID NO: 127 and light chain SEQ ID NO: 128) in hIgG4 S228P format, on BSSLs enzymatic activity was explored. Comparisons were made with the AS20 CDR graft (heavy chain SEQ ID NO: 131 and light chain SEQ ID NO: 132), chimeric AS20 (heavy chain SEQ ID NO: 129 and light chain SEQ ID NO: 130) and the isotype control anti-NP antibody (heavy chain SEQ ID NO: 133 and light chain SEQ ID NO: 134) in the hIgG4 S228P format.

### Methods and Materials

Preincubation of BSSL with Prenominated Antibody Candidates

Native human BSSL (SEQ ID NO: 138; Table 2) was used in this Example. The BSSL stock solution was diluted 10×in MilliQ (MQ)-H₂O to a concentration of 0.42 mg/ml.

The antibodies were diluted to 0.3 ₄/μ1 starting concentration in MQ-H20, followed by a 1:1 serial dilution in MQ-H20, resulting in 6-points concentrations ranging from 0.3 μg/μl to 0.009 μg/μl From each antibody dilution, 20 μl was added to 2.4 μl BSSL (1 μg) and the antibody/BSSL mixtures were incubated at +4° C. for 1 h. After incubation, 20 μl MQ-H₂O was added to each BSSL/antibody reaction.

### Triglyceride Hydrolysis Assay

A triglyceride (TG) emulsion was prepared by mixing 25 mg unlabeled triolein (Sigma cat. #92860) with 50 μl ³H-labeled triolein (triolein [9,10-3H(N)], 91 Cl/mmol) NET431001MC Perkin Elmer (Waltham, Mass.) in a round-bottomed, 30 mm diameter glass vessel suitable for sonication; evaporating the solvent under nitrogen gas (N₂) at room temperature; adding 1.0 ml 10% gum Arabic (Sigma cat. #G-9752), 1.25 ml 1.0 M Tris-HCl pH 9.0 and 2.0 ml MQ-H₂O to the vessel; chilling the vessel in ice water and sonicating for 10 min in a 50%-pulse mode using a Soniprep 150 (MSE, UK) with a 9 mm diameter flat-tipped probe at medium setting placed a few mm below the surface of the liquid until an emulsion was obtained; and adding 2.5 ml 18.7% BSA (Sigma cat. #A7906), 2.5 ml 1.0 M NaCl, and 3.25 ml MQ-H₂O to said emulsion. The emulsion was used on the same day it was prepared. Next, 10 μl of preincubated BSSL/antibody solution (see above) was mixed with 150 μl TG emulsion and 10 mM sodium cholate (Sigma cat. #C-1254) and MQ-H₂O in a total volume of 200 μl in 13×100 mm glass tubes. Samples were prepared in duplicates. The tubes were incubated at 37° C. for 15 min and the reactions were subsequently stopped by addition of 3.25 ml methanol/chloroform/heptane (vol/vol/vol, 760/680/540) and 1.0 ml 0.1 M sodium carbonate pH 10.5, followed by centrifugation at 3500×g for 10 min. From the upper water-soluble phase, containing the hydrolyzed free fatty acids, 1400 μl was withdrawn and mixed with 2.0 ml Optiphase Hisafe 3 scintillation cocktail (Perkin Elmer, Waltham, Mass.) in 6 ml polyethylene vials (Perkin Elmer) and the amount of hydrolyzed ³H-labelled free fatty acids was measured by liquid scintillation counting on a WinSpectral 1414 (Wallac, Turku, Finland).

### Cholesterol Ester Hydrolysis Assay

A cholesterol ester (CE) emulsion was prepared by adding 40 μl ¹⁴C-labeled cholesteryl oleate (oleate-1-14C, NEC6380050UC, Perkin Elmer) to a round-bottomed, 30 mm diameter glass vessel suitable for sonication and evaporating the solvent under nitrogen gas at room temperature; adding 2.0 ml 0.2 M Tris-HCl pH 7.5 and 0.85 ml MQ-H₂O to the vessel; chilling the vessel in ice water and sonicating for 10 min in a 50%-pulse mode using Soniprep 150 (MSE) with a 9 mm diameter flat-tipped probe at medium setting placed a few mm below the surface of the liquid until an emulsion was obtained; and adding 1.65 ml 200 mM sodium cholate and 1.0 ml MQ-H₂O to the emulsion. The emulsion was used on the same day it was prepared. Next, 10 μl of preincubated BSSL/antibody solution (see above) was mixed with 100 μl CE emulsion and MQ-H₂O to a total volume of 200 μl in 13×100 mm glass tubes. Samples were prepared in duplicates. The tubes were incubated at 37° C. for 30 min and the reaction was subsequently stopped by addition of 3.25 ml methanol/chloroform/heptane (vol/vol/vol, 760/680/540) and 1.0 ml 0.1 M sodium carbonate pH 10.5, followed by centrifugation at 3500×g for 10 minutes. From the upper water-soluble phase, containing the hydrolyzed free fatty acids, 400 μl was withdrawn and mixed with 2.0 ml Optiphase Hisafe 3 scintillation cocktail (Perkin Elmer) in 6 ml polyethylene vials (Perkin Elmer) and the amount of hydrolyzed ¹⁴C-labelled free fatty acids was measured by liquid scintillation counting on a WinSpectral 1414 (Wallac).

### Results

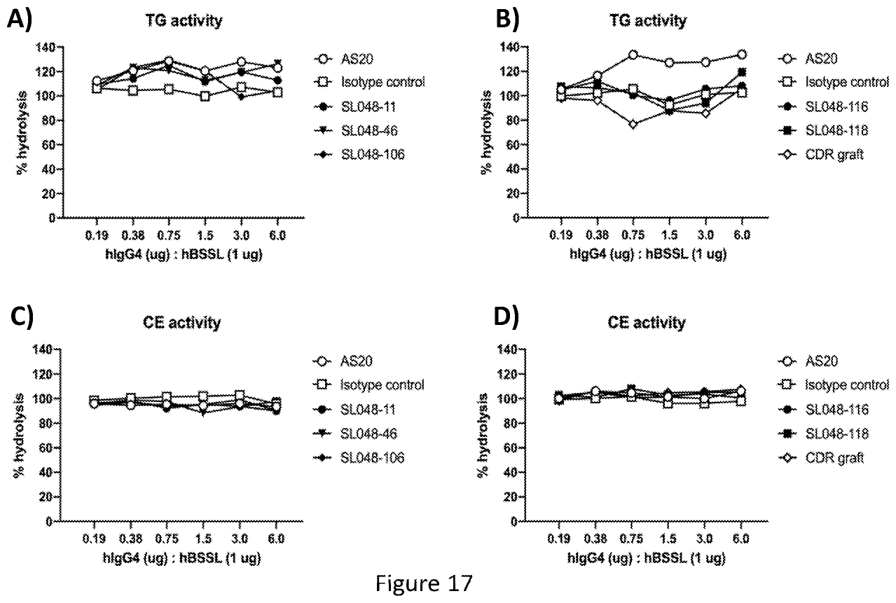

Enzymatic activity was evaluated by measuring release of free fatty acids (radioactively labelled) after 15 min incubation (triglyceride hydrolysis assay) or 30 min incubation (cholesterol ester hydrolysis assay), respectively, see FIG. 17. For technical reasons, the samples had to be analyzed in two consecutive sets, but two antibodies (AS20 and the isotype control anti-NP antibody) were included in both sets. Data are expressed as relative values with the value obtained without antibody added to the reaction set to 100% (Table 23 and 24).

TABLE 23
The effect of five BSSL-specific hIgG4 S228P and control antibodies of hIgG4
S228P subtype on BSSL enzymatic activity (hydrolysis of triglycerides)

| | | Effect of pre-nominated antibodies on | | | | | |
|---|---|---|---|---|---|---|---|
| | | BSSL hydrolysis of triglycerides | | | | | |
| | | (relative values; values obtained without | | | | | |
| | | antibody in reaction is set to 100%) | | | | | |
| Antibody | Set | 0.19 μg | 0.38 μg | 0.75 μg | 1.5 μg | 3.0 μg | 6.0 μg |
| S-SL048-11 | 1 | 109.4 ± 4.0 | 114.3 ± 1.1 | 125.0 ± 8.8 | 111.6 ± 6.8 | 119.8 ± 4.4 | 112.9 ± 0.2 |
| S-SL048-46 | 1 | 104.9 ± 5.0 | 123.0 ± 1.6 | 120.8 ± 4.2 | 112.7 ± 0.9 | 119.3 ± 2.8 | 126.3 ± 4.6 |
| S-SL048-106 | 1 | 108.7 ± 1.0 | 123.2 ± 4.0 | 129.4 ± 0.0 | 120.5 ± 0.8 | 99.2 ± 2.4 | 104.3 ± 7.4 |
| S-SL048-116 | 2 | 104.7 ± 6.3 | 111.8 ± 0.7 | 100.4 ± 3.2 | 96.2 ± 0.5 | 105.6 ± 2.1 | 108.1 ± 3.2 |
| S-SL048-118 | 2 | 107.3 ± 1.4 | 106.5 ± 4.1 | 102.2 ± 1.0 | 88.0 ± 0.1 | 93.9 ± 2.8 | 119.4 ± 0.2 |
| AS20 (set 1) | 1 | 112.4 ± 3.4 | 120.6 ± 2.9 | 128.6 ± 2.5 | 120.4 ± 9.8 | 128.0 ± 5.1 | 123.0 ± 3.4 |
| AS20 (set 2) | 2 | 105.0 ± 0.1 | 116.4 ± 0.9 | 133.5 ± 0.3 | 127.1 ± 1.4 | 127.4 ± 1.5 | 133.8 ± 4.9 |
| AS20 CDR graft | 2 | 98.0 ± 0.4 | 96.0 ± 0.6 | 76.7 ± 3.6 | 87.8 ± 0.1 | 85.6 ± 3.8 | 106.0 ± 1.6 |
| Anti-NP (set 1) | 1 | 106.3 ± 0.9 | 104.5 ± 3.2 | 105.5 ± 2.7 | 99.8 ± 4.1 | 107.2 ± 2.0 | 103.1 ± 4.5 |
| Anti-NP (set 2) | 2 | 99.8 ± 2.4 | 102.2 ± 0.8 | 105.5 ± 2.3 | 92.5 ± 2.8 | 101.1 ± 2.2 | 102.6 ± 7.1 |

TABLE 24
The effect of five BSSL-specific hIgG4 S228P and control antibodies of hIgG4
S228P subtype on BSSL enzymatic activity (hydrolysis of cholesterol esters)

| | | Effect of pre-nominated antibodies on | | | | | |
|---|---|---|---|---|---|---|---|
| | | BSSL hydrolysis of cholesterol esters | | | | | |
| | | (relative values; values obtained without | | | | | |
| | | antibody in reaction is set to 100%) | | | | | |
| Antibody | Set | 0.19 μg | 0.38 μg | 0.75 μg | 1.5 μg | 3.0 μg | 6.0 μg |
| S-SL048-11 | 1 | 96.2 ± 3.0 | 98.2 ± 4.4 | 92.2 ± 1.3 | 95.2 ± 0.0 | 93.7 ± 2.1 | 90.2 ± 1.3 |
| S-SL048-46 | 1 | 95.7 ± 2.5 | 99.0 ± 0.5 | 97.6 ± 0.4 | 88.3 ± 1.3 | 93.5 ± 0.2 | 97.7 ± 0.3 |
| S-SL048-106 | 1 | 94.8 ± 2.0 | 97.2 ± 6.1 | 93.9 ± 2.4 | 95.3 ± 0.4 | 99.0 ± 2.6 | 90.5 ± 0.2 |
| S-SL048-116 | 2 | 104.1 ± 3.2 | 107.2 ± 3.9 | 102.4 ± 0.9 | 102.8 ± 2.5 | 107.0 ± 1.3 | 107.1 ± 1.2 |
| S-SL048-118 | 2 | 104.6 ± 4.5 | 102.6 ± 7.4 | 110.3 ± 2.5 | 104.8 ± 5.8 | 105.6 ± 1.4 | 102.0 ± 3.1 |
| AS20 (set 1) | 1 | 96.0 ± 1.4 | 94.7 ± 0.9 | 95.4 ± 0.1 | 94.3 ± 0.5 | 96.1 ± 1.3 | 93.5 ± 2.9 |
| AS20 (set 2) | 2 | 101.7 ± 2.1 | 108.2 ± 4.1 | 106.6 ± 0.7 | 103.0 ± 7.0 | 101.5 ± 1.8 | 108.3 ± 0.7 |
| AS20 CDR graft | 2 | 99.8 ± 0.9 | 108.4 ± 1.6 | 103.7 ± 0.4 | 106.7 ± 0.9 | 107.7 ± 0.3 | 109.6 ± 1.8 |
| Anti-NP (set 1) | 1 | 98.5 ± 5.4 | 100.4 ± 3.4 | 101.5 ± 0.5 | 102.0 ± 0.15 | 102.8 ± 0.0 | 95.7 ± 2.5 |
| Anti-NP (set 2) | 2 | 100.7 ± 1.5 | 101.8 ± 2.7 | 103.2 ± 1.2 | 97.2 ± 1.6 | 97.2 ± 0.7 | 99.2 ± 4.4 |

### Conclusion

None of the antibodies tested, i.e., the five pre-nominated candidate drugs, AS20, AS20 CDR graft and anti-NP in hIgG4 S228P format, showed any significant effect on the enzymatic activity of BSSL.

### Example 21—Epitope Mapping of AS20 by X-ray Crystallography

X-ray crystallography was used to determine the three-dimensional structure of AS20 in complex with hBSSL. For this experiment the antibody was cleaved to produce Fab fragments and a new C-terminally truncated hBSSL (t-hBSSL) construct was made corresponding to amino acids 1 to 530 of hBSSL followed by AHHHHHH (SEQ ID NO: 146).

### Material and Methods

Cloning, Expression and Purification of t-hBSSL

Human BSSL has previously been crystallized in a truncated form lacking the flexible C-terminal part. Thus, to be able to produce crystals of AS20 Fab and hBSSL a new truncated BSSL construct was made including a C-terminal his tag for purification. The construct gp67-BSSL-6×H consisted of amino acids 1-530 +AHHHHHH (BSSL numbering based on the sequence after the signal peptide is removed), was ordered from GeneArt and cloned into a pFastBac tGFP Dual vector that had been prepared for ligation-independent cloning (LIC). LIC was performed using the InFusion cloning kit and transformed to Stellar competent cells.

Recombinant bacmid DNA was generated in DH10Bac E. coli cells. Transfection of bacmid into Sf9 cells was performed for 120 h at 27° C. P1 virus was harvested from the growth medium and subsequently used to generate P2 virus stock. After 96 h, P2 virus stock was harvested by centrifugation. 400 ml Sf9 cells (1.5×10⁶ cells/mi) were infected with 2 ml P2 virus stock and grown for 72 h post infection. The medium (P3 stock) was harvested by centrifugation and filtered. For large scale expression, 35 ml P3 stock virus was added to 2130 ml Sf9 cells (1.6×10⁶ cells/ml) in a Thomson Optimum Growth Flask (5 L). Expression was performed at 27° C. for 72 h. At harvest, cell density was 2.34×10⁶ cells/mi, with 75% expressing GFP, and 89% viability. The culture was centrifuged to remove cells. t-hBSSL was purified from the medium by batch IMAC using Ni Sepharose Fast Flow resin and eluted with 50 mM Tris pH 7.5, 500 mM NaCl, 500 mM imidazole. It was further purified by size exclusion chromatography using a Superdex 200 16/60 column in 50 mM Hepes pH 7.0, 500 mM NaCl. The protein eluted as a single monomeric peak, which was pooled and concentrated.

Complex Formation of AS20 Fab Fragment and t-hBSSL

Production of the AS20 antibody was performed at Absolute Antibody (Oxford, UK). Fab fragments were generated by following the papain digestion protocol from the immobilized papain supplier (Thermo Scientific, product no. 20341). AS20, 15 mg in total, was concentrated to 17 mg/ml and the buffer was exchanged to 20 mM Na phosphate, 10 mM EDTA pH 7, 20 mM cysteine. The antibody was incubated with immobilized papain (0.6 ml slurry) at 37° C. for 3 hours, followed by incubation at 4° C. overnight. Since cleavage was not complete the sample was incubated at 37° C. for an additional 3 hours, and then at RT over the weekend. The antibody was eluted with 10 mM Tris pH 7.5 and purified t-hBSSL was added. The mixture was incubated for ca 30 min at RT, after which it was loaded on a Superdex 200 column in 50 mM Hepes pH 7.0, 500 mM NaCl. The first major peak at around 60 ml elution volume contained the complex of t-hBSSL and AS20 Fab fragment. The relevant fractions were concentrated (10K MWCO) and salt concentration reduced to 250 mM by dilution with 50 mM Hepes pH 7.0 buffer.

Crystallization of AS20 Fab in Complex with t-hBSSL and Structure Solution

After purification of the Fab t-hBSSL complex the sample was kept on ice at 4° C. for approximately 36 h while transported between laboratories. The complex was then concentrated using a 500 μL Vivaspin with 30 000 MWCO (Vivaspin 500 from Sartorius, VS0122) from 4 mg/mL to a final concentration of 17.3 mg/mL. The concentration was measured at A280 on a Nanodrop using the extinction coefficient 174,375 and Mw of 106.242 kDa.

Crystallization experiments were set up using a Mosquito liquid handling robot in Swiss CI XTAL SD-3 3-well plates with 35 μL reservoir solution and drops of 150+50, 100+100, 50+150 nL protein+reservoir. The screen Morpheus from Molecular Dimensions gave crystals at 20 degrees in conditions B9 and B10. Crystals, of rod-like morphology, appeared in the first 12 h and grew larger during the next 24 h. These were cryo-cooled, after a quick transfer to reservoir solution containing an additional 10% glycerol, by plunging into liquid nitrogen. Data were collected at the Biomax beamline at MaxIV. The auto-processed data file was used for solving the structure by molecular replacement in Phaser with 1f6w.pdb (hBSSL) and 4n0y.pdb (Fab fragment) as search models.

Water molecules were removed as was the very C-terminal part of BSSL and for 4n0y.pdb only the Fab chains, made into a poly-alanine model, were kept. Manual model building was performed in Coot and the model was refined with Refmac 5 (all of the CCP4 suit).

### Results

Crystallization and Structure Determination of AS20 Fab t-hBSSL

AS20 Fab fragment could be crystallized together with t-hBSSL in the Morpheus condition B9, which contains 30 mM NaBr, 30 mM NaFI, 30 mM Nal, 0.1 M Tris (base), 0.1 M Bicine (buffer set to pH 8.5 with these two buffers), 20% PEG550 MME and 10% PEG 20K. The crystals belonged to the space group C2 with unit cell dimensions 323, 67, 123, 90, 101.7, 90 and diffracted to 2.5 Å. The structure was solved by molecular replacement and two complete complexes were present in the asymmetric unit. The crystal packing around the two copies is different resulting in small variations of the t-hBSSL structures. Especially the last residues including the 6-Histag are seen in molecule A but not in molecule B due to B having less crystal contacts in this region and more room for the C-terminal to be flexible. The region between residues 272 and 283 is also highly disordered in B and can't be modeled. Apart from these discrepancies the two t-hBSSL models superimpose very nicely.

Analysis of the Interaction Interface of AS20 Fab and t-hBSSL

The BSSL structure has been described as a having a large core region consisting of a twisted, 11-stranded beta-sheet surrounded by alpha helices and connecting loops [9]. At the N-terminus there is a smaller 3-stranded beta-sheet. The structure has been likened to a left-handed oven-glove with the palm containing the active site triad close to the “thumb”. With this likeness, the small N-terminal beta-sheet is located on the back of the hand close to the “little finger”, see FIG. 12. The part of the BSSL structure, which interacts with the Fab molecule is located to the small N-terminal beta sheet and the C-terminal part of alpha C [17], the third alpha helix in the structure. In other words, the binding region for the antibody is not close to the active site but on the opposite side of the antigen. FIG. 13 shows how the variable chains of AS20 bind to t-hBSSL with the epitope sequence highlighted in light grey.

The epitope regions are listed in Table 25 and comprise residues 7-12 (strand 1 and 2, SEQ ID NO: 1), 42-55 (loop region leading into strand 3 of the sheet, part of SEQ ID NO: 2), and 174-180 (the C-terminal end of alpha C, SEQ ID NO: 5). The epitope is rather flat with only a few characteristic residues sticking out, namely Tyr7, Phe12 and GIn52 (the main interactions listed in Table 245The loop region of 47-54 is well defined and forms a uniform surface. Proline 47 is important for a stacking interaction with Tyr31 of the Fab but as a whole the surface is flat here. Many of the residues within the epitope sequence are important for the BSSL fold but do not interact in a specific manner with AS20.

TABLE 25
A list of the BSSL residues involved in the interaction between
BSSL and Fab AS20 ranked with the most important interactions
in the top row. BSSL residues that are important for the
BSSL fold in the epitope area are also listed.

| | |
|---|---|
| Minimum main | Y7, E9, F12 |
| interactions | P47, Q52, T54 |
| | R176 |
| Main interactions | A5, Y7, E9, G10, G11, F12, E14 |
| and residues | P47, H48, P49, G50, W51, Q52, G53, T54, K56 |
| important | W173, V174, K175, R176, N177, A180 |
| for BSSL fold | |
| Residues important | A5, V6, Y7, T8, E9, G10, G11, |
| for interaction and | F12, V13, E14 |
| BSSL structure | Q46, P47, H48, P49, G50, W51, Q52, |
| | G53, T54, L55, K56 |
| | W173, V174, K175, R176, N177, |
| | I178, A179, A180 |
| Extended residues | A5, V6, Y7, T8, E9, G10, G11, |
| of importance | F12, V13, E14 |
| | Q46, P47, H48, P49, G50, W51, Q52, |
| | G53, T54, L55, K56 |
| | H168, M169,1171, A172, W173, V174, |
| | K175, R176, N177, I178, A179, A180 |

### Conclusion

This Example describes the crystallization and structure solution of AS20 Fab and t-hBSSL. The structure reveals that the epitope region is located to the same area as previously identified by HDX-MS, described in Example 13. It is a three-dimensional epitope consisting of a small beta sheet, a well-ordered loop region leading into strand three of the sheet and the C-terminal part of an adjacent helix. The sequences of the epitope are 7-12 (YTEGGF, SEQ ID NO: 1), 42-55 (LENPQPHPGWQGTL, SEQ ID NO: 2) and 174-180 (VKRNIAA, SEQ ID NO: 5); spread in sequence but coming close together in the structure. The most defining residues are Tyr7, Phe12 and GIn52, which protrude out from the surface.

The sequences of the five prenominated antibodies were analyzed in the context of the AS20 Fab t-hBSSL structure. Sequence differences, which are mainly conservative, and results from HDX-MS mapping (Example 14) indicate that all five antibodies would bind the same epitope on BSSL.

### Example 22—Complex Formation, Crystallization, Structure Determination and Epitope Analysis of S-SL048-116 Fab-BSSL Complex

### Materials and Methods

S-SL048-116 Cleavage with FabRICATOR

S-SL048-116 antibody (heavy chain SEQ ID NO: 125 and light chain SEQ ID NO: 126) in PBS was cleaved and the F(ab′)₂ purified using the FraglT Kit (Genovis) following the manufacturer's instructions.

### Reduction of the F(ab′)₂ Fragment and Purification of the Fab′ Fragment

A large-scale reduction of the purified F(ab′)₂ fragment was done using cysteamine at a final concentration of 50 mM at room temperature for 2 hours in PBS pH 7.2 containing 5 mM EDTA. Fourfifths of the resulting sample was purified on a HiLoad 26/60 Superdex 200 prep grade (GE Healthcare) with PBS pH 7.2, 2 mM EDTA as mobile phase. Fractions from the peak of interest were pooled, concentrated and stored at −80° C. for downstream applications. The total yield was 7.2 mg.

### Alkylation of the Fab′ Fragment and Subsequent Purification

The remaining one fifth of the reduced sample was treated with an equal volume of 375 mM iodoacetamide at room temperature for 30 min to block the free cysteines by alkylation. The resulting alkylated sample was purified exactly like the non-alkylated sample. The total yield was 2 mg.

### Expression and Purification of BSSL

The P2 virus stock used for expression was obtained from SciLifeLab (Stockholm, Sweden). The ExpiSf9 insect cell line was used in ExpiSf CD medium. Expression using the ExpiSf protein expression kit (ThermoFisher Scientific) was done according to the manufacturers recommendation using the viral load recommended by SciLife Lab.

Culture supernatant from a 2 L expression was supplemented with EDTA-free protease inhibitor tablets (Merck Millipore), NiSO₄ and imidazole (final concentration 15 mM). Any particles were removed by centrifugation at 13000×g. The supernatant was loaded on a 5 ml HisTrap Excel column (GE Healthcare) overnight at 4° C. at a flow rate of 2 ml/min. The column was washed with 6 column volumes (CV) IMAC buffer A (50 mM Tris-HCl, 500 mM NaCl; pH 7.5) containing 15 mM imidazole. The column was again washed with 10 CV of IMAC buffer A containing 25 mM imidazole. Bound protein was eluted with a 20 CV linear gradient to 100% IMAC buffer B (50 mM Tris-HCl, 500 mM NaCl, 0.5 M imidazole, pH 7.5). The eluted protein was concentrated, centrifuged and run on a HiLoad 26/600 Superdex 200 prep grade gel filtration column (GE Healthcare) pre-equilibrated with SEC buffer (50 mM HEPES, 500 mM NaCl, 2 mM EDTA; pH 7). Fractions from the peak of interest (1E6-1F8) were pooled and concentrated. The total yield was 6.2 mg and the sample was about 85% pure.

### BSSL:Fab′ Complex Formation and Purification

Before the complex generation, BSSL was evaluated by SEC on a HiLoad 26/600 Superdex 200 prep grade column to establish that there was no concentration/storage-induced oligomerization. The complex was mixed at a molar ratio of 1:1.3 BSSL:Fab′ and incubated on ice for 1 hour. The incubated complex was run on the same SEC column using the same buffer as for BSSL. Fractions of interest (B3-C4) were pooled and buffer exchanged to 50 mM HEPES, 250 mM NaCl, 2 mM EDTA; pH 7. The buffer-exchanged sample was concentrated to 17 mg/ml, flash-frozen in liquid nitrogen and stored at −80° C. ready for crystallization. The purity of the sample was >90% as estimated from SDS-PAGE analysis.

### BSSL:Alk-Fab′ Complex Formation and Purification

The complex was mixed at a molar ratio of 1.1:1 BSSL:Alk-Fab′ and incubated on ice for one hour. The incubated complex was run on the same SEC column using the same buffer as for the BSSL:Fab′ complex. Fractions of interest were pooled and buffer exchanged to 50 mM HEPES, 250 mM NaCl, 2 mM EDTA; pH 7. The buffer exchanged sample was concentrated to 14.5 mg/ml, flash frozen in liquid nitrogen and stored at −80° C. ready for crystallization.

### Crystallisation and Freezing of Fab-BSSL Complex

The best-diffracting crystals were grown at 20° C. from 14.5 mg/ml Fab-BSSL complex in buffer (50 mM HEPES, 250 mM NaCl, 2 mM EDTA, pH 7.0) and mixed with reservoir (16% (w/v) PEG 4000, 0.1 M sodium citrate pH 5 and 6% (v/v) ethanol) and seed solution as below:

150 nl complex+37 nl seed (crushed crystals in 0.1 M sodium citrate pH 5.0, 20% (w/v) PEG 4000 and 0.2 M ammonium sulphate)+113 nl reservoir

The sitting drop was pipetted on a MRC plate with 40 μl reservoir. The crystal appeared within a few days and was frozen in a cryo-solution containing 20% (v/v) glycerol, 16% (w/v) PEG 4000, 0.1 M sodium citrate pH 5.0 and 3% (v/v) ethanol.

### Data Collection

A data set was collected at 100K at BioMAX station, MAX IV, Lund, Sweden (A =0.97625 A) equipped with an Eiger 16M hybrid pixel detector. The data set was collected using an exposure time of 0.011 s and an oscillation of 0.1° per image, collecting 360° in total. The data were processed using the autoPROC pipeline to 2.5 A in space group P21.

### Results

### Structure Determination

The Fab-BSSL structure was determined using molecular replacement with the Phaser software with the 2.3 Å structure of the catalytic domain of the human bile salt activated lipase BSSL (from PDB: 1F6W) and a homologous 2.86 Å Fab structure (from PDB: 3NFP) as templates. One complex was found in the asymmetric unit. The structure was refined in Refmac5 followed by Buster and model building was carried out in Coot. The final model included three protein chains with BSSL (chain A) and the heavy chain (H) and light chain (L) of the S-SL048-116 Fab (FIG. 22). The final model included amino acids 1-531 in chain A except for a flexible loop not seen in the electron density (amino acids 117 -123). The model included amino acids 1-227 in chain H and amino acids 1 -211 in chain L. The first amino acid in chain H, Gln 1 has been modelled as PCA, pyroglutamic acid to best fit the electron density. In chain H, two free cysteines (Cys 133 and Cys 225) have been modelled as non-alkylated cysteines although alkylation treatment of the S-SL048-116 Fab was necessary for crystallization. No extra density was present to fit the methylamide group supposedly attached to the sulphur atom of the cysteine. Probably the cysteine residues werere at least partially alkylated but the attached atoms were so flexible that they were not clearly seen in the electron density map. In addition, 87 water molecules have been modelled.

### Epitope Analysis

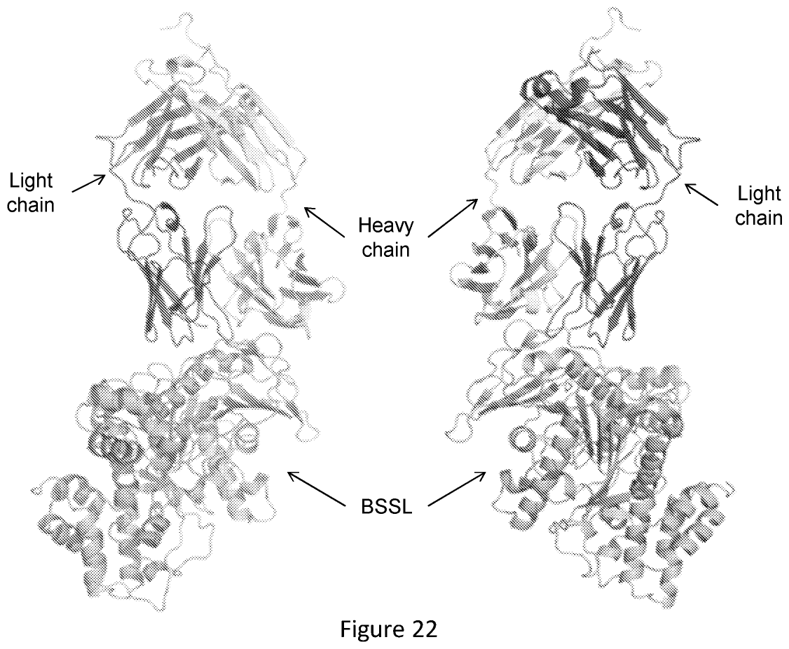

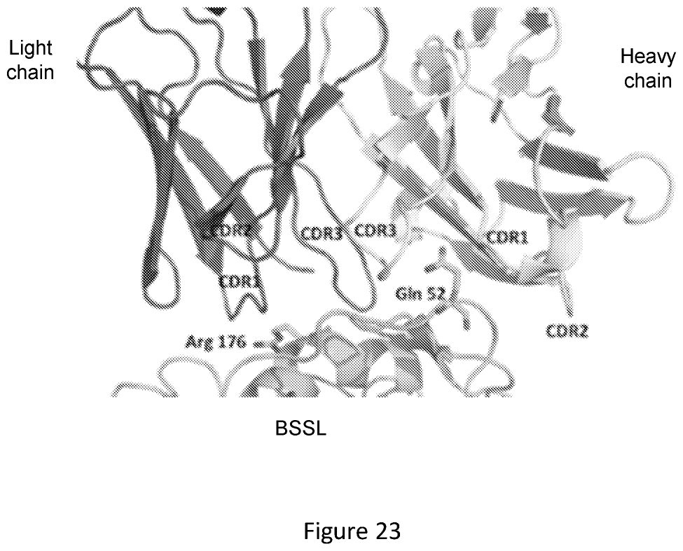

The epitope analysis was performed using the coordinates of the Fab-BSSL complex. The analysis was done using the CONTACT software in the CCP4 suite of programs. The S-SL048-116-Fab binds to BSSL through both the heavy and the light chains. In the heavy chain all three CDR loops are involved (CDR1, CDR2 and CDR3) whereas in the light chain only CDR1 and CDR3 make contact to BSSL, (FIG. 23, Table 26). Also Ile 2 close to the N-terminus of the light chain makes hydrophobic van der Waal's interaction to BSSL. A network of hydrogen bonds stabilises the interaction between the heavy chain and BSSL (altogether 7 hydrogen bonds) and BSSL and the light chain (altogether 5 hydrogen bonds) calculated using PISA (QT) analysis. The PISA (QT) analysis reveals that that 426 Å² of solvent accessible area are buried in the interface between BSSL and the heavy chain and 468 Å² are buried in the interface of BSSL and the light chain.

TABLE 26
A summary of all interacting residues between
S-SL048-116-Fab and BSSL between 0 and 4 Å

| CDR | Fab residue | BSSL residue |
|---|---|---|
| HC-CDR1 | Ser 31 | Gln 52 |
| | Tyr 32 | Gln 52 |
| | Asn 33 | Gln 52, Gly 53 |
| | His 35 | Tyr 7 |
| HC-CDR2 | Val 50 | Tyr 7, Phe 12 |
| | Asn 52 | Phe 12, Gly 53, Thr 54 |
| | Asp 55 | Thr 54 |
| | Ala 57 | Ala 5, Phe 12, Thr 54 |
| | Thr 58 | Phe 12 |
| | Ser 59 | Tyr 7 |
| HC-CDR3 | Asp 99 | Gln 52 |
| | Gly 102 | Pro 49 |
| | Ser 103 | Pro 49, Gly 50, Trp 51, Gln 52 |
| LC | Ile 2 | Arg 176 |
| LC-CDR1 | Pro 27 | Lys 175 |
| | Ser 28 | Ala 172, Arg 176 |
| | Ile 29 | Arg 176 |
| | Ser 30 | Glu 9, Pro 47, Arg 176 |
| | Tyr 31 | Gln 46, Pro 47, His 48 |
| LC-CDR3 | Arg 90 | Tyr 7, Glu 9 |
| | Ser 91 | Tyr 7, Thr 8, Glu 9 |
| | Ser 92 | Tyr 7, Arg 176, Asn 177, Ala 180 |

### Conclusion

The results obtained in the present Example for S-SCL048-116 is in agreement with the results obtained from the HDX-MS study in Example 14.

### Example 23—Efficacy Validation of S-SL048-116 in a Mouse Model of Rheumatoid Arthritis

In this Example, the effect of S-SL048-116 (heavy chain SEQ ID NO: 125 and light chain SEQ ID NO: 126) was investigated in an in vivo mouse model of rheumatoid arthritis (RA), i.e., collagen antibody induced arthritis (CAIA). The CAIA model is described in Example 18. The study was approved by the local animal ethic committee Malmo/Lund, Sweden (02896-20).

### Material and Methods

### Disease Induction

DBA/1 mice (males, 8 weeks) were injected i.v. with 2 mg/mouse of a cocktail of monoclonal anti-CII antibodies (CIA-MAB-50, MD Bioproducts) on day 0. Day 5 the mice were injected with LPS (50 μg/mouse) i.p., in order to boost the disease.

### Experimental Groups and Administration of Test Items

S-SL048-116 was delivered at a concentration of 5 mg/ml and further diluted in vehicle (25 mM Histidine, 150 mM NaCl, 0.02% P80, pH 6.0). Test items (antibodies and vehicle) were administered i.p. every 4^{th} day starting one day prior to disease induction (day −1) and then day 3, 7, 11 and 15. S-SL048-116 was administered at three different doses, i.e., 10, 30 and 90 mg/kg based on mean weight of the animals at day −2. The relatively high doses were chosen to compensate for S-SL048-116's low affinity for mouse BSSL (K_{D}=40 nM) compared to human BSSL (K_{D}=0.5 nM), as described in Example 11. The experimental groups are outlined in Table 27. The first administration at day −1 was a bolus dose, i.e., test items were given as double doses divided into two injections, the first injection was given in the morning and the second injection in the afternoon. The following administrations (day 3, 7, 11 and 15) were given as single doses. The animals were weighed before each administration and the dose volume, 20 ml/kg was based on the individual weight of the animals.

TABLE 27
Experimental groups

| | | Dose | Admin | Termination | No. of |
|---|---|---|---|---|---|
| Group | Treatment | (mg/kg) | route | day | animals |
| 1 | Vehicle | N/A | i.p. | 19 | 15 |
| 2 | S-SL048-116 | 90 | i.p. | 19 | 15 |
| 3 | S-SL048-116 | 30 | i.p. | 19 | 15 |
| 4 | S-SL048-116 | 10 | i.p. | 19 | 15 |
| 5 | Naive | N/A | N/A | 20 | 15 |

### Disease Evaluation

Disease was evaluated daily from day 3 in a blinded fashion using a macroscopic scoring system of the four limbs ranging from 0 to 15 (1 point for each swollen or red toe, 1 point for a swollen or red mid foot digit or knuckle, 5 points for a swollen ankle) resulting in a maximum total score of 60 for each mouse. Due to ethical restrictions, animals with a score exceeding 45 were removed from the experiment.

### Results

### Arthritis Severity

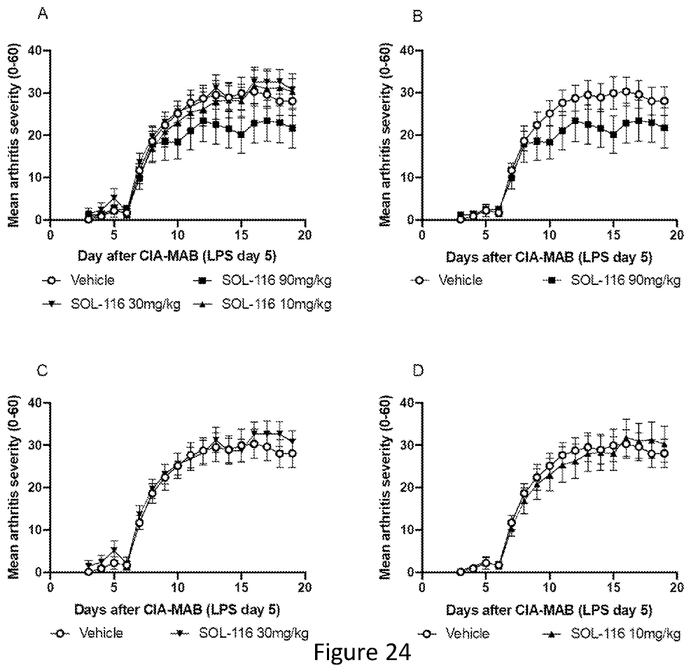

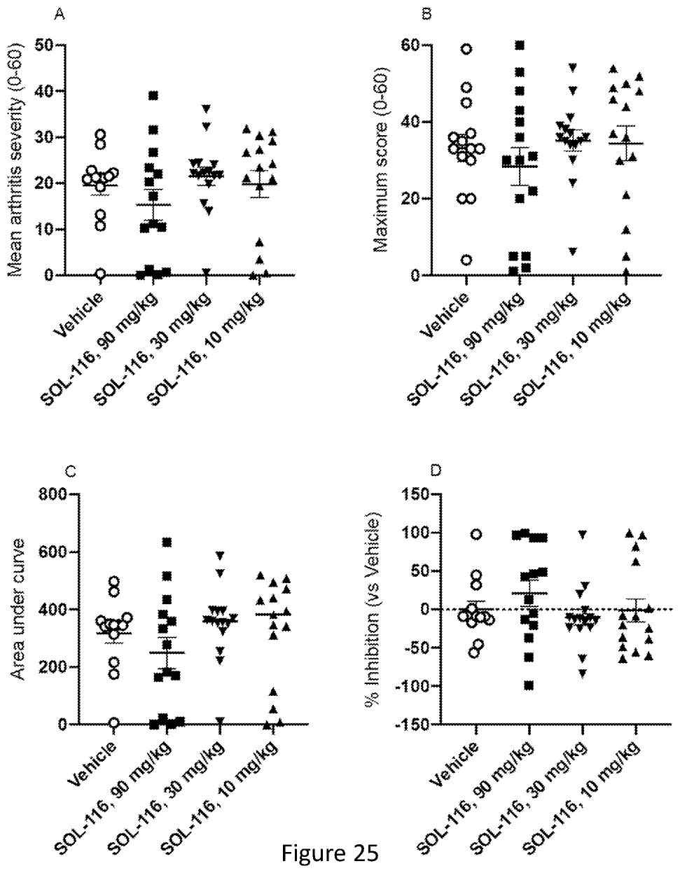

The CAIA induced and vehicle treated animals developed a moderate to severe disease with 100% incidence. The efficacy of S-SL048-116 (SOL-116), dosed i.p. every fourth day from day −1 until termination was evaluated in three doses (10, 30 and 90 mg/kg). S-SL048-116 dosed at 90 mg/kg showed an ameliorating effect on disease severity compared with vehicle (FIGS. 24 and 25).

Two animals were removed pre-termination for ethical reasons (high score), one in the vehicle group (day 15) and one in the 90 mg/kg group (day 12). These animals are included in the maximum score but excluded from mean, AUC and % inhibition (FIG. 25, Table 28). One mouse in the vehicle group did not recover from the LPS boost, i.e., was hypothermic, had a kyphotic posture and very low weight. This mouse was removed before onset of disease (day 7) and is therefore excluded from all results.

Disease parameter statistics are outlined in Table 28.

TABLE 28
CAIA severity parameter statistics

| Group | Incidence¹ | MES² | Max | AUC³ | % Inhibition⁴ |
|---|---|---|---|---|---|
| Vehicle | 100% | 19.5 ± 2.1 | 33.2 ± 3.6 | 317.9 ± 34.9 | N/A |
| S-SL048-116, 90 mg/kg | 80% | 15.3 ± 3.4 | 28.4 ± 4.9 | 249.0 ± 54.5 | 21.2 ± 17.0 |
| S-SL048-116, 30 mg/kg | 93% | 21.5 ± 2.1 | 35.1 ± 2.7 | 349.8 ± 33.4 | −10.0 ± 10.5 |
| S-SL048-116, 10 mg/kg | 87% | 19.8 ± 2.9 | 34.4 ± 4.5 | 321.8 ± 47.7 | −1.23 ± 15.0 |

¹Cumulative incidence. A mouse was considered to have developed disease if scored a point of 1 or higher on two consecutive scoring days.
²Mean CAIA score for all scoring time points.
³Area under curve.
⁴Percent change in overall disease burden in each animal relative to vehicle Ctrl treated group. Calculated by determining the difference between the vehicle Ctrl group mean AUC and the AUC for each individual animal, divided by the vehicle Ctrl group AUC and multiplied by 100 *(−1).

### Health Evaluation

General health assessment was performed daily in conjunction with evaluation of disease from day 3 until termination. The mice were weighed two times weekly as part of the general health assessment. No adverse effects from treatments were observed in the animals.

### Conclusion

The CAIA induced vehicle treated mice developed a moderate to severe disease with 100% incidence. The efficacy of S-SL048-116, at three different doses, i.e. 10, 30 and 90 mg/kg doses i.p., with treatment starting day −1, was evaluated. An ameliorating effect on disease severity was seen with S-SL048-116 dosed at 90 mg/kg when comparing with vehicle. No adverse effects from treatments with S-SL048-116 or vehicle alone were observed.

### Example 24—Analysis of Cellularity in Blood, Spleen and Mesenteric Lymph Nodes Comparing BSSL Knockout (KO) and Wild Type (WT) Mice

In this Example, the quantity of different leukocyte subsets in blood, spleen and mesenteric lymph nodes (MLN) collected from BSSL deficient knockout (KO) mice and wildtype littermates was investigated by fluorescence-activated cell sorting (FACS).

### Material and Methods

Leukocytes were isolated for analysis from blood, spleen and MLN from 10 BSSL KO mice and 10 wildtype littermates (15-19 weeks). Total leukocyte counts were determined for spleen and MLN using manual counting in a Burcher chamber. Cells isolated from the blood, spleen and MLN were incubated with FC-Block (CD16/C032 clone 2.4G2) followed by two different antibody cocktails, Staining A and Staining B.

### Staining A

Antibodies/clones/fluorochromes used for Stain A were: CD19 (ID3/BB515), γδTCR (GL3/PE), CD8a (53-6.7/PerCP-Cy5.5), CD45.2 (104/PE-Cy7), NK1.1 (PK136/APC), CD4 (GK1.5/APC-H7), TCRβ (H57-597/BV421) and fixable viability dye (FVD) (Horizon 510).

Cell populations identified by Staining A were:

αβ T cells: CD45.2+, TCRβ30

CD4 αβ T cells: CD45.2+, TCRIβ+, CD4+

CD8 αβ T cells: CD45.2+, TCRIβ+, CD8+

γδT cells: CD45.2+, TCRγδ+

NKT cells: CD45.2+, TCRIβ+, NK1.1+

B cells: CD45.2+, CD19+

NK cells: CD45.2+, TCRβ−, TCRγδ−, NK1.1+

### Staining B

Antibodies/clones/fluorochromes used for Staining B were: MHC II (MS/114.15.2/Alexa488), FcεRlα (MAR-1/PE), Siglec F (E50-2440/PE-CF594), Ly6G (1A8/PerCP-Cy5.5), CD45.2 (104/PE-Cy7), Ly6C (AL-21/APC), CD11b (M1/70/APC-Cy7), CD117 (2B8/BV421) and fixable viability dye (FVD) (Horizon 510).

Cell populations identified by Staining B were:

Myeloid cells: CD45.2+, CD11b+

Neutrophils: CD45.2+, CD11b+, Ly6G+

Eosinophils: CD45.2+, CD11b+, Ly6G⁻, Siglec F+, SSChigh

Monocytes: CD45.2+, CD11b+, Ly6G⁻, Siglec F−, SSC^{low}, Ly6C+, MHCII−

Macrophages: CD45.2+, CD11b+, Ly6G−, Siglec F−, SSC^{low}, Ly6C−, MHCII+

Mast cells: CD45.2+, CD11 b+, FcεRIα+, CD117+

Basophils: CD45.2+, CD11b+, FcεRIα+, CD117−

After incubation (20 min on ice), cells were washed, resuspended in FACS buffer and different cellular subsets were identified by FACS.

### Results

### Total Number of Leukocytes in Spleen and MLN

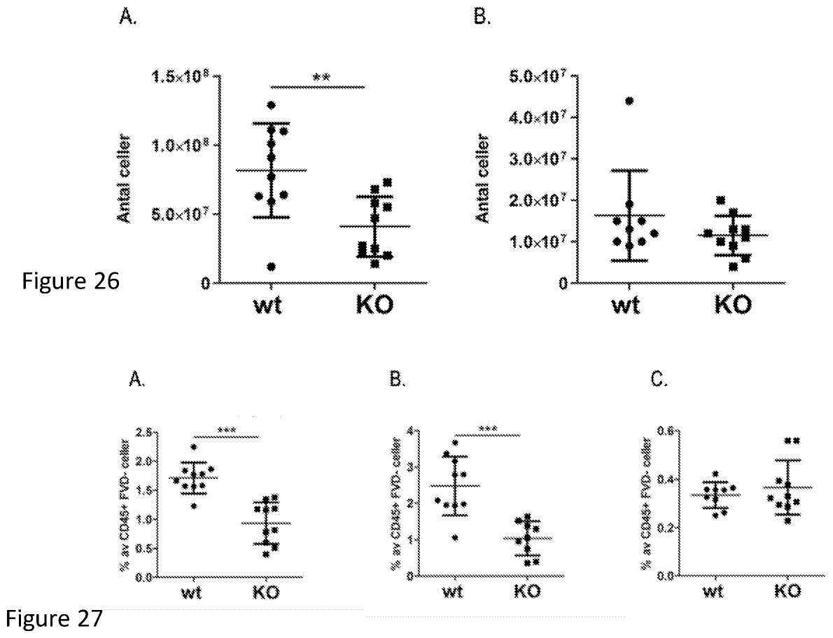

The number of leukocytes in spleen of KO mice was found to be significantly reduced to roughly 50% of that in the WT mice. No significant difference in the number of leukocytes was observed in MLN between KO and WT mice (FIG. 26).

### αβ T Cells

The total number of αβ T cells in the spleen was found to be reduced in the KO mice compared to WT mice. The reduction is similar to that observed for total CD45+ leukocytes. No significant difference in the number of αβ T cells was observed in MLN. The percentage αβ T cells out of total CD45+ cells was found to be higher in KO spleen and blood compared to WT mice but no difference was observed in MLN. In spleen, the CD4+/CD8+ ratio was higher in KO mice compared with WT mice.

γδ T cells The total number of γδ T cells in the spleen was found to be reduced in KO mice compared to

WT mice. The reduction was similar to that observed for total CD45+ leukocytes. In MLN this reduction was even more pronounced than in spleen. The proportion of γδ cells out of CD45+ cells in MLN was also found to be lower in KO mice compared to WT mice. No such difference was observed in spleen and blood.

### NKT Cells

The total number of NKT cells in the spleen was found to be reduced in the KO mice compared with the WI mice. The reduction was similar to that observed for total CD45+ leukocytes. No significant difference in the number of NKT cells was observed in MLN. The proportion of NKT cells out of CD45+ cells in blood was found to be lower in KO mice compared with WI mice. No such difference was observed in spleen and MLN.

### B Cells

The total number of B cells in the spleen was found to be reduced in the KO mice compared to WT mice. The reduction was similar to that observed for total CD45+ leukocytes. No significant difference in the number of B cells was observed in MLN. The proportion of B cells out of CD45+cells in spleen and blood was found to be lower in KO mice compared with WT mice. No such difference was observed in MLN.

### NK Cells

The total number of NK cells in the spleen was found to be reduced in the KO mice compared with WT mice. The reduction was found to be more pronounced for NK cells compared with that observed for total CD45+ leukocytes and other lymphoid subsets. No significant difference in the number of NK cells was observed in MLN. The proportion of NK cells out of CD45+ cells in spleen and blood was found to be lower in KO mice compared with WT mice. No such difference was observed in MLN (FIG. 27).

### Myeloid Cells

The total number of myeloid cells was reduced in KO mice compared to WT mice both in the spleen and in the MLN. The proportion of myeloid cells out of CD45+ cells in spleen and MLN was found to be lower in KO mice compared with WT mice. No such difference was observed in blood.

### Neutrophils

No significant difference in the number of neutrophils was observed in spleen or MLN comparing KO and WT mice. The proportion of neutrophils in the blood was higher in KO mice but no significant difference was noted in spleen and MLN.

### Eosinophils

No significant difference in the number of eosinophils was observed in spleen or MLN comparing KO and WT mice. The proportion of eosinophils in the blood was found to be higher in the KO but no significant difference was noted in spleen and MLN.

### Monocytes

The total number of monocytes was found to be lower in KO compared to WT mice both in the spleen and MLN. No significant difference in the proportion of monocytes was observed between KO and WT mice in any of the organs studied.

### Macrophages

The total number of macrophages was found to be lower in KO compared to WT mice both in the spleen and MLN. No significant difference in the proportion of macrophages was observed between KO and WT mice in any of the organs studied.

### Mast Cells

No significant difference in the number of mast cells in spleen and MLN was observed between KO and WT mice. The proportion of mast cells in the spleen was higher in KO mice compared with WT mice. No difference was observed in blood or MLN.

### Basophils

The total number of basophils in the spleen was found to be lower in KO compared to WT mice. No difference in basophil numbers was observed in MLN. The proportion of basophils in the blood was found to be higher in KO compared with WT mice. No such difference was observed in spleen or MLN.

### Conclusion

In addition to the overall effect on CD45+ leukocytes, the data suggest additional effects specifically on NK cells, i.e., both the total number and the proportion of NK cells out of CD45+ cells were lower in blood and spleen of naïve BSSL deficient mice compared with wildtype littermates. These data support the finding that treatment with AS20 hIgG1 LALA-PG (90 mg/kg dosage) significantly reduced the total number of NK cells and the proportion of T cells, NKT cells and NK cells out of CD45+ cells in spleen from mice with CAIA (Example 19).

TABLE 29
Listing of the sequences and SEQ ID NO:
s used in the present document

| SEQ | | |
|---|---|---|
| ID | | |
| NO: | Nomenclature | Sequence |
| 1 | BSSL Epitope | YTEGGF |
| | 7-12 | |
| 2 | BSSL Epitope | LENPQPHPGWQGTL |
| | 42-55 | |
| 3 | BSSL Epitope | AKLGAVYTEGGF |
| | 1-12 | |
| 4 | BSSL Epitope | NIWVPQGRKQVSRDLPVM |
| | 84-101 (clone | |
| | 46) | |
| 5 | BSSL Epitope | VKRNIAA |
| | 174-180 (clone | |
| | 116) | |
| 6 | BSSL Epitope | HYVGFVPVIDGDF |
| | 283-295 (clone | |
| | 11) | |
| 7 | HCDR1 (clone | GYTFTSYN |
| | 11, 46, 106, | |
| | 116, 118 + | |
| | AS20) | |
| 8 | HCDR2 (clone | INPGDGAT |
| | 46, 116, 118) | |
| 9 | HCDR3 (clone | ARDYYGSSPLGY |
| | 11, 46, 106, | |
| | 116, 118 + | |
| | AS20) | |
| 10 | LCDR1 (clone | PSISY |
| | 11, 116, 118) | |
| 11 | LCDR3 (clone | HQRSSSPT |
| | 46, 116, 118) | |
| 12 | eHCDR2 (clone | MGVINPGDGATSYAQKFK |
| | 116 och 118) | |
| 13 | BSSL Epitope | AWVKRNIAA |
| | 172-180 (AS20) | |
| 14 | eLCDR1 (clone | RASPSISYMN |
| | 116) | |
| 15 | eLCDR2 (clone | ATSSLA |
| | 11, 116) | |
| 16 | eLCDR1 (clone | SASPSISYMN |
| | 11, 118) | |
| 17 | eLCDR2 (clone | ATSSLP |
| | 118) | |
| 18 | HCDR2 (clone | IYPGDGAT |
| | 11) | |
| 19 | HCDR2 (clone | IYPGDGST |
| | 106) | |
| 20 | LCDR1 (clone | SSISY |
| | 46, 106) | |
| 21 | LCDR3 (clone | HQRSSYPT |
| | 11 + AS20) | |
| 22 | LCDR3 (clone | HQRSSTPT |
| | 106) | |
| 23 | eHCDR2 (clone | MGVIYPGDGATSYAQKFK |
| | 11) | |
| 24 | eHCDR2 (clone | MGVINPGDGATSYNQKFQ |
| | 46) | |
| 25 | eHCDR2 (clone | IGVIYPGDGSTSYNQKFQ |
| | 106) | |
| 26 | eLCDR1 (clone | SASSSISYMN |
| | 106) | |
| 27 | eLCDR1 (clone | RASSSISYLN |
| | 46) | |
| 28 | eLCDR2 (clone | ATSKLP |
| | 106) | |
| 29 | eLCDR2 (clone | AASSLA |
| | 46) | |
| 30 | S-SL048-11 | QVQLVQSGAEVKKPGASVKVSCKASGYTFTSYNMHWVRQAPGQGL |
| | HCVR | EWMGVIYPGDGATSYAQKFKGRVTMTRDTSTSTVYMELSSLRSED |
| | | TAVYYCARDYYGSSPLGYWGQGTLVTVSS |
| 31 | S-SL048-11 | DIQMTQSPSSLSASVGDRVTITCSASPSISYMNWYQQKPGKAPKL |
| | LCVR | LIYATSSLASGVPSRFSGSGSGTDFTLTISSLQPEDFATYYCHQR |
| | | SSYPTFGQGTKLEIK |
| 32 | S-SL048-46 | QVQLVQSGAEVKKPGASVKVSCKASGYTFTSYNMHWVRQAPGQGL |
| | HCVR | EWMGVINPGDGATSYNQKFQGRVTMTRDTSTSTVYMELSSLRSED |
| | | TAVYYCARDYYGSSPLGYWGQGTLVTVSS |
| 33 | S-SL048-46 | DIQMTQSPSSLSASVGDRVTITCRASSSISYLNWYQQKPGKAPKL |
| | LCVR | LIYAASSLASGVPSRFSGSGSGTDFTLTISSLQPEDFATYYCHQR |
| | | SSSPTFGQGTKLEIK |
| 34 | S-SL048-106 | QVQLVQSGAEVKKPGASVKVSCKASGYTFTSYNMHWVRQAPGQGL |
| | HCVR | EWIGVIYPGDGSTSYNQKFQGRVTMTRDTSTSTVYMELSSLRSED |
| | | TAVYYCARDYYGSSPLGYWGQGTLVTVSS |
| 35 | S-SL048-106 | DIQMTQSPSSLSASVGDRVTITCSASSSISYMNWYQQKPGKAPKL |
| | LCVR | LIYATSKLPSGVPSRFSGSGSGTDFTLTISSLQPEDFATYYCHQR |
| | | SSTPTFGQGTKLEIK |
| 36 | S-SL048-116 | QVQLVQSGAEVKKPGASVKVSCKASGYTFTSYNMHWVRQAPGQGL |
| | and 118 HCVR | EWMGVINPGDGATSYAQKFKGRVTMTRDTSTSTVYMELSSLRSED |
| | | TAVYYCARDYYGSSPLGYWGQGTLVTVSS |
| 37 | S-SL048-116 | DIQMTQSPSSLSASVGDRVTITCRASPSISYMNWYQQKPGKAPKL |
| | LCVR | LIYATSSLASGVPSRFSGSGSGTDFTLTISSLQPEDFATYYCHQR |
| | | SSSPTFGQGTKLEIK |
| 38 | S-SL048-118 | DIQMTQSPSSLSASVGDRVTITCSASPSISYMNWYQQKPGKAPKL |
| | LCVR | LIYATSSLPSGVPSRFSGSGSGTDFTLTISSLQPEDFATYYCHQR |
| | | SSSPTFGQGTKLEIK |
| 39 | ZH1 | QVQLVQSGAEVKKPGASVKVSCKAS |
| 40 | ZH2 | MHWVRQAPGQGLEW |
| 41 | ZH3 | GRVTMTRDTSTSTVYMELSSLRSEDTAVYYC |
| 42 | ZH4 | WGQGTLVTVSS |
| 43 | ZL1 | DIQMTQSPSSLSASVGDRVTITC |
| 44 | ZL2 | WYQQKPGKAPKLLIY |
| 45 | ZL3 | SGVPSRFSGSGSGTDFTLTISSLQPEDFATYYC |
| 46 | ZL4 | FGQGTKLEIK |
| 47 | S-SL048-10 VH | QVQLVQSGAEVKKPGASVKVSCKASGYTFTSYNMHWVRQAPGQGL |
| | | EWIGVINPSDGYTSYAQKFQGRVTMTRDTSTSTVYMELSSLRSED |
| | | TAVYYCARDYYGSSPLGYWGQGTLVTVSS |
| 48 | S-SL048-12 VH | QVQLVQSGAEVKKPGASVKVSCKASGYTFTSYNMHWVRQAPGQGL |
| | | EWMGVINPSDGATSYTQKFQGRVTMTRDTSTSTVYMELSSLRSED |
| | | TAVYYCARDYYGSSPLGYWGQGTLVTVSS |
| 49 | S-SL048-13 VH | QVQLVQSGAEVKKPGASVKVSCKASGYTFTSYNMHWVRQAPGQGL |
| | | EWIGVINPSDGATSYTQKFQGRVTMTRDTSTSTVYMELSSLRSED |
| | | TAVYYCARDYYGSSPLGYWGQGTLVTVSS |
| 50 | S-SL048-14 VH | QVQLVQSGAEVKKPGASVKVSCKASGYTFTSYNMHWVRQAPGQGL |
| | | EWMGVINPGDGATSYAQKFKGRVTMTRDTSTSTVYMELSSLRSED |
| | | TAVYYCARDYYGSSPLGYWGQGTLVTVSS |
| 51 | S-SL048-17 VH | QVQLVQSGAEVKKPGASVKVSCKASGYTFTSYNMHWVRQAPGQGL |
| | | EWMGVINPGDGYTSYTQKFKGRVTMTRDTSTSTVYMELSSLRSED |
| | | TAVYYCARDYYGSSPLGYWGQGTLVTVSS |
| 52 | S-SL048-18 VH | QVQLVQSGAEVKKPGASVKVSCKASGYTFTSYNMHWVRQAPGQGL |
| | | EWMGVINPSDGATSYTQKFKGRVTMTRDTSTSTVYMELSSLRSED |
| | | TAVYYCARDYYGSSPLGYWGQGTLVTVSS |
| 53 | S-SL048-38 VH | QVQLVQSGAEVKKPGASVKVSCKASGYTFTSYNMHWVRQAPGQGL |
| | | EWMGVINPGDGATSYAQKFQGRVTMTRDTSTSTVYMELSSLRSED |
| | | TAVYYCARDYYGSSPLGYWGQGTLVTVSS |
| 54 | S-SL048-40 VH | QVQLVQSGAEVKKPGASVKVSCKASGYTFTSYNMHWVRQAPGQGL |
| | | EWIGVINPSDGATSYAQKFQGRVTMTRDTSTSTVYMELSSLRSED |
| | | TAVYYCARDYYGSSPLGYWGQGTLVTVSS |
| 55 | S-SL048-41 VH | QVQLVQSGAEVKKPGASVKVSCKASGYTFTSYNMHWVRQAPGQGL |
| | | EWIGVIYPGDGATSYAQKFKGRVTMTRDTSTSTVYMELSSLRSED |
| | | TAVYYCARDYYGSSPLGYWGQGTLVTVSS |
| 56 | S-SL048-43 VH | QVQLVQSGAEVKKPGASVKVSCKASGYTFTSYNMHWVRQAPGQGL |
| | | EWMGVINPGDGATSYTQKFQGRVTMTRDTSTSTVYMELSSLRSED |
| | | TAVYYCARDYYGSSPLGYWGQGTLVTVSS |
| 57 | S-SL048-45 VH | QVQLVQSGAEVKKPGASVKVSCKASGYTFTSYNMHWVRQAPGQGL |
| | | EWMGVINPSDGATSYTQKFQGRVTMTRDTSTSTVYMELSSLRSED |
| | | TAVYYCARDYYGSSPLGYWGQGTLVTVSS |
| 58 | S-SL048-47 VH | QVQLVQSGAEVKKPGASVKVSCKASGYTFTSYNMHWVRQAPGQGL |
| | | EWIGVINPGGGYTSYTQKFQGRVTMTRDTSTSTVYMELSSLRSED |
| | | TAVYYCARDYYGSSPLGYWGQGTLVTVSS |
| 59 | S-SL048-48 VH | QVQLVQSGAEVKKPGASVKVSCKASGYTFTSYNMHWVRQAPGQGL |
| | | EWIGVINPGDGATSYAQKFQGRVTMTRDTSTSTVYMELSSLRSED |
| | | TAVYYCARDYYGSSPLGYWGQGTLVTVSS |
| 60 | S-SL048-65 VH | QVQLVQSGAEVKKPGASVKVSCKASGYTFTSYNMHWVRQAPGQGL |
| | | EWIGVINPGDGATSYAQKFKGRVTMTRDTSTSTVYMELSSLRSED |
| | | TAVYYCARDYYGSSPLGYWGQGTLVTVSS |
| 61 | S-SL048-66 VH | QVQLVQSGAEVKKPGASVKVSCKASGYTFTSYNMHWVRQAPGQGL |
| | | EWMGVINPGDGATSYNQKFKGRVTMTRDTSTSTVYMELSSLRSED |
| | | TAVYYCARDYYGSSPLGYWGQGTLVTVSS |
| 62 | S-SL048-74 VH | QVQLVQSGAEVKKPGASVKVSCKASGYTFTSYNMHWVRQAPGQGL |
| | | EWIGVINPGDGATSYAQKFQGRVTMTRDTSTSTVYMELSSLRSED |
| | | TAVYYCARDYYGSSPLGYWGQGTLVTVSS |
| 63 | S-SL048-75 VH | QVQLVQSGAEVKKPGASVKVSCKASGYTFTSYNMHWVRQAPGQGL |
| | | EWMGVIYPGGGYTSYAQKFKGRVTMTRDTSTSTVYMELSSLRSED |
| | | TAVYYCARDYYGSSPLGYWGQGTLVTVSS |
| 64 | S-SL048-79 VH | QVQLVQSGAEVKKPGASVKVSCKASGYTFTSYNMHWVRQAPGQGL |
| | | EWMGVINPSDGATSYAQKFQGRVTMTRDTSTSTVYMELSSLRSED |
| | | TAVYYCARDYYGSSPLGYWGQGTLVTVSS |
| 65 | S-SL048-81 VH | QVQLVQSGAEVKKPGASVKVSCKASGYTFTSYNMHWVRQAPGQGL |
| | | EWIGVINPGDGATSYAQKFQGRVTMTRDTSTSTVYMELSSLRSED |
| | | TAVYYCARDYYGSSPLGYWGQGTLVTVSS |
| 66 | S-SL048-86 VH | QVQLVQSGAEVKKPGASVKVSCKASGYTFTSYNMHWVRQAPGQGL |
| | | EWMGVINPGDGATSYAQKFQGRVTMTRDTSTSTVYMELSSLRSED |
| | | TAVYYCARDYYGSSPLGYWGQGTLVTVSS |
| 67 | S-SL048-89 VH | QVQLVQSGAEVKKPGASVKVSCKASGYTFTSYNMHWVRQAPGQGL |
| | | EWMGVINPSSGSTSYTQKFQGRVTMTRDTSTSTVYMELSSLRSED |
| | | TAVYYCARDYYGSSPLGYWGQGTLVTVSS |
| 68 | S-SL048-103 | QVQLVQSGAEVKKPGASVKVSCKASGYTFTSYNMHWVRQAPGQGL |
| | VH | EWMGVINPGDGATSYAQKFKGRVTMTRDTSTSTVYMELSSLRSED |
| | | TAVYYCARDYYGSSPLGYWGQGTLVTVSS |
| 69 | S-SL048-104 | QVQLVQSGAEVKKPGASVKVSCKASGYTFTSYNMHWVRQAPGQGL |
| | VH | EWMGVINPGDGATSYDQKFKGRVTMTRDTSTSTVYMELSSLRSED |
| | | TAVYYCARDYYGSSPLGYWGQGTLVTVSS |
| 70 | S-SL048-105 | QVQLVQSGAEVKKPGASVKVSCKASGYTFTSYNMHWVRQAPGQGL |
| | VH | EWMGVINPSDGATSYAQKFQGRVTMTRDTSTSTVYMELSSLRSED |
| | | TAVYYCARDYYGSSPLGYWGQGTLVTVSS |
| 71 | S-SL048-107 | QVQLVQSGAEVKKPGASVKVSCKASGYTFTSYNMHWVRQAPGQGL |
| | VH | EWMGVINPGDGATSYNQKFQGRVTMTRDTSTSTVYMELSSLRSED |
| | | TAVYYCARDYYGSSPLGYWGQGTLVTVSS |
| 72 | S-SL048-108 | QVQLVQSGAEVKKPGASVKVSCKASGYTFTSYNMHWVRQAPGQGL |
| | VH | EWIGVINPSDGATSYAQKFKGRVTMTRDTSTSTVYMELSSLRSED |
| | | TAVYYCARDYYGSSPLGYWGQGTLVTVSS |
| 73 | S-SL048-109 | QVQLVQSGAEVKKPGASVKVSCKASGYTFTSYNMHWVRQAPGQGL |
| | VH | EWMGVINPSDGATSYAQKFKGRVTMTRDTSTSTVYMELSSLRSED |
| | | TAVYYCARDYYGSSPLGYWGQGTLVTVSS |
| 74 | S-SL048-110 | QVQLVQSGAEVKKPGASVKVSCKASGYTFTSYNMHWVRQAPGQGL |
| | VH | EWMGVINPGDGATSYNQKFKGRVTMTRDTSTSTVYMELSSLRSED |
| | | TAVYYCARDYYGSSPLGYWGQGTLVTVSS |
| 75 | S-SL048-112 | QVQLVQSGAEVKKPGASVKVSCKASGYTFTSYNMHWVRQAPGQGL |
| | VH | EWIGIIYPGDGATSYNQKFKGRVTMTRDTSTSTVYMELSSLRSED |
| | | TAVYYCARDYYGSSPLGYWGQGTLVTVSS |
| 76 | S-SL048-115 | QVQLVQSGAEVKKPGASVKVSCKASGYTFTSYNMHWVRQAPGQGL |
| | VH | EWMGVINPSDGATSYAQKFKGRVTMTRDTSTSTVYMELSSLRSED |
| | | TAVYYCARDYYGSSPLGYWGQGTLVTVSS |
| 77 | S-SL048-125 | QVQLVQSGAEVKKPGASVKVSCKASGYTFTSYNMHWVRQAPGQGL |
| | VH | EWIGVINPGDGATSYAQKFQGRVTMTRDTSTSTVYMELSSLRSED |
| | | TAVYYCARDYYGSSPLGYWGQGTLVTVSS |
| 78 | S-SL048-131 | QVQLVQSGAEVKKPGASVKVSCKASGYTFTSYNMHWVRQAPGQGL |
| | VH | EWIGVINPGDGATSYAQKFKGRVTMTRDTSTSTVYMELSSLRSED |
| | | TAVYYCARDYYGSSPLGYWGQGTLVTVSS |
| 79 | S-SL048-134 | QVQLVQSGAEVKKPGASVKVSCKASGYTFTSYNMHWVRQAPGQGL |
| | VH | EWMGVINPSDGATSYAQKFKGRVTMTRDTSTSTVYMELSSLRSED |
| | | TAVYYCARDYYGSSPLGYWGQGTLVTVSS |
| 80 | AS20 VH | QVQLQQPGAELVKPGASVKMSCKASGYTFTSYNMHWVKQTPGQGL |
| | | EWIGVIYPGNGDTSYNQKFKGKATLTADKSSSTAYMQLSSLTSED |
| | | SAVYYCARDYYGSSPLGYWGQGTTLTVSS |
| 81 | S-SL048-10 VL | DIQMTQSPSSLSASVGDRVTITCRASPSISYLNWYQQKPGKAPKL |
| | | LIYATSRLPSGVPSRFSGSGSGTDFTLTISSLQPEDFATYYCQQR |
| | | SSSPTFGQGTKLEIK |
| 82 | S-SL048-12 VL | DIQMTQSPSSLSASVGDRVTITCSASSSISYMHWYQQKPGKAPKL |
| | | LIYAASRLASGVPSRFSGSGSGTDFTLTISSLQPEDFATYYCHQR |
| | | SSTSTFGQGTKLEIK |
| 83 | S-SL048-13 VL | DIQMTQSPSSLSASVGDRVTITCSASPSISYLNWYQQKPGKAPKL |
| | | LIYATSSLASGVPSRFSGSGSGTDFTLTISSLQPEDFATYYCQQR |
| | | SSSPTFGQGTKLEIK |
| 84 | S-SL048-14 VL | DIQMTQSPSSLSASVGDRVTITCRASPSISYLNWYQQKPGKAPKL |
| | | LIYATSRLPSGVPSRFSGSGSGTDFTLTISSLQPEDFATYYCQQR |
| | | SSSPTFGQGTKLEIK |
| 85 | S-SL048-17 VL | DIQMTQSPSSLSASVGDRVTITCRASQSISYLNWYQQKPGKAPKL |
| | | LIYAASKLPSGVPSRFSGSGSGTDFTLTISSLQPEDFATYYCQQR |
| | | SSSPTFGQGTKLEIK |
| 86 | S-SL048-18 VL | DIQMTQSPSSLSASVGDRVTITCRASSSISYLNWYQQKPGKAPKL |
| | | LIYATSRLQSGVPSRFSGSGSGTDFTLTISSLQPEDFATYYCHQR |
| | | SSTPTFGQGTKLEIK |
| 87 | S-SL048-38 VL | DIQMTQSPSSLSASVGDRVTITCRASPSISYLNWYQQKPGKAPKL |
| | | LIYATSSLASGVPSRFSGSGSGTDFTLTISSLQPEDFATYYCQQR |
| | | SSSPTFGQGTKLEIK |
| 88 | S-SL048-40 VL | DIQMTQSPSSLSASVGDRVTITCRASSSISYLNWYQQKPGKAPKL |
| | | LIYAASRLPSGVPSRFSGSGSGTDFTLTISSLQPEDFATYYCQQR |
| | | SSSPTFGQGTKLEIK |
| 89 | S-SL048-41 VL | DIQMTQSPSSLSASVGDRVTITCRASSSISYMNWYQQKPGKAPKL |
| | | LIYATSRLPSGVPSRFSGSGSGTDFTLTISSLQPEDFATYYCHQR |
| | | SSSPTFGQGTKLEIK |
| 90 | S-SL048-43 VL | DIQMTQSPSSLSASVGDRVTITCRASPSISYMNWYQQKPGKAPKL |
| | | LIYAASSLPSGVPSRFSGSGSGTDFTLTISSLQPEDFATYYCQQR |
| | | SSSPTFGQGTKLEIK |
| 91 | S-SL048-45 VL | DIQMTQSPSSLSASVGDRVTITCRASQSISYLHWYQQKPGKAPKL |
| | | LIYAASRLASGVPSRFSGSGSGTDFTLTISSLQPEDFATYYCHQR |
| | | SSTPTFGQGTKLEIK |
| 92 | S-SL048-47 VL | DIQMTQSPSSLSASVGDRVTITCSASPSISYMNWYQQKPGKAPKL |
| | | LIYATSRLQSGVPSRFSGSGSGTDFTLTISSLQPEDFATYYCQQR |
| | | SSTPTFGQGTKLEIK |
| 93 | S-SL048-48 VL | DIQMTQSPSSLSASVGDRVTITCRASPSISYMNWYQQKPGKAPKL |
| | | LIYATSRLQSGVPSRFSGSGSGTDFTLTISSLQPEDFATYYCHQR |
| | | SSSPTFGQGTKLEIK |
| 94 | S-SL048-65 VL | DIQMTQSPSSLSASVGDRVTITCSASQSISYLNWYQQKPGKAPKL |
| | | LIYDASSLPSGVPSRFSGSGSGTDFTLTISSLQPEDFATYYCHQR |
| | | SSSPTFGQGTKLEIK |
| 95 | S-SL048-66 VL | DIQMTQSPSSLSASVGDRVTITCSASQSISYMNWYQQKPGKAPKL |
| | | LIYDASSLASGVPSRFSGSGSGTDFTLTISSLQPEDFATYYCHQR |
| | | SSTPTFGQGTKLEIK |
| 96 | S-SL048-74 VL | DIQMTQSPSSLSASVGDRVTITCRASQSISYLNWYQQKPGKAPKL |
| | | LIYAASSLASGVPSRFSGSGSGTDFTLTISSLQPEDFATYYCHQR |
| | | SSSPTFGQGTKLEIK |
| 97 | S-SL048-75 VL | DIQMTQSPSSLSASVGDRVTITCRASQSISYLNWYQQKPGKAPKL |
| | | LIYATSNLQSGVPSRFSGSGSGTDFTLTISSLQPEDFATYYCHQR |
| | | SSSPTFGQGTKLEIK |
| 98 | S-SL048-79 VL | DIQMTQSPSSLSASVGDRVTITCSASQSISYLNWYQQKPGKAPKL |
| | | LIYAASRLPSGVPSRFSGSGSGTDFTLTISSLQPEDFATYYCHQR |
| | | SSYPTFGQGTKLEIK |
| 99 | S-SL048-81 VL | DIQMTQSPSSLSASVGDRVTITCRASSSISYMHWYQQKPGKAPKL |
| | | LIYAASSLQSGVPSRFSGSGSGTDFTLTISSLQPEDFATYYCHQR |
| | | SSSPTFGQGTKLEIK |
| 100 | S-SL048-86 VL | DIQMTQSPSSLSASVGDRVTITCRASQSISYLHWYQQKPGKAPKL |
| | | LIYATSSLQSGVPSRFSGSGSGTDFTLTISSLQPEDFATYYCHQR |
| | | SSYPTFGQGTKLEIK |
| 101 | S-SL048-89 VL | DIQMTQSPSSLSASVGDRVTITCRASPSISYLNWYQQKPGKAPKL |
| | | LIYATSRLPSGVPSRFSGSGSGTDFTLTISSLQPEDFATYYCQQR |
| | | SSNPTFGQGTKLEIK |
| 102 | S-SL048-103 | DIQMTQSPSSLSASVGDRVTITCRASSSISYMHWYQQKPGKAPKL |
| | VL | LIYATSSLASGVPSRFSGSGSGTDFTLTISSLQPEDFATYYCHQR |
| | | SSSPTFGQGTKLEIK |
| 103 | S-SL048-104 | DIQMTQSPSSLSASVGDRVTITCSASSSISYLHWYQQKPGKAPKL |
| | VL | LIYAASRLASGVPSRFSGSGSGTDFTLTISSLQPEDFATYYCHQR |
| | | SSTPTFGQGTKLEIK |
| 104 | S-SL048-105 | DIQMTQSPSSLSASVGDRVTITCSASQSISYLHWYQQKPGKAPKL |
| | VL | LIYAASKLPSGVPSRFSGSGSGTDFTLTISSLQPEDFATYYCHQR |
| | | SSYPTFGQGTKLEIK |
| 105 | S-SL048-107 | DIQMTQSPSSLSASVGDRVTITCRASPSISYLNWYQQKPGKAPKL |
| | VL | LIYATSSLPSGVPSRFSGSGSGTDFTLTISSLQPEDFATYYCQQR |
| | | SSSPTFGQGTKLEIK |
| 106 | S-SL048-108 | DIQMTQSPSSLSASVGDRVTITCSASPSISYLNWYQQKPGKAPKL |
| | VL | LIYATSSLASGVPSRFSGSGSGTDFTLTISSLQPEDFATYYCQQR |
| | | SSSPTFGQGTKLEIK |
| 107 | S-SL048-109 | DIQMTQSPSSLSASVGDRVTITCRASPSISYLNWYQQKPGKAPKL |
| | VL | LIYATSRLASGVPSRFSGSGSGTDFTLTISSLQPEDFATYYCQQR |
| | | SSSPTFGQGTKLEIK |
| 108 | S-SL048-110 | DIQMTQSPSSLSASVGDRVTITCSASQSISYLHWYQQKPGKAPKL |
| | VL | LIYAASRLASGVPSRFSGSGSGTDFTLTISSLQPEDFATYYCHQR |
| | | SSTPTFGQGTKLEIK |
| 109 | S-SL048-112 | DIQMTQSPSSLSASVGDRVTITCRASSSISYMHWYQQKPGKAPKL |
| | VL | LIYATSRLASGVPSRFSGSGSGTDFTLTISSLQPEDFATYYCHQR |
| | | SSTPTFGQGTKLEIK |
| 110 | S-SL048-115 | DIQMTQSPSSLSASVGDRVTITCRASQSISYMHWYQQKPGKAPKL |
| | VL | LIYAASKLPSGVPSRFSGSGSGTDFTLTISSLQPEDFATYYCHQR |
| | | SSSPTFGQGTKLEIK |
| 111 | S-SL048-125 | DIQMTQSPSSLSASVGDRVTITCRASSSISYLHWYQQKPGKAPKL |
| | VL | LIYAASRLPSGVPSRFSGSGSGTDFTLTISSLQPEDFATYYCHQR |
| | | SSYPTFGQGTKLEIK |
| 112 | S-SL048-131 | DIQMTQSPSSLSASVGDRVTITCRASPSISYLNWYQQKPGKAPKL |
| | VL | LIYAASKLASGVPSRFSGSGSGTDFTLTISSLQPEDFATYYCHQR |
| | | SSSPTFGQGTKLEIK |
| 113 | S-SL048-134 | DIQMTQSPSSLSASVGDRVTITCRASPSISYLNWYQQKPGKAPKL |
| | VL | LIYATSKLPSGVPSRFSGSGSGTDFTLTISSLQPEDFATYYCQQR |
| | | SSYPTFGQGTKLEIK |
| 114 | AS20 VL | QIVLTQSPAVMSASPGEKVTMTCSASSSISYMHWYQQKPGTSPKR |
| | | WIYDTSKLASGVPARFSGSGSGTSYSLTISSMEAEDAATYYCHQR |
| | | SSYPTFGGGTKLEIK |
| 115 | AS20 hIgG1 | QVQLQQPGAELVKPGASVKMSCKASGYTFTSYNMHWVKQTPGQGL |
| | LALA-PG HC | EWIGVIYPGNGDTSYNQKFKGKATLTADKSSSTAYMQLSSLTSED |
| | | SAVYYCARDYYGSSPLGYWGQGTTLTVSSASTKGPSVFPLAPSSK |
| | | SISGGTAALGCLVKDYFPEPVTVSWNSGALISGVHTFPAVLQSSG |
| | | LYSLSSVVTVPSSSLGTQTYICNVNHKPSNTKVDKKVEPKSCDKT |
| | | HTCPPCPAPEAAGGPSVFLFPPKPKDTLMISRTPEVTCVVVDVSH |
| | | EDPEVKFNWYVDGVEVHNAKTKPREEQYNSTYRVVSVLTVLHQDW |
| | | LNGKEYKCKVSNKALGAPIEKTISKAKGQPREPQVYTLPPSRDEL |
| | | TKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| 116 | AS20 hIgG1 | QIVLTQSPAVMSASPGEKVTMTCSASSSISYMHWYQQKPGTSPKR |
| | LALA-PG LC | WIYDTSKLASGVPARFSGSGSGTSYSLTISSMEAEDAATYYCHQR |
| | | SSYPTFGGGTKLEIKRTVAAPSVFIFPPSDEQLKSGTASVVCLLN |
| | | NFYPREAKVQWKVDNALQSGNSQESVTEQDSKDSTYSLSSTLTLS |
| | | KADYEKHKVYACEVTHQGLSSPVTKSFNRGEC |
| 117 | Anti-NP hIgG1 | QVQLQQPGAELVKPGASVKLSCKASGYTFTSYWMHWVKQRPGRGL |
| | LALA-PG HC | EWIGRIDPNSGGTKYNEKFKSKATLTVDKPSSTAYMQLSSLTSED |
| | | SAVYYCARYDYYGSSYFDYWGQGTTVTVSSASTKGPSVFPLAPSS |
| | | KSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDK |
| | | THTCPPCPAPEAAGGPSVFLFPPKPKDTLMISRTPEVTCVVVDVS |
| | | HEDPEVKFNWYVDGVEVHNAKTKPREEQYNSTYRVVSVLTVLHQD |
| | | WLNGKEYKCKVSNKALGAPIEKTISKAKGQPREPQVYTLPPSRDE |
| | | LTKNQVSLTCLVKGFYPSDIAVEWESNGQPENNYKTTPPVLDSDG |
| | | SFFLYSKLTVDKSRWQQGNVFSCSVMHEALHNHYTQKSLSLSPGK |
| 118 | Anti-NP hIgG1 | QAVVTQESALTTSPGETVTLTCRSSTGAVTTSNYANWVQEKPDHL |
| | LALA-PG LC | FTGLIGGTNNRAPGVPARFSGSLIGNKAALTITGAQTEDEAIYFC |
| | | ALWYSNHWVFGGGTKLTVLQPKAAPSVTLFPPSSEELQANKATLV |
| | | CLISDFYPGAVTVAWKADSSPVKAGVETTTPSKQSNNKYAASSYL |
| | | SLIPEQWKSHRSYSCQVTHEGSTVEKTVAPTEC |
| 119 | S-SL048-11 | QVQLVQSGAEVKKPGASVKVSCKASGYTFTSYNMHWVRQAPGQGL |
| | hIgG4 S228P | EWMGVIYPGDGATSYAQKFKGRVTMTRDTSTSTVYMELSSLRSED |
| | HC | TAVYYCARDYYGSSPLGYWGQGTLVTVSSASTKGPSVFPLAPCSR |
| | | STSESTAALGCLVKDYFPEPVTVSWNSGALISGVHTFPAVLQSSG |
| | | LYSLSSVVTVPSSSLGTKTYTCNVDHKPSNTKVDKRVESKYGPPC |
| | | PPCPAPEFLGGPSVFLFPPKPKDTLMISRTPEVTCVVVDVSQEDP |
| | | EVQFNWYVDGVEVHNAKTKPREEQFNSTYRVVSVLTVLHQDWLNG |
| | | KEYKCKVSNKGLPSSIEKTISKAKGQPREPQVYTLPPSQEEMTKN |
| | | QVSLTCLVKGFYPSDIAVEWESNGQPENNYKTTPPVLDSDGSFFL |
| | | YSRLTVDKSRWQEGNVFSCSVMHEALHNHYTQKSLSLSLGK |
| 120 | S-SL048-11 | DIQMTQSPSSLSASVGDRVTITCSASPSISYMNWYQQKPGKAPKL |
| | hIgG4 S228P | LIYATSSLASGVPSRFSGSGSGTDFTLTISSLQPEDFATYYCHQR |
| | LC | SSYPTFGQGTKLEIKRTVAAPSVFIFPPSDEQLKSGTASVVCLLN |
| | | NFYPREAKVQWKVDNALQSGNSQESVTEQDSKDSTYSLSSTLTLS |
| | | KADYEKHKVYACEVTHQGLSSPVTKSFNRGEC |
| 121 | S-SL048-46 | QVQLVQSGAEVKKPGASVKVSCKASGYTFTSYNMHWVRQAPGQGL |
| | hIgG4 S228P | EWMGVINPGDGATSYNQKFQGRVTMTRDTSTSTVYMELSSLRSED |
| | HC | TAVYYCARDYYGSSPLGYWGQGTLVTVSSASTKGPSVFPLAPCSR |
| | | STSESTAALGCLVKDYFPEPVTVSWNSGALISGVHTFPAVLQSSG |
| | | LYSLSSVVTVPSSSLGTKTYTCNVDHKPSNTKVDKRVESKYGPPC |
| | | PPCPAPEFLGGPSVFLFPPKPKDTLMISRTPEVTCVVVDVSQEDP |
| | | EVQFNWYVDGVEVHNAKTKPREEQFNSTYRVVSVLTVLHQDWLNG |
| | | KEYKCKVSNKGLPSSIEKTISKAKGQPREPQVYTLPPSQEEMTKN |
| | | QVSLTCLVKGFYPSDIAVEWESNGQPENNYKTTPPVLDSDGSFFL |
| | | YSRLTVDKSRWQEGNVFSCSVMHEALHNHYTQKSLSLSLGK |
| 122 | S-SL048-46 | DIQMTQSPSSLSASVGDRVTITCRASSSISYLNWYQQKPGKAPKL |
| | hIgG4 S228P | LIYAASSLASGVPSRFSGSGSGTDFTLTISSLQPEDFATYYCHQR |
| | LC | SSSPTFGQGTKLEIKRTVAAPSVFIFPPSDEQLKSGTASVVCLLN |
| | | NFYPREAKVQWKVDNALQSGNSQESVTEQDSKDSTYSLSSTLTLS |
| | | KADYEKHKVYACEVTHQGLSSPVTKSFNRGEC |
| 123 | S-SL048-106 | QVQLVQSGAEVKKPGASVKVSCKASGYTFTSYNMHWVRQAPGQGL |
| | hIgG4 S228P | EWIGVIYPGDGSTSYNQKFQGRVTMTRDTSTSTVYMELSSLRSED |
| | HC | TAVYYCARDYYGSSPLGYWGQGTLVTVSSASTKGPSVFPLAPCSR |
| | | STSESTAALGCLVKDYFPEPVTVSWNSGALISGVHTFPAVLQSSG |
| | | LYSLSSVVTVPSSSLGTKTYTCNVDHKPSNTKVDKRVESKYGPPC |
| | | PPCPAPEFLGGPSVFLFPPKPKDTLMISRTPEVTCVVVDVSQEDP |
| | | EVQFNWYVDGVEVHNAKTKPREEQFNSTYRVVSVLTVLHQDWLNG |
| | | KEYKCKVSNKGLPSSIEKTISKAKGQPREPQVYTLPPSQEEMTKN |
| | | QVSLTCLVKGFYPSDIAVEWESNGQPENNYKTTPPVLDSDGSFFL |
| | | YSRLTVDKSRWQEGNVFSCSVMHEALHNHYTQKSLSLSLGK |
| 124 | S-SL048-106 | DIQMTQSPSSLSASVGDRVTITCSASSSISYMNWYQQKPGKAPKL |
| | hIgG4 S228P | LIYATSKLPSGVPSRFSGSGSGTDFTLTISSLQPEDFATYYCHQR |
| | LC | SSTPTFGQGTKLEIKRTVAAPSVFIFPPSDEQLKSGTASVVCLLN |
| | | NFYPREAKVQWKVDNALQSGNSQESVTEQDSKDSTYSLSSTLTLS |
| | | KADYEKHKVYACEVTHQGLSSPVTKSFNRGEC |
| 125 | S-SL048-116 | QVQLVQSGAEVKKPGASVKVSCKASGYTFTSYNMHWVRQAPGQGL |
| | hIgG4 S228P | EWMGVINPGDGATSYAQKFKGRVTMTRDTSTSTVYMELSSLRSED |
| | HC | TAVYYCARDYYGSSPLGYWGQGTLVTVSSASTKGPSVFPLAPCSR |
| | | STSESTAALGCLVKDYFPEPVTVSWNSGALTSGVHTFPAVLQSSG |
| | | LYSLSSVVTVPSSSLGTKTYTCNVDHKPSNTKVDKRVESKYGPPC |
| | | PPCPAPEFLGGPSVFLFPPKPKDTLMISRTPEVTCVVVDVSQEDP |
| | | EVQFNWYVDGVEVHNAKTKPREEQFNSTYRVVSVLTVLHQDWLNG |
| | | KEYKCKVSNKGLPSSIEKTISKAKGQPREPQVYTLPPSQEEMTKN |
| | | QVSLTCLVKGFYPSDIAVEWESNGQPENNYKTTPPVLDSDGSFFL |
| | | YSRLTVDKSRWQEGNVFSCSVMHEALHNHYTQKSLSLSLGK |
| 126 | S-SL048-116 | DIQMTQSPSSLSASVGDRVTITCRASPSISYMNWYQQKPGKAPKL |
| | hIgG4S228P | LIYATSSLASGVPSRFSGSGSGTDFTLTISSLQPEDFATYYCHQR |
| | LC | SSSPTFGQGTKLEIKRTVAAPSVFIFPPSDEQLKSGTASVVCLLN |
| | | NFYPREAKVQWKVDNALQSGNSQESVTEQDSKDSTYSLSSTLTLS |
| | | KADYEKHKVYACEVTHQGLSSPVTKSFNRGEC |
| 127 | S-SL048-118 | QVQLVQSGAEVKKPGASVKVSCKASGYTFTSYNMHWVRQAPGQGL |
| | hIgG4 S228P | EWMGVINPGDGATSYAQKFKGRVTMTRDTSTSTVYMELSSLRSED |
| | HC | TAVYYCARDYYGSSPLGYWGQGTLVTVSSASTKGPSVFPLAPCSR |
| | | STSESTAALGCLVKDYFPEPVTVSWNSGALISGVHTFPAVLQSSG |
| | | LYSLSSVVTVPSSSLGTKTYTCNVDHKPSNTKVDKRVESKYGPPC |
| | | PPCPAPEFLGGPSVFLFPPKPKDTLMISRTPEVTCVVVDVSQEDP |
| | | EVQFNWYVDGVEVHNAKTKPREEQFNSTYRVVSVLTVLHQDWLNG |
| | | KEYKCKVSNKGLPSSIEKTISKAKGQPREPQVYTLPPSQEEMTKN |
| | | QVSLTCLVKGFYPSDIAVEWESNGQPENNYKTTPPVLDSDGSFFL |
| | | YSRLTVDKSRWQEGNVFSCSVMHEALHNHYTQKSLSLSLGK |
| 128 | S-SL048-118 | DIQMTQSPSSLSASVGDRVTITCSASPSISYMNWYQQKPGKAPKL |
| | hIgG4 S228P | LIYATSSLPSGVPSRFSGSGSGTDFTLTISSLQPEDFATYYCHQR |
| | LC | SSSPTFGQGTKLEIKRTVAAPSVFIFPPSDEQLKSGTASVVCLLN |
| | | NFYPREAKVQWKVDNALQSGNSQESVTEQDSKDSTYSLSSTLTLS |
| | | KADYEKHKVYACEVTHQGLSSPVTKSFNRGEC |
| 129 | AS20 hIgG4 | QVQLQQPGAELVKPGASVKMSCKASGYTFTSYNMHWVKQTPGQGL |
| | S228P HC | EWIGVIYPGNGDTSYNQKFKGKATLTADKSSSTAYMQLSSLTSED |
| | | SAVYYCARDYYGSSPLGYWGQGTTLTVSSASTKGPSVFPLAPCSR |
| | | STSESTAALGCLVKDYFPEPVTVSWNSGALTSGVHTFPAVLQSSG |
| | | LYSLSSVVTVPSSSLGTKTYTCNVDHKPSNTKVDKRVESKYGPPC |
| | | PPCPAPEFLGGPSVFLFPPKPKDTLMISRTPEVTCVVVDVSQEDP |
| | | EVQFNWYVDGVEVHNAKTKPREEQFNSTYRVVSVLTVLHQDWLNG |
| | | KEYKCKVSNKGLPSSIEKTISKAKGQPREPQVYTLPPSQEEMTKN |
| | | QVSLTCLVKGFYPSDIAVEWESNGQPENNYKTTPPVLDSDGSFFL |
| | | YSRLTVDKSRWQEGNVFSCSVMHEALHNHYTQKSLSLSLGK |
| 130 | AS20 hIgG4 | QIVLTQSPAVMSASPGEKVTMTCSASSSISYMHWYQQKPGTSPKR |
| | S228P LC | WIYDTSKLASGVPARFSGSGSGTSYSLTISSMEAEDAATYYCHQR |
| | | SSYPTFGGGTKLEIKRTVAAPSVFIFPPSDEQLKSGTASVVCLLN |
| | | NFYPREAKVQWKVDNALQSGNSQESVTEQDSKDSTYSLSSTLTLS |
| | | KADYEKHKVYACEVTHQGLSSPVTKSFNRGEC |
| 131 | CDR graft | QVQLVQSGAEVKKPGASVKVSCKASGYTFTSYNMHWVRQAPGQGL |
| | hIgG4 S228P | EWIGVIYPGNGDTSYNQKFKGRVTMTRDTSTSTVYMELSSLRSED |
| | HC | TAVYYCARDYYGSSPLGYWGQGTLVTVSSASTKGPSVFPLAPCSR |
| | | STSESTAALGCLVKDYFPEPVTVSWNSGALISGVHTFPAVLQSSG |
| | | LYSLSSVVTVPSSSLGTKTYTCNVDHKPSNTKVDKRVESKYGPPC |
| | | PPCPAPEFLGGPSVFLFPPKPKDTLMISRTPEVTCVVVDVSQEDP |
| | | EVQFNWYVDGVEVHNAKTKPREEQFNSTYRVVSVLTVLHQDWLNG |
| | | KEYKCKVSNKGLPSSIEKTISKAKGQPREPQVYTLPPSQEEMTKN |
| | | QVSLTCLVKGFYPSDIAVEWESNGQPENNYKTTPPVLDSDGSFFL |
| | | YSRLTVDKSRWQEGNVFSCSVMHEALHNHYTQKSLSLSLGK |
| 132 | CDR graft | DIQMTQSPSSLSASVGDRVTITCSASSSISYMHWYQQKPGKAPKL |
| | hIgG4 S228P | LIYDTSKLASGVPSRFSGSGSGTDFTLTISSLQPEDFATYYCHQR |
| | LC | SSYPTFGQGTKLEIKRTVAAPSVFIFPPSDEQLKSGTASVVCLLN |
| | | NFYPREAKVQWKVDNALQSGNSQESVTEQDSKDSTYSLSSTLTLS |
| | | KADYEKHKVYACEVTHQGLSSPVTKSFNRGEC |
| 133 | Anti-NP hIgG4 | QVQLQQPGAELVKPGASVKLSCKASGYTFTSYWMHWVKQRPGRGL |
| | S228P HC | EWIGRIDPNSGGTKYNEKFKSKATLTVDKPSSTAYMQLSSLTSED |
| | | SAVYYCARYDYYGSSYFDYWGQGTTVTVSSASTKGPSVFPLAPCS |
| | | RSTSESTAALGCLVKDYFPEPVTVSWNSGALISGVHTFPAVLQSS |
| | | GLYSLSSVVTVPSSSLGTKTYTCNVDHKPSNTKVDKRVESKYGPP |
| | | CPPCPAPEFLGGPSVFLFPPKPKDTLMISRTPEVTCVVVDVSQED |
| | | PEVQFNWYVDGVEVHNAKTKPREEQFNSTYRVVSVLTVLHQDWLN |
| | | GKEYKCKVSNKGLPSSIEKTISKAKGQPREPQVYTLPPSQEEMTK |
| | | NQVSLTCLVKGFYPSDIAVEWESNGQPENNYKTTPPVLDSDGSFF |
| | | LYSRLTVDKSRWQEGNVFSCSVMHEALHNHYTQKSLSLSLGK |
| 134 | Anti-NPh IgG4 | QAVVTQESALTTSPGETVTLTCRSSTGAVTTSNYANWVQEKPDHL |
| | S228 PLC | FTGLIGGTNNRAPGVPARFSGSLIGNKAALTITGAQTEDEAIYFC |
| | | ALWYSNHWVFGGGTKLTVLRTVAAPSVFIFPPSDEQLKSGTASVV |
| | | CLLNNFYPREAKVQWKVDNALQSGNSQESVTEQDSKDSTYSLSST |
| | | LTLSKADYEKHKVYACEVTHQGLSSPVTKSFNRGEC |
| 135 | AS20 IgG HC | MGWSCIILFLVATATGVHSQVQLQQPGAELVKPGASVKMSCKASG |
| | | YTFTSYNMHWVKQTPGQGLEWIGVIYPGNGDTSYNQKFKGKATLT |
| | | ADKSSSTAYMQLSSLTSEDSAVYYCARDYYGSSPLGYWGQGTTLT |
| | | VSS |
| 136 | AS20 IgG LC | MDFQVQIFSFLLISASVILSRGQIVLTQSPAVMSASPGEKVTMTC |
| | | SASSSISYMHWYQQKPGTSPKRWIYDTSKLASGVPARFSGSGSGT |
| | | SYSLTISSMEAEDAATYYCHQRSSYPTFGGGTKLEIK |
| 137 | mBSSL | AKLGAVYTEGGFVEGVNKKLSLLGGDSVDIFKGIPFATAKTLENP |
| | | QRHPGWQGTLKATNFKKRCLQATITQDNTYGQEDCLYLNIWVPQG |
| | | RKQVSHNLPVMVWIYGGAFLMGSGQGANFLKNYLYDGEEIATRGN |
| | | VIVVTFNYRVGPLGFLSTGDANLPGNFGLRDQHMAIAWVKRNIAA |
| | | FGGDPDNITIFGESAGAASVSLQTLSPYNKGLIRRAISQSGMALS |
| | | PWAIQKNPLFWAKTIAKKVGCPTEDTGKMAACLKITDPRALTLAY |
| | | KLPVKKQEYPVVHYLAFIPVIDGDFIPDDPINLYNNTADIDYIAG |
| | | INNMDGHLFATIDVPAVDKTKQTVTEEDFYRLVSGHTVAKGLKGA |
| | | QATFDIYTESWAQDPSQENMKKTVVAFETDVLFLIPTEIALAQHK |
| | | AHAKSAKTYSYLFSHPSRMPIYPKWMGADHADDLQYVFGKPFATP |
| | | LGYRPQDRAVSKAMIAYWTNFARSGDPNMGNSPVPTHWYPYTLEN |
| | | GNYLDITKTITSASMKEHLREKFLKFWAVTFEVLPTVTGDQDTLT |
| | | PPEDDSEVAPDPPSDDSQVVPVPPTDDSVEAQMPATIGF |
| 138 | hBSSL | AKLGAVYTEG GFVEGVNKKL GLLGDSVDIF KGIPFAAPTK |
| | | ALENPQPHPG WQGTLKAKNF KKRCLQATIT QDSTYGDEDC |
| | | LYLNIWVPQG RKQVSRDLPV MIWIYGGAFL MGSGHGANFL |
| | | NNYLYDGEEI ATRGNVIVVT FNYRVGPLGF LSTGDANLPG |
| | | NYGLRDQHMA IAWVKRNIAA FGGDPNNITL FGESAGGASV |
| | | SLQTLSPYNK GLIRRAISQS GVALSPWVIQ KNPLFWAKKV |
| | | AEKVGCPVGD AARMAQCLKV TDPRALTLAY KVPLAGLEYP |
| | | MLHYVGFVPV IDGDFIPADP INLYANAADI DYIAGTNNMD |
| | | GHIFASIDMP AINKGNKKVT EEDFYKLVSE FTITKGLRGA |
| | | KTTFDVYTES WAQDPSQENK KKTVVDFETD VLFLVPTEIA |
| | | LAQHRANAKS AKTYAYLFSH PSRMPVYPKW VGADHADDIQ |
| | | YVFGKPFATP TGYRPQDRTV SKAMIAYWTN FAKTGDPNMG |
| | | DSAVPTHWEP YTTENSGYLE ITKKMGSSSM KRSLRTNFLR |
| | | YWTLTYLALP TVTDQEATPV PPTGDSEATP VPPTGDSETA |
| | | PVPPTGDSGA PPVPPTGDSG APPVPPTGDS GAPPVPPTGD |
| | | SGAPPVPPTG DSGAPPVPPT GDSGAPPVPP TGDSGAPPVP |
| | | PTGDAGPPPV PPTGDSGAPP VPPTGDSGAP PVTPTGDSET |
| | | APVPPTGDSG APPVPPTGDS EAAPVPPTDD SKEAQMPAVIRF |
| 139 | chimeric AS20 | QVQLQQPGAELVKPGASVKMSCKASGYTFTSYNMHWVKQTPGQGL |
| | IgG4 HC | EWIGVIYPGNGDTSYNQKFKGKATLTADKSSSTAYMQLSSLTSED |
| | | SAVYYCARDYYGSSPLGYWGQGTTLTVSSASTKGPSVFPLAPCSR |
| | | STSESTAALGCLVKDYFPEPVTVSWNSGALISGVHTFPAVLQSSG |
| | | LYSLSSVVTVPSSSLGTKTYTCNVDHKPSNTKVDKRVESKYGPPC |
| | | PSCPAPEFLGGPSVFLFPPKPKDTLMISRTPEVTCVVVDVSQEDP |
| | | EVQFNWYVDGVEVHNAKTKPREEQFNSTYRVVSVLTVLHQDWLNG |
| | | KEYKCKVSNKGLPSSIEKTISKAKGQPREPQVYTLPPSQEEMTKN |
| | | QVSLTCLVKGFYPSDIAVEWESNGQPENNYKTTPPVLDSDGSFFL |
| | | YSRLTVDKSRWQEGNVFSCSVMHEALHNHYTQKSLSLSLGK |
| 140 | chimeric AS20 | QIVLTQSPAVMSASPGEKVTMTCSASSSISYMHWYQQKPGTSPKR |
| | IgG4 LC | WIYDTSKLASGVPARFSGSGSGTSYSLTISSMEAEDAATYYCHQR |
| | | SSYPTFGGGTKLEIKRTVAAPSVFIFPPSDEQLKSGTASVVCLLN |
| | | NFYPREAKVQWKVDNALQSGNSQESVTEQDSKDSTYSLSSTLTLS |
| | | KADYEKHKVYACEVTHQGLSSPVTKSFNRGEC |
| 141 | eHCDR2 of | IGVIYPGNGDTSYNQKFK |
| | AS20 | |
| 142 | eLCDR1 of | SASSSISYMH |
| | AS20 | |
| 143 | eLCDR2of | DTSKLA |
| | AS20 | |
| 144 | CDR graft | QVQLVQSGAEVKKPGASVKVSCKASGYTFTSYNMHWVRQAPGQGL |
| | HCVR | EWIGVIYPGNGDTSYNQKFKGRVTMTRDTSTSTVYMELSSLRSED |
| | | TAVYYCARDYYGSSPLGYWGQGTLVTVSS |
| 145 | CDR graft | DIQMTQSPSSLSASVGDRVTITCSASSSISYMHWYQQKPGKAPKL |
| | LCVR | LIYDTSKLASGVPSRFSGSGSGTDFTLTISSLQPEDFATYYCHQR |
| | | SSYPTFGQGTKLEIK |
| 146 | t-hBSSL | AKLGAVYTEGGFVEGVNKKLGLLGDSVDIFKGIPFAAPTKALENP |
| | | QPHPGWQGTLKAKNFKKRCLQATITQDSTYGDEDCLYLNIWVPQG |
| | | RKQVSRDLPVMIWIYGGAFLMGSGHGANFLNNYLYDGEEIATRGN |
| | | VIVVTFNYRVGPLGFLSTGDANLPGNYGLRDQHMAIAWVKRNIAA |
| | | FGGDPNNITLFGESAGGASVSLQTLSPYNKGLIRRAISQSGVALS |
| | | PWVIQKNPLFWAKKVAEKVGCPVGDAARMAQCLKVTDPRALTLAY |
| | | KVPLAGLEYPMLHYVGFVPVIDGDFIPADPINLYANAADIDYIAG |
| | | TNNMDGHIFASIDMPAINKGNKKVTEEDFYKLVSEFTITKGLRGA |
| | | KTTFDVYTESWAQDPSQENKKKTVVDFETDVLFLVPTEIALAQHR |
| | | ANAKSAKTYAYLFSHPSRMPVYPKWVGADHADDIQYVFGKPFATP |
| | | TGYRPQDRTVSKAMIAYWTNFAKTGDPNMGDSAVPTHWEPYTTEN |
| | | SGYLEITKKMGSSSMKRSLRTNFLRYWTLTYLALPAHHHHHH |
| 147 | BSSL epitope | YTEGG |
| | aa 7-11 | |
| 148 | BSSL epitope | AKLGAVY |
| | 1-7 | |
| 149 | BSSL epitope | AKLGAVYTEGG |
| | 1-11 | |
| 150 | BSSL epitope | AKLGAVYTEGGFVEGVNKKLGLLGD |
| | 1-25 | |
| 151 | BSSL epitope | AKLGAVYTEGGFVEGVNKKLGLLGDS |
| | 1-26 | |
| 152 | BSSL epitope | AKLGAVYTEGGFVEGVNKKLGLLGDSVD |
| | 1-28 | |
| 153 | iTope ™ | VKPGASVKM |
| | residues AS20 | |
| 154 | iTopeTM | YNMHWVRQA |
| | residues | |
| | S-SL048-11, -46, | |
| | -106, -118 | |
| 155 | iTopeTM | YAQKFKGRV |
| | residues | |
| | S-SL048-11, -116, | |
| | -118 | |
| 156 | iTopeTM | WGQGTTLTV |
| | residues AS20 | |
| 157 | iTopeTM | VTMTCSASS |
| | residues AS20 | |
| 158 | iTopeTM | VTITCRASS |
| | residues | |
| | S-SL048-46 | |
| 159 | iTopeTM | VTITCSASS |
| | residues | |
| | S-SL048-106 | |
| 160 | iTopeTM | LLIYATSKL |
| | residues | |
| | S-SL048-106 | |
| 161 | iTopeTM | IYATSSLAS |
| | residues | |
| | S-SL048-11, -116 | |
| 162 | iTopeTM | IYAASSLAS |
| | residues | |
| | S-SL048-46 | |
| 163 | iTopeTM | FGGGTKLEI |
| | residues AS20 | |
| 164 | SL048_hum_AS20_H1 | GATACACCTTCACCAGCTACWATATGCACTGGGTGCG |
| 165 | SL048_hum_AS20_H2 | GACAAGGGCTTGAGTGGATRGGARTAATCWACCCTRGTRRTGGTK |
| | | MCACAAGCTACRMTCAGAAGTTCMAGGGCCGCGTCACC |
| 166 | SL048_hum_AS20_L1 | CGTCACCATCACCTGCAGKGCAAGTYMGAGCATTAGCTATWTGMA |
| | | TTGGTATCAGCAGAAAC |
| 167 | SL048_hum_AS20_L2 | CCTAAGCTCCTGATCTATGMTRCATCCARSTTGSMAAGTGGGGTC |
| | | CCATCAC |
| 168 | SL048_hum_AS20_L3 | GATTTTGCAACTTATTACTGTCASCAGAGKTMTAGTWMTYHCACT |
| | | TTTGGCCAGGGG |
| 169 | HVCR part 2 | XGVXPGDGXTSYXQKFX |
| 170 | LVCR part 1 | XASXSISYXN |
| 171 | LVCR part 2 | AXSXLX |
| 172 | LVCR part 3 | HQRSSXPT |
| 173 | consensus | QQSYSTPT |
| | sequence | |
| 174 | nt S-SL048-11 | CAGGTGCAGCTGGTGCAGTCTGGGGCTGAGGTGAAGAAGCCTGGG |
| | HC | GCCTCAGTGAAGGTTTCCTGCAAGGCATCTGGATACACCTTCACC |
| | | AGCTACAATATGCACTGGGTGCGACAGGCCCCTGGACAAGGGCTT |
| | | GAGTGGATGGGAGTAATCTACCCTGGTGATGGTGCCACAAGCTAC |
| | | GCTCAGAAGTTCAAGGGCCGCGTCACCATGACCCGCGACACGTCC |
| | | ACGAGCACAGTCTACATGGAGCTGAGCAGCCTGCGCTCTGAGGAC |
| | | ACGGCTGTGTATTACTGTGCGAGAGATTACTACGGTAGTAGCCCC |
| | | CTTGGCTACTGGGGCCAAGGAACCCTGGTCACCGTCTCCTCA |
| 175 | nt S-SL048-11 | GACATCCAGATGACCCAGTCTCCATCCTCCCTGAGCGCATCTGTA |
| | LC | GGAGACCGCGTCACCATCACCTGCAGTGCAAGTCCGAGCATTAGC |
| | | TATATGAATTGGTATCAGCAGAAACCAGGGAAAGCCCCTAAGCTC |
| | | CTGATCTATGCTACATCCAGCTTGGCAAGTGGGGTCCCATCACGT |
| | | TTCAGTGGCAGTGGAAGCGGGACAGATTTCACTCTCACCATCAGC |
| | | AGTCTGCAACCTGAAGATTTTGCAACTTATTACTGTCACCAGAGG |
| | | TCTAGTTATCCCACTTTTGGCCAGGGGACCAAGCTGGAGATCAAA |
| 176 | nt S-SL048-46 | CAGGTGCAGCTGGTGCAGTCTGGGGCTGAGGTGAAGAAGCCTGGG |
| | HC | GCCTCAGTGAAGGTTTCCTGCAAGGCATCTGGATACACCTTCACC |
| | | AGCTACAATATGCACTGGGTGCGACAGGCCCCTGGACAAGGGCTT |
| | | GAGTGGATGGGAGTAATCAACCCTGGTGATGGTGCCACAAGCTAC |
| | | AATCAGAAGTTCCAGGGCCGCGTCACCATGACCCGCGACACGTCC |
| | | ACGAGCACAGTCTACATGGAGCTGAGCAGCCTGCGCTCTGAGGAC |
| | | ACGGCTGTGTATTACTGTGCGAGAGATTACTACGGTAGTAGCCCC |
| | | CTTGGCTACTGGGGCCAAGGAACCCTGGTCACCGTCTCCTCA |
| 177 | nt S-SL048-46 | GACATCCAGATGACCCAGTCTCCATCCTCCCTGAGCGCATCTGTA |
| | LC | GGAGACCGCGTCACCATCACCTGCAGGGCAAGTTCGAGCATTAGC |
| | | TATTTGAATTGGTATCAGCAGAAACCAGGGAAAGCCCCTAAGCTC |
| | | CTGATCTATGCTGCATCCAGCTTGGCAAGTGGGGTCCCATCACGT |
| | | TTCAGTGGCAGTGGAAGCGGGACAGATTTCACTCTCACCATCAGC |
| | | AGTCTGCAACCTGAAGATTTTGCAACTTATTACTGTCACCAGAGG |
| | | TCTAGTTCTCCCACTTTTGGCCAGGGGACCAAGCTGGAGATCAAA |
| 178 | nt S-SL048-106 | CAGGTGCAGCTGGTGCAGTCTGGGGCTGAGGTGAAGAAGCCTGGG |
| | HC | GCCTCAGTGAAGGTTTCCTGCAAGGCATCTGGATACACCTTCACC |
| | | AGCTACAATATGCACTGGGTGCGACAGGCCCCTGGACAAGGGCTT |
| | | GAGTGGATAGGAGTAATCTACCCTGGTGATGGTTCCACAAGCTAC |
| | | AATCAGAAGTTCCAGGGCCGCGTCACCATGACCCGCGACACGTCC |
| | | ACGAGCACAGTCTACATGGAGCTGAGCAGCCTGCGCTCTGAGGAC |
| | | ACGGCTGTGTATTACTGTGCGAGAGATTACTACGGTAGTAGCCCC |
| | | CTTGGCTACTGGGGCCAAGGAACCCTGGTCACCGTCTCCTCA |
| 179 | nt S-SL048-106 | GACATCCAGATGACCCAGTCTCCATCCTCCCTGAGCGCATCTGTA |
| | LC | GGAGACCGCGTCACCATCACCTGCAGTGCAAGTTCGAGCATTAGC |
| | | TATATGAATTGGTATCAGCAGAAACCAGGGAAAGCCCCTAAGCTC |
| | | CTGATCTATGCTACATCCAAGTTGCCAAGTGGGGTCCCATCACGT |
| | | TTCAGTGGCAGTGGAAGCGGGACAGATTTCACTCTCACCATCAGC |
| | | AGTCTGCAACCTGAAGATTTTGCAACTTATTACTGTCACCAGAGG |
| | | TCTAGTACTCCCACTTTTGGCCAGGGGACCAAGCTGGAGATCAAA |
| 180 | nt S-SL048-116 | CAGGTGCAGCTGGTGCAGTCTGGGGCTGAGGTGAAGAAGCCTGGG |
| | and 118 HC | GCCTCAGTGAAGGTTTCCTGCAAGGCATCTGGATACACCTTCACC |
| | | AGCTACAATATGCACTGGGTGCGACAGGCCCCTGGACAAGGGCTT |
| | | GAGTGGATGGGAGTAATCAACCCTGGTGATGGTGCCACAAGCTAC |
| | | GCTCAGAAGTTCAAGGGCCGCGTCACCATGACCCGCGACACGTCC |
| | | ACGAGCACAGTCTACATGGAGCTGAGCAGCCTGCGCTCTGAGGAC |
| | | ACGGCTGTGTATTACTGTGCGAGAGATTACTACGGTAGTAGCCCC |
| | | CTTGGCTACTGGGGCCAAGGAACCCTGGTCACCGTCTCCTCA |
| 181 | nt S-SL048-116 | GACATCCAGATGACCCAGTCTCCATCCTCCCTGAGCGCATCTGTA |
| | LC | GGAGACCGCGTCACCATCACCTGCAGGGCAAGTCCGAGCATTAGC |
| | | TATATGAATTGGTATCAGCAGAAACCAGGGAAAGCCCCTAAGCTC |
| | | CTGATCTATGCTACATCCAGCTTGGCAAGTGGGGTCCCATCACGT |
| | | TTCAGTGGCAGTGGAAGCGGGACAGATTTCACTCTCACCATCAGC |
| | | AGTCTGCAACCTGAAGATTTTGCAACTTATTACTGTCACCAGAGG |
| | | TCTAGTTCTCCCACTTTTGGCCAGGGGACCAAGCTGGAGATCAAA |
| 181 | nt S-SL048-118 | GACATCCAGATGACCCAGTCTCCATCCTCCCTGAGCGCATCTGTA |
| | LC | GGAGACCGCGTCACCATCACCTGCAGTGCAAGTCCGAGCATTAGC |
| | | TATATGAATTGGTATCAGCAGAAACCAGGGAAAGCCCCTAAGCTC |
| | | CTGATCTATGCTACATCCAGCTTGCCAAGTGGGGTCCCATCACGT |
| | | TTCAGTGGCAGTGGAAGCGGGACAGATTTCACTCTCACCATCAGC |
| | | AGTCTGCAACCTGAAGATTTTGCAACTTATTACTGTCACCAGAGG |
| | | TCTAGTTCTCCCACTTTTGGCCAGGGGACCAAGCTGGAGATCAAA |
| 183 | nt AS20 HC | CAAGTTCAGCTGCAGCAGCCCGGTGCCGAGCTGGTGAAACCCGGT |
| | | GCCTCTGTGAAGATGAGCTGCAAGGCCAGCGGCTACACCTTTACC |
| | | AGCTACAACATGCACTGGGTGAAGCAGACACCCGGACAAGGTTTA |
| | | GAGTGGATCGGCGTGATCTACCCCGGCAACGGCGACACCTCTTAC |
| | | AACCAGAAGTTCAAGGGCAAGGCCACACTGACCGCCGACAAGAGC |
| | | AGCAGCACCGCCTACATGCAGCTGAGCTCTTTAACCAGCGAGGAC |
| | | TCCGCCGTGTACTACTGCGCTCGTGATTACTACGGCAGCAGCCCT |
| | | TTAGGCTATTGGGGACAAGGTACCACTTTAACCGTCTCGAGCGCC |
| | | TCAACCAAAGGACCCTCCGTGTTTCCCCTCGCCCCCTGTTCCCGC |
| | | TCCACATCCGAGTCAACCGCGGCGCTGGGCTGCCTCGTGAAGGAC |
| | | TACTTCCCTGAGCCCGTCACTGTGTCGTGGAACTCCGGGGCCCTG |
| | | ACCTCCGGCGTGCACACCTTCCCTGCCGTGCTTCAATCCTCCGGA |
| | | CTGTACTCCCTGTCCTCGGTGGTCACCGTGCCGTCGAGCTCGTTG |
| | | GGAACCAAGACTTACACTTGCAACGTGGACCACAAGCCAAGCAAC |
| | | ACCAAAGTGGACAAGAGAGTCGAATCTAAGTACGGACCGCCCTGC |
| | | CCGCCTTGCCCCGCCCCTGAGTTTCTCGGCGGTCCTAGCGTGTTC |
| | | CTGTTCCCACCCAAGCCCAAGGACACTCTGATGATCTCCCGGACC |
| | | CCTGAAGTGACCTGTGTGGTCGTGGACGTGTCGCAGGAAGATCCG |
| | | GAGGTCCAGTTCAATTGGTACGTGGATGGGGTGGAGGTCCACAAC |
| | | GCCAAGACGAAGCCGAGAGAAGAACAGTTCAACTCAACTTACCGG |
| | | GTGGTGTCCGTGCTGACCGTGCTGCATCAGGATTGGCTCAACGGA |
| | | AAGGAGTACAAGTGCAAAGTGTCCAACAAGGGCCTGCCTAGCTCA |
| | | ATCGAAAAGACCATTTCCAAGGCCAAGGGCCAGCCGAGGGAACCA |
| | | CAGGTCTATACTCTGCCACCGAGCCAAGAAGAGATGACCAAGAAC |
| | | CAAGTGTCCCTGACTTGCCTGGTCAAGGGGTTCTACCCGTCGGAC |
| | | ATCGCAGTGGAGTGGGAGAGCAACGGACAGCCTGAAAACAATTAC |
| | | AAGACCACCCCGCCCGTGCTGGATAGCGACGGTTCCTTCTTCCTT |
| | | TACTCGCGCCTCACCGTCGACAAGAGCCGGTGGCAGGAGGGCAAC |
| | | GTGTTCTCCTGCTCCGTGATGCACGAAGCTCTGCATAACCACTAC |
| | | ACTCAGAAGTCCTTGTCGCTGAGCCTCGGAAAG |
| 184 | nt AS20 LC | CAGATCGTGCTGACCCAGAGCCCCGCTGTGATGAGCGCCTCTCCC |
| | | GGTGAGAAGGTGACCATGACTTGTAGCGCCAGCAGCAGCATCAGC |
| | | TACATGCACTGGTACCAGCAGAAGCCCGGCACCAGCCCTAAGAGG |
| | | TGGATCTACGACACCAGCAAGCTGGCCAGCGGCGTGCCCGCTAGG |
| | | TTCAGCGGAAGCGGCAGCGGCACCAGCTACTCTTTAACCATCAGC |
| | | AGCATGGAGGCCGAGGATGCCGCCACCTACTACTGCCACCAGAGA |
| | | AGCAGCTACCCCACCTTCGGCGGCGGCACCAAGCTCGAGATCAAG |
| | | AGAACTGTGGCCGCGCCGTCAGTGTTTATCTTCCCTCCATCGGAT |
| | | GAACAGCTTAAGTCCGGCACGGCGTCTGTGGTCTGCCTGCTCAAT |
| | | ACTTTTACCCTAGGGAAGCTAAAGTCCAATGGAAAGTGGATAAC |
| | | GCCCTGCAGTCAGGAAACAGCCAGGAATCGGTTACCGAACAGGAC |
| | | AGCAAGGACAGCACTTACTCCTTGTCGTCGACTCTTACTCTGAGC |
| | | AAGGCCGATTACGAGAAGCACAAGGTCTACGCCTGCGAGGTCACC |
| | | CATCAGGGACTCTCGTCCCCGGTGACCAAATCCTTCAATAGAGGC |
| | | GAATGC |
| 185 | nt S-SL048-11 | CTCAAGTTCAGCTCGTGCAGAGCGGTGCTGAAGTGAAGAAGCCCG |
| | HC | GTGCCTCTGTGAAGGTGAGCTGCAAGGCCAGCGGCTACACCTTCA |
| | | CCAGCTACAACATGCACTGGGTGAGACAAGCTCCCGGTCAAGGTT |
| | | TAGAGTGGATGGGCGTGATCTACCCCGGTGATGGTGCTACCAGCT |
| | | ACGCCCAGAAGTTCAAGGGTCGTGTGACCATGACCAGAGACACCA |
| | | GCACCAGCACCGTGTACATGGAGCTGAGCTCTTTAAGGAGCGAGG |
| | | ACACCGCCGTGTACTACTGCGCTCGTGACTACTACGGCAGCAGCC |
| | | CTTTAGGCTATTGGGGACAAGGTACTTTAGTGACCGTCTCGAGCG |
| | | CCTCAACCAAAGGACCCTCCGTGTTTCCCCTCGCCCCCTGTTCCC |
| | | GCTCCACATCCGAGTCAACCGCGGCGCTGGGCTGCCTCGTGAAGG |
| | | ACTACTTCCCTGAGCCCGTCACTGTGTCGTGGAACTCCGGGGCCC |
| | | TGACCTCCGGCGTGCACACCTTCCCTGCCGTGCTTCAATCCTCCG |
| | | GACTGTACTCCCTGTCCTCGGTGGTCACCGTGCCGTCGAGCTCGT |
| | | TGGGAACCAAGACTTACACTTGCAACGTGGACCACAAGCCAAGCA |
| | | ACACCAAAGTGGACAAGAGAGTCGAATCTAAGTACGGACCGCCCT |
| | | GCCCGCCTTGCCCCGCCCCTGAGTTTCTCGGCGGTCCTAGCGTGT |
| | | TCCTGTTCCCACCCAAGCCCAAGGACACTCTGATGATCTCCCGGA |
| | | CCCCTGAAGTGACCTGTGTGGTCGTGGACGTGTCGCAGGAAGATC |
| | | CGGAGGTCCAGTTCAATTGGTACGTGGATGGGGTGGAGGTCCACA |
| | | ACGCCAAGACGAAGCCGAGAGAAGAACAGTTCAACTCAACTTACC |
| | | GGGTGGTGTCCGTGCTGACCGTGCTGCATCAGGATTGGCTCAACG |
| | | GAAAGGAGTACAAGTGCAAAGTGTCCAACAAGGGCCTGCCTAGCT |
| | | CAATCGAAAAGACCATTTCCAAGGCCAAGGGCCAGCCGAGGGAAC |
| | | CACAGGTCTATACTCTGCCACCGAGCCAAGAAGAGATGACCAAGA |
| | | ACCAAGTGTCCCTGACTTGCCTGGTCAAGGGGTTCTACCCGTCGG |
| | | ACATCGCAGTGGAGTGGGAGAGCAACGGACAGCCTGAAAACAATT |
| | | ACAAGACCACCCCGCCCGTGCTGGATAGCGACGGTTCCTTCTTCC |
| | | TTTACTCGCGCCTCACCGTCGACAAGAGCCGGTGGCAGGAGGGCA |
| | | ACGTGTTCTCCTGCTCCGTGATGCACGAAGCTCTGCATAACCACT |
| | | ACACTCAGAAGTCCTTGTCGCTGAGCCTCGGAAAG |
| 186 | nt S-SL048-11 | GACATCCAGATGACCCAGAGCCCTAGCTCTTTAAGCGCCTCTGTG |
| | LC | GGCGATCGTGTGACCATCACTTGTAGCGCCAGCCCCAGCATCAGC |
| | | TACATGAACTGGTACCAGCAGAAGCCCGGCAAGGCCCCCAAGCTG |
| | | CTGATCTACGCCACAAGCTCTTTAGCCAGCGGCGTGCCTAGCAGA |
| | | TTTAGCGGCAGCGGTAGCGGCACAGACTTCACTTTAACCATCAGC |
| | | TCTTTACAGCCAGAAGACTTCGCCACCTACTACTGCCACCAGAGG |
| | | AGCAGCTACCCCACCTTCGGCCAAGGTACCAAGCTCGAGATCAAG |
| | | AGAACTGTGGCCGCGCCGTCAGTGTTTATCTTCCCTCCATCGGAT |
| | | GAACAGCTTAAGTCCGGCACGGCGTCTGTGGTCTGCCTGCTCAAT |
| | | ACTTTTTACCCTAGGGAAGCTAAAGTCCAATGGAAAGTGGATAAC |
| | | GCCCTGCAGTCAGGAAACAGCCAGGAATCGGTTACCGAACAGGAC |
| | | AGCAAGGACAGCACTTACTCCTTGTCGTCGACTCTTACTCTGAGC |
| | | AAGGCCGATTACGAGAAGCACAAGGTCTACGCCTGCGAGGTCACC |
| | | CATCAGGGACTCTCGTCCCCGGTGACCAAATCCTTCAATAGAGGC |
| | | GAATGC |
| 187 | nt S-SL048-46 | CAAGTTCAGCTCGTGCAGAGCGGTGCTGAAGTGAAGAAGCCCGGT |
| | HC | GCCTCTGTGAAGGTGAGCTGCAAGGCCAGCGGCTACACCTTCACC |
| | | AGCTACAACATGCACTGGGTGAGACAAGCTCCCGGTCAAGGTTTA |
| | | GAGTGGATGGGCGTGATCAACCCCGGTGATGGTGCTACCAGCTAC |
| | | AACCAGAAGTTCCAGGGTCGTGTGACCATGACCAGAGACACCAGC |
| | | ACCAGCACCGTGTACATGGAGCTGAGCTCTTTAAGGAGCGAGGAC |
| | | ACCGCCGTGTACTACTGCGCTCGTGACTACTACGGCAGCAGCCCT |
| | | TTAGGCTATTGGGGACAAGGTACTTTAGTGACCGTCTCGAGCGCC |
| | | TCAACCAAAGGACCCTCCGTGTTTCCCCTCGCCCCCTGTTCCCGC |
| | | TCCACATCCGAGTCAACCGCGGCGCTGGGCTGCCTCGTGAAGGAC |
| | | TACTTCCCTGAGCCCGTCACTGTGTCGTGGAACTCCGGGGCCCTG |
| | | ACCTCCGGCGTGCACACCTTCCCTGCCGTGCTTCAATCCTCCGGA |
| | | CTGTACTCCCTGTCCTCGGTGGTCACCGTGCCGTCGAGCTCGTTG |
| | | GGAACCAAGACTTACACTTGCAACGTGGACCACAAGCCAAGCAAC |
| | | ACCAAAGTGGACAAGAGAGTCGAATCTAAGTACGGACCGCCCTGC |
| | | CCGCCTTGCCCCGCCCCTGAGTTTCTCGGCGGTCCTAGCGTGTTC |
| | | CTGTTCCCACCCAAGCCCAAGGACACTCTGATGATCTCCCGGACC |
| | | CCTGAAGTGACCTGTGTGGTCGTGGACGTGTCGCAGGAAGATCCG |
| | | GAGGTCCAGTTCAATTGGTACGTGGATGGGGTGGAGGTCCACAAC |
| | | GCCAAGACGAAGCCGAGAGAAGAACAGTTCAACTCAACTTACCGG |
| | | GTGGTGTCCGTGCTGACCGTGCTGCATCAGGATTGGCTCAACGGA |
| | | AAGGAGTACAAGTGCAAAGTGTCCAACAAGGGCCTGCCTAGCTCA |
| | | ATCGAAAAGACCATTTCCAAGGCCAAGGGCCAGCCGAGGGAACCA |
| | | CAGGTCTATACTCTGCCACCGAGCCAAGAAGAGATGACCAAGAAC |
| | | CAAGTGTCCCTGACTTGCCTGGTCAAGGGGTTCTACCCGTCGGAC |
| | | ATCGCAGTGGAGTGGGAGAGCAACGGACAGCCTGAAAACAATTAC |
| | | AAGACCACCCCGCCCGTGCTGGATAGCGACGGTTCCTTCTTCCTT |
| | | TACTCGCGCCTCACCGTCGACAAGAGCCGGTGGCAGGAGGGCAAC |
| | | GTGTTCTCCTGCTCCGTGATGCACGAAGCTCTGCATAACCACTAC |
| | | ACTCAGAAGTCCTTGTCGCTGAGCCTCGGAAAG |
| 188 | nt S-SL048-46 | GACATCCAGATGACCCAGAGCCCTAGCTCTTTAAGCGCCTCTGTG |
| | LC | GGCGATCGTGTGACCATCACTTGTCGTGCCAGCAGCAGCATCAGC |
| | | TATTTAAACTGGTACCAGCAGAAGCCCGGCAAGGCCCCCAAGCTG |
| | | CTGATCTACGCCGCCTCTTCTTTAGCCTCTGGCGTGCCTTCTCGT |
| | | TTCAGCGGAAGCGGCAGCGGCACCGACTTCACTTTAACCATCAGC |
| | | TCTTTACAGCCAGAAGACTTCGCCACCTACTACTGCCACCAGAGG |
| | | AGCAGCAGCCCCACCTTCGGACAAGGTACCAAGCTCGAGATCAAG |
| | | AGAACTGTGGCCGCGCCGTCAGTGTTTATCTTCCCTCCATCGGAT |
| | | GAACAGCTTAAGTCCGGCACGGCGTCTGTGGTCTGCCTGCTCAAT |
| | | AACTTTTACCCTAGGGAAGCTAAAGTCCAATGGAAAGTGGATAAC |
| | | GCCCTGCAGTCAGGAAACAGCCAGGAATCGGTTACCGAACAGGAC |
| | | AGCAAGGACAGCACTTACTCCTTGTCGTCGACTCTTACTCTGAGC |
| | | AAGGCCGATTACGAGAAGCACAAGGTCTACGCCTGCGAGGTCACC |
| | | CATCAGGGACTCTCGTCCCCGGTGACCAAATCCTTCAATAGAGGC |
| | | GAATGC |
| 189 | nt S-SL048-106 | CAAGTTCAGCTGGTGCAGAGCGGAGCCGAGGTGAAAAAGCCCGGT |
| | HC | GCCTCTGTGAAGGTGAGCTGCAAGGCCAGCGGCTACACCTTTACC |
| | | AGCTACAACATGCACTGGGTGAGGCAAGCTCCCGGTCAAGGTCTG |
| | | GAGTGGATCGGCGTGATCTACCCCGGCGACGGCAGCACCTCTTAC |
| | | AACCAGAAGTTCCAAGGTCGTGTGACCATGACTCGTGACACCAGC |
| | | ACCAGCACCGTGTACATGGAGCTGAGCTCTTTAAGGAGCGAGGAT |
| | | ACCGCCGTGTACTACTGCGCTCGTGACTACTACGGCAGCAGCCCT |
| | | CTGGGCTATTGGGGCCAAGGTACTTTAGTGACCGTCTCGAGCGCC |
| | | TCAACCAAAGGACCCTCCGTGTTTCCCCTCGCCCCCTGTTCCCGC |
| | | TCCACATCCGAGTCAACCGCGGCGCTGGGCTGCCTCGTGAAGGAC |
| | | TACTTCCCTGAGCCCGTCACTGTGTCGTGGAACTCCGGGGCCCTG |
| | | ACCTCCGGCGTGCACACCTTCCCTGCCGTGCTTCAATCCTCCGGA |
| | | CTGTACTCCCTGTCCTCGGTGGTCACCGTGCCGTCGAGCTCGTTG |
| | | GGAACCAAGACTTACACTTGCAACGTGGACCACAAGCCAAGCAAC |
| | | ACCAAAGTGGACAAGAGAGTCGAATCTAAGTACGGACCGCCCTGC |
| | | CCGCCTTGCCCCGCCCCTGAGTTTCTCGGCGGTCCTAGCGTGTTC |
| | | CTGTTCCCACCCAAGCCCAAGGACACTCTGATGATCTCCCGGACC |
| | | CCTGAAGTGACCTGTGTGGTCGTGGACGTGTCGCAGGAAGATCCG |
| | | GAGGTCCAGTTCAATTGGTACGTGGATGGGGTGGAGGTCCACAAC |
| | | GCCAAGACGAAGCCGAGAGAAGAACAGTTCAACTCAACTTACCGG |
| | | GTGGTGTCCGTGCTGACCGTGCTGCATCAGGATTGGCTCAACGGA |
| | | AAGGAGTACAAGTGCAAAGTGTCCAACAAGGGCCTGCCTAGCTCA |
| | | ATCGAAAAGACCATTTCCAAGGCCAAGGGCCAGCCGAGGGAACCA |
| | | CAGGTCTATACTCTGCCACCGAGCCAAGAAGAGATGACCAAGAAC |
| | | CAAGTGTCCCTGACTTGCCTGGTCAAGGGGTTCTACCCGTCGGAC |
| | | ATCGCAGTGGAGTGGGAGAGCAACGGACAGCCTGAAAACAATTAC |
| | | AAGACCACCCCGCCCGTGCTGGATAGCGACGGTTCCTTCTTCCTT |
| | | TACTCGCGCCTCACCGTCGACAAGAGCCGGTGGCAGGAGGGCAAC |
| | | GTGTTCTCCTGCTCCGTGATGCACGAAGCTCTGCATAACCACTAC |
| | | ACTCAGAAGTCCTTGTCGCTGAGCCTCGGAAAG |
| 190 | nt S-SL048-106 | GACATCCAGATGACCCAGAGCCCTAGCTCTTTAAGCGCCTCTGTG |
| | LC | GGCGATCGTGTGACCATCACTTGTAGCGCCAGCAGCAGCATCAGC |
| | | TACATGAACTGGTACCAGCAGAAGCCCGGCAAGGCCCCCAAGCTG |
| | | CTGATCTACGCCACCAGCAAGCTGCCTAGCGGCGTGCCCTCCAGA |
| | | TTTTCTGGCAGCGGCTCTGGCACCGACTTTACTTTAACCATCAGC |
| | | TCTTTACAGCCAGAAGACTTCGCCACCTACTACTGCCACCAGAGG |
| | | AGCAGCACCCCTACCTTCGGCCAAGGTACCAAGCTCGAGATCAAG |
| | | AGAACTGTGGCCGCGCCGTCAGTGTTTATCTTCCCTCCATCGGAT |
| | | GAACAGCTTAAGTCCGGCACGGCGTCTGTGGTCTGCCTGCTCAAT |
| | | AACTTTTACCCTAGGGAAGCTAAAGTCCAATGGAAAGTGGATAAC |
| | | GCCCTGCAGTCAGGAAACAGCCAGGAATCGGTTACCGAACAGGAC |
| | | AGCAAGGACAGCACTTACTCCTTGTCGTCGACTCTTACTCTGAGC |
| | | AAGGCCGATTACGAGAAGCACAAGGTCTACGCCTGCGAGGTCACC |
| | | CATCAGGGACTCTCGTCCCCGGTGACCAAATCCTTCAATAGAGGC |
| | | GAATGC |
| 191 | nt S-SL048-116 | CAAGTTCAGCTCGTGCAGAGCGGTGCTGAAGTGAAGAAGCCCGGT |
| | and 118 HC | GCCTCTGTGAAGGTGAGCTGCAAGGCCAGCGGCTACACCTTCACC |
| | | AGCTACAACATGCACTGGGTGAGACAAGCTCCCGGTCAAGGTTTA |
| | | GAGTGGATGGGCGTGATCAACCCCGGTGATGGTGCTACCAGCTAC |
| | | GCCCAGAAGTTCAAGGGTCGTGTGACCATGACCAGAGACACCAGC |
| | | ACCAGCACCGTGTACATGGAGCTGAGCTCTTTAAGGAGCGAGGAC |
| | | ACCGCCGTGTACTACTGCGCTCGTGACTACTACGGCAGCAGCCCT |
| | | TTAGGCTATTGGGGACAAGGTACTTTAGTGACCGTCTCGAGCGCC |
| | | TCAACCAAAGGACCCTCCGTGTTTCCCCTCGCCCCCTGTTCCCGC |
| | | TCCACATCCGAGTCAACCGCGGCGCTGGGCTGCCTCGTGAAGGAC |
| | | TACTTCCCTGAGCCCGTCACTGTGTCGTGGAACTCCGGGGCCCTG |
| | | ACCTCCGGCGTGCACACCTTCCCTGCCGTGCTTCAATCCTCCGGA |
| | | CTGTACTCCCTGTCCTCGGTGGTCACCGTGCCGTCGAGCTCGTTG |
| | | GGAACCAAGACTTACACTTGCAACGTGGACCACAAGCCAAGCAAC |
| | | ACCAAAGTGGACAAGAGAGTCGAATCTAAGTACGGACCGCCCTGC |
| | | CCGCCTTGCCCCGCCCCTGAGTTTCTCGGCGGTCCTAGCGTGTTC |
| | | CTGTTCCCACCCAAGCCCAAGGACACTCTGATGATCTCCCGGACC |
| | | CCTGAAGTGACCTGTGTGGTCGTGGACGTGTCGCAGGAAGATCCG |
| | | GAGGTCCAGTTCAATTGGTACGTGGATGGGGTGGAGGTCCACAAC |
| | | GCCAAGACGAAGCCGAGAGAAGAACAGTTCAACTCAACTTACCGG |
| | | GTGGTGTCCGTGCTGACCGTGCTGCATCAGGATTGGCTCAACGGA |
| | | AAGGAGTACAAGTGCAAAGTGTCCAACAAGGGCCTGCCTAGCTCA |
| | | ATCGAAAAGACCATTTCCAAGGCCAAGGGCCAGCCGAGGGAACCA |
| | | CAGGTCTATACTCTGCCACCGAGCCAAGAAGAGATGACCAAGAAC |
| | | CAAGTGTCCCTGACTTGCCTGGTCAAGGGGTTCTACCCGTCGGAC |
| | | ATCGCAGTGGAGTGGGAGAGCAACGGACAGCCTGAAAACAATTAC |
| | | AAGACCACCCCGCCCGTGCTGGATAGCGACGGTTCCTTCTTCCTT |
| | | TACTCGCGCCTCACCGTCGACAAGAGCCGGTGGCAGGAGGGCAAC |
| | | GTGTTCTCCTGCTCCGTGATGCACGAAGCTCTGCATAACCACTAC |
| | | ACTCAGAAGTCCTTGTCGCTGAGCCTCGGAAAG |
| 192 | nt S-SL048-116 | GACATCCAGATGACCCAGAGCCCTAGCTCTTTAAGCGCCAGCGTG |
| | LC | GGAGATCGTGTGACCATCACTTGTCGTGCCAGCCCCAGCATCAGC |
| | | TACATGAACTGGTACCAGCAGAAGCCCGGCAAGGCCCCCAAGCTG |
| | | CTGATCTACGCCACCAGCTCTTTAGCCTCTGGCGTGCCTAGCAGA |
| | | TTCAGCGGCAGCGGAAGCGGCACCGACTTCACTTTAACCATCAGC |
| | | TCTTTACAGCCAGAAGACTTCGCCACCTACTACTGCCACCAGAGG |
| | | AGCAGCAGCCCTACCTTCGGCCAAGGTACCAAGCTCGAGATCAAG |
| | | AGAACTGTGGCCGCGCCGTCAGTGTTTATCTTCCCTCCATCGGAT |
| | | GAACAGCTTAAGTCCGGCACGGCGTCTGTGGTCTGCCTGCTCAAT |
| | | AACTTTTACCCTAGGGAAGCTAAAGTCCAATGGAAAGTGGATAAC |
| | | GCCCTGCAGTCAGGAAACAGCCAGGAATCGGTTACCGAACAGGAC |
| | | AGCAAGGACAGCACTTACTCCTTGTCGTCGACTCTTACTCTGAGC |
| | | AAGGCCGATTACGAGAAGCACAAGGTCTACGCCTGCGAGGTCACC |
| | | CATCAGGGACTCTCGTCCCCGGTGACCAAATCCTTCAATAGAGGC |
| | | GAATGC |
| 193 | nt S-SL048-118 | GACATCCAGATGACCCAGAGCCCTAGCTCTTTAAGCGCCTCTGTG |
| | LC | GGCGATCGTGTGACCATCACTTGTAGCGCCAGCCCCAGCATCAGC |
| | | TACATGAACTGGTACCAGCAGAAGCCCGGCAAGGCCCCCAAGCTG |
| | | CTGATCTACGCCACAAGCTCTTTACCCAGCGGCGTGCCTAGCAGA |
| | | TTCAGCGGCAGCGGAAGCGGCACCGACTTCACTTTAACCATCAGC |
| | | TCTTTACAGCCAGAAGACTTCGCCACCTACTACTGCCACCAGAGA |
| | | AGCAGCAGCCCCACCTTCGGCCAAGGTACAAAGCTCGAGATCAAG |
| | | AGAACTGTGGCCGCGCCGTCAGTGTTTATCTTCCCTCCATCGGAT |
| | | GAACAGCTTAAGTCCGGCACGGCGTCTGTGGTCTGCCTGCTCAAT |
| | | AACTTTTACCCTAGGGAAGCTAAAGTCCAATGGAAAGTGGATAAC |
| | | GCCCTGCAGTCAGGAAACAGCCAGGAATCGGTTACCGAACAGGAC |
| | | AGCAAGGACAGCACTTACTCCTTGTCGTCGACTCTTACTCTGAGC |
| | | AAGGCCGATTACGAGAAGCACAAGGTCTACGCCTGCGAGGTCACC |
| | | CATCAGGGACTCTCGTCCCCGGTGACCAAATCCTTCAATAGAGGC |
| | | GAATGC |
| 194 | nt CDR graft | CAAGTTCAGCTGGTGCAGAGCGGTGCTGAGGTGAAAAAACCCGGT |
| | HC | GCTTCCGTGAAGGTGAGCTGCAAGGCCAGCGGCTACACCTTTACC |
| | | AGCTACAACATGCACTGGGTGAGACAAGCCCCCGGTCAAGGTTTA |
| | | GAGTGGATCGGCGTGATCTACCCCGGCAACGGCGACACCTCTTAC |
| | | AACCAGAAGTTCAAGGGTCGTGTGACCATGACTCGTGACACCTCC |
| | | ACCAGCACCGTGTACATGGAGCTGAGCTCTTTAAGGAGCGAGGAC |
| | | ACAGCCGTGTACTACTGCGCTCGTGACTACTACGGCAGCAGCCCT |
| | | CTGGGCTATTGGGGCCAAGGTACTTTAGTGACCGTCTCGAGCGCC |
| | | TCAACCAAAGGACCCTCCGTGTTTCCCCTCGCCCCCTGTTCCCGC |
| | | TCCACATCCGAGTCAACCGCGGCGCTGGGCTGCCTCGTGAAGGAC |
| | | TACTTCCCTGAGCCCGTCACTGTGTCGTGGAACTCCGGGGCCCTG |
| | | ACCTCCGGCGTGCACACCTTCCCTGCCGTGCTTCAATCCTCCGGA |
| | | CTGTACTCCCTGTCCTCGGTGGTCACCGTGCCGTCGAGCTCGTTG |
| | | GGAACCAAGACTTACACTTGCAACGTGGACCACAAGCCAAGCAAC |
| | | ACCAAAGTGGACAAGAGAGTCGAATCTAAGTACGGACCGCCCTGC |
| | | CCGCCTTGCCCCGCCCCTGAGTTTCTCGGCGGTCCTAGCGTGTTC |
| | | CTGTTCCCACCCAAGCCCAAGGACACTCTGATGATCTCCCGGACC |
| | | CCTGAAGTGACCTGTGTGGTCGTGGACGTGTCGCAGGAAGATCCG |
| | | GAGGTCCAGTTCAATTGGTACGTGGATGGGGTGGAGGTCCACAAC |
| | | GCCAAGACGAAGCCGAGAGAAGAACAGTTCAACTCAACTTACCGG |
| | | GTGGTGTCCGTGCTGACCGTGCTGCATCAGGATTGGCTCAACGGA |
| | | AAGGAGTACAAGTGCAAAGTGTCCAACAAGGGCCTGCCTAGCTCA |
| | | ATCGAAAAGACCATTTCCAAGGCCAAGGGCCAGCCGAGGGAACCA |
| | | CAGGTCTATACTCTGCCACCGAGCCAAGAAGAGATGACCAAGAAC |
| | | CAAGTGTCCCTGACTTGCCTGGTCAAGGGGTTCTACCCGTCGGAC |
| | | ATCGCAGTGGAGTGGGAGAGCAACGGACAGCCTGAAAACAATTAC |
| | | AAGACCACCCCGCCCGTGCTGGATAGCGACGGTTCCTTCTTCCTT |
| | | TACTCGCGCCTCACCGTCGACAAGAGCCGGTGGCAGGAGGGCAAC |
| | | GTGTTCTCCTGCTCCGTGATGCACGAAGCTCTGCATAACCACTAC |
| | | ACTCAGAAGTCCTTGTCGCTGAGCCTCGGAAAG |
| 195 | nt CDR graft LC | GACATCCAGATGACCCAGAGCCCTAGCTCTTTAAGCGCCTCTGTG |
| | | GGCGATCGTGTGACCATCACTTGTAGCGCCAGCAGCAGCATCAGC |
| | | TACATGCACTGGTACCAGCAGAAGCCCGGCAAGGCCCCCAAGCTG |
| | | CTGATCTACGACACCAGCAAGCTGGCCAGCGGCGTGCCTAGCAGA |
| | | TTCAGCGGCAGCGGAAGCGGCACCGACTTCACTTTAACCATCAGC |
| | | TCTTTACAGCCAGAAGACTTCGCCACCTACTACTGCCACCAGAGG |
| | | AGCAGCTACCCCACCTTCGGCCAAGGTACCAAGCTCGAGATCAAG |
| | | AGAACTGTGGCCGCGCCGTCAGTGTTTATCTTCCCTCCATCGGAT |
| | | GAACAGCTTAAGTCCGGCACGGCGTCTGTGGTCTGCCTGCTCAAT |
| | | AACTTTTACCCTAGGGAAGCTAAAGTCCAATGGAAAGTGGATAAC |
| | | GCCCTGCAGTCAGGAAACAGCCAGGAATCGGTTACCGAACAGGAC |
| | | AGCAAGGACAGCACTTACTCCTTGTCGTCGACTCTTACTCTGAGC |
| | | AAGGCCGATTACGAGAAGCACAAGGTCTACGCCTGCGAGGTCACC |
| | | CATCAGGGACTCTCGTCCCCGGTGACCAAATCCTTCAATAGAGGC |
| | | GAATGC |
| 196 | nt S-SL048-11 | CAAGTGCAGCTCGTGCAGTCCGGAGCCGAAGTCAAGAAGCCCGGA |
| | HC codon | GCGTCAGTGAAAGTGTCCTGCAAGGCCTCGGGCTACACTTTCACA |
| | optimized | AGCTACAACATGCACTGGGTCAGACAGGCACCTGGGCAGGGTCTG |
| | | GAGTGGATGGGAGTGATCTACCCGGGCGACGGCGCCACTTCCTAC |
| | | GCCCAAAAGTTCAAGGGCCGCGTGACCATGACTAGGGACACCTCG |
| | | ACCTCAACCGTGTACATGGAACTGAGCTCCCTGCGGTCCGAGGAT |
| | | ACCGCCGTGTACTATTGTGCTCGGGACTACTACGGGTCCAGCCCA |
| | | CTGGGATACTGGGGACAGGGTACCCTTGTCACGGTGTCGTCAGCT |
| | | TCCACCAAGGGCCCATCCGTCTTCCCCCTGGCGCCCTGCTCCAGG |
| | | AGCACCTCCGAGAGCACAGCCGCCCTGGGCTGCCTGGTCAAGGAC |
| | | TACTTCCCCGAACCGGTGACGGTGTCGTGGAACTCAGGCGCCCTG |
| | | ACCAGCGGCGTGCACACCTTCCCGGCTGTCCTACAGTCCTCAGGA |
| | | CTCTACTCCCTCAGCAGCGTGGTGACCGTGCCCTCCAGCAGCTTG |
| | | GGCACGAAGACCTACACCTGCAATGTAGATCACAAGCCCAGCAAC |
| | | ACCAAGGTGGACAAGAGAGTTGAGTCCAAATATGGTCCCCCATGC |
| | | CCACCATGCCCAGCACCTGAGTTCCTGGGGGGACCATCAGTCTTC |
| | | CTGTTCCCCCCAAAACCCAAGGACACTCTCATGATCTCCCGGACC |
| | | CCTGAGGTCACGTGCGTGGTGGTGGACGTGAGCCAGGAAGACCCC |
| | | GAGGTCCAGTTCAACTGGTACGTGGATGGCGTGGAGGTGCATAAT |
| | | GCCAAGACAAAGCCGCGGGAGGAGCAGTTCAACAGCACGTACCGT |
| | | GTGGTCAGCGTCCTCACCGTCCTGCACCAGGACTGGCTGAACGGC |
| | | AAGGAGTACAAGTGCAAGGTCTCCAACAAAGGCCTCCCGTCCTCC |
| | | ATCGAGAAAACCATCTCCAAAGCCAAAGGGCAGCCCCGAGAGCCA |
| | | CAGGTGTACACCCTGCCCCCATCCCAGGAGGAGATGACCAAGAAC |
| | | CAGGTCAGCCTGACCTGCCTGGTCAAAGGCTTCTACCCCAGCGAC |
| | | ATCGCCGTGGAGTGGGAGAGCAATGGGCAGCCGGAGAACAACTAC |
| | | AAGACCACGCCTCCCGTGCTGGACTCCGACGGCTCCTTCTTCCTC |
| | | TACAGCAGGCTAACCGTGGACAAGAGCAGGTGGCAGGAGGGGAAT |
| | | GTCTTCTCATGCTCCGTGATGCATGAGGCTCTGCACAACCACTAC |
| | | ACACAGAAGAGCCTCTCCCTGTCTCTGGGTAAA |
| 197 | nt S-SL048-11 | GACATCCAAATGACTCAGTCCCCGTCATCCCTGTCGGCATCCGTG |
| | LC codon | GGAGACAGAGTCACCATTACGTGCAGCGCGAGCCCGAGCATTTCC |
| | optimized | TATATGAACTGGTACCAGCAGAAGCCCGGGAAAGCCCCTAAGCTG |
| | | TTGATCTACGCCACTTCCTCACTGGCTTCCGGCGTGCCATCCCGG |
| | | TTCTCGGGTTCCGGCTCGGGAACCGATTTTACCCTTACTATCTCG |
| | | TCCCTGCAACCCGAGGACTTCGCCACCTACTACTGTCACCAGCGC |
| | | TCTAGCTACCCTACATTCGGACAGGGCACCAAGCTCGAAATCAAA |
| | | CGAACTGTGGCTGCACCATCTGTCTTCATCTTCCCGCCATCTGAT |
| | | GAGCAGTTGAAATCTGGAACTGCCTCTGTTGTGTGCCTGCTGAAT |
| | | AACTTCTATCCCAGAGAGGCCAAAGTACAGTGGAAGGTGGATAAC |
| | | GCCCTCCAATCGGGTAACTCCCAGGAGAGTGTCACAGAGCAGGAC |
| | | AGCAAGGACAGCACCTACAGCCTCAGCAGCACCCTGACGCTGAGC |
| | | AAAGCAGACTACGAGAAACACAAAGTCTACGCCTGCGAAGTCACC |
| | | CATCAGGGCCTGAGCTCGCCCGTCACAAAGAGCTTCAACAGGGGA |
| | | GAGTGT |
| 198 | nt S-SL048-106 | CAAGTGCAGCTCGTGCAGTCCGGAGCCGAAGTCAAGAAGCCCGGA |
| | HC codon | GCGTCAGTGAAAGTGTCCTGCAAGGCCTCGGGCTACACTTTCACA |
| | optimized | AGCTACAACATGCACTGGGTCAGACAGGCACCTGGGCAGGGTCTG |
| | | GAGTGGATTGGAGTGATCTACCCGGGCGACGGCTCCACTTCCTAC |
| | | AACCAAAAGTTCCAGGGCCGCGTGACCATGACTAGGGACACCTCG |
| | | ACCTCAACCGTGTACATGGAACTGAGCTCCCTGCGGTCCGAGGAT |
| | | ACCGCCGTGTACTATTGTGCTCGGGACTACTACGGGTCCAGCCCA |
| | | CTGGGATACTGGGGACAGGGTACCCTTGTCACGGTGTCGTCAGCT |
| | | TCCACCAAGGGCCCATCCGTCTTCCCCCTGGCGCCCTGCTCCAGG |
| | | AGCACCTCCGAGAGCACAGCCGCCCTGGGCTGCCTGGTCAAGGAC |
| | | TACTTCCCCGAACCGGTGACGGTGTCGTGGAACTCAGGCGCCCTG |
| | | ACCAGCGGCGTGCACACCTTCCCGGCTGTCCTACAGTCCTCAGGA |
| | | CTCTACTCCCTCAGCAGCGTGGTGACCGTGCCCTCCAGCAGCTTG |
| | | GGCACGAAGACCTACACCTGCAATGTAGATCACAAGCCCAGCAAC |
| | | ACCAAGGTGGACAAGAGAGTTGAGTCCAAATATGGTCCCCCATGC |
| | | CCACCATGCCCAGCACCTGAGTTCCTGGGGGGACCATCAGTCTTC |
| | | CTGTTCCCCCCAAAACCCAAGGACACTCTCATGATCTCCCGGACC |
| | | CCTGAGGTCACGTGCGTGGTGGTGGACGTGAGCCAGGAAGACCCC |
| | | GAGGTCCAGTTCAACTGGTACGTGGATGGCGTGGAGGTGCATAAT |
| | | GCCAAGACAAAGCCGCGGGAGGAGCAGTTCAACAGCACGTACCGT |
| | | GTGGTCAGCGTCCTCACCGTCCTGCACCAGGACTGGCTGAACGGC |
| | | AAGGAGTACAAGTGCAAGGTCTCCAACAAAGGCCTCCCGTCCTCC |
| | | ATCGAGAAAACCATCTCCAAAGCCAAAGGGCAGCCCCGAGAGCCA |
| | | CAGGTGTACACCCTGCCCCCATCCCAGGAGGAGATGACCAAGAAC |
| | | CAGGTCAGCCTGACCTGCCTGGTCAAAGGCTTCTACCCCAGCGAC |
| | | ATCGCCGTGGAGTGGGAGAGCAATGGGCAGCCGGAGAACAACTAC |
| | | AAGACCACGCCTCCCGTGCTGGACTCCGACGGCTCCTTCTTCCTC |
| | | TACAGCAGGCTAACCGTGGACAAGAGCAGGTGGCAGGAGGGGAAT |
| | | GTCTTCTCATGCTCCGTGATGCATGAGGCTCTGCACAACCACTAC |
| | | ACACAGAAGAGCCTCTCCCTGTCTCTGGGTAAA |
| 199 | nt S-SL048-106 | GACATCCAAATGACTCAGTCCCCGTCATCCCTGTCGGCATCCGTG |
| | LC codon | GGAGACAGAGTCACCATTACGTGCAGCGCGAGCTCAAGCATTTCC |
| | optimized | TATATGAACTGGTACCAGCAGAAGCCCGGGAAAGCCCCTAAGCTG |
| | | TTGATCTACGCCACTTCCAAGCTGCCGTCCGGCGTGCCATCCCGG |
| | | TTCTCGGGTTCCGGCTCGGGAACCGATTTTACCCTTACTATCTCG |
| | | TCCCTGCAACCCGAGGACTTCGCCACCTACTACTGTCACCAGCGC |
| | | TCTAGCACCCCTACATTCGGACAGGGCACCAAGCTCGAAATCAAA |
| | | CGAACTGTGGCTGCACCATCTGTCTTCATCTTCCCGCCATCTGAT |
| | | GAGCAGTTGAAATCTGGAACTGCCTCTGTTGTGTGCCTGCTGAAT |
| | | AACTTCTATCCCAGAGAGGCCAAAGTACAGTGGAAGGTGGATAAC |
| | | GCCCTCCAATCGGGTAACTCCCAGGAGAGTGTCACAGAGCAGGAC |
| | | AGCAAGGACAGCACCTACAGCCTCAGCAGCACCCTGACGCTGAGC |
| | | AAAGCAGACTACGAGAAACACAAAGTCTACGCCTGCGAAGTCACC |
| | | CATCAGGGCCTGAGCTCGCCCGTCACAAAGAGCTTCAACAGGGGA |
| | | GAGTGT |
| 200 | nt S-SL048-116 | CAAGTGCAGCTCGTGCAGTCCGGAGCCGAAGTCAAGAAGCCCGGA |
| | and 118 HC | GCGTCAGTGAAAGTGTCCTGCAAGGCCTCGGGCTACACTTTCACA |
| | codon | AGCTACAACATGCACTGGGTCAGACAGGCACCTGGGCAGGGTCTG |
| | optimized | GAGTGGATGGGAGTGATCAACCCGGGCGACGGCGCCACTTCCTAC |
| | | GCCCAAAAGTTCAAGGGCCGCGTGACCATGACTAGGGACACCTCG |
| | | ACCTCAACCGTGTACATGGAACTGAGCTCCCTGCGGTCCGAGGAT |
| | | ACCGCCGTGTACTATTGTGCTCGGGACTACTACGGGTCCAGCCCA |
| | | CTGGGATACTGGGGACAGGGTACCCTTGTCACGGTGTCGTCAGCT |
| | | TCCACCAAGGGCCCATCCGTCTTCCCCCTGGCGCCCTGCTCCAGG |
| | | AGCACCTCCGAGAGCACAGCCGCCCTGGGCTGCCTGGTCAAGGAC |
| | | TACTTCCCCGAACCGGTGACGGTGTCGTGGAACTCAGGCGCCCTG |
| | | ACCAGCGGCGTGCACACCTTCCCGGCTGTCCTACAGTCCTCAGGA |
| | | CTCTACTCCCTCAGCAGCGTGGTGACCGTGCCCTCCAGCAGCTTG |
| | | GGCACGAAGACCTACACCTGCAATGTAGATCACAAGCCCAGCAAC |
| | | ACCAAGGTGGACAAGAGAGTTGAGTCCAAATATGGTCCCCCATGC |
| | | CCACCATGCCCAGCACCTGAGTTCCTGGGGGGACCATCAGTCTTC |
| | | CTGTTCCCCCCAAAACCCAAGGACACTCTCATGATCTCCCGGACC |
| | | CCTGAGGTCACGTGCGTGGTGGTGGACGTGAGCCAGGAAGACCCC |
| | | GAGGTCCAGTTCAACTGGTACGTGGATGGCGTGGAGGTGCATAAT |
| | | GCCAAGACAAAGCCGCGGGAGGAGCAGTTCAACAGCACGTACCGT |
| | | GTGGTCAGCGTCCTCACCGTCCTGCACCAGGACTGGCTGAACGGC |
| | | AAGGAGTACAAGTGCAAGGTCTCCAACAAAGGCCTCCCGTCCTCC |
| | | ATCGAGAAAACCATCTCCAAAGCCAAAGGGCAGCCCCGAGAGCCA |
| | | CAGGTGTACACCCTGCCCCCATCCCAGGAGGAGATGACCAAGAAC |
| | | CAGGTCAGCCTGACCTGCCTGGTCAAAGGCTTCTACCCCAGCGAC |
| | | ATCGCCGTGGAGTGGGAGAGCAATGGGCAGCCGGAGAACAACTAC |
| | | AAGACCACGCCTCCCGTGCTGGACTCCGACGGCTCCTTCTTCCTC |
| | | TACAGCAGGCTAACCGTGGACAAGAGCAGGTGGCAGGAGGGGAAT |
| | | GTCTTCTCATGCTCCGTGATGCATGAGGCTCTGCACAACCACTAC |
| | | ACACAGAAGAGCCTCTCCCTGTCTCTGGGTAAA |
| 201 | nt S-SL048-116 | GACATCCAAATGACTCAGTCCCCGTCATCCCTGTCGGCATCCGTG |
| | LC codon | GGAGACAGAGTCACCATTACGTGCCGCGCGAGCCCGAGCATTTCC |
| | optimized | TATATGAACTGGTACCAGCAGAAGCCCGGGAAAGCCCCTAAGCTG |
| | | TTGATCTACGCCACTTCCTCACTGGCTTCCGGCGTGCCATCCCGG |
| | | TTCTCGGGTTCCGGCTCGGGAACCGATTTTACCCTTACTATCTCG |
| | | TCCCTGCAACCCGAGGACTTCGCCACCTACTACTGTCACCAGCGC |
| | | TCTAGCAGCCCTACATTCGGACAGGGCACCAAGCTCGAAATCAAA |
| | | CGAACTGTGGCTGCACCATCTGTCTTCATCTTCCCGCCATCTGAT |
| | | GAGCAGTTGAAATCTGGAACTGCCTCTGTTGTGTGCCTGCTGAAT |
| | | AACTTCTATCCCAGAGAGGCCAAAGTACAGTGGAAGGTGGATAAC |
| | | GCCCTCCAATCGGGTAACTCCCAGGAGAGTGTCACAGAGCAGGAC |
| | | AGCAAGGACAGCACCTACAGCCTCAGCAGCACCCTGACGCTGAGC |
| | | AAAGCAGACTACGAGAAACACAAAGTCTACGCCTGCGAAGTCACC |
| | | CATCAGGGCCTGAGCTCGCCCGTCACAAAGAGCTTCAACAGGGGA |
| | | GAGTGT |
| 202 | nt S-SL048-118 | GACATCCAAATGACTCAGTCCCCGTCATCCCTGTCGGCATCCGTG |
| | LC codon | GGAGACAGAGTCACCATTACGTGCAGCGCGAGCCCGAGCATTTCC |
| | optimized | TATATGAACTGGTACCAGCAGAAGCCCGGGAAAGCCCCTAAGCTG |
| | | TTGATCTACGCCACTTCCTCACTGCCGTCCGGCGTGCCATCCCGG |
| | | TTCTCGGGTTCCGGCTCGGGAACCGATTTTACCCTTACTATCTCG |
| | | TCCCTGCAACCCGAGGACTTCGCCACCTACTACTGTCACCAGCGC |
| | | TCTAGCAGCCCTACATTCGGACAGGGCACCAAGCTCGAAATCAAA |
| | | CGAACTGTGGCTGCACCATCTGTCTTCATCTTCCCGCCATCTGAT |
| | | GAGCAGTTGAAATCTGGAACTGCCTCTGTTGTGTGCCTGCTGAAT |
| | | AACTTCTATCCCAGAGAGGCCAAAGTACAGTGGAAGGTGGATAAC |
| | | GCCCTCCAATCGGGTAACTCCCAGGAGAGTGTCACAGAGCAGGAC |
| | | AGCAAGGACAGCACCTACAGCCTCAGCAGCACCCTGACGCTGAGC |
| | | AAAGCAGACTACGAGAAACACAAAGTCTACGCCTGCGAAGTCACC |
| | | CATCAGGGCCTGAGCTCGCCCGTCACAAAGAGCTTCAACAGGGGA |
| | | GAGTGT |

### REFERENCES

1 European Journal of Biochemistry (1981) 116(2): 221-225
2 U.S. Patent Application No. 2010/0124555
3 U.S. Pat. No. 8,597,650
U.S. Patent No. 9,168,299
Developmental and Comparative Immunology (2003) 27(1): 55-77
Nucleic Acids Research (1994) 22: 4673-4680
Protein Engineering, Design and Selection (2016) 29(10): 457-466
8 The Journal of Biological Chemistry (2015) 290(9): 5462-5469
Journal of Molecular Biology (2001) 312: 511-523
MAbs (2013) 5(3): 445-470
Journal of Molecular Biology (2007) 373: 924-940
Protein Engineering, Design and Selection (2016) 29: 427-437
13 Molecular Immunoogy. (1993) 30(1): 105-1058
PNAS (2001) 98(3): 944-949
The Journal of Immunology (2012) 189(7): 3430-3438
The Journal of Biological Chemistry (2017) 292(9): 3900-3908
Protein Science (2000) 9: 1783-1790

1.-49. (canceled)

50. An isolated antibody, or antigen-binding fragment thereof, specifically binding to a bile salt stimulated lipase (BSSL), preferably human BSSL (hBSSL), and comprising:
three complementarity determining regions (CDRs) of a heavy chain variable region (HCVR) (HCDRs); and
three CDRs of a light chain variable region (LCVR) (LCDRs), wherein
the first HCDR comprises an amino acid sequence according to SEQ ID NO: 7, or an amino acid sequence having at least 87% identity to SEQ ID NO: 7;
the second HCDR comprises an amino acid sequence according to SEQ ID NO: 8, or an amino acid sequence having at least 75% identity to SEQ ID NO: 8;
the third HCDR comprises an amino acid sequence according to SEQ ID NO: 9, or an amino acid sequence having at least 83% identity to SEQ ID NO: 9;
the first LCDR comprises an amino acid sequence according to SEQ ID NO: 10, or an amino acid sequence having at least 80% identity to SEQ ID NO: 10;
the second LCDR comprises the amino acid sequence ATS, or an amino acid sequence having at least 66% identity to the amino acid sequence ATS, preferably AAS; and
the third LCDR comprises an amino acid sequence according to SEQ ID NO: 11, or an amino acid sequence having at least 87% identity to SEQ ID NO: 11.

51. The isolated antibody, or antigen-binding fragment thereof, according to claim 50, wherein
the first HCDR comprises the amino acid sequence according to SEQ ID NO: 7;
the second HCDR comprises an amino acid sequence selected from the group consisting of SEQ ID NO: 8, SEQ ID NO: 18 and SEQ ID NO: 19;
the third HCDR comprises the amino acid sequence according to SEQ ID NO: 9;
the first LCDR comprises an amino acid sequence selected from the group consisting of SEQ ID NO: 10 and SEQ ID NO: 20;
the second LCDR comprises an amino acid sequence selected from the group consisting of ATS and AAS; and
the third LCDR comprises an amino acid sequence selected from the group consisting of SEQ ID NO: 11, SEQ ID NO: 21 and SEQ ID NO: 22.

52. The isolated antibody, or antigen-binding fragment thereof, according to claim 51, wherein
the first HCDR comprises the amino acid sequence according to SEQ ID NO: 7;
the second HCDR comprises the amino acid sequence according to SEQ ID NO: 8;
the third HCDR comprises the amino acid sequence according to SEQ ID NO: 9;
the first LCDR comprises the amino acid sequence according to SEQ ID NO: 10;
the second LCDR comprises the amino acid sequence ATS; and
the third LCDR comprises the amino acid sequence according to SEQ ID NO: 11.

53. The isolated antibody, or antigen-binding fragment thereof, according to claim 52, wherein the isolated antibody, or antigen-binding fragment thereof, comprises:
an extended second HCDR comprising the amino acid sequence according to SEQ ID NO: 12;
an extended first LCDR comprising the amino acid sequence according to SEQ ID NO: 14; and
an extended second LCDR comprising the amino acid sequence according to SEQ ID NO: 15.

54. The isolated antibody, or antigen-binding fragment thereof, according to claim 52, wherein
the HCVR comprises an amino acid sequence according to SEQ ID NO: 36; and/or
the LCVR comprises an amino acid sequence according to SEQ ID NO: 37.

55. The isolated antibody, or antigen-binding fragment thereof, according to claim 52, wherein the isolated antibody, or antigen-binding fragment thereof, comprises:
an extended second HCDR comprising the amino acid sequence according to SEQ ID NO: 12;
an extended first LCDR comprising the amino acid sequence according to SEQ ID NO: 16; and
an extended second LCDR comprising the amino acid sequence according to SEQ ID NO: 17.

56. The isolated antibody, or antigen-binding fragment thereof, according to claim 52, wherein
the HCVR comprises an amino acid sequence according to SEQ ID NO: 36; and/or
the LCVR comprises an amino acid sequence according to SEQ ID NO: 38.

57. The isolated antibody, or antigen-binding fragment thereof, according to claim 51, wherein
the first HCDR comprises the amino acid sequence according to SEQ ID NO: 7;
the second HCDR comprises the amino acid sequence according to SEQ ID NO: 18;
the third HCDR comprises the amino acid sequence according to SEQ ID NO: 9;
the first LCDR comprises the amino acid sequence according to SEQ ID NO: 10;
the second LCDR comprises the amino acid sequence ATS; and
the third LCDR comprises the amino acid sequence according to SEQ ID NO: 21.

58. The isolated antibody, or antigen-binding fragment thereof, according to claim 57, wherein the isolated antibody, or antigen-binding fragment thereof, comprises:
an extended second HCDR comprising the amino acid sequence according to SEQ ID NO: 23;
an extended first LCDR comprising the amino acid sequence according to SEQ ID NO: 16; and
an extended second LCDR comprising the amino acid sequence according to SEQ ID NO: 15.

59. The isolated antibody, or antigen-binding fragment thereof, according to claim 57, wherein
the HCVR comprises, preferably consists of, an amino acid sequence according to SEQ ID NO: 30; and/or
the LCVR comprises, preferably consists of, an amino acid sequence according to SEQ ID NO: 31.

60. The isolated antibody, or antigen-binding fragment thereof, according to claim 51, wherein
the first HCDR comprises the amino acid sequence according to SEQ ID NO: 7;
the second HCDR comprises the amino acid sequence according to SEQ ID NO: 8;
the third HCDR comprises the amino acid sequence according to SEQ ID NO: 9;
the first LCDR comprises the amino acid sequence according to SEQ ID NO: 20;
the second LCDR comprises the amino acid sequence AAS; and
the third LCDR comprises the amino acid sequence according to SEQ ID NO: 11.

61. The isolated antibody, or antigen-binding fragment thereof, according to claim 60, wherein the isolated antibody, or antigen-binding fragment thereof, comprises:
an extended second HCDR comprising the amino acid sequence according to SEQ ID NO: 24;
an extended first LCDR comprising the amino acid sequence according to SEQ ID NO: 27; and
an extended second LCDR comprising the amino acid sequence according to SEQ ID NO: 29.

62. The isolated antibody, or antigen-binding fragment thereof, according to claim 60, wherein
the HCVR comprises an amino acid sequence according to SEQ ID NO: 32; and/or
the LCVR comprises an amino acid sequence according to SEQ ID NO: 33.

63. The isolated antibody, or antigen-binding fragment thereof, according to claim 51, wherein
the first HCDR comprises the amino acid sequence according to SEQ ID NO: 7;
the second HCDR comprises the amino acid sequence according to SEQ ID NO: 19;
the third HCDR comprises the amino acid sequence according to SEQ ID NO: 9;
the first LCDR comprises the amino acid sequence according to SEQ ID NO: 20;
the second LCDR comprises the amino acid sequence ATS; and
the third LCDR comprises the amino acid sequence according to SEQ ID NO: 22.

64. The isolated antibody, or antigen-binding fragment thereof, according to claim 63, wherein the isolated antibody, or antigen-binding fragment thereof, comprises:
an extended second HCDR comprising the amino acid sequence according to SEQ ID NO: b 25;
an extended first LCDR comprising the amino acid sequence according to SEQ ID NO: 26; and
an extended second LCDR comprising the amino acid sequence according to SEQ ID NO: 28.

65. The isolated antibody, or antigen-binding fragment thereof, according to claim 63, wherein
the HCVR comprises an amino acid sequence according to SEQ ID NO: 34; and/or
the LCVR comprises an amino acid sequence according to SEQ ID NO: 35.

66. The isolated antibody, or antigen-binding fragment thereof, according to claim 50, wherein the HCVR comprises, preferably consists of, an amino acid sequence selected from the group consisting of SEQ ID NO: 30, SEQ ID NO: 32, SEQ ID NO: 34, and SEQ ID NO: 36.

67. The isolated antibody, or antigen-binding fragment thereof, according to claim 50, wherein the LCVR comprises, preferably consists of, an amino acid sequence selected from the group consisting of SEQ ID NO: 31, SEQ ID NO: 33, SEQ ID NO: 35, SEQ ID NO: 37 and SEQ ID NO: 38.

68. The isolated antibody, or antigen-binding fragment thereof, according to claim 50, wherein the antibody, or antigen-binding fragment thereof, is selected from the group consisting of a human antibody, a humanized antibody and a chimeric antibody, or an antigen-binding fragment thereof.

69. The isolated antibody, or antigen-binding fragment thereof, according to claim 50, wherein the antigen-binding fragment is selected from the group consisting of a single chain fragment variable (scFv), a Fab fragment, F(ab′)₂ fragment, a F(ab′)₃ fragment, a Fab′ fragment, a Fd fragment, a Fv fragment, a dAb fragment, an isolated complementarity determining region (CDR) and a nanobody, preferably a scFv.

70. The isolated antibody, or antigen-binding fragment thereof according to claim 50, wherein the isolated antibody, or antigen-binding fragment thereof, is a monoclonal antibody, or an antigen-binding fragment thereof.

71. The isolated antibody, or antigen-binding fragment thereof, according to claim 50, wherein the isolated antibody, or antigen-binding fragment thereof, hs an isotype class selected from the group consisting of IgG, IgA, IgM, IgD and IgE.

72. The isolated antibody, or antigen-binding fragment thereof, according to claim 50, wherein the isolated antibody, or antigen-binding fragment thereof, comprises at least one Fc silencing mutation inhibiting interaction with Fc receptors.

73. The isolated antibody, or antigen-binding fragment thereof, according to claim 72, wherein isolated antibody, or antigen-binding fragment thereof, is of IgG isotype class and the at least one Fc silencing mutation is selected from the group consisting of L234A, L235A and P329G.

74. The isolated antibody, or antigen-binding fragment thereof, according to claim 50, wherein the isolated antibody, or antigen-binding fragment thereof comprises, at least one stabilizing mutation which prevents or reduces in vivo Fab arm exchange.

75. The isolated antibody, or antigen-binding fragment thereof, according to claim 74, wherein the isolated antibody, or antigen-binding fragment thereof, is of IgG4 isotype subclass and the at least one stabilizing mutation is S228P.

76. A pharmaceutical composition comprising an isolated antibody and/or an antigen-binding fragment thereof according to claim 50 and a pharmaceutically acceptable carrier or excipient.

77. A method for treating and/or ameliorating and/or preventing and/or prophylaxis of an inflammatory disease, the method comprises administering a therapeutically effective amount of an isolated antibody, or an antigen-binding fragment thereof, according to claim 50 to a subject in need thereof.

78. The method according to claim 77, wherein the inflammatory disease is selected from the group consisting of a chronic inflammatory disease, a systemic inflammatory disease, an autoimmune disease, an autoinflammatory disease, a natural killer (NK) cell mediated inflammatory disease, rheumatoid arthritis (RA), juvenile idiopathic arthritis (JIA), psoriatic arthritis, Crohn's disease, ulcerative colitis (UC), and hepatic steatosis.

79. A polynucleotide encoding an antibody, or antigen-binding fragment thereof, according to claim 50.

80. An expression vector comprising a polynucleotide according to claim 79.

81. A cell comprising an expression vector according to claim 80.

82. A method of producing an antibody, or an antigen-binding fragment thereof, the method comprising:
culturing a cell according to claim 81 under conditions where the antibody, or antigen-binding fragment thereof, is expressed by the cell.

83. A method for detecting the presence or absence of a bile salt stimulated lipase (BSSL) and/or quantifying the amount of BSSL in a sample, the method comprising:
contacting the sample with an isolated antibody, or an antigen-binding fragment thereof, according to claim 50; and
detecting the presence or absence of BSSL in the sample and/or quantifying the amount of BSSL in the sample based on an amount of isolated antibody, or antigen-binding fragment thereof bound to BSSL.

84. A method for diagnosis of a bile salt stimulated lipase (BSSL) related disorder, the method comprising:
a) contacting a sample from a subject with an isolated antibody, or an antigen-binding fragment thereof, according to claim 50;
b) detecting the presence or absence of BSSL and/or quantifying the amount of BSSL in the sample based on an amount of isolated antibody, or antigen-binding fragment thereof bound to BSSL; and
c) concluding, based on the results in step b), whether the subject is suffering from a BSSL-related disorder or not.

85. An isolated antibody, or antigen-binding fragment thereof, specifically binding to a bile salt stimulated lipase (BSSL), preferably human BSSL (hBSSL), and comprising:
a heavy chain variable region (HCVR) consisting of an amino acid sequence selected from ZH1-[GYTFTSYN]-ZH2-[X₅₃GVIX₅₇PGDGX₆₄TSYX₆₈QKFX₇₂]-ZH3-[ARDYYGSSPLGY]-ZH4, wherein
each of ZH1, ZH2, ZH3 and ZH4 independently represents zero, one or several independently selected amino acid residues,
X₅₃ is selected from I and M;
X₅₇ is selected from N and Y;
X₆₄ is selected from A and S;
X₆₈ is selected from A and N; and
X₇₂ is selected from K and Q; and
a light chain variable region (LCVR) consisting of an amino acid sequence selected from ZL1-[X₂₄ASX₂₇SISYX₃₉N]-ZL2-[AX₅₇SX₆₆LX₆₈]-ZL3-[HQRSSX₁₁₅PT]-ZL4, wherein
each of ZL1, ZL2, ZL3 and ZL4 independently represents zero, one or several independently selected amino acid residues,
X₂₄ is selected from S and R;
X₂₇ is selected from S and P;
X₃₉ is selected from M and L;
X₅₇ is selected from A and T;
X₆₆ is selected from K and S;
X₆₈ is selected from A and P; and
X₁₁₅ is selected from S, T and Y.

86. The isolated antibody, or antigen-binding fragment thereof, according to claim 85, wherein
ZH1 comprises an amino acid sequence according to SEQ ID NO: 39, or an amino acid sequence having at least 90% identity to SEQ ID NO: 39;
ZH2 comprises an amino acid sequence according to SEQ ID NO: 40, or an amino acid sequence having at least 90% identity to SEQ ID NO: 40;
ZH3 comprises an amino acid sequence according to SEQ ID NO: 41, or an amino acid sequence having at least 90% identity to SEQ ID NO: 41;
ZH4 comprises an amino acid sequence according to SEQ ID NO: 42, or an amino acid sequence having at least 90% identity to SEQ ID NO: 42;
ZL1 comprises an amino acid sequence according to SEQ ID NO: 43, or an amino acid sequence having at least 90% identity to SEQ ID NO: 43;
ZL2 comprises an amino acid sequence according to SEQ ID NO: 44, or an amino acid sequence having at least 90% identity to SEQ ID NO: 44;
ZL3 comprises an amino acid sequence according to SEQ ID NO: 45, or an amino acid sequence having at least 90% identical to SEQ ID NO: 45; and/or
ZL4 comprises an amino acid sequence according to SEQ ID NO: 46, or an amino acid sequence having at least 90% identity to SEQ ID NO:46.

87. An isolated antibody, or an antigen-binding fragment thereof, that specifically binds to an epitope of a bile salt stimulated lipase (BSSL), wherein the epitope comprises:
a first surface comprising an amino acid sequence according to SEQ ID NO: 1, or an amino acid sequence having at least 80%, preferably at least 83%, identity to SEQ ID NO: 1; and
a second surface comprising an amino acid sequence according to SEQ ID NO: 2, or an amino acid sequence having at least 80%, preferably at least 85% or at least 92%, identity to SEQ ID NO: 2.

88. The isolated antibody, or antigen-binding fragment thereof, according to claim 87, wherein the first surface comprises an amino acid sequence according to SEQ ID NO: 3, or an amino acid sequence having at least 80%, preferably at least 83%, and more preferably at least 91%, identity to SEQ ID NO: 3.

89. The isolated antibody, or antigen-binding fragment thereof, according to claim 87, wherein the isolated antibody, or antigen-binding fragment thereof, further specifically binds to a surface selected from the group consisting of:
an amino acid sequence according to SEQ ID NO: 5, or an amino acid sequence having at least 80%, preferably at least 85%, identity to SEQ ID NO: 5;
an amino acid sequence according to SEQ ID NO: 4, or an amino acid sequence having at least 80%, preferably at least 83%, more preferably at least 88%, such as at least 94%, identity to SEQ ID NO: 4; and
an amino acid sequence according to SEQ ID NO: 6, or an amino acid sequence having at least 80%, preferably at least 84%, and more preferably at least 92%, identity to SEQ ID NO: 6.

90. A bile salt stimulated lipase (BSSL) epitope comprising:
a first surface comprising an amino acid sequence according to SEQ ID NO: 1, or an amino acid sequence having at least 80%, preferably at least 83%, identity to SEQ ID NO: 1; and
a second surface comprising an amino acid sequence according to SEQ ID NO: 2, or an amino acid sequence having at least 80%, preferably at least 85% or at least 92%, identity to SEQ ID NO: 2.

91. The BSSL epitope according to claim 90, wherein the first surface comprises an amino acid sequence according to SEQ ID NO: 3.

92. The BSSL epitope according to claim 90, further comprising a surface comprising, preferably consisting of, an amino acid sequence selected from the group consisting of SEQ ID NO: 4, SEQ ID NO: 5 and SEQ ID NO: 6.


SEQUENCE LISTING (202 sequences)

SEQ ID NO: 1 - protein, 6 aa, Homo sapiens
YTEGGF

SEQ ID NO: 2 - protein, 14 aa, Homo sapiens
LENPQPHPGWQGTL

SEQ ID NO: 3 - protein, 12 aa, Homo sapiens
AKLGAVYTEGGF

SEQ ID NO: 4 - protein, 18 aa, Homo sapiens
NIWVPQGRKQVSRDLPVM

SEQ ID NO: 5 - protein, 7 aa, Homo sapiens
VKRNIAA

SEQ ID NO: 6 - protein, 13 aa, Homo sapiens
HYVGFVPVIDGDF

SEQ ID NO: 7 - protein, 8 aa, Artificial Sequence
  HCDR1
GYTFTSYN

SEQ ID NO: 8 - protein, 8 aa, Artificial Sequence
  HCDR2
INPGDGAT

SEQ ID NO: 9 - protein, 12 aa, Artificial Sequence
  HCDR3
ARDYYGSSPLGY

SEQ ID NO: 10 - protein, 5 aa, Artificial Sequence
  LCDR1
PSISY

SEQ ID NO: 11 - protein, 8 aa, Artificial Sequence
  LCDR3
HQRSSSPT

SEQ ID NO: 12 - protein, 18 aa, Artificial Sequence
  Extended HCDR2
MGVINPGDGATSYAQKFK

SEQ ID NO: 13 - protein, 9 aa, Homo sapiens
AWVKRNIAA

SEQ ID NO: 14 - protein, 10 aa, Artificial Sequence
  Extended LCDR1
RASPSISYMN

SEQ ID NO: 15 - protein, 6 aa, Artificial Sequence
  Extended LCDR2
ATSSLA

SEQ ID NO: 16 - protein, 10 aa, Artificial Sequence
  Extended LCDR1
SASPSISYMN

SEQ ID NO: 17 - protein, 6 aa, Artificial Sequence
  Extended LCDR2
ATSSLP

SEQ ID NO: 18 - protein, 8 aa, Artificial Sequence
  HCDR2
IYPGDGAT

SEQ ID NO: 19 - protein, 8 aa, Artificial Sequence
  HCDR2
IYPGDGST

SEQ ID NO: 20 - protein, 5 aa, Artificial Sequence
  LCDR1
SSISY

SEQ ID NO: 21 - protein, 8 aa, Artificial Sequence
  LCDR3
HQRSSYPT

SEQ ID NO: 22 - protein, 8 aa, Artificial Sequence
  LCDR3
HQRSSTPT

SEQ ID NO: 23 - protein, 18 aa, Artificial Sequence
  Extended HCDR2
MGVIYPGDGATSYAQKFK

SEQ ID NO: 24 - protein, 18 aa, Artificial Sequence
  Extended HCDR2
MGVINPGDGATSYNQKFQ

SEQ ID NO: 25 - protein, 18 aa, Artificial Sequence
  Extended HCDR2
IGVIYPGDGSTSYNQKFQ

SEQ ID NO: 26 - protein, 10 aa, Artificial Sequence
  Extended LCDR1
SASSSISYMN

SEQ ID NO: 27 - protein, 10 aa, Artificial Sequence
  Extended LCDR1
RASSSISYLN

SEQ ID NO: 28 - protein, 6 aa, Artificial Sequence
  Extended LCDR2
ATSKLP

SEQ ID NO: 29 - protein, 6 aa, Artificial Sequence
  Extended LCDR2
AASSLA

SEQ ID NO: 30 - protein, 119 aa, Artificial Sequence
  HVCR
QVQLVQSGAEVKKPGASVKVSCKASGYTFTSYNMHWVRQAPGQGLEWMGVIYPGDGATSYAQKFKGRVTMTRDTSTSTVYMELSSLRSEDTAVYYCARDYYGSSPLGYWGQGTLVTVSS

SEQ ID NO: 31 - protein, 105 aa, Artificial Sequence
  LVCR
DIQMTQSPSSLSASVGDRVTITCSASPSISYMNWYQQKPGKAPKLLIYATSSLASGVPSRFSGSGSGTDFTLTISSLQPEDFATYYCHQRSSYPTFGQGTKLEIK

SEQ ID NO: 32 - protein, 119 aa, Artificial Sequence
  HVCR
QVQLVQSGAEVKKPGASVKVSCKASGYTFTSYNMHWVRQAPGQGLEWMGVINPGDGATSYNQKFQGRVTMTRDTSTSTVYMELSSLRSEDTAVYYCARDYYGSSPLGYWGQGTLVTVSS

SEQ ID NO: 33 - protein, 105 aa, Artificial Sequence
  LCVR
DIQMTQSPSSLSASVGDRVTITCRASSSISYLNWYQQKPGKAPKLLIYAASSLASGVPSRFSGSGSGTDFTLTISSLQPEDFATYYCHQRSSSPTFGQGTKLEIK

SEQ ID NO: 34 - protein, 119 aa, Artificial Sequence
  HVCR
QVQLVQSGAEVKKPGASVKVSCKASGYTFTSYNMHWVRQAPGQGLEWIGVIYPGDGSTSYNQKFQGRVTMTRDTSTSTVYMELSSLRSEDTAVYYCARDYYGSSPLGYWGQGTLVTVSS

SEQ ID NO: 35 - protein, 105 aa, Artificial Sequence
  LCVR
DIQMTQSPSSLSASVGDRVTITCSASSSISYMNWYQQKPGKAPKLLIYATSKLPSGVPSRFSGSGSGTDFTLTISSLQPEDFATYYCHQRSSTPTFGQGTKLEIK

SEQ ID NO: 36 - protein, 119 aa, Artificial Sequence
  HVCR
QVQLVQSGAEVKKPGASVKVSCKASGYTFTSYNMHWVRQAPGQGLEWMGVINPGDGATSYAQKFKGRVTMTRDTSTSTVYMELSSLRSEDTAVYYCARDYYGSSPLGYWGQGTLVTVSS

SEQ ID NO: 37 - protein, 105 aa, Artificial Sequence
  LCVR
DIQMTQSPSSLSASVGDRVTITCRASPSISYMNWYQQKPGKAPKLLIYATSSLASGVPSRFSGSGSGTDFTLTISSLQPEDFATYYCHQRSSSPTFGQGTKLEIK

SEQ ID NO: 38 - protein, 105 aa, Artificial Sequence
  LCVR
DIQMTQSPSSLSASVGDRVTITCSASPSISYMNWYQQKPGKAPKLLIYATSSLPSGVPSRFSGSGSGTDFTLTISSLQPEDFATYYCHQRSSSPTFGQGTKLEIK

SEQ ID NO: 39 - protein, 25 aa, Artificial Sequence [sequence omitted]

SEQ ID NO: 40 - protein, 14 aa, Artificial Sequence
  ZH2
MHWVRQAPGQGLEW

SEQ ID NO: 41 - protein, 31 aa, Artificial Sequence
  ZH3
GRVTMTRDTSTSTVYMELSSLRSEDTAVYYC

SEQ ID NO: 42 - protein, 11 aa, Artificial Sequence
  ZH4
WGQGTLVTVSS

SEQ ID NO: 43 - protein, 23 aa, Artificial Sequence
  ZL1
DIQMTQSPSSLSASVGDRVTITC

SEQ ID NO: 44 - protein, 15 aa, Artificial Sequence
  ZL2
WYQQKPGKAPKLLIY

SEQ ID NO: 45 - protein, 33 aa, Artificial Sequence
  ZL3
SGVPSRFSGSGSGTDFTLTISSLQPEDFATYYC

SEQ ID NO: 46 - protein, 10 aa, Artificial Sequence
  ZL4
FGQGTKLEIK

SEQ ID NO: 47 - protein, 119 aa, Artificial Sequence
  VH
QVQLVQSGAEVKKPGASVKVSCKASGYTFTSYNMHWVRQAPGQGLEWIGVINPSDGYTSYAQKFQGRVTMTRDTSTSTVYMELSSLRSEDTAVYYCARDYYGSSPLGYWGQGTLVTVSS

SEQ ID NO: 48 - protein, 119 aa, Artificial Sequence
  VH
QVQLVQSGAEVKKPGASVKVSCKASGYTFTSYNMHWVRQAPGQGLEWMGVINPSDGATSYTQKFQGRVTMTRDTSTSTVYMELSSLRSEDTAVYYCARDYYGSSPLGYWGQGTLVTVSS

SEQ ID NO: 49 - protein, 119 aa, Artificial Sequence
  VH
QVQLVQSGAEVKKPGASVKVSCKASGYTFTSYNMHWVRQAPGQGLEWIGVINPSDGATSYTQKFQGRVTMTRDTSTSTVYMELSSLRSEDTAVYYCARDYYGSSPLGYWGQGTLVTVSS

SEQ ID NO: 50 - protein, 119 aa, Artificial Sequence; identical to SEQ ID NO: 36
  VH

SEQ ID NO: 51 - protein, 119 aa, Artificial Sequence
  VH
QVQLVQSGAEVKKPGASVKVSCKASGYTFTSYNMHWVRQAPGQGLEWMGVINPGDGYTSYTQKFKGRVTMTRDTSTSTVYMELSSLRSEDTAVYYCARDYYGSSPLGYWGQGTLVTVSS

SEQ ID NO: 52 - protein, 119 aa, Artificial Sequence
  VH
QVQLVQSGAEVKKPGASVKVSCKASGYTFTSYNMHWVRQAPGQGLEWMGVINPSDGATSYTQKFKGRVTMTRDTSTSTVYMELSSLRSEDTAVYYCARDYYGSSPLGYWGQGTLVTVSS

SEQ ID NO: 53 - protein, 119 aa, Artificial Sequence
  VH
QVQLVQSGAEVKKPGASVKVSCKASGYTFTSYNMHWVRQAPGQGLEWMGVINPGDGATSYAQKFQGRVTMTRDTSTSTVYMELSSLRSEDTAVYYCARDYYGSSPLGYWGQGTLVTVSS

SEQ ID NO: 54 - protein, 119 aa, Artificial Sequence
  VH
QVQLVQSGAEVKKPGASVKVSCKASGYTFTSYNMHWVRQAPGQGLEWIGVINPSDGATSYAQKFQGRVTMTRDTSTSTVYMELSSLRSEDTAVYYCARDYYGSSPLGYWGQGTLVTVSS

SEQ ID NO: 55 - protein, 119 aa, Artificial Sequence
  VH
QVQLVQSGAEVKKPGASVKVSCKASGYTFTSYNMHWVRQAPGQGLEWIGVIYPGDGATSYAQKFKGRVTMTRDTSTSTVYMELSSLRSEDTAVYYCARDYYGSSPLGYWGQGTLVTVSS

SEQ ID NO: 56 - protein, 119 aa, Artificial Sequence
  VH
QVQLVQSGAEVKKPGASVKVSCKASGYTFTSYNMHWVRQAPGQGLEWMGVINPGDGATSYTQKFQGRVTMTRDTSTSTVYMELSSLRSEDTAVYYCARDYYGSSPLGYWGQGTLVTVSS

SEQ ID NO: 57 - protein, 119 aa, Artificial Sequence; identical to SEQ ID NO: 48
  VH

SEQ ID NO: 58 - protein, 119 aa, Artificial Sequence
  VH
QVQLVQSGAEVKKPGASVKVSCKASGYTFTSYNMHWVRQAPGQGLEWIGVINPGGGYTSYTQKFQGRVTMTRDTSTSTVYMELSSLRSEDTAVYYCARDYYGSSPLGYWGQGTLVTVSS

SEQ ID NO: 59 - protein, 119 aa, Artificial Sequence
  VH
QVQLVQSGAEVKKPGASVKVSCKASGYTFTSYNMHWVRQAPGQGLEWIGVINPGDGATSYAQKFQGRVTMTRDTSTSTVYMELSSLRSEDTAVYYCARDYYGSSPLGYWGQGTLVTVSS

SEQ ID NO: 60 - protein, 119 aa, Artificial Sequence
  VH
QVQLVQSGAEVKKPGASVKVSCKASGYTFTSYNMHWVRQAPGQGLEWIGVINPGDGATSYAQKFKGRVTMTRDTSTSTVYMELSSLRSEDTAVYYCARDYYGSSPLGYWGQGTLVTVSS

SEQ ID NO: 61 - protein, 119 aa, Artificial Sequence
  VH
QVQLVQSGAEVKKPGASVKVSCKASGYTFTSYNMHWVRQAPGQGLEWMGVINPGDGATSYNQKFKGRVTMTRDTSTSTVYMELSSLRSEDTAVYYCARDYYGSSPLGYWGQGTLVTVSS

SEQ ID NO: 62 - protein, 119 aa, Artificial Sequence; identical to SEQ ID NO: 59
  VH

SEQ ID NO: 63 - protein, 119 aa, Artificial Sequence
  VH
QVQLVQSGAEVKKPGASVKVSCKASGYTFTSYNMHWVRQAPGQGLEWMGVIYPGGGYTSYAQKFKGRVTMTRDTSTSTVYMELSSLRSEDTAVYYCARDYYGSSPLGYWGQGTLVTVSS

SEQ ID NO: 64 - protein, 119 aa, Artificial Sequence
  VH
QVQLVQSGAEVKKPGASVKVSCKASGYTFTSYNMHWVRQAPGQGLEWMGVINPSDGATSYAQKFQGRVTMTRDTSTSTVYMELSSLRSEDTAVYYCARDYYGSSPLGYWGQGTLVTVSS

SEQ ID NO: 65 - protein, 119 aa, Artificial Sequence; identical to SEQ ID NO: 59
  VH

SEQ ID NO: 66 - protein, 119 aa, Artificial Sequence; identical to SEQ ID NO: 53
  VH

SEQ ID NO: 67 - protein, 119 aa, Artificial Sequence
  VH
QVQLVQSGAEVKKPGASVKVSCKASGYTFTSYNMHWVRQAPGQGLEWMGVINPSSGSTSYTQKFQGRVTMTRDTSTSTVYMELSSLRSEDTAVYYCARDYYGSSPLGYWGQGTLVTVSS

SEQ ID NO: 68 - protein, 119 aa, Artificial Sequence; identical to SEQ ID NO: 36
  VH

SEQ ID NO: 69 - protein, 119 aa, Artificial Sequence
  VH
QVQLVQSGAEVKKPGASVKVSCKASGYTFTSYNMHWVRQAPGQGLEWMGVINPGDGATSYDQKFKGRVTMTRDTSTSTVYMELSSLRSEDTAVYYCARDYYGSSPLGYWGQGTLVTVSS

SEQ ID NO: 70 - protein, 119 aa, Artificial Sequence; identical to SEQ ID NO: 64
  VH

SEQ ID NO: 71 - protein, 119 aa, Artificial Sequence; identical to SEQ ID NO: 32
  VH

SEQ ID NO: 72 - protein, 119 aa, Artificial Sequence
  VH
QVQLVQSGAEVKKPGASVKVSCKASGYTFTSYNMHWVRQAPGQGLEWIGVINPSDGATSYAQKFKGRVTMTRDTSTSTVYMELSSLRSEDTAVYYCARDYYGSSPLGYWGQGTLVTVSS

SEQ ID NO: 73 - protein, 119 aa, Artificial Sequence
  VH
QVQLVQSGAEVKKPGASVKVSCKASGYTFTSYNMHWVRQAPGQGLEWMGVINPSDGATSYAQKFKGRVTMTRDTSTSTVYMELSSLRSEDTAVYYCARDYYGSSPLGYWGQGTLVTVSS

SEQ ID NO: 74 - protein, 119 aa, Artificial Sequence; identical to SEQ ID NO: 61
  VH

SEQ ID NO: 75 - protein, 119 aa, Artificial Sequence
  VH
QVQLVQSGAEVKKPGASVKVSCKASGYTFTSYNMHWVRQAPGQGLEWIGIIYPGDGATSYNQKFKGRVTMTRDTSTSTVYMELSSLRSEDTAVYYCARDYYGSSPLGYWGQGTLVTVSS

SEQ ID NO: 76 - protein, 119 aa, Artificial Sequence; identical to SEQ ID NO: 73
  VH

SEQ ID NO: 77 - protein, 119 aa, Artificial Sequence; identical to SEQ ID NO: 59
  VH

SEQ ID NO: 78 - protein, 119 aa, Artificial Sequence; identical to SEQ ID NO: 60
  VH

SEQ ID NO: 79 - protein, 119 aa, Artificial Sequence; identical to SEQ ID NO: 73
  VH

SEQ ID NO: 80 - protein, 119 aa, Artificial Sequence
  VH
QVQLQQPGAELVKPGASVKMSCKASGYTFTSYNMHWVKQTPGQGLEWIGVIYPGNGDTSYNQKFKGKATLTADKSSSTAYMQLSSLTSEDSAVYYCARDYYGSSPLGYWGQGTTLTVSS

SEQ ID NO: 81 - protein, 105 aa, Artificial Sequence
  VL
DIQMTQSPSSLSASVGDRVTITCRASPSISYLNWYQQKPGKAPKLLIYATSRLPSGVPSRFSGSGSGTDFTLTISSLQPEDFATYYCQQRSSSPTFGQGTKLEIK

SEQ ID NO: 82 - protein, 105 aa, Artificial Sequence
  VL
DIQMTQSPSSLSASVGDRVTITCSASSSISYMHWYQQKPGKAPKLLIYAASRLASGVPSRFSGSGSGTDFTLTISSLQPEDFATYYCHQRSSTSTFGQGTKLEIK

SEQ ID NO: 83 - protein, 105 aa, Artificial Sequence
  VL
DIQMTQSPSSLSASVGDRVTITCSASPSISYLNWYQQKPGKAPKLLIYATSSLASGVPSRFSGSGSGTDFTLTISSLQPEDFATYYCQQRSSSPTFGQGTKLEIK

SEQ ID NO: 84 - protein, 105 aa, Artificial Sequence; identical to SEQ ID NO: 81
  VL

SEQ ID NO: 85 - protein, 105 aa, Artificial Sequence
  VL
DIQMTQSPSSLSASVGDRVTITCRASQSISYLNWYQQKPGKAPKLLIYAASKLPSGVPSRFSGSGSGTDFTLTISSLQPEDFATYYCQQRSSSPTFGQGTKLEIK

SEQ ID NO: 86 - protein, 105 aa, Artificial Sequence
  VL
DIQMTQSPSSLSASVGDRVTITCRASSSISYLNWYQQKPGKAPKLLIYATSRLQSGVPSRFSGSGSGTDFTLTISSLQPEDFATYYCHQRSSTPTFGQGTKLEIK

SEQ ID NO: 87 - protein, 105 aa, Artificial Sequence
  VL
DIQMTQSPSSLSASVGDRVTITCRASPSISYLNWYQQKPGKAPKLLIYATSSLASGVPSRFSGSGSGTDFTLTISSLQPEDFATYYCQQRSSSPTFGQGTKLEIK

SEQ ID NO: 88 - protein, 105 aa, Artificial Sequence
  VL
DIQMTQSPSSLSASVGDRVTITCRASSSISYLNWYQQKPGKAPKLLIYAASRLPSGVPSRFSGSGSGTDFTLTISSLQPEDFATYYCQQRSSSPTFGQGTKLEIK

SEQ ID NO: 89 - protein, 105 aa, Artificial Sequence
  VL
DIQMTQSPSSLSASVGDRVTITCRASSSISYMNWYQQKPGKAPKLLIYATSRLPSGVPSRFSGSGSGTDFTLTISSLQPEDFATYYCHQRSSSPTFGQGTKLEIK

SEQ ID NO: 90 - protein, 105 aa, Artificial Sequence
  VL
DIQMTQSPSSLSASVGDRVTITCRASPSISYMNWYQQKPGKAPKLLIYAASSLPSGVPSRFSGSGSGTDFTLTISSLQPEDFATYYCQQRSSSPTFGQGTKLEIK

SEQ ID NO: 91 - protein, 105 aa, Artificial Sequence
  VL
DIQMTQSPSSLSASVGDRVTITCRASQSISYLHWYQQKPGKAPKLLIYAASRLASGVPSRFSGSGSGTDFTLTISSLQPEDFATYYCHQRSSTPTFGQGTKLEIK

SEQ ID NO: 92 - protein, 105 aa, Artificial Sequence
  VL
DIQMTQSPSSLSASVGDRVTITCSASPSISYMNWYQQKPGKAPKLLIYATSRLQSGVPSRFSGSGSGTDFTLTISSLQPEDFATYYCQQRSSTPTFGQGTKLEIK

SEQ ID NO: 93 - protein, 105 aa, Artificial Sequence
  VL
DIQMTQSPSSLSASVGDRVTITCRASPSISYMNWYQQKPGKAPKLLIYATSRLQSGVPSRFSGSGSGTDFTLTISSLQPEDFATYYCHQRSSSPTFGQGTKLEIK

SEQ ID NO: 94 - protein, 105 aa, Artificial Sequence
  VL
DIQMTQSPSSLSASVGDRVTITCSASQSISYLNWYQQKPGKAPKLLIYDASSLPSGVPSRFSGSGSGTDFTLTISSLQPEDFATYYCHQRSSSPTFGQGTKLEIK

SEQ ID NO: 95 - protein, 105 aa, Artificial Sequence
  VL
DIQMTQSPSSLSASVGDRVTITCSASQSISYMNWYQQKPGKAPKLLIYDASSLASGVPSRFSGSGSGTDFTLTISSLQPEDFATYYCHQRSSTPTFGQGTKLEIK

SEQ ID NO: 96 - protein, 105 aa, Artificial Sequence
  VL
DIQMTQSPSSLSASVGDRVTITCRASQSISYLNWYQQKPGKAPKLLIYAASSLASGVPSRFSGSGSGTDFTLTISSLQPEDFATYYCHQRSSSPTFGQGTKLEIK

SEQ ID NO: 97 - protein, 105 aa, Artificial Sequence
  VL
DIQMTQSPSSLSASVGDRVTITCRASQSISYLNWYQQKPGKAPKLLIYATSNLQSGVPSRFSGSGSGTDFTLTISSLQPEDFATYYCHQRSSSPTFGQGTKLEIK

SEQ ID NO: 98 - protein, 105 aa, Artificial Sequence
  VL
DIQMTQSPSSLSASVGDRVTITCSASQSISYLNWYQQKPGKAPKLLIYAASRLPSGVPSRFSGSGSGTDFTLTISSLQPEDFATYYCHQRSSYPTFGQGTKLEIK

SEQ ID NO: 99 - protein, 105 aa, Artificial Sequence
  VL
DIQMTQSPSSLSASVGDRVTITCRASSSISYMHWYQQKPGKAPKLLIYAASSLQSGVPSRFSGSGSGTDFTLTISSLQPEDFATYYCHQRSSSPTFGQGTKLEIK

SEQ ID NO: 100 - protein, 105 aa, Artificial Sequence
  VL
DIQMTQSPSSLSASVGDRVTITCRASQSISYLHWYQQKPGKAPKLLIYATSSLQSGVPSRFSGSGSGTDFTLTISSLQPEDFATYYCHQRSSYPTFGQGTKLEIK

SEQ ID NO: 101 - protein, 105 aa, Artificial Sequence
  VL
DIQMTQSPSSLSASVGDRVTITCRASPSISYLNWYQQKPGKAPKLLIYATSRLPSGVPSRFSGSGSGTDFTLTISSLQPEDFATYYCQQRSSNPTFGQGTKLEIK

SEQ ID NO: 102 - protein, 105 aa, Artificial Sequence
  VL
DIQMTQSPSSLSASVGDRVTITCRASSSISYMHWYQQKPGKAPKLLIYATSSLASGVPSRFSGSGSGTDFTLTISSLQPEDFATYYCHQRSSSPTFGQGTKLEIK

SEQ ID NO: 103 - protein, 105 aa, Artificial Sequence
  VL
DIQMTQSPSSLSASVGDRVTITCSASSSISYLHWYQQKPGKAPKLLIYAASRLASGVPSRFSGSGSGTDFTLTISSLQPEDFATYYCHQRSSTPTFGQGTKLEIK

SEQ ID NO: 104 - protein, 105 aa, Artificial Sequence
  VL
DIQMTQSPSSLSASVGDRVTITCSASQSISYLHWYQQKPGKAPKLLIYAASKLPSGVPSRFSGSGSGTDFTLTISSLQPEDFATYYCHQRSSYPTFGQGTKLEIK

SEQ ID NO: 105 - protein, 105 aa, Artificial Sequence
  VL
DIQMTQSPSSLSASVGDRVTITCRASPSISYLNWYQQKPGKAPKLLIYATSSLPSGVPSRFSGSGSGTDFTLTISSLQPEDFATYYCQQRSSSPTFGQGTKLEIK

SEQ ID NO: 106 - protein, 105 aa, Artificial Sequence; identical to SEQ ID NO: 83
  VL

SEQ ID NO: 107 - protein, 105 aa, Artificial Sequence
  VL
DIQMTQSPSSLSASVGDRVTITCRASPSISYLNWYQQKPGKAPKLLIYATSRLASGVPSRFSGSGSGTDFTLTISSLQPEDFATYYCQQRSSSPTFGQGTKLEIK

SEQ ID NO: 108 - protein, 105 aa, Artificial Sequence
  VL
DIQMTQSPSSLSASVGDRVTITCSASQSISYLHWYQQKPGKAPKLLIYAASRLASGVPSRFSGSGSGTDFTLTISSLQPEDFATYYCHQRSSTPTFGQGTKLEIK

SEQ ID NO: 109 - protein, 105 aa, Artificial Sequence
  VL
DIQMTQSPSSLSASVGDRVTITCRASSSISYMHWYQQKPGKAPKLLIYATSRLASGVPSRFSGSGSGTDFTLTISSLQPEDFATYYCHQRSSTPTFGQGTKLEIK

SEQ ID NO: 110 - protein, 105 aa, Artificial Sequence
  VL
DIQMTQSPSSLSASVGDRVTITCRASQSISYMHWYQQKPGKAPKLLIYAASKLPSGVPSRFSGSGSGTDFTLTISSLQPEDFATYYCHQRSSSPTFGQGTKLEIK

SEQ ID NO: 111 - protein, 105 aa, Artificial Sequence
  VL
DIQMTQSPSSLSASVGDRVTITCRASSSISYLHWYQQKPGKAPKLLIYAASRLPSGVPSRFSGSGSGTDFTLTISSLQPEDFATYYCHQRSSYPTFGQGTKLEIK

SEQ ID NO: 112 - protein, 105 aa, Artificial Sequence
  VL
DIQMTQSPSSLSASVGDRVTITCRASPSISYLNWYQQKPGKAPKLLIYAASKLASGVPSRFSGSGSGTDFTLTISSLQPEDFATYYCHQRSSSPTFGQGTKLEIK

SEQ ID NO: 113 - protein, 105 aa, Artificial Sequence
  VL
DIQMTQSPSSLSASVGDRVTITCRASPSISYLNWYQQKPGKAPKLLIYATSKLPSGVPSRFSGSGSGTDFTLTISSLQPEDFATYYCQQRSSYPTFGQGTKLEIK

SEQ ID NO: 114 - protein, 105 aa, Artificial Sequence
  VL
QIVLTQSPAVMSASPGEKVTMTCSASSSISYMHWYQQKPGTSPKRWIYDTSKLASGVPARFSGSGSGTSYSLTISSMEAEDAATYYCHQRSSYPTFGGGTKLEIK

SEQ ID NO: 115 - protein, 449 aa, Artificial Sequence
  IgG1 LALA-PG HC
QVQLQQPGAELVKPGASVKMSCKASGYTFTSYNMHWVKQTPGQGLEWIGVIYPGNGDTSYNQKFKGKATLTADKSSSTAYMQLSSLTSEDSAVYYCARDYYGSSPLGYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 212 aa, Artificial Sequence
  hIgG1 LALA-PG LC
QIVLTQSPAVMSASPGEKVTMTCSASSSISYMHWYQQKPGTSPKRWIYDTSKLASGVPARFSGSGSGTSYSLTISSMEAEDAATYYCHQRSSYPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 450 aa, Artificial Sequence
  hIgG1 LALA-PG HC
QVQLQQPGAELVKPGASVKLSCKASGYTFTSYWMHWVKQRPGRGLEWIGRIDPNSGGTKYNEKFKSKATLTVDKPSSTAYMQLSSLTSEDSAVYYCARYDYYGSSYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 213 aa, Artificial Sequence
  hIgG1 LALA-PG  LC
QAVVTQESALTTSPGETVTLTCRSSTGAVTTSNYANWVQEKPDHLFTGLIGGTNNRAPGVPARFSGSLIGNKAALTITGAQTEDEAIYFCALWYSNHWVFGGGTKLTVLQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 119 - protein, 446 aa, Artificial Sequence
  hIgG4 S228P HC
QVQLVQSGAEVKKPGASVKVSCKASGYTFTSYNMHWVRQAPGQGLEWMGVIYPGDGATSYAQKFKGRVTMTRDTSTSTVYMELSSLRSEDTAVYYCARDYYGSSPLGYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 120 - protein, 212 aa, Artificial Sequence
  hIgG4 S228P  LC
DIQMTQSPSSLSASVGDRVTITCSASPSISYMNWYQQKPGKAPKLLIYATSSLASGVPSRFSGSGSGTDFTLTISSLQPEDFATYYCHQRSSYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 446 aa, Artificial Sequence
  hIgG4 S228P HC
QVQLVQSGAEVKKPGASVKVSCKASGYTFTSYNMHWVRQAPGQGLEWMGVINPGDGATSYNQKFQGRVTMTRDTSTSTVYMELSSLRSEDTAVYYCARDYYGSSPLGYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 122 - protein, 212 aa, Artificial Sequence
  hIgG4 S228P  LC
DIQMTQSPSSLSASVGDRVTITCRASSSISYLNWYQQKPGKAPKLLIYAASSLASGVPSRFSGSGSGTDFTLTISSLQPEDFATYYCHQRSSSPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 446 aa, Artificial Sequence
  hIgG4 S228P HC
QVQLVQSGAEVKKPGASVKVSCKASGYTFTSYNMHWVRQAPGQGLEWIGVIYPGDGSTSYNQKFQGRVTMTRDTSTSTVYMELSSLRSEDTAVYYCARDYYGSSPLGYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 124 - protein, 212 aa, Artificial Sequence
  hIgG4 S228P  LC
DIQMTQSPSSLSASVGDRVTITCSASSSISYMNWYQQKPGKAPKLLIYATSKLPSGVPSRFSGSGSGTDFTLTISSLQPEDFATYYCHQRSSTPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 446 aa, Artificial Sequence
  hIgG4 S228P HC
QVQLVQSGAEVKKPGASVKVSCKASGYTFTSYNMHWVRQAPGQGLEWMGVINPGDGATSYAQKFKGRVTMTRDTSTSTVYMELSSLRSEDTAVYYCARDYYGSSPLGYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 126 - protein, 212 aa, Artificial Sequence
  hIgG4 S228P  LC
DIQMTQSPSSLSASVGDRVTITCRASPSISYMNWYQQKPGKAPKLLIYATSSLASGVPSRFSGSGSGTDFTLTISSLQPEDFATYYCHQRSSSPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 446 aa, Artificial Sequence; identical to SEQ ID NO: 125
  hIgG4 S228P HC

SEQ ID NO: 128 - protein, 212 aa, Artificial Sequence
  hIgG4 S228P  LC
DIQMTQSPSSLSASVGDRVTITCSASPSISYMNWYQQKPGKAPKLLIYATSSLPSGVPSRFSGSGSGTDFTLTISSLQPEDFATYYCHQRSSSPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 446 aa, Artificial Sequence
  hIgG4 S228P HC
QVQLQQPGAELVKPGASVKMSCKASGYTFTSYNMHWVKQTPGQGLEWIGVIYPGNGDTSYNQKFKGKATLTADKSSSTAYMQLSSLTSEDSAVYYCARDYYGSSPLGYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 130 - protein, 212 aa, Artificial Sequence; identical to SEQ ID NO: 116
  hIgG4 S228P  LC

SEQ ID NO: 131 - protein, 446 aa, Artificial Sequence
  hIgG4 S228P HC
QVQLVQSGAEVKKPGASVKVSCKASGYTFTSYNMHWVRQAPGQGLEWIGVIYPGNGDTSYNQKFKGRVTMTRDTSTSTVYMELSSLRSEDTAVYYCARDYYGSSPLGYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 132 - protein, 212 aa, Artificial Sequence
  hIgG4 S228P  LC
DIQMTQSPSSLSASVGDRVTITCSASSSISYMHWYQQKPGKAPKLLIYDTSKLASGVPSRFSGSGSGTDFTLTISSLQPEDFATYYCHQRSSYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 447 aa, Artificial Sequence
  hIgG4 S228P HC
QVQLQQPGAELVKPGASVKLSCKASGYTFTSYWMHWVKQRPGRGLEWIGRIDPNSGGTKYNEKFKSKATLTVDKPSSTAYMQLSSLTSEDSAVYYCARYDYYGSSYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 134 - protein, 216 aa, Artificial Sequence
  hIgG4 S228P  LC
QAVVTQESALTTSPGETVTLTCRSSTGAVTTSNYANWVQEKPDHLFTGLIGGTNNRAPGVPARFSGSLIGNKAALTITGAQTEDEAIYFCALWYSNHWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 138 aa, Artificial Sequence
  IgG HC
MGWSCIILFLVATATGVHSQVQLQQPGAELVKPGASVKMSCKASGYTFTSYNMHWVKQTPGQGLEWIGVIYPGNGDTSYNQKFKGKATLTADKSSSTAYMQLSSLTSEDSAVYYCARDYYGSSPLGYWGQGTTLTVSS

SEQ ID NO: 136 - protein, 127 aa, Artificial Sequence
  IgG LC
MDFQVQIFSFLLISASVILSRGQIVLTQSPAVMSASPGEKVTMTCSASSSISYMHWYQQKPGTSPKRWIYDTSKLASGVPARFSGSGSGTSYSLTISSMEAEDAATYYCHQRSSYPTFGGGTKLEIK

SEQ ID NO: 137 - protein, 579 aa, Mus musculus
AKLGAVYTEGGFVEGVNKKLSLLGGDSVDIFKGIPFATAKTLENPQRHPGWQGTLKATNFKKRCLQATITQDNTYGQEDCLYLNIWVPQGRKQVSHNLPVMVWIYGGAFLMGSGQGANFLKNYLYDGEEIATRGNVIVVTFNYRVGPLGFLSTGDANLPGNFGLRDQHMAIAWVKRNIAAFGGDPDNITIFGESAGAASVSLQTLSPYNKGLIRRAISQSGMALSPWAIQKNPLFWAKTIAKKVGCPTEDTGKMAACLKITDPRALTLAYKLPVKKQEYPVVHYLAFIPVIDGDFIPDDPINLYNNTADIDYIAGINNMDGHLFATIDVPAVDKTKQTVTEEDFYRLVSGHTVAKGLKGAQATFDIYTESWAQDPSQENMKKTVVAFETDVLFLIPTEIALAQHKAHAKSAKTYSYLFSHPSRMPIYPKWMGADHADDLQYVFGKPFATPLGYRPQDRAVSKAMIAYWTNFARSGDPNMGNSPVPTHWYPYTLENGNYLDITKTITSASMKEHLREKFLKFWAVTFEVLPTVTGDQDTLTPPEDDSEVAPDPPSDDSQVVPVPPTDDSVEAQMPATIGF

SEQ ID NO: 138 - protein, 722 aa, Homo sapiens
AKLGAVYTEGGFVEGVNKKLGLLGDSVDIFKGIPFAAPTKALENPQPHPGWQGTLKAKNFKKRCLQATITQDSTYGDEDCLYLNIWVPQGRKQVSRDLPVMIWIYGGAFLMGSGHGANFLNNYLYDGEEIATRGNVIVVTFNYRVGPLGFLSTGDANLPGNYGLRDQHMAIAWVKRNIAAFGGDPNNITLFGESAGGASVSLQTLSPYNKGLIRRAISQSGVALSPWVIQKNPLFWAKKVAEKVGCPVGDAARMAQCLKVTDPRALTLAYKVPLAGLEYPMLHYVGFVPVIDGDFIPADPINLYANAADIDYIAGTNNMDGHIFASIDMPAINKGNKKVTEEDFYKLVSEFTITKGLRGAKTTFDVYTESWAQDPSQENKKKTVVDFETDVLFLVPTEIALAQHRANAKSAKTYAYLFSHPSRMPVYPKWVGADHADDIQYVFGKPFATPTGYRPQDRTVSKAMIAYWTNFAKTGDPNMGDSAVPTHWEPYTTENSGYLEITKKMGSSSMKRSLRTNFLRYWTLTYLALPTVTDQEATPVPPTGDSEATPVPPTGDSETAPVPPTGDSGAPPVPPTGDSGAPPVPPTGDSGAPPVPPTGDSGAPPVPPTGDSGAPPVPPTGDSGAPPVPPTGDSGAPPVPPTGDAGPPPVPPTGDSGAPPVPPTGDSGAPPVTPTGDSETAPVPPTGDSGAPPVPPTGDSEAAPVPPTDDSKEAQMPAVIRF

SEQ ID NO: 139 - protein, 446 aa, Artificial Sequence
  IgG4 HC
QVQLQQPGAELVKPGASVKMSCKASGYTFTSYNMHWVKQTPGQGLEWIGVIYPGNGDTSYNQKFKGKATLTADKSSSTAYMQLSSLTSEDSAVYYCARDYYGSSPLGYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 140 - protein, 212 aa, Artificial Sequence; identical to SEQ ID NO: 116
  IgG4 LC

SEQ ID NO: 141 - protein, 18 aa, Artificial Sequence
  Extended HCDR2
IGVIYPGNGDTSYNQKFK

SEQ ID NO: 142 - protein, 10 aa, Artificial Sequence
  Extended LCDR1
SASSSISYMH

SEQ ID NO: 143 - protein, 6 aa, Artificial Sequence
  Extended LCDR2
DTSKLA

SEQ ID NO: 144 - protein, 119 aa, Artificial Sequence
  HCVR
QVQLVQSGAEVKKPGASVKVSCKASGYTFTSYNMHWVRQAPGQGLEWIGVIYPGNGDTSYNQKFKGRVTMTRDTSTSTVYMELSSLRSEDTAVYYCARDYYGSSPLGYWGQGTLVTVSS

SEQ ID NO: 145 - protein, 105 aa, Artificial Sequence
  LCVR
DIQMTQSPSSLSASVGDRVTITCSASSSISYMHWYQQKPGKAPKLLIYDTSKLASGVPSRFSGSGSGTDFTLTISSLQPEDFATYYCHQRSSYPTFGQGTKLEIK

SEQ ID NO: 146 - protein, 537 aa, Artificial Sequence
  t-hBSSL
AKLGAVYTEGGFVEGVNKKLGLLGDSVDIFKGIPFAAPTKALENPQPHPGWQGTLKAKNFKKRCLQATITQDSTYGDEDCLYLNIWVPQGRKQVSRDLPVMIWIYGGAFLMGSGHGANFLNNYLYDGEEIATRGNVIVVTFNYRVGPLGFLSTGDANLPGNYGLRDQHMAIAWVKRNIAAFGGDPNNITLFGESAGGASVSLQTLSPYNKGLIRRAISQSGVALSPWVIQKNPLFWAKKVAEKVGCPVGDAARMAQCLKVTDPRALTLAYKVPLAGLEYPMLHYVGFVPVIDGDFIPADPINLYANAADIDYIAGTNNMDGHIFASIDMPAINKGNKKVTEEDFYKLVSEFTITKGLRGAKTTFDVYTESWAQDPSQENKKKTVVDFETDVLFLVPTEIALAQHRANAKSAKTYAYLFSHPSRMPVYPKWVGADHADDIQYVFGKPFATPTGYRPQDRTVSKAMIAYWTNFAKTGDPNMGDSAVPTHWEPYTTENSGYLEITKKMGSSSMKRSLRTNFLRYWTLTYLALPAHHHHHH

SEQ ID NO: 147 - protein, 5 aa, Homo sapiens
YTEGG

SEQ ID NO: 148 - protein, 7 aa, Homo sapiens
AKLGAVY

SEQ ID NO: 149 - protein, 11 aa, Homo sapiens
AKLGAVYTEGG

SEQ ID NO: 150 - protein, 25 aa, Homo sapiens
AKLGAVYTEGGFVEGVNKKLGLLGD

SEQ ID NO: 151 - protein, 26 aa, Homo sapiens
AKLGAVYTEGGFVEGVNKKLGLLGDS

SEQ ID NO: 152 - protein, 28 aa, Homo sapiens
AKLGAVYTEGGFVEGVNKKLGLLGDSVD

SEQ ID NO: 153 - protein, 9 aa, Artificial Sequence
  iTope residues
VKPGASVKM

SEQ ID NO: 154 - protein, 9 aa, Artificial Sequence
  iTope residues
YNMHWVRQA

SEQ ID NO: 155 - protein, 9 aa, Artificial Sequence
  iTope residues
YAQKFKGRV

SEQ ID NO: 156 - protein, 9 aa, Artificial Sequence
  iTope residues
WGQGTTLTV

SEQ ID NO: 157 - protein, 9 aa, Artificial Sequence
  iTope residues
VTMTCSASS

SEQ ID NO: 158 - protein, 9 aa, Artificial Sequence
  iTope residues
VTITCRASS

SEQ ID NO: 159 - protein, 9 aa, Artificial Sequence
  iTope residues
VTITCSASS

SEQ ID NO: 160 - protein, 9 aa, Artificial Sequence
  iTope residues
LLIYATSKL

SEQ ID NO: 161 - protein, 9 aa, Artificial Sequence
  iTope residues
IYATSSLAS

SEQ ID NO: 162 - protein, 9 aa, Artificial Sequence
  iTope residues
IYAASSLAS

SEQ ID NO: 163 - protein, 9 aa, Artificial Sequence
  iTope residues
FGGGTKLEI

SEQ ID NO: 164 - DNA, 37 bp, Artificial Sequence
  Oligonucleotide primer
gatacaccttcaccagctacwatatgcactgggtgcg

SEQ ID NO: 165 - protein, 83 aa, Artificial Sequence
  Oligonucleotide primer
GACAAGGGCTTGAGTGGATRGGARTAATCWACCCTRGTRRTGGTKMCACAAGCTACRMTCAGAAGTTCMAGGGCCGCGTCACC

SEQ ID NO: 166 - DNA, 62 bp, Artificial Sequence
  Oligonucleotide primer
cgtcaccatcacctgcagkgcaagtymgagcattagctatwtgmattggtatcagcagaaac

SEQ ID NO: 167 - DNA, 52 bp, Artificial Sequence
  Oligonucleotide primer
cctaagctcctgatctatgmtrcatccarsttgsmaagtggggtcccatcac

SEQ ID NO: 168 - DNA, 57 bp, Artificial Sequence
  Oligonucleotide primer
gattttgcaacttattactgtcascagagktmtagtwmtyhcacttttggccagggg

SEQ ID NO: 169 - protein, 18 aa, Artificial Sequence
  HVCR part 2
  MISC_FEATURE (1)..(1): X=I or M
  MISC_FEATURE (5)..(5): X=N or Y
  MISC_FEATURE (10)..(10): X=A or S
  MISC_FEATURE (14)..(14): X=A or N
  MISC_FEATURE (18)..(18): X=K or Q
XGVIXPGDGXTSYXQKFX

SEQ ID NO: 170 - protein, 10 aa, Artificial Sequence
  LVCR part 1
  MISC_FEATURE (1)..(1): X=S or R
  MISC_FEATURE (4)..(4): X=S or P
  MISC_FEATURE (9)..(9): X=M or L
XASXSISYXN

SEQ ID NO: 171 - protein, 6 aa, Artificial Sequence
  LVCR part 2
  MISC_FEATURE (2)..(2): X=A or T
  MISC_FEATURE (4)..(4): X=K or S
  MISC_FEATURE (6)..(6): X=A or P
AXSXLX

SEQ ID NO: 172 - protein, 8 aa, Artificial Sequence
  LVCR part 3
  MISC_FEATURE (6)..(6): X=S, T or Y
HQRSSXPT

SEQ ID NO: 173 - protein, 8 aa, Homo sapiens
QQSYSTPT

SEQ ID NO: 174 - DNA, 357 bp, Artificial Sequence
  HC
caggtgcagctggtgcagtctggggctgaggtgaagaagcctggggcctcagtgaaggtttcctgcaaggcatctggatacaccttcaccagctacaatatgcactgggtgcgacaggcccctggacaagggcttgagtggatgggagtaatctaccctggtgatggtgccacaagctacgctcagaagttcaagggccgcgtcaccatgacccgcgacacgtccacgagcacagtctacatggagctgagcagcctgcgctctgaggacacggctgtgtattactgtgcgagagattactacggtagtagcccccttggctactggggccaaggaaccctggtcaccgtctcctca

SEQ ID NO: 175 - DNA, 315 bp, Artificial Sequence
  LC
gacatccagatgacccagtctccatcctccctgagcgcatctgtaggagaccgcgtcaccatcacctgcagtgcaagtccgagcattagctatatgaattggtatcagcagaaaccagggaaagcccctaagctcctgatctatgctacatccagcttggcaagtggggtcccatcacgtttcagtggcagtggaagcgggacagatttcactctcaccatcagcagtctgcaacctgaagattttgcaacttattactgtcaccagaggtctagttatcccacttttggccaggggaccaagctggagatcaaa

SEQ ID NO: 176 - DNA, 357 bp, Artificial Sequence
  HC
caggtgcagctggtgcagtctggggctgaggtgaagaagcctggggcctcagtgaaggtttcctgcaaggcatctggatacaccttcaccagctacaatatgcactgggtgcgacaggcccctggacaagggcttgagtggatgggagtaatcaaccctggtgatggtgccacaagctacaatcagaagttccagggccgcgtcaccatgacccgcgacacgtccacgagcacagtctacatggagctgagcagcctgcgctctgaggacacggctgtgtattactgtgcgagagattactacggtagtagcccccttggctactggggccaaggaaccctggtcaccgtctcctca

SEQ ID NO: 177 - DNA, 315 bp, Artificial Sequence
  LC
gacatccagatgacccagtctccatcctccctgagcgcatctgtaggagaccgcgtcaccatcacctgcagggcaagttcgagcattagctatttgaattggtatcagcagaaaccagggaaagcccctaagctcctgatctatgctgcatccagcttggcaagtggggtcccatcacgtttcagtggcagtggaagcgggacagatttcactctcaccatcagcagtctgcaacctgaagattttgcaacttattactgtcaccagaggtctagttctcccacttttggccaggggaccaagctggagatcaaa

SEQ ID NO: 178 - DNA, 357 bp, Artificial Sequence
  HC
caggtgcagctggtgcagtctggggctgaggtgaagaagcctggggcctcagtgaaggtttcctgcaaggcatctggatacaccttcaccagctacaatatgcactgggtgcgacaggcccctggacaagggcttgagtggataggagtaatctaccctggtgatggttccacaagctacaatcagaagttccagggccgcgtcaccatgacccgcgacacgtccacgagcacagtctacatggagctgagcagcctgcgctctgaggacacggctgtgtattactgtgcgagagattactacggtagtagcccccttggctactggggccaaggaaccctggtcaccgtctcctca

SEQ ID NO: 179 - DNA, 315 bp, Artificial Sequence
  LC
gacatccagatgacccagtctccatcctccctgagcgcatctgtaggagaccgcgtcaccatcacctgcagtgcaagttcgagcattagctatatgaattggtatcagcagaaaccagggaaagcccctaagctcctgatctatgctacatccaagttgccaagtggggtcccatcacgtttcagtggcagtggaagcgggacagatttcactctcaccatcagcagtctgcaacctgaagattttgcaacttattactgtcaccagaggtctagtactcccacttttggccaggggaccaagctggagatcaaa

SEQ ID NO: 180 - DNA, 357 bp, Artificial Sequence
  HC
caggtgcagctggtgcagtctggggctgaggtgaagaagcctggggcctcagtgaaggtttcctgcaaggcatctggatacaccttcaccagctacaatatgcactgggtgcgacaggcccctggacaagggcttgagtggatgggagtaatcaaccctggtgatggtgccacaagctacgctcagaagttcaagggccgcgtcaccatgacccgcgacacgtccacgagcacagtctacatggagctgagcagcctgcgctctgaggacacggctgtgtattactgtgcgagagattactacggtagtagcccccttggctactggggccaaggaaccctggtcaccgtctcctca

SEQ ID NO: 181 - DNA, 315 bp, Artificial Sequence
  LC
gacatccagatgacccagtctccatcctccctgagcgcatctgtaggagaccgcgtcaccatcacctgcagggcaagtccgagcattagctatatgaattggtatcagcagaaaccagggaaagcccctaagctcctgatctatgctacatccagcttggcaagtggggtcccatcacgtttcagtggcagtggaagcgggacagatttcactctcaccatcagcagtctgcaacctgaagattttgcaacttattactgtcaccagaggtctagttctcccacttttggccaggggaccaagctggagatcaaa

SEQ ID NO: 182 - DNA, 315 bp, Artificial Sequence
  LC
gacatccagatgacccagtctccatcctccctgagcgcatctgtaggagaccgcgtcaccatcacctgcagtgcaagtccgagcattagctatatgaattggtatcagcagaaaccagggaaagcccctaagctcctgatctatgctacatccagcttgccaagtggggtcccatcacgtttcagtggcagtggaagcgggacagatttcactctcaccatcagcagtctgcaacctgaagattttgcaacttattactgtcaccagaggtctagttctcccacttttggccaggggaccaagctggagatcaaa

SEQ ID NO: 183 - DNA, 1338 bp, Artificial Sequence
  HC
caagttcagctgcagcagcccggtgccgagctggtgaaacccggtgcctctgtgaagatgagctgcaaggccagcggctacacctttaccagctacaacatgcactgggtgaagcagacacccggacaaggtttagagtggatcggcgtgatctaccccggcaacggcgacacctcttacaaccagaagttcaagggcaaggccacactgaccgccgacaagagcagcagcaccgcctacatgcagctgagctctttaaccagcgaggactccgccgtgtactactgcgctcgtgattactacggcagcagccctttaggctattggggacaaggtaccactttaaccgtctcgagcgcctcaaccaaaggaccctccgtgtttcccctcgccccctgttcccgctccacatccgagtcaaccgcggcgctgggctgcctcgtgaaggactacttccctgagcccgtcactgtgtcgtggaactccggggccctgacctccggcgtgcacaccttccctgccgtgcttcaatcctccggactgtactccctgtcctcggtggtcaccgtgccgtcgagctcgttgggaaccaagacttacacttgcaacgtggaccacaagccaagcaacaccaaagtggacaagagagtcgaatctaagtacggaccgccctgcccgccttgccccgcccctgagtttctcggcggtcctagcgtgttcctgttcccacccaagcccaaggacactctgatgatctcccggacccctgaagtgacctgtgtggtcgtggacgtgtcgcaggaagatccggaggtccagttcaattggtacgtggatggggtggaggtccacaacgccaagacgaagccgagagaagaacagttcaactcaacttaccgggtggtgtccgtgctgaccgtgctgcatcaggattggctcaacggaaaggagtacaagtgcaaagtgtccaacaagggcctgcctagctcaatcgaaaagaccatttccaaggccaagggccagccgagggaaccacaggtctatactctgccaccgagccaagaagagatgaccaagaaccaagtgtccctgacttgcctggtcaaggggttctacccgtcggacatcgcagtggagtgggagagcaacggacagcctgaaaacaattacaagaccaccccgcccgtgctggatagcgacggttccttcttcctttactcgcgcctcaccgtcgacaagagccggtggcaggagggcaacgtgttctcctgctccgtgatgcacgaagctctgcataaccactacactcagaagtccttgtcgctgagcctcggaaag

SEQ ID NO: 184 - DNA, 636 bp, Artificial Sequence
  LC
cagatcgtgctgacccagagccccgctgtgatgagcgcctctcccggtgagaaggtgaccatgacttgtagcgccagcagcagcatcagctacatgcactggtaccagcagaagcccggcaccagccctaagaggtggatctacgacaccagcaagctggccagcggcgtgcccgctaggttcagcggaagcggcagcggcaccagctactctttaaccatcagcagcatggaggccgaggatgccgccacctactactgccaccagagaagcagctaccccaccttcggcggcggcaccaagctcgagatcaagagaactgtggccgcgccgtcagtgtttatcttccctccatcggatgaacagcttaagtccggcacggcgtctgtggtctgcctgctcaataacttttaccctagggaagctaaagtccaatggaaagtggataacgccctgcagtcaggaaacagccaggaatcggttaccgaacaggacagcaaggacagcacttactccttgtcgtcgactcttactctgagcaaggccgattacgagaagcacaaggtctacgcctgcgaggtcacccatcagggactctcgtccccggtgaccaaatccttcaatagaggcgaatgc

SEQ ID NO: 185 - DNA, 1340 bp, Artificial Sequence
  HC
ctcaagttcagctcgtgcagagcggtgctgaagtgaagaagcccggtgcctctgtgaaggtgagctgcaaggccagcggctacaccttcaccagctacaacatgcactgggtgagacaagctcccggtcaaggtttagagtggatgggcgtgatctaccccggtgatggtgctaccagctacgcccagaagttcaagggtcgtgtgaccatgaccagagacaccagcaccagcaccgtgtacatggagctgagctctttaaggagcgaggacaccgccgtgtactactgcgctcgtgactactacggcagcagccctttaggctattggggacaaggtactttagtgaccgtctcgagcgcctcaaccaaaggaccctccgtgtttcccctcgccccctgttcccgctccacatccgagtcaaccgcggcgctgggctgcctcgtgaaggactacttccctgagcccgtcactgtgtcgtggaactccggggccctgacctccggcgtgcacaccttccctgccgtgcttcaatcctccggactgtactccctgtcctcggtggtcaccgtgccgtcgagctcgttgggaaccaagacttacacttgcaacgtggaccacaagccaagcaacaccaaagtggacaagagagtcgaatctaagtacggaccgccctgcccgccttgccccgcccctgagtttctcggcggtcctagcgtgttcctgttcccacccaagcccaaggacactctgatgatctcccggacccctgaagtgacctgtgtggtcgtggacgtgtcgcaggaagatccggaggtccagttcaattggtacgtggatggggtggaggtccacaacgccaagacgaagccgagagaagaacagttcaactcaacttaccgggtggtgtccgtgctgaccgtgctgcatcaggattggctcaacggaaaggagtacaagtgcaaagtgtccaacaagggcctgcctagctcaatcgaaaagaccatttccaaggccaagggccagccgagggaaccacaggtctatactctgccaccgagccaagaagagatgaccaagaaccaagtgtccctgacttgcctggtcaaggggttctacccgtcggacatcgcagtggagtgggagagcaacggacagcctgaaaacaattacaagaccaccccgcccgtgctggatagcgacggttccttcttcctttactcgcgcctcaccgtcgacaagagccggtggcaggagggcaacgtgttctcctgctccgtgatgcacgaagctctgcataaccactacactcagaagtccttgtcgctgagcctcggaaag

SEQ ID NO: 186 - DNA, 636 bp, Artificial Sequence
  LC
gacatccagatgacccagagccctagctctttaagcgcctctgtgggcgatcgtgtgaccatcacttgtagcgccagccccagcatcagctacatgaactggtaccagcagaagcccggcaaggcccccaagctgctgatctacgccacaagctctttagccagcggcgtgcctagcagatttagcggcagcggtagcggcacagacttcactttaaccatcagctctttacagccagaagacttcgccacctactactgccaccagaggagcagctaccccaccttcggccaaggtaccaagctcgagatcaagagaactgtggccgcgccgtcagtgtttatcttccctccatcggatgaacagcttaagtccggcacggcgtctgtggtctgcctgctcaataacttttaccctagggaagctaaagtccaatggaaagtggataacgccctgcagtcaggaaacagccaggaatcggttaccgaacaggacagcaaggacagcacttactccttgtcgtcgactcttactctgagcaaggccgattacgagaagcacaaggtctacgcctgcgaggtcacccatcagggactctcgtccccggtgaccaaatccttcaatagaggcgaatgc

SEQ ID NO: 187 - DNA, 1338 bp, Artificial Sequence
  HC
caagttcagctcgtgcagagcggtgctgaagtgaagaagcccggtgcctctgtgaaggtgagctgcaaggccagcggctacaccttcaccagctacaacatgcactgggtgagacaagctcccggtcaaggtttagagtggatgggcgtgatcaaccccggtgatggtgctaccagctacaaccagaagttccagggtcgtgtgaccatgaccagagacaccagcaccagcaccgtgtacatggagctgagctctttaaggagcgaggacaccgccgtgtactactgcgctcgtgactactacggcagcagccctttaggctattggggacaaggtactttagtgaccgtctcgagcgcctcaaccaaaggaccctccgtgtttcccctcgccccctgttcccgctccacatccgagtcaaccgcggcgctgggctgcctcgtgaaggactacttccctgagcccgtcactgtgtcgtggaactccggggccctgacctccggcgtgcacaccttccctgccgtgcttcaatcctccggactgtactccctgtcctcggtggtcaccgtgccgtcgagctcgttgggaaccaagacttacacttgcaacgtggaccacaagccaagcaacaccaaagtggacaagagagtcgaatctaagtacggaccgccctgcccgccttgccccgcccctgagtttctcggcggtcctagcgtgttcctgttcccacccaagcccaaggacactctgatgatctcccggacccctgaagtgacctgtgtggtcgtggacgtgtcgcaggaagatccggaggtccagttcaattggtacgtggatggggtggaggtccacaacgccaagacgaagccgagagaagaacagttcaactcaacttaccgggtggtgtccgtgctgaccgtgctgcatcaggattggctcaacggaaaggagtacaagtgcaaagtgtccaacaagggcctgcctagctcaatcgaaaagaccatttccaaggccaagggccagccgagggaaccacaggtctatactctgccaccgagccaagaagagatgaccaagaaccaagtgtccctgacttgcctggtcaaggggttctacccgtcggacatcgcagtggagtgggagagcaacggacagcctgaaaacaattacaagaccaccccgcccgtgctggatagcgacggttccttcttcctttactcgcgcctcaccgtcgacaagagccggtggcaggagggcaacgtgttctcctgctccgtgatgcacgaagctctgcataaccactacactcagaagtccttgtcgctgagcctcggaaag

SEQ ID NO: 188 - DNA, 636 bp, Artificial Sequence
  LC
gacatccagatgacccagagccctagctctttaagcgcctctgtgggcgatcgtgtgaccatcacttgtcgtgccagcagcagcatcagctatttaaactggtaccagcagaagcccggcaaggcccccaagctgctgatctacgccgcctcttctttagcctctggcgtgccttctcgtttcagcggaagcggcagcggcaccgacttcactttaaccatcagctctttacagccagaagacttcgccacctactactgccaccagaggagcagcagccccaccttcggacaaggtaccaagctcgagatcaagagaactgtggccgcgccgtcagtgtttatcttccctccatcggatgaacagcttaagtccggcacggcgtctgtggtctgcctgctcaataacttttaccctagggaagctaaagtccaatggaaagtggataacgccctgcagtcaggaaacagccaggaatcggttaccgaacaggacagcaaggacagcacttactccttgtcgtcgactcttactctgagcaaggccgattacgagaagcacaaggtctacgcctgcgaggtcacccatcagggactctcgtccccggtgaccaaatccttcaatagaggcgaatgc

SEQ ID NO: 189 - DNA, 1338 bp, Artificial Sequence
  HC
caagttcagctggtgcagagcggagccgaggtgaaaaagcccggtgcctctgtgaaggtgagctgcaaggccagcggctacacctttaccagctacaacatgcactgggtgaggcaagctcccggtcaaggtctggagtggatcggcgtgatctaccccggcgacggcagcacctcttacaaccagaagttccaaggtcgtgtgaccatgactcgtgacaccagcaccagcaccgtgtacatggagctgagctctttaaggagcgaggataccgccgtgtactactgcgctcgtgactactacggcagcagccctctgggctattggggccaaggtactttagtgaccgtctcgagcgcctcaaccaaaggaccctccgtgtttcccctcgccccctgttcccgctccacatccgagtcaaccgcggcgctgggctgcctcgtgaaggactacttccctgagcccgtcactgtgtcgtggaactccggggccctgacctccggcgtgcacaccttccctgccgtgcttcaatcctccggactgtactccctgtcctcggtggtcaccgtgccgtcgagctcgttgggaaccaagacttacacttgcaacgtggaccacaagccaagcaacaccaaagtggacaagagagtcgaatctaagtacggaccgccctgcccgccttgccccgcccctgagtttctcggcggtcctagcgtgttcctgttcccacccaagcccaaggacactctgatgatctcccggacccctgaagtgacctgtgtggtcgtggacgtgtcgcaggaagatccggaggtccagttcaattggtacgtggatggggtggaggtccacaacgccaagacgaagccgagagaagaacagttcaactcaacttaccgggtggtgtccgtgctgaccgtgctgcatcaggattggctcaacggaaaggagtacaagtgcaaagtgtccaacaagggcctgcctagctcaatcgaaaagaccatttccaaggccaagggccagccgagggaaccacaggtctatactctgccaccgagccaagaagagatgaccaagaaccaagtgtccctgacttgcctggtcaaggggttctacccgtcggacatcgcagtggagtgggagagcaacggacagcctgaaaacaattacaagaccaccccgcccgtgctggatagcgacggttccttcttcctttactcgcgcctcaccgtcgacaagagccggtggcaggagggcaacgtgttctcctgctccgtgatgcacgaagctctgcataaccactacactcagaagtccttgtcgctgagcctcggaaag

SEQ ID NO: 190 - DNA, 636 bp, Artificial Sequence
  LC
gacatccagatgacccagagccctagctctttaagcgcctctgtgggcgatcgtgtgaccatcacttgtagcgccagcagcagcatcagctacatgaactggtaccagcagaagcccggcaaggcccccaagctgctgatctacgccaccagcaagctgcctagcggcgtgccctccagattttctggcagcggctctggcaccgactttactttaaccatcagctctttacagccagaagacttcgccacctactactgccaccagaggagcagcacccctaccttcggccaaggtaccaagctcgagatcaagagaactgtggccgcgccgtcagtgtttatcttccctccatcggatgaacagcttaagtccggcacggcgtctgtggtctgcctgctcaataacttttaccctagggaagctaaagtccaatggaaagtggataacgccctgcagtcaggaaacagccaggaatcggttaccgaacaggacagcaaggacagcacttactccttgtcgtcgactcttactctgagcaaggccgattacgagaagcacaaggtctacgcctgcgaggtcacccatcagggactctcgtccccggtgaccaaatccttcaatagaggcgaatgc

SEQ ID NO: 191 - DNA, 1338 bp, Artificial Sequence
  HC
caagttcagctcgtgcagagcggtgctgaagtgaagaagcccggtgcctctgtgaaggtgagctgcaaggccagcggctacaccttcaccagctacaacatgcactgggtgagacaagctcccggtcaaggtttagagtggatgggcgtgatcaaccccggtgatggtgctaccagctacgcccagaagttcaagggtcgtgtgaccatgaccagagacaccagcaccagcaccgtgtacatggagctgagctctttaaggagcgaggacaccgccgtgtactactgcgctcgtgactactacggcagcagccctttaggctattggggacaaggtactttagtgaccgtctcgagcgcctcaaccaaaggaccctccgtgtttcccctcgccccctgttcccgctccacatccgagtcaaccgcggcgctgggctgcctcgtgaaggactacttccctgagcccgtcactgtgtcgtggaactccggggccctgacctccggcgtgcacaccttccctgccgtgcttcaatcctccggactgtactccctgtcctcggtggtcaccgtgccgtcgagctcgttgggaaccaagacttacacttgcaacgtggaccacaagccaagcaacaccaaagtggacaagagagtcgaatctaagtacggaccgccctgcccgccttgccccgcccctgagtttctcggcggtcctagcgtgttcctgttcccacccaagcccaaggacactctgatgatctcccggacccctgaagtgacctgtgtggtcgtggacgtgtcgcaggaagatccggaggtccagttcaattggtacgtggatggggtggaggtccacaacgccaagacgaagccgagagaagaacagttcaactcaacttaccgggtggtgtccgtgctgaccgtgctgcatcaggattggctcaacggaaaggagtacaagtgcaaagtgtccaacaagggcctgcctagctcaatcgaaaagaccatttccaaggccaagggccagccgagggaaccacaggtctatactctgccaccgagccaagaagagatgaccaagaaccaagtgtccctgacttgcctggtcaaggggttctacccgtcggacatcgcagtggagtgggagagcaacggacagcctgaaaacaattacaagaccaccccgcccgtgctggatagcgacggttccttcttcctttactcgcgcctcaccgtcgacaagagccggtggcaggagggcaacgtgttctcctgctccgtgatgcacgaagctctgcataaccactacactcagaagtccttgtcgctgagcctcggaaag

SEQ ID NO: 192 - DNA, 636 bp, Artificial Sequence
  LC
gacatccagatgacccagagccctagctctttaagcgccagcgtgggagatcgtgtgaccatcacttgtcgtgccagccccagcatcagctacatgaactggtaccagcagaagcccggcaaggcccccaagctgctgatctacgccaccagctctttagcctctggcgtgcctagcagattcagcggcagcggaagcggcaccgacttcactttaaccatcagctctttacagccagaagacttcgccacctactactgccaccagaggagcagcagccctaccttcggccaaggtaccaagctcgagatcaagagaactgtggccgcgccgtcagtgtttatcttccctccatcggatgaacagcttaagtccggcacggcgtctgtggtctgcctgctcaataacttttaccctagggaagctaaagtccaatggaaagtggataacgccctgcagtcaggaaacagccaggaatcggttaccgaacaggacagcaaggacagcacttactccttgtcgtcgactcttactctgagcaaggccgattacgagaagcacaaggtctacgcctgcgaggtcacccatcagggactctcgtccccggtgaccaaatccttcaatagaggcgaatgc

SEQ ID NO: 193 - DNA, 636 bp, Artificial Sequence
  LC
gacatccagatgacccagagccctagctctttaagcgcctctgtgggcgatcgtgtgaccatcacttgtagcgccagccccagcatcagctacatgaactggtaccagcagaagcccggcaaggcccccaagctgctgatctacgccacaagctctttacccagcggcgtgcctagcagattcagcggcagcggaagcggcaccgacttcactttaaccatcagctctttacagccagaagacttcgccacctactactgccaccagagaagcagcagccccaccttcggccaaggtacaaagctcgagatcaagagaactgtggccgcgccgtcagtgtttatcttccctccatcggatgaacagcttaagtccggcacggcgtctgtggtctgcctgctcaataacttttaccctagggaagctaaagtccaatggaaagtggataacgccctgcagtcaggaaacagccaggaatcggttaccgaacaggacagcaaggacagcacttactccttgtcgtcgactcttactctgagcaaggccgattacgagaagcacaaggtctacgcctgcgaggtcacccatcagggactctcgtccccggtgaccaaatccttcaatagaggcgaatgc

SEQ ID NO: 194 - DNA, 1338 bp, Artificial Sequence
  HC
caagttcagctggtgcagagcggtgctgaggtgaaaaaacccggtgcttccgtgaaggtgagctgcaaggccagcggctacacctttaccagctacaacatgcactgggtgagacaagcccccggtcaaggtttagagtggatcggcgtgatctaccccggcaacggcgacacctcttacaaccagaagttcaagggtcgtgtgaccatgactcgtgacacctccaccagcaccgtgtacatggagctgagctctttaaggagcgaggacacagccgtgtactactgcgctcgtgactactacggcagcagccctctgggctattggggccaaggtactttagtgaccgtctcgagcgcctcaaccaaaggaccctccgtgtttcccctcgccccctgttcccgctccacatccgagtcaaccgcggcgctgggctgcctcgtgaaggactacttccctgagcccgtcactgtgtcgtggaactccggggccctgacctccggcgtgcacaccttccctgccgtgcttcaatcctccggactgtactccctgtcctcggtggtcaccgtgccgtcgagctcgttgggaaccaagacttacacttgcaacgtggaccacaagccaagcaacaccaaagtggacaagagagtcgaatctaagtacggaccgccctgcccgccttgccccgcccctgagtttctcggcggtcctagcgtgttcctgttcccacccaagcccaaggacactctgatgatctcccggacccctgaagtgacctgtgtggtcgtggacgtgtcgcaggaagatccggaggtccagttcaattggtacgtggatggggtggaggtccacaacgccaagacgaagccgagagaagaacagttcaactcaacttaccgggtggtgtccgtgctgaccgtgctgcatcaggattggctcaacggaaaggagtacaagtgcaaagtgtccaacaagggcctgcctagctcaatcgaaaagaccatttccaaggccaagggccagccgagggaaccacaggtctatactctgccaccgagccaagaagagatgaccaagaaccaagtgtccctgacttgcctggtcaaggggttctacccgtcggacatcgcagtggagtgggagagcaacggacagcctgaaaacaattacaagaccaccccgcccgtgctggatagcgacggttccttcttcctttactcgcgcctcaccgtcgacaagagccggtggcaggagggcaacgtgttctcctgctccgtgatgcacgaagctctgcataaccactacactcagaagtccttgtcgctgagcctcggaaag

SEQ ID NO: 195 - DNA, 636 bp, Artificial Sequence
  LC
gacatccagatgacccagagccctagctctttaagcgcctctgtgggcgatcgtgtgaccatcacttgtagcgccagcagcagcatcagctacatgcactggtaccagcagaagcccggcaaggcccccaagctgctgatctacgacaccagcaagctggccagcggcgtgcctagcagattcagcggcagcggaagcggcaccgacttcactttaaccatcagctctttacagccagaagacttcgccacctactactgccaccagaggagcagctaccccaccttcggccaaggtaccaagctcgagatcaagagaactgtggccgcgccgtcagtgtttatcttccctccatcggatgaacagcttaagtccggcacggcgtctgtggtctgcctgctcaataacttttaccctagggaagctaaagtccaatggaaagtggataacgccctgcagtcaggaaacagccaggaatcggttaccgaacaggacagcaaggacagcacttactccttgtcgtcgactcttactctgagcaaggccgattacgagaagcacaaggtctacgcctgcgaggtcacccatcagggactctcgtccccggtgaccaaatccttcaatagaggcgaatgc

SEQ ID NO: 196 - DNA, 1338 bp, Artificial Sequence
  HC
caagtgcagctcgtgcagtccggagccgaagtcaagaagcccggagcgtcagtgaaagtgtcctgcaaggcctcgggctacactttcacaagctacaacatgcactgggtcagacaggcacctgggcagggtctggagtggatgggagtgatctacccgggcgacggcgccacttcctacgcccaaaagttcaagggccgcgtgaccatgactagggacacctcgacctcaaccgtgtacatggaactgagctccctgcggtccgaggataccgccgtgtactattgtgctcgggactactacgggtccagcccactgggatactggggacagggtacccttgtcacggtgtcgtcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaatgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 197 - DNA, 636 bp, Artificial Sequence
  LC
gacatccaaatgactcagtccccgtcatccctgtcggcatccgtgggagacagagtcaccattacgtgcagcgcgagcccgagcatttcctatatgaactggtaccagcagaagcccgggaaagcccctaagctgttgatctacgccacttcctcactggcttccggcgtgccatcccggttctcgggttccggctcgggaaccgattttacccttactatctcgtccctgcaacccgaggacttcgccacctactactgtcaccagcgctctagctaccctacattcggacagggcaccaagctcgaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 198 - DNA, 1338 bp, Artificial Sequence
  HC
caagtgcagctcgtgcagtccggagccgaagtcaagaagcccggagcgtcagtgaaagtgtcctgcaaggcctcgggctacactttcacaagctacaacatgcactgggtcagacaggcacctgggcagggtctggagtggattggagtgatctacccgggcgacggctccacttcctacaaccaaaagttccagggccgcgtgaccatgactagggacacctcgacctcaaccgtgtacatggaactgagctccctgcggtccgaggataccgccgtgtactattgtgctcgggactactacgggtccagcccactgggatactggggacagggtacccttgtcacggtgtcgtcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaatgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 199 - DNA, 635 bp, Artificial Sequence
  LC
acatccaaatgactcagtccccgtcatccctgtcggcatccgtgggagacagagtcaccattacgtgcagcgcgagctcaagcatttcctatatgaactggtaccagcagaagcccgggaaagcccctaagctgttgatctacgccacttccaagctgccgtccggcgtgccatcccggttctcgggttccggctcgggaaccgattttacccttactatctcgtccctgcaacccgaggacttcgccacctactactgtcaccagcgctctagcacccctacattcggacagggcaccaagctcgaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 200 - DNA, 1338 bp, Artificial Sequence
  HC
caagtgcagctcgtgcagtccggagccgaagtcaagaagcccggagcgtcagtgaaagtgtcctgcaaggcctcgggctacactttcacaagctacaacatgcactgggtcagacaggcacctgggcagggtctggagtggatgggagtgatcaacccgggcgacggcgccacttcctacgcccaaaagttcaagggccgcgtgaccatgactagggacacctcgacctcaaccgtgtacatggaactgagctccctgcggtccgaggataccgccgtgtactattgtgctcgggactactacgggtccagcccactgggatactggggacagggtacccttgtcacggtgtcgtcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaatgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 201 - DNA, 636 bp, Artificial Sequence
  LC
gacatccaaatgactcagtccccgtcatccctgtcggcatccgtgggagacagagtcaccattacgtgccgcgcgagcccgagcatttcctatatgaactggtaccagcagaagcccgggaaagcccctaagctgttgatctacgccacttcctcactggcttccggcgtgccatcccggttctcgggttccggctcgggaaccgattttacccttactatctcgtccctgcaacccgaggacttcgccacctactactgtcaccagcgctctagcagccctacattcggacagggcaccaagctcgaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 202 - DNA, 636 bp, Artificial Sequence
  LC
gacatccaaatgactcagtccccgtcatccctgtcggcatccgtgggagacagagtcaccattacgtgcagcgcgagcccgagcatttcctatatgaactggtaccagcagaagcccgggaaagcccctaagctgttgatctacgccacttcctcactgccgtccggcgtgccatcccggttctcgggttccggctcgggaaccgattttacccttactatctcgtccctgcaacccgaggacttcgccacctactactgtcaccagcgctctagcagccctacattcggacagggcaccaagctcgaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt